SEQUENCE LISTING (81 sequences)

SEQ ID NO: 1 - protein, 1054 aa, Artificial Sequence
  Description of Artificial Sequence Synthetic polypeptide
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTS

SEQ ID NO: 2 - protein, 17 aa, Artificial Sequence
  Description of Artificial Sequence Synthetic peptide
SEFFSGEETYTICVHHL

SEQ ID NO: 3 - protein, 6 aa, Artificial Sequence
  Description of Artificial Sequence Synthetic peptide
DPADPE

SEQ ID NO: 4 - protein, 6 aa, Artificial Sequence
  Description of Artificial Sequence Synthetic peptide
RQECSA

SEQ ID NO: 5 - protein, 9 aa, Artificial Sequence
  Description of Artificial Sequence Synthetic peptide
KKNKKKEIV

SEQ ID NO: 6 - protein, 6 aa, Artificial Sequence
  Description of Artificial Sequence Synthetic peptide
VRGKSL

SEQ ID NO: 7 - protein, 12 aa, Artificial Sequence
  Description of Artificial Sequence Synthetic peptide
ALNASKNNPISD

SEQ ID NO: 8 - protein, 13 aa, Artificial Sequence
  Description of Artificial Sequence Synthetic peptide
LKWRSDRKSNIPC

SEQ ID NO: 9 - protein, 11 aa, Artificial Sequence
  Description of Artificial Sequence Synthetic peptide
STEQEKQQQDI

SEQ ID NO: 10 - protein, 7 aa, Artificial Sequence
  Description of Artificial Sequence Synthetic peptide
YGGTASD

SEQ ID NO: 11 - protein, 9 aa, Artificial Sequence
  Description of Artificial Sequence Synthetic peptide
SASIGESYY

SEQ ID NO: 12 - protein, 12 aa, Artificial Sequence
  Description of Artificial Sequence Synthetic peptide
SNLKEIPKNVAP

SEQ ID NO: 13 - protein, 9 aa, Artificial Sequence
  Description of Artificial Sequence Synthetic peptide
KDGQKEFDL

SEQ ID NO: 14 - protein, 12 aa, Artificial Sequence
  Description of Artificial Sequence Synthetic peptide
GRQKGTLQIGDR

SEQ ID NO: 15 - protein, 25 aa, Artificial Sequence
  Description of Artificial Sequence Synthetic peptide
CGSLYTSHQDPLVHRNPDKAMKCRW

SEQ ID NO: 16 - protein, 7 aa, Artificial Sequence
  Description of Artificial Sequence Synthetic peptide
GTGEKED

SEQ ID NO: 17 - protein, 6 aa, Artificial Sequence
  Description of Artificial Sequence Synthetic peptide
KATKET

SEQ ID NO: 18 - protein, 8 aa, Artificial Sequence
  Description of Artificial Sequence Synthetic peptide
SKERDWCC

SEQ ID NO: 19 - protein, 6 aa, Artificial Sequence
  Description of Artificial Sequence Synthetic peptide
RQECSA

SEQ ID NO: 20 - protein, 13 aa, Artificial Sequence
  Description of Artificial Sequence Synthetic peptide
AQRNDRPNSLDLR

SEQ ID NO: 21 - protein, 6 aa, Artificial Sequence
  Description of Artificial Sequence Synthetic peptide
HPDGRW

SEQ ID NO: 22 - protein, 16 aa, Artificial Sequence
  Description of Artificial Sequence Synthetic peptide
IYAAGNSPVDTCQFRT

SEQ ID NO: 23 - protein, 12 aa, Artificial Sequence
  Description of Artificial Sequence Synthetic peptide
VKEGQYHKELGC

SEQ ID NO: 24 - protein, 6 aa, Artificial Sequence
  Description of Artificial Sequence Synthetic peptide
GNKEAV

SEQ ID NO: 25 - protein, 6 aa, Artificial Sequence
  Description of Artificial Sequence Synthetic peptide
VFDQKQ

SEQ ID NO: 26 - protein, 5 aa, Artificial Sequence
  Description of Artificial Sequence Synthetic peptide
GGGGS

SEQ ID NO: 27 - protein, 30 aa, Artificial Sequence
  Description of Artificial Sequence Synthetic polypeptide
GGGGSGGGGSGGGGSGGGGSGGGGSGGGGS

SEQ ID NO: 28 - protein, 15 aa, Artificial Sequence
  Description of Artificial Sequence Synthetic peptide
GGGGSGGGGSGGGGS

SEQ ID NO: 29 - protein, 4 aa, Artificial Sequence
  Description of Artificial Sequence Synthetic peptide
GSSG

SEQ ID NO: 30 - protein, 8 aa, Artificial Sequence
  Description of Artificial Sequence Synthetic peptide
GSSGGSSG

SEQ ID NO: 31 - protein, 12 aa, Artificial Sequence
  Description of Artificial Sequence Synthetic peptide
GSSGGSSGGSSG

SEQ ID NO: 32 - protein, 16 aa, Artificial Sequence
  Description of Artificial Sequence Synthetic peptide
GSSGGSSGGSSGGSSG

SEQ ID NO: 33 - protein, 3 aa, Artificial Sequence
  Description of Artificial Sequence Synthetic peptide
GSG

SEQ ID NO: 34 - protein, 12 aa, Artificial Sequence
  Description of Artificial Sequence Synthetic peptide
GSGGSGGSGGSG

SEQ ID NO: 35 - protein, 4 aa, Artificial Sequence
  Description of Artificial Sequence Synthetic peptide
GGGS

SEQ ID NO: 36 - RNA, 34 nt, Artificial Sequence
  Description of Artificial Sequence Synthetic oligonucleotide
gcaacaccuaagaaauccgucuuucauugacggg

SEQ ID NO: 37 - RNA, 36 nt, Artificial Sequence
  Description of Artificial Sequence Synthetic oligonucleotide
guugcaaaacccaagaaauccgucuuucauugacgg

SEQ ID NO: 38 - RNA, 23 nt, Artificial Sequence
  Description of Artificial Sequence Synthetic oligonucleotide
agaaauccgucuuucauugacgg

SEQ ID NO: 39 - protein, 1053 aa, Artificial Sequence
  Description of Artificial Sequence Synthetic polypeptide
MSSAIKSYKSVLRPNERKNQLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGRWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 40 - protein, 1054 aa, Artificial Sequence
  Description of Artificial Sequence Synthetic polypeptide
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 41 - protein, 1054 aa, Artificial Sequence
  Description of Artificial Sequence Synthetic polypeptide
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGGRIKLQLTS

SEQ ID NO: 42 - protein, 1054 aa, Artificial Sequence
  Description of Artificial Sequence Synthetic polypeptide
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 43 - protein, 1053 aa, Artificial Sequence; identical to SEQ ID NO: 39
  Description of Artificial Sequence Synthetic polypeptide

SEQ ID NO: 44 - protein, 15 aa, Artificial Sequence
  Description of Artificial Sequence Synthetic peptide
GSSGSSGSSGSSGSS

SEQ ID NO: 45 - protein, 1070 aa, Artificial Sequence
  Description of Artificial Sequence Synthetic polypeptide
MECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTSGGSGGSGGSGGSGGSMSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQ

SEQ ID NO: 46 - protein, 1070 aa, Artificial Sequence
  Description of Artificial Sequence Synthetic polypeptide
MKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTSGGSGGSGGSGGSGGSMSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNK

SEQ ID NO: 47 - protein, 1070 aa, Artificial Sequence
  Description of Artificial Sequence Synthetic polypeptide
MQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTSGGSGGSGGSGGSGGSMSSAIKSYKSVLRPNERKNQLLKSTIQCLEDGSAFFFKMLQGLFGGITPEIVRFSTEQEK

SEQ ID NO: 48 - protein, 1070 aa, Artificial Sequence
  Description of Artificial Sequence Synthetic polypeptide
MTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTSGGSGGSGGSGGSGGSMSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGG

SEQ ID NO: 49 - protein, 1070 aa, Artificial Sequence
  Description of Artificial Sequence Synthetic polypeptide
MESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTSGGSGGSGGSGGSGGSMSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIG

SEQ ID NO: 50 - protein, 1070 aa, Artificial Sequence
  Description of Artificial Sequence Synthetic polypeptide
MIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTSGGSGGSGGSGGSGGSMSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKE

SEQ ID NO: 51 - protein, 1070 aa, Artificial Sequence
  Description of Artificial Sequence Synthetic polypeptide
MEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTSGGSGGSGGSGGSGGSMSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQK

SEQ ID NO: 52 - protein, 1070 aa, Artificial Sequence
  Description of Artificial Sequence Synthetic polypeptide
MPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTSGGSGGSGGSGGSGGSMSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRN

SEQ ID NO: 53 - RNA, 46 nt, Artificial Sequence
  Description of Artificial Sequence Synthetic oligonucleotide
agacauguguccucagugacacggauggcgacuucaggcacaggau

SEQ ID NO: 54 - DNA, 24 bp, Artificial Sequence
  Description of Artificial Sequence Synthetic oligonucleotide
ggatggcgacttcaggcacaggat

SEQ ID NO: 55 - RNA, 46 nt, Artificial Sequence
  Description of Artificial Sequence Synthetic oligonucleotide
agacauguguccucagugacacagcaagggacuauucagggaugaa

SEQ ID NO: 56 - DNA, 24 bp, Artificial Sequence
  Description of Artificial Sequence Synthetic oligonucleotide
agcaagggactattcagggatgaa

SEQ ID NO: 57 - RNA, 46 nt, Artificial Sequence
  Description of Artificial Sequence Synthetic oligonucleotide
agacauguguccucagugacacaaaauugagcaaucuacccugguc

SEQ ID NO: 58 - DNA, 24 bp, Artificial Sequence
  Description of Artificial Sequence Synthetic oligonucleotide
aaaattgagcaatctaccctggtc

SEQ ID NO: 59 - RNA, 46 nt, Artificial Sequence
  Description of Artificial Sequence Synthetic oligonucleotide
agacauguguccucagugacacuuaaacucuccauggaccaggcuc

SEQ ID NO: 60 - DNA, 24 bp, Artificial Sequence
  Description of Artificial Sequence Synthetic oligonucleotide
ttaaactctccatggaccaggctc

SEQ ID NO: 61 - protein, 16 aa, Artificial Sequence
  Description of Artificial Sequence Synthetic peptide
KRPAATKKAGQAKKKK

SEQ ID NO: 62 - protein, 19 aa, Artificial Sequence
  Description of Artificial Sequence Synthetic peptide
MKRTADGSEFESPKKKRKV

SEQ ID NO: 63 - protein, 20 aa, Artificial Sequence
  Description of Artificial Sequence Synthetic peptide
MKRTADGSEFESPKKKRKVE

SEQ ID NO: 64 - protein, 18 aa, Artificial Sequence
  Description of Artificial Sequence Synthetic peptide
KRTADGSEFESPKKKRKV

SEQ ID NO: 65 - protein, 19 aa, Artificial Sequence
  Description of Artificial Sequence Synthetic peptide
KRTADGSEFESPKKKRKVE

SEQ ID NO: 66 - protein, 14 aa, Artificial Sequence
  Description of Artificial Sequence Synthetic peptide
MKIEEGKGHHHHHH

SEQ ID NO: 67 - protein, 13 aa, Artificial Sequence
  Description of Artificial Sequence Synthetic peptide
KIEEGKGHHHHHH

SEQ ID NO: 68 - protein, 1089 aa, Artificial Sequence
  Description of Artificial Sequence Synthetic polypeptide
MKIEEGKGHHHHHHMSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTSGSKRTADGSEFESPKKKRKVE

SEQ ID NO: 69 - protein, 1088 aa, Artificial Sequence
  Description of Artificial Sequence Synthetic polypeptide
MKIEEGKGHHHHHHMSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTSGSKRTADGSEFESPKKKRKV

SEQ ID NO: 70 - protein, 3 aa, Artificial Sequence
  Description of Artificial Sequence Synthetic peptide
GSS

SEQ ID NO: 71 - protein, 15 aa, Artificial Sequence
  Description of Artificial Sequence Synthetic peptide
GGSGGSGGSGGSGGS

SEQ ID NO: 72 - protein, 3 aa, Artificial Sequence
  Description of Artificial Sequence Synthetic peptide
GGS

SEQ ID NO: 73 - protein, 1075 aa, Artificial Sequence
  Description of Artificial Sequence Synthetic polypeptide
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTSGSKRTADGSEFESPKKKRKVE

SEQ ID NO: 74 - protein, 1074 aa, Artificial Sequence
  Description of Artificial Sequence Synthetic polypeptide
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTSGSKRTADGSEFESPKKKRKV

SEQ ID NO: 75 - protein, 4 aa, Artificial Sequence
  Description of Artificial Sequence Synthetic peptide
IRQE

SEQ ID NO: 76 - protein, 4 aa, Artificial Sequence
  Description of Artificial Sequence Synthetic peptide
YGGT

SEQ ID NO: 77 - protein, 45 aa, Artificial Sequence
  Description of Artificial Sequence Synthetic polypeptide
  SITE (1)..(45): This sequence may encompass 0-15 “Gly Ser Gly” repeating units
GSGGSGGSGGSGGSGGSGGSGGSGGSGGSGGSGGSGGSGGSGGSG

SEQ ID NO: 78 - protein, 60 aa, Artificial Sequence
  Description of Artificial Sequence Synthetic polypeptide
  SITE (1)..(60): This sequence may encompass 0-15 “Gly Gly Gly Ser” repeating units
GGGSGGGSGGGSGGGSGGGSGGGSGGGSGGGSGGGSGGGSGGGSGGGSGGGSGGGSGGGS

SEQ ID NO: 79 - protein, 60 aa, Artificial Sequence
  Description of Artificial Sequence Synthetic polypeptide
  SITE (1)..(60): This sequence may encompass 0-15 “Gly Ser Ser Gly” repeating units
GSSGGSSGGSSGGSSGGSSGGSSGGSSGGSSGGSSGGSSGGSSGGSSGGSSGGSSGGSSG

SEQ ID NO: 80 - protein, 45 aa, Artificial Sequence
  Description of Artificial Sequence Synthetic polypeptide
  SITE (1)..(45): This sequence may encompass 0-15 “Gly Ser Ser” repeating units
GSSGSSGSSGSSGSSGSSGSSGSSGSSGSSGSSGSSGSSGSSGSS

SEQ ID NO: 81 - protein, 75 aa, Artificial Sequence
  Description of Artificial Sequence Synthetic polypeptide
  SITE (1)..(75): This sequence may encompass 0-15 “Gly Gly Gly Gly Ser” repeating units
GGGGSGGGGSGGGGSGGGGSGGGGSGGGGSGGGGSGGGGSGGGGSGGGGSGGGGSGGGGSGGGGSGGGGSGGGGS

(19) United States
(12) Patent Application Publication
(10) Pub. No.: US 2024/0301446 A1
(43) Pub. Date: Sep. 12, 2024

(54) CAS12I2 FUSION MOLECULES AND USES THEREOF

(71) Applicant: Arbor Biotechnologies, Inc., Cambridge, MA (US)

(72) Inventors: Brendan Jay Hilbert, Natick, MA (US); Noah Michael Jakimo, Cambridge, MA (US); David A. Scott, San Francisco, CA (US); Colin Alexander McGaw, Boston, MA (US); Jason Michael Carte, Boston, MA (US)

(21) Appl. No.: 18/262,086

(22) PCT Filed: Jan. 20, 2022
(86) PCT No.: PCT/US2022/013133

§ 371 Date: Jul. 19, 2023

Related U.S. Application Data
(60) Provisional application No. 63/270,512, filed on Oct. 21, 2021.
(60) Provisional application No. 63/227,404, filed on Jul. 30, 2021.
(60) Provisional application No. 63/139,651, filed on Jan. 20, 2021.

(51) Int. Cl. C12N 15/90; C12N 9/22; C12N 15/11

(52) U.S. Cl. CPC C12N 15/907; C12N 9/22; C12N 15/11; C12N 2310/20; C12N 2800/80

(57) ABSTRACT

The disclosure describes Cas12i2 fusion proteins, methods, and compositions for the manipulation of nucleic acids in a targeted fashion. The disclosure describes non-naturally occurring, engineered Cas12i2 fusion proteins, components, and methods for targeted modification of nucleic acids. Each system, includes one or more protein components and one or more nucleic acid components that together target nucleic acids.

### RELATED APPLICATIONS

This application claims priority to U.S. Provisional Application 63/139,651 filed on Jan. 20, 2021, U.S. Provisional Application 63/227,404 filed on Jul. 30, 2021, U.S. Provisional Application 63/270,512 filed on Oct. 21, 2021, the entire contents of each of which are hereby incorporated by reference.

### SEQUENCE LISTING

The instant application contains a Sequence Listing which has been submitted electronically in ASCII format and is hereby incorporated by reference in its entirety. Said ASCII copy, created on Jan. 19, 2022 is named A2186-7040WO_SL.txt and is 184,784 bytes in size.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY OF THE INVENTION

It is against the above background that the present invention provides certain advantages and advancements over the prior art.

The disclosure provides Cas12i2 fusion proteins, compositions, systems, and methods of using the Cas12i2 fusion proteins. In particular, such Cas12i2 fusion proteins contain one or more domains, wherein at least one of the domains includes a portion of a Cas12i2 domain and one or more heterologous sequences. The heterologous sequences in the Cas12i2 fusion proteins may include a fusion domain (e.g., a base editing domain, a ssDNA binding domain, an NLS, a poly-basic domain, a restriction endonuclease, or a CRISPR nuclease). The Cas12i2 domain (e.g., at least a portion of SEQ ID NO: 1 or any of SEQ ID NOs: 39-43) in the Cas12i2 fusion proteins may contact (e.g., associate with, recognize, or bind) a target nucleic acid at a position specified by an RNA guide. While the amino acid numbering system used herein is in relation to SEQ ID NO: 1 or any one of SEQ ID NOs: 39-43, other Cas12i2 sequences can be used. One of ordinary skill in the art can identify the corresponding amino acid positions in another Cas12i2 sequences using available tools, such as sequence alignment algorithms.

In one aspect, the disclosure provides a Cas12i2 fusion protein comprising:
a) a first portion comprising amino acids 1-n of SEQ ID NO: 1 or any one of SEQ ID NOs: 39-43, or a sequence having at least 80%, 85%, 90%, 95%, 96%, 97%, 98%, or 99% identity thereto;
b) a second portion comprising amino acids m-1054 of SEQ ID NO: 1 or any one of SEQ ID NOs: 39-43, or a sequence having at least 80%, 85%, 90%, 95%, 96%, 97%, 98%, or 99% identity thereto; and
c) a heterologous sequence disposed between the first portion and the second portion,
wherein n and m are each independently a number between:
i) 342-358 (e.g., 342, 343, 344, 345, 346, 347, 348, 349, 350, 351, 352, 353, 354, 355, 356, 357, or 358);
ii) 373-378 (e.g., 373, 374, 375, 376, 377, or 378);
iii) 408-413 (e.g., 408, 409, 410, 411, 412, or 413);
iv) 677-685 (e.g., 677, 678, 679, 680, 681, 682, 683, 684, or 685);
v) 718-723 (e.g., 718, 719, 720, 721, 722, or 723);
vi) 771-782 (e.g., 771, 772, 773, 774, 775, 776, 777, 778, 779, 780, 781, or 782);
vii) 953-965 (e.g., 953, 954, 955, 956, 957, 958, 959, 960, 961, 962, 963, 964, or 965);
viii) 55-65 (e.g., 55, 56, 57, 58, 59, 60, 61, 62, 63, 64, or 65);
ix) 99-105 (e.g., 99, 100, 101, 102, 103, 104, or 105);
x) 112-120 (e.g., 112, 113, 114, 115, 116, 117, 118, 119, or 120);
xi) 195-206 (e.g., 195, 196, 197, 198, 199, 200, 201, 202, 203, 204, 205, or 206);
xii) 241-250 (e.g., 241, 242, 243, 244, 245, 246, 247, 248, 249, or 250);
xiii) 583-594 (e.g., 583, 584, 585, 586, 587, 588, 589, 590, 591, 592, 593, or 594);
xiv) 877-901 (e.g., 877, 878, 879, 880, 881, 882, 883, 884, 885, 886, 887, 888, 889, 890, 891, 892, 893, 894, 895, 896, 897, 898, 899, 900, or 901);
xv) 173-179 (e.g., 173, 174, 175, 176, 177, 178, or 179);
xvi) 216-221 (e.g., 216, 217, 218, 219, 220, or 221);
xvii) 265-272 (e.g., 265, 266, 267, 268, 269, 270, 271, or 272);
xviii) 456-468 (e.g., 456, 457, 458, 459, 460, 461, 462, 463, 464, 465, 466, 467, or 468);
xix) 476-482 (e.g., 476, 477, 478, 479, 480, 481, or 482);
xx) 498-513 (e.g., 498, 499, 500, 501, 502, 503, 504, 505, 506, 507, 508, 509, 510, 511, 512, or 513);
xxi) 614-625 (e.g., 614, 615, 616, 617, 618, 619, 620, 621, 622, 623, 624, or 625);
xxii) 977-982 (e.g., 977, 978, 979, 980, 981, or 982); or
xxiii) 1007-1012 (e.g., 1007, 1008, 1009, 1010, 1011, or 1012).

In some embodiments, n<m. In some embodiment, m=n+1.

In some embodiments, the Cas12i2 fusion protein is a Cas12i2 fusion protein as described herein (e.g., in section Cas12i2 fusion proteins of the detailed description).

In some embodiments, the heterologous sequence comprises a fusion domain (e.g., a base editing domain, a ssDNA binding domain, an NLS, a poly-basis domain, a restriction endonuclease, or a CRISPR nuclease). In certain embodiments, the heterologous sequence comprises at least one linker sequence. In some embodiments, the heterologous sequence comprises a first linker (e.g., a first peptide linker) and a second linker (e.g., a second peptide linker). In certain embodiments, the first linker and the second linker each independently comprise between 3 and 60 amino acid residues. In some embodiments, the first linker and the second linker each independently comprise one or more Gly residues and one or more Ser residues. In certain embodiments, the first linker and the second peptide linker each independently comprise (GSG)ₓ, (GGGS)ₓ, or (GSSG)ₓ, wherein x is an integer between 0 and 15 (e.g., 0, 1, 2, 3, 4, 5, 6, 7, 8, 9, 10, 11, 12, 13, 14, or 15). In some embodiments, the first linker is N-terminal of the fusion domain and the second linker is C-terminal of the fusion domain. In certain embodiments, the first linker and the second linker are the same. In some embodiments, the first linker and the second linker are different.

In one aspect, the disclosure provides a Cas12i2 fusion protein comprising:
a) a Cas12i2 domain comprising an amino acid sequence of SEQ ID NO: 1 or any one of SEQ ID NOs: 39-43, or a sequence having at least 80%, 85%, 90%, 95%, 96%, 97%, 98%, or 99% identity thereto;
b) a first heterologous sequence disposed N-terminal of the Cas12i2 domain; and
c) a second heterologous sequence disposed C-terminal of the Cas12i2 domain, wherein the first heterologous sequence comprises a dimerization domain, the second heterologous sequence comprises a dimerization domain, or the first heterologous sequence comprises a first dimerization domain and the second heterologous sequences comprises a second, compatible dimerization domain.

In some embodiments, the heterologous sequence further comprises a fusion domain. In some embodiments, the Cas12i2 fusion protein is a Cas12i2 fusion protein as described herein (e.g., in section Fusion proteins with dimerization domains of the detailed description).

In one aspect, the disclosure provides a Cas12i2 fusion protein comprising:
a) a Cas12i2 domain comprising an amino acid sequence of SEQ ID NO: 1 or any one of SEQ ID NOs: 39-43, or a sequence having at least 80%, 85%, 90%, 95%, 96%, 97%, 98%, or 99% identity thereto;
b) a first heterologous sequence disposed N-terminal of the Cas12i2 domain, wherein the first heterologous sequence comprises a first portion of a split fusion domain; and
c) a second heterologous sequence disposed C-terminal of the Cas12i2 domain, wherein the second heterologous sequence comprises a second portion of a split fusion domain, wherein the second portion of the split fusion domain can bind the first portion of the split fusion domain.

In some embodiments, the Cas12i2 fusion protein is a Cas12i2 fusion protein as described herein (e.g., in section N-terminal and C-terminal split fusion).

In another aspect, the disclosure provides an engineered, non-naturally occurring Cas12i2 fusion protein comprising:
a) a first portion comprising an amino acid sequence of an N-terminal portion of SEQ ID NO: 1 or any one of SEQ ID NOs: 39-43, or a sequence having at least 80%, 85%, 90%, 95%, 96%, 97%, 98%, or 99% identity thereto; and
b) a second portion comprising an amino acid sequence of a C-terminal portion of SEQ ID NO: 1 or any one of SEQ ID NOs: 39-43, or a sequence having at least 80%, 85%, 90%, 95%, 96%, 97%, 98%, or 99% identity thereto,
wherein the second portion is N-terminal of the first portion,
wherein the first portion and second portion together bind to an RNA guide comprising a direct repeat sequence and a spacer sequence.

In some embodiments, the Cas12i2 fusion protein is capable of specifically binding or contacting a target nucleic acid (e.g., a target nucleic acid complementary to the spacer sequence). In some embodiments, the first portion and the second portion are linked by a heterologous sequence. In certain embodiments, the heterologous sequence comprises one or more of:
a) a first linker (e.g., a first peptide linker);
b) a second linker (e.g., a second peptide linker); and
c) a fusion domain.

In some embodiments, the C-terminal most amino acid of the first portion is any amino acid residue of SEQ ID NO: 1 or any one of SEQ ID NOs: 39-43 chosen from residues:
i) 342-358 (e.g., residue 342, 343, 344, 345, 346, 347, 348, 349, 350, 351, 352, 353, 354, 355, 356, 357, or 358);
ii) 373-378 (e.g., residue 373, 374, 375, 376, 377, or 378);
iii) 408-413 (e.g., residue 408, 409, 410, 411, 412, or 413);
iv) 677-685 (e.g., residue 677, 678, 679, 680, 681, 682, 683, 684, or 685);
v) 718-723 (e.g., residue 718, 719, 720, 721, 722, or 723);
vi) 771-782 (e.g., residue 771, 772, 773, 774, 775, 776, 777, 778, 779, 780, 781, or 782);
vii) 953-965 (e.g., residue 953, 954, 955, 956, 957, 958, 959, 960, 961, 962, 963, 964, or 965);
viii) 55-65 (e.g., residue 55, 56, 57, 58, 59, 60, 61, 62, 63, 64, or 65);
ix) 99-105 (e.g., residue 99, 100, 101, 102, 103, 104, or 105);
x) 112-120 (e.g., residue 112, 113, 114, 115, 116, 117, 118, 119, or 120);
xi) 195-206 (e.g., residue 195, 196, 197, 198, 199, 200, 201, 202, 203, 204, 205, or 206);
xii) 241-250 (e.g., residue 241, 242, 243, 244, 245, 246, 247, 248, 249, or 250);
xiii) 583-594 (e.g., residue 583, 584, 585, 586, 587, 588, 589, 590, 591, 592, 593, or 594);
xiv) 877-901 (e.g., residue 877, 878, 879, 880, 881, 882, 883, 884, 885, 886, 887, 888, 889, 890, 891, 892, 893, 894, 895, 896, 897, 898, 899, 900, or 901);
xv) 173-179 (e.g., residue 173, 174, 175, 176, 177, 178, or 179);
xvi) 216-221 (e.g., residue 216, 217, 218, 219, 220, or 221);
xvii) 265-272 (e.g., residue 265, 266, 267, 268, 269, 270, 271, or 272);
xviii) 456-468 (e.g., residue 456, 457, 458, 459, 460, 461, 462, 463, 464, 465, 466, 467, or 468);
xix) 476-482 (e.g., residue 476, 477, 478, 479, 480, 481, or 482);
xx) 498-513 (e.g., residue 498, 499, 500, 501, 502, 503, 504, 505, 506, 507, 508, 509, 510, 511, 512, or 513);
xxi) 614-625 (e.g., residue 614, 615, 616, 617, 618, 619, 620, 621, 622, 623, 624, or 625);
xxii) 977-982 (e.g., residue 977, 978, 979, 980, 981, or 982);
xxiii) 1007-1012 (e.g., residue 1007, 1008, 1009, 1010, 1011, or 1012);
xxiv) 386-397 (e.g., residue 386, 387, 388, 389, 390, 391, 392, 393, 394, 395, 396, or 397); or
xxv) 831-844 (e.g., residue 831, 832, 833, 834, 835, 836, 837, 838, 839, 840, 841, 842, 843, or 844).

In any of the embodiments described herein, the Cas12i2 fusion protein further comprises a second heterologous sequence at its N-terminus. In some embodiments, the Cas12i2 fusion protein further comprises an additional heterologous sequence at its C-terminus. In some embodiments, the second heterologous sequence and/or the additional heterologous sequence is chosen from a purification tag, stability tag, or restriction endonuclease or domain thereof. In some embodiments, the heterologous sequence comprises a FokI nuclease domain (e.g., a catalytically active FokI nuclease or a catalytically inactive FokI nuclease domain). In certain embodiments, the N-terminal Met residue of SEQ ID NO: 1, 39-43, 73, or 74 is absent.

In any of the embodiments described herein, the first portion further comprises a fusion domain, the second portion comprises a fusion domain, or the first portion and the second portion comprise a fusion domain. In certain embodiments, a) the first portion comprises a catalytically active FokI nuclease domain and the second portion comprises a catalytically inactive FokI nuclease domain; or
b) the first portion comprises a catalytically inactive FokI nuclease domain and the second portion comprises a catalytically active FokI nuclease domain.

In some embodiments, the Cas12i2 fusion protein is capable of binding an RNA guide, wherein the RNA guide comprises a direct repeat sequence and a spacer sequence, wherein the spacer is capable of hybridizing to a target nucleic acid, e.g., a target strand (i.e., non-PAM strand) of a target nucleic acid.

In any of the aspects described herein, the Cas12i2 fusion protein comprises a catalytic residue (e.g., D599, E833, and D1019). In certain embodiments, the Cas12i2 fusion protein comprises a mutation at any one of amino acid residue D599, E833, or D1019 of SEQ ID NO: 1. In certain embodiments, the Cas12i2 fusion protein is a deadCas12i2 fusion protein (e.g., a variant Cas12i2 fusion protein comprising D599A, E833A, and/or D1019A). In some embodiments, the Cas12i2 fusion protein comprises a catalytically inactive RuvC domain. In certain embodiments, the Cas12i2 fusion protein comprises nickase activity. In some embodiments, the Cas12i2 fusion protein is capable of binding an RNA guide comprising a direct repeat sequence and a spacer sequence, wherein the spacer sequence is capable of hybridizing to a target nucleic acid.

In some embodiments, the heterologous sequence comprises a peptide tag, a fluorescent protein, a base-editing domain, a DNA methylation domain, a histone residue modification domain, a localization factor (e.g., an NLS), a transcription modification factor, a light-gated control factor, a chemically inducible factor, a chromatin visualization factor, restriction endonuclease, or a CRISPR nuclease. In some embodiments, the Cas12i2 fusion protein comprises a fusion domain having an amino acid sequence of SEQ ID NO: 66 or SEQ ID NO: 67, or a sequence having at least 80%, 85%, 90%, 95%, 96%, 97%, 98%, or 99% identity thereto. In some embodiments, the fusion domain is situated at the N-terminus or C-terminus of the Cas12i2 fusion protein. In certain embodiments, the fusion domain comprises an NLS. In some embodiments, the NLS comprises an amino acid sequence of any one of SEQ ID NOs: 61-65, or a sequence having at least 80%, 85%, 90%, 95%, 96%, 97%, 98%, or 99% identity thereto. In certain embodiments, the Cas12i2 protein comprises an amino acid sequence of SEQ ID NO: 68, SEQ ID NO: 69, SEQ ID NO: 73, or SEQ ID NO: 74, or a sequence having at least 80%, 85%, 90%, 95%, 96%, 97%, 98%, or 99% identity thereto. In some embodiments, the heterologous sequence is about 1-5, 5-10, 10-20, 20-30, 30-40, 40-50, 50-100, 100-200, 200-300, 300-400, 400-500, 500-600, 600-700, 700-800, 800-900, 900-1100, 1100-1400, 1400-1600, 1600-1800, or 1800-2000 amino acids in length.

In another aspect, the disclosure provides a system comprising:
(a) a Cas12i2 fusion protein of any aspect described herein, and
(b) an RNA guide comprising a direct repeat sequence and a spacer sequence, wherein the spacer sequence is capable of hybridizing to a target nucleic acid, e.g., to a target strand (i.e., non-PAM strand) of a target nucleic acid.

In another aspect, the disclosure provides a nucleic acid encoding the Cas12i2 fusion protein or the system described herein.

In one aspect, the disclosure provides a composition comprising: a first nucleic acid encoding the Cas12i2 fusion protein of any aspect described herein and a second nucleic acid comprising or encoding an RNA guide comprising a direct repeat sequence and a spacer sequence, wherein the spacer sequence is capable of hybridizing to a target nucleic acid, e.g., to a target strand (i.e., non-PAM strand) of a target nucleic acid.

In another aspect, the disclosure provides a vector comprising:
(a) a nucleic acid encoding the Cas12i2 fusion protein or a system described herein; or
(b) one or both of a first nucleic acid encoding the Cas12i2 fusion protein of any aspect described herein and a second nucleic acid comprising or encoding an RNA guide comprising a direct repeat sequence and a spacer sequence, wherein the spacer sequence is capable of hybridizing to a target nucleic acid, e.g., to a target strand (i.e., non-PAM strand) of a target nucleic acid.

Another aspect of the invention provides a cell comprising the Cas12i2 fusion protein of any aspect described herein or the system of any aspect described herein. In some embodiments, the cell is a eukaryotic cell. In some embodiments, the cell is a prokaryotic cell.

In another aspect, the disclosure provides a cell comprising the Cas12i2 fusion protein, the system, the nucleic acid, or the vector of any aspect described herein. In some embodiments, the cell is a eukaryotic cell. In some embodiments, the cell is a prokaryotic cell.

In another aspect, the disclosure provides a method of binding or contacting the Cas12i2 fusion protein of any aspect described herein, or any system described herein with a target nucleic acid in a cell comprising:
(a) providing the Cas12i2 fusion protein or the system;
(b) providing an RNA guide comprising a direct repeat sequence and a spacer sequence, wherein the spacer sequence is capable of hybridizing to the target nucleic acid, e.g., to a target strand (i.e., non-PAM strand) of the target nucleic acid, wherein the Cas12i2 fusion protein is capable of binding to the RNA guide; and
(c) delivering (i) the Cas12i2 fusion protein or the system and (ii) the RNA guide to the cell,
wherein the cell comprises the target nucleic acid, wherein the Cas12i2 fusion protein binds to the RNA guide, and wherein the spacer sequence binds to the target nucleic acid, e.g., to a target strand (i.e., non-PAM strand) of the target nucleic acid.

In some embodiments, the target nucleic acid is a double-stranded DNA.

In another aspect, the disclosure provides a method of modifying a target nucleic acid, the method comprising delivering to the target nucleic acid (i) a Cas12i2 fusion protein of aspect described herein, or any system described herein and (ii) an RNA guide comprising a direct repeat sequence and a spacer sequence, wherein the spacer sequence is capable of hybridizing to the target nucleic acid, e.g., to a target strand (i.e., non-PAM strand) of the target nucleic acid, wherein the Cas12i2 fusion protein is capable of binding to the RNA guide, wherein recognition of the target nucleic acid by the CRISPR-associated protein and RNA guide results in a modification of the target nucleic acid. In some embodiments, the modification comprises DNA methylation, epigenetic modification, or DNA cleavage (e.g., single stranded cleavage, double stranded cleavage, or nicking). In some embodiments, the target nucleic acid comprises a target strand and a non-target strand, and the system modifies the target strand. In certain embodiments, the Cas12i2 fusion protein is any Cas12i2 protein comprising a heterologous sequence disposed between any one of residues i) 342-358 (e.g., 342, 343, 344, 345, 346, 347, 348, 349, 350, 351, 352, 353, 354, 355, 356, 357, or 358); ii) 373-378 (e.g., 373, 374, 375, 376, 377, or 378); iii) 408-413 (e.g., 408, 409, 410, 411, 412, or 413); iv) 677-685 (e.g., 677, 678, 679, 680, 681, 682, 683, 684, or 685); v) 718-723 (e.g., 718, 719, 720, 721, 722, or 723); vi) 771-782 (e.g., 771, 772, 773, 774, 775, 776, 777, 778, 779, 780, 781, or 782); or vii) 953-965 (e.g., 953, 954, 955, 956, 957, 958, 959, 960, 961, 962, 963, 964, or 965) of SEQ ID NO: 1 or any one of SEQ ID NOs: 39-43.

In some embodiments, the target nucleic acid comprises a target strand and a non-target strand, and the system modifies the non-target strand. In certain embodiments, the Cas12i2 fusion protein is any Cas12i2 protein comprising a heterologous sequence disposed between any one of residues viii) 55-65 (e.g., 55, 56, 57, 58, 59, 60, 61, 62, 63, 64, or 65); ix) 99-105 (e.g., 99, 100, 101, 102, 103, 104, or 105); x) 112-120 (e.g., 112, 113, 114, 115, 116, 117, 118, 119, or 120); xi) 195-206 (e.g., 195, 196, 197, 198, 199, 200, 201, 202, 203, 204, 205, or 206); xii) 241-250 (e.g., 241, 242, 243, 244, 245, 246, 247, 248, 249, or 250); xiii) 583-594 (e.g., 583, 584, 585, 586, 587, 588, 589, 590, 591, 592, 593, or 594); xiv) 877-901 (e.g., 877, 878, 879, 880, 881, 882, 883, 884, 885, 886, 887, 888, 889, 890, 891, 892, 893, 894, 895, 896, 897, 898, 899, 900, or 901) of SEQ ID NO: 1 or any one of SEQ ID NOs: 39-43.

In some embodiments of any of the compositions described herein, the system is present in a delivery composition comprising a nanoparticle, a liposome, an exosome, a microvesicle, or a gene-gun.

In some embodiments of any of the compositions described herein, the compositions are within a cell. In some embodiments, the cell is a eukaryotic cell. In some embodiments, the cell is a mammalian cell. In some embodiments, the cell is a human cell. In some embodiments, the cell is a prokaryotic cell.

Other features and advantages of the invention will be apparent from the following detailed description and from the claims.

### BRIEF DESCRIPTION OF THE DRAWINGS

The figures are a series of schematics that represent exemplary Cas12i2 fusion proteins.

FIG. 1 depicts a schematic representation of the initial nucleotide cleavage step of a non-target strand (NTS) by a Cas12i2 complex. The complex comprises a Class 2 type V-I CRISPR Cas12i2 polypeptide comprising a Wedge (Wed) domain, a RuvC domain, a nuclease domain (Nuc), a recognition domain 1 (Rec1), Rec2, and a PAM Interaction domain (PI). The CRISPR RNA (crRNA) binds to the target strand (TS) while the Cas12i2 fusion protein first cleaves the non-target strand (NTS).

FIG. 2 depicts a schematic representation of a Cas12i2 fusion protein comprising a heterologous sequence N-terminal of the Cas12i2 domain. The heterologous sequence includes a linker and a fusion domain. The fusion domain of the exemplary Cas12i2 fusion protein can interact with the ssDNA of the NTS.

FIG. 3 depicts a schematic representation of a Cas12i2 fusion protein comprising a heterologous sequence C-terminal of the Cas12i2 domain. The heterologous sequence includes a linker and a fusion domain. The fusion domain of the exemplary Cas12i2 fusion protein can interact with the ssDNA of the NTS.

FIG. 4 depicts a schematic representation of a Cas12i2 fusion protein comprising a split fusion domain. A first portion of the split fusion domain is located N-terminal of the Cas12i2 domain. A second portion of the split fusion domain is located C-terminal of the Cas12i2 domain. The first portion and the second portion of the split fusion domain are linked the Cas12i2 domain by way of a linker. Alternatively, the first portion of the split fusion domain can be located C-terminal of the Cas12i2 domain, and the second portion of the split fusion domain can be located N-terminal of the Cas12i2 domain. The split fusion domain of the Cas12i2 fusion protein of FIG. 4 can interact at or near the ssDNA of the NTS forming an active fusion domain for acting on the NTS.

FIG. 5 depicts a schematic representation of a Cas12i2 fusion protein comprising a first heterologous sequence located N-terminal of the Cas12i2 domain and a second heterologous sequence located C-terminal of the Cas12i2 domain. In this exemplary Cas12i2 fusion protein, the first heterologous sequence comprises a fusion domain and a first dimerization domain located C-terminal to the fusion domain. In some instances, such as this, the fusion domain and the first dimerization domain are linked by way of a linker. The first heterologous sequence further comprises a linker N-terminal of the fusion domain. The second heterologous sequence comprises a second, compatible dimerization domain. The first dimerization domain and the second dimerization domain of the Cas12i2 fusion protein can dimerize. The fusion domain can interact with the ssDNA of the NTS for acting on the NTS.

FIG. 6 depicts a schematic representation of a circularly permuted, non-naturally occurring Cas12i2 protein, wherein the non-naturally occurring Cas12i2 protein comprises a first portion comprising an amino acid sequence of an N-terminal portion of a Cas12i2 protein, and a second portion comprising an amino acid sequence of a C-terminal portion of a Cas12i2 protein, wherein the second portion is N-terminal of the first portion, and wherein the first portion and the second portion together bind to an RNA guide comprising a direct repeat sequence and a spacer sequence. In this exemplary schematic, the N-terminus and the C-terminus of the Cas12i2 protein are linked by way of a heterologous sequence, and a new N-terminus and C-terminus are located at a loop of interest. In some instances, the heterologous sequence comprises a linker.

FIG. 7 depicts a schematic representation of a Cas12i2 fusion protein, comprising a Cas12i2 domain comprising the circularly permuted, non-naturally occurring Cas12i2 protein depicted in FIG. 6, wherein the heterologous sequence of the non-naturally occurring Cas12i2 protein of FIG. 6 comprises a fusion domain.

FIGS. 8A and 8B depict schematic representations of a Cas12i2 fusion protein comprising a fusion domain. FIGS. 8A and 8B depict Cas12i2 fusion proteins comprising a fusion domain inserted at a loop (e.g., a loop described herein), optionally, having an N-terminal and C-terminal linkers connecting the fusion domain to the Cas12i2 domain(s). In these exemplary depictions, the fusion domain is inlaid within the Rec2 domain. FIG. 8A depicts a Cas12i2 fusion protein with a fusion domain accessing ssDNA of the TS. FIG. 8B depicts a Cas12i2 fusion protein with a fusion domain accessing a ssDNA of the NTS.

FIGS. 9A and 9B depict schematic representations of a Cas12i2 fusion protein comprising a fusion domain. FIGS. 9A and 9B depict Cas12i2 fusion proteins comprising a fusion domain inserted at a loop (e.g., a loop described herein), optionally, having an N-terminal and C-terminal linkers connecting the fusion domain to the Cas12i2 domain(s). In these exemplary depictions, the fusion domain is inlaid within the Nuc domain. FIG. 9A depicts a Cas12i2 fusion protein with a fusion domain accessing ssDNA of the TS. FIG. 9B depicts a Cas12i2 fusion protein with a fusion domain accessing a ssDNA of the NTS.

FIG. 10 depicts a schematic representation of a Cas12i2 fusion protein comprising a surface exposed heterologous sequence. In some instances, the heterologous sequence comprises a linker. In this exemplary schematic, the heterologous sequence comprises a linker(s) and a peptide, such as an NLS peptide.

FIG. 11 depicts a schematic representation of a Cas12i2 fusion protein comprising a FokI nuclease domain. In some instances, the FokI nuclease domain is a heterodimeric FokI nuclease domain. In this exemplary schematic, the heterodimeric FokI nuclease domain comprises a catalytically active FokI nuclease domain and a catalytically inactive FokI nuclease domain.

FIGS. 12A, 12B, 12C, and 12D depict flexible loops of the Cas12i2 protein in proximity to target DNA. FIG. 12A depicts the positions of flexible loops in the Helical II domain (loops at residues 342-358, 373-378, and 386-397), the Helical III domain (loops at residues 677-685 and 771-782), the RuvC II motif (loop at residues 831-844), and the Nuc domain (loop at residues 953-965). FIG. 12B depicts the positions of the loops at residues 373-378, 677-685, and 953-965. FIG. 12C depicts the positions of the loops at residues 342-358 and 386-397. In some embodiments, a FokI nuclease domain is introduced by way of linker in the loop at residues 342-358 and in the loop at residues 386-397. For example, in some embodiments, a catalytically active FokI nuclease domain is introduced into the loop at residues 342-358 and a catalytically inactive FokI nuclease domain is introduced into the loop at residues 386-397. In another example, in some embodiments, a catalytically inactive FokI nuclease domain is introduced into the loop at residues 342-358 and a catalytically active FokI nuclease domain is introduced into the loop at residues 386-397. FIG. 12D depicts the positions of the loops at residues 342-358 and 386-397 as well as the helices between the two loops. In some instances, a circular permutation is introduced at any one of the indicated loops. In some instances, the portion of the Helical II domain positioned from about residue 342 to about 397 is deleted.

FIG. 13A depicts a schematic representation for the engineering a circularly permuted, non-naturally occurring Cas12i2 protein. The top panel depicts the domains of a reference Cas12i2 protein. In the middle panel of this exemplary schematic, the N-terminus and the C-terminus of the Cas12i2 protein are linked by way of a heterologous sequence (e.g., a linker), and a new N-terminus and C-terminus are located at a loop of interest (e.g., a loop within the Helical II domain). In some instances, the new N-terminus and/or C-terminus comprise a fusion domain. In some instances, the fusion domain is a FokI nuclease domain. As depicted in this exemplary schematic, the new N-terminus can be fused to a dead FokI nuclease domain, and the new C-terminus can be fused to an active FokI nuclease domain.

FIG. 13B depicts a schematic representation for the engineering a circularly permuted, non-naturally occurring Cas12i2 protein. The top panel depicts the domains of a reference Cas12i2 protein and a portion of the Helical II domain that can be mutated or deleted (see asterisk). In the middle panel of this exemplary schematic, the N-terminus and the C-terminus of the Cas12i2 protein are linked by way of a heterologous sequence (e.g., a linker), a portion of the Helical II domain is deleted (e.g., the portion from about residue 342 to about 397), and a new N-terminus and C-terminus are located within the Helical II domain. In some instances, the new N-terminus and/or C-terminus comprise a fusion domain. In some instances, the fusion domain is a FokI nuclease domain. As depicted in this exemplary schematic, the new N-terminus can be fused to a dead FokI nuclease domain, and the new C-terminus can be fused to an active FokI nuclease domain.

FIG. 14A shows indel activity of the variant Cas12i2 polypeptide of SEQ ID NO: 40 and the circularly permuted Cas12i2 polypeptides of SEQ ID NOs: 45-52 on four mammalian targets. FIG. 14B shows indel activity of the variant Cas12i2 polypeptide of SEQ ID NO: 40 and the circularly permuted Cas12i2 polypeptides of SEQ ID NOs: 45-52 averaged across four mammalian targets. The data shown is an average of two bioreplicates.

### DETAILED DESCRIPTION

The present disclosure relates to novel Cas12i2 fusion proteins and methods of use thereof. In some aspects, a composition comprising a Cas12i2 fusion protein having one or more characteristics is described herein. In some aspects, a method of producing a Cas12i2 fusion protein is described. In some aspects, a method of delivering a composition comprising a Cas12i2 fusion protein is described.

The term “base editing domain,” as described herein refers to an agent comprising a polypeptide that is capable of making a chemical modification to a base (e.g., A, T, C, G, or U) within a nucleic acid sequence (e.g., DNA or RNA). In some embodiments, a base editing domain changes a first canonical base into a second canonical base. In some embodiments, a base editing domain changes a canonical base into a non-canonical base.

As used herein, a “biologically active portion” of a polypeptide is a portion of a polypeptide that maintains a function (e.g. completely, partially, or minimally) of the polypeptide (e.g., a Cas12i2 domain (e.g., a “minimal” or “core” domain) or a fusion domain).

As used herein, the term “catalytic residue” refers to an amino acid that activates catalysis. A catalytic residue is an amino acid that is involved (e.g., directly involved) in catalysis.

The term “Cas12i2 fusion protein,” as used herein, refers to a polypeptide having: i) one or more domains, wherein at least one of the domains includes a portion of a Cas12i2 domain and ii) a heterologous sequence, wherein the Cas12i2 fusion protein comes into contact with a target nucleic acid specified by an RNA guide. In some embodiments, the Cas12i2 fusion protein has enzymatic (e.g., nuclease) activity. In some embodiments, an enzymatic activity (e.g., nuclease activity) can be carried out by the Cas12i2 domain. In some instances, the Cas12i2 domain comprises an amino acid sequence having at least 80% (e.g., 80%, 85%, 90%, 95%, 96%, 97%, 98%, or 99%) sequence identity to any one of SEQ ID NOs: 1 or 39-43 or a portion thereof. In some instances, the Cas12i2 domain has the sequence of SEQ ID NO: 1 or a portion thereof. In some instances, the Cas12i2 domain includes a first portion and a second portion, wherein the first portion and the second portion together bind to an RNA guide comprising a direct repeat sequence and a spacer sequence. Optionally, the first and second portions are not directly adjacent to each other. For example, in some instances, a heterologous sequence is adjacent to the first portion and to the second portion. In some instances, a heterologous sequence is C-terminal of the first portion and N-terminal of the second portion. In some instances, the heterologous sequence is N-terminal of the first portion and C-terminal of the second portion. While the amino acid numbering system used herein is in relation to SEQ ID NO: 1, other Cas12i2 sequences can be used. One of ordinary skill in the art can identify the corresponding amino acid positions in another Cas12i2 sequences using available tools, such as sequence alignment algorithms.

As used herein, the term “dimerization domain,” refers to a polypeptide domain capable of specifically binding a separate, and compatible, polypeptide domain (e.g., a second compatible dimerization domain). In some embodiments, the dimer is formed by a non-covalent bond between the first dimerization domain and the second compatible dimerization domain. In some embodiments, the first dimerization domain and the second compatible dimerization domain are identical (e.g., a homodimer). In some embodiments, the first dimerization domain and the second dimerization domain are not identical (e.g., a heterodimer). In some embodiments, a dimerization domain is a leucine zipper. In some instances, the dimerization domain is a chemically inducible dimerization domain (e.g., a rapamycin sensitive dimerization domain) that can be regulated by the presence of a small molecule.

As used herein, the terms “domain” and “protein domain” refer to a distinct functional and/or structural unit of a polypeptide. In some embodiments, a domain may comprise a conserved amino acid sequence. As used herein, the term “RuvC domain” refers to a conserved domain or motif of amino acids having nuclease (e.g., endonuclease) activity. As used herein, a protein having a split RuvC domain refers to a protein having two or more RuvC motifs, at sequentially disparate sites within a sequence, that interact in a tertiary structure to form a RuvC domain.

The term “fusion domain,” as used herein, refers to a polypeptide domain that is operably linked to a second, heterologous domain. In some embodiments, the fusion domain is about 1-5, 10-20, 20-50, 50-100, or 100-200 amino acids in length.

The term “heterologous,” when used to describe a first element in reference to a second element means that the first element and second element do not exist in nature disposed as described. For example, a heterologous polypeptide sequence refers to (a) a polypeptide, or portion of a polypeptide that is operably linked to a second polypeptide sequence to which it is not operably linked in nature, (b) a polypeptide or portion of a polypeptide that is not native to a cell in which it is expressed, (c) a polypeptide or portion of a polypeptide that has been altered or mutated relative to its native state, or (d) a polypeptide with an altered expression as compared to the native expression levels under similar conditions. As an example, a heterologous sequence of a polypeptide may be a different sequence or from a different source, relative to other domains or portions of a polypeptide. In some instances, the heterologous sequence includes a fusion domain and at least one linker sequence.

As used herein, the term “insertion” refers to a gain of residues in an amino acid sequence.

As used herein, the term “nuclease” refers to an enzyme capable of cleaving a phosphodiester bond. A nuclease hydrolyzes phosphodiester bonds in a nucleic acid backbone. As used herein, the term “endonuclease” refers to an enzyme capable of cleaving a phosphodiester bond between nucleotides.

As used herein, the terms “parent,” “parent polypeptide,” and “parent sequence” refer to an original polypeptide (e.g., starting polypeptide) to which an alteration is made to produce a variant polypeptide. In some embodiments, the parent is an Cas12i2 having an identical amino acid sequence of the variant at one or more of specified positions. The parent may be a naturally occurring (wild-type) polypeptide. In a particular embodiment, the parent is a polypeptide with at least 60%, at least 61%, at least 62%, at least 63%, at least 64%, at least 65%, at least 70%, at least 72%, at least 73%, at least 74%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% identity to a polypeptide described herein of any one of SEQ ID NO: 1 and SEQ ID NOs: 39-43.

The term “basic domain,” as used herein refers to a polypeptide domain comprising a plurality of basic amino acids (e.g., histidine, lysine, arginine, or any combination thereof). In some embodiments, the basic domain can bind to a nucleic acid. Optionally, a basic domain can comprise one or more non-basic (e.g., polar, nonpolar, or acidic) amino acids dispersed throughout. For example, in some embodiments, the basic domain comprises a plurality of lysine residues but no histidine or arginine residues. In other embodiments, the basic domain may comprise a plurality of lysine residues and one or both of histidine and arginine residues.

The term “poly-basic domain,” as used herein refers to a polypeptide domain comprising a combination of histidine, lysine, and/or arginine that can bind a nucleic acid, e.g., by interacting with the negatively charged phosphate backbone or DNA through electrostatic interactions, and, optionally, one or more non-basic (e.g., polar, nonpolar, or acidic) amino acids dispersed throughout. In some instances, the poly-basic domain comprises between 5 and 50 (e.g., between 5-10, 10-20, 20-30, 30-40, or 40-50) arginine residues. In some instances, the poly-basic domain comprises between 5 and 50 (e.g., between 5-10, 10-20, 20-30, 30-40, or 40-50) lysine residues. In some instances, the poly-basic domain comprises between 5 and 50 (e.g., between 5-10, 10-20, 20-30, 30-40, or 40-50) histidine residues. In some instances, the poly-basic domain comprises one or more polar amino acids (e.g., Q, N, and/or S) located between a two poly-basic sequences each independently between 5 and 25 (e.g., between 5-10, 10-15, 15-20, or 20-25) residues in length.

The term “polypeptide linker,” as used herein refers to a linker that comprises amino acids and links together two amino acid sequences (e.g., domains). In some embodiments, the polypeptide linker comprises glycine and/or serine residues used alone or in combination. In some embodiments, the peptide linker connects two portions of the Cas12i2 fusion protein together.

As used herein, the term “protospacer adjacent motif” or “PAM” refers to a DNA sequence adjacent to a target sequence to which a complex comprising a CRISPR nuclease (e.g., a Cas12i2 fusion protein) and an RNA guide binds. In some embodiments, a PAM is required for binding of a Cas12i2 fusion protein and an RNA guide to a target nucleic acid. As used herein, the term “adjacent” includes instances in which an RNA guide of the complex specifically binds, interacts, or associates with a target sequence that is immediately adjacent to a PAM. In such instances, there are no nucleotides between the target sequence and the PAM. The term “adjacent” also includes instances in which there are a small number (e.g., 1, 2, 3, 4, or 5) of nucleotides between the target sequence, to which the targeting moiety binds, and the PAM.

As used herein, the terms “reference composition,” “reference sequence,” and “reference” refer to a control, such as a negative control or a parent (e.g., a parent sequence, a parent protein, a wild-type protein, or a complex comprising a parent sequence).

As used herein, the terms “RNA guide” or “RNA guide sequence” refer to any RNA molecule that facilitates the targeting of a polypeptide described herein (e.g., a Cas12i2 fusion protein) to a target nucleic acid. For example, an RNA guide can be a molecule that recognizes (e.g., binds to) a target nucleic acid. An RNA guide may be designed to be complementary to a target nucleic acid, e.g., a target strand (i.e., non-PAM strand) of a target nucleic acid sequence. An RNA guide comprises a DNA targeting sequence and a direct repeat (DR) sequence. The terms CRISPR RNA (crRNA), pre-crRNA, mature crRNA, and gRNA are also used herein to refer to an RNA guide. As used herein, the term “pre-crRNA” refers to an unprocessed RNA molecule comprising a DR-spacer-DR sequence. As used herein, the term “mature crRNA” refers to a processed form of a pre-crRNA; a mature crRNA may comprise a DR-spacer sequence, wherein the DR is a truncated form of the DR of a pre-crRNA and/or the spacer is a truncated form of the spacer of a pre-crRNA.

The term “split fusion domain,” as used herein refers to: (i) a first portion (e.g., an N-terminal portion, a C-terminal portion, or a central portion) of a reference polypeptide, and (ii) a second portion of the reference polypeptide; wherein (i) and (ii) are non-contiguous (e.g., are present on a single polypeptide chain but separated by a Cas12i2 domain or are present on different polypeptide chains); and wherein (i) and (ii) bound together have one or more activity of the reference polypeptide.

The term, “ssDNA binding domain” as used herein refers to a polypeptide domain that binds a single stranded DNA molecule (e.g., an unwound portion of a largely double stranded DNA molecule). In some instances, the ssDNA binding domain comprises a single-stranded DNA binding protein (SSB) found in E. coli (see, e.g., Oakley A. J. Nucleic Acid Research 42(4): 2750-2757, 2014).

As used herein, the term “substantially identical” refers to a sequence, polynucleotide, or polypeptide, that has a certain degree of identity to a reference sequence.

As used herein, the terms “target nucleic acid” and “target sequence” refer to a nucleic acid sequence to which a targeting moiety (e.g., RNA guide) specifically binds. In some embodiments, the DNA targeting sequence of an RNA guide binds to a target nucleic acid. The target nucleic acid is typically a double-stranded molecule, wherein one strand comprises the target sequence adjacent to the PAM and is referred to as the “PAM strand” (i.e., the non-target strand or the non-spacer-complementary strand), and the other, complementary strand is referred to as the “non-PAM strand” (i.e., the target strand or the spacer-complementary strand).

### Cas12i2 Fusion Proteins

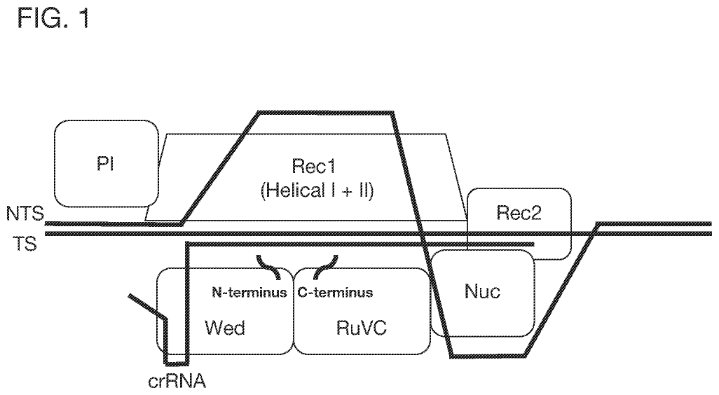

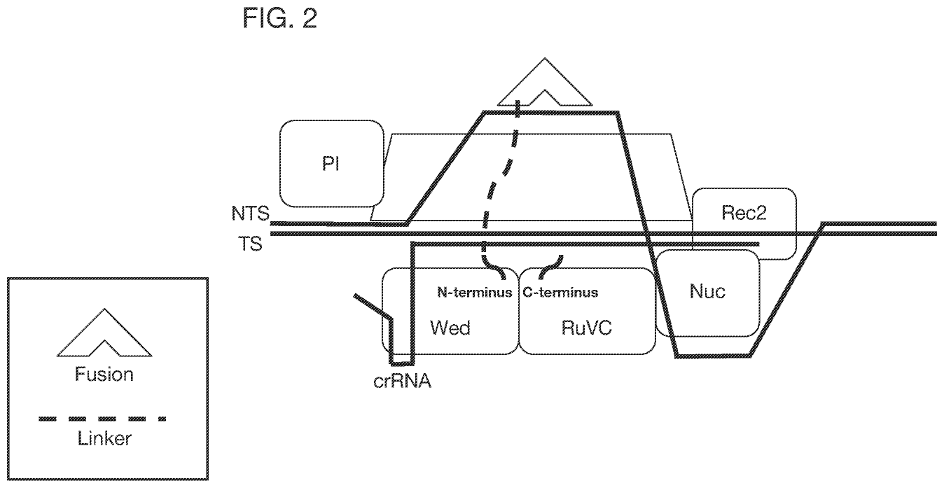

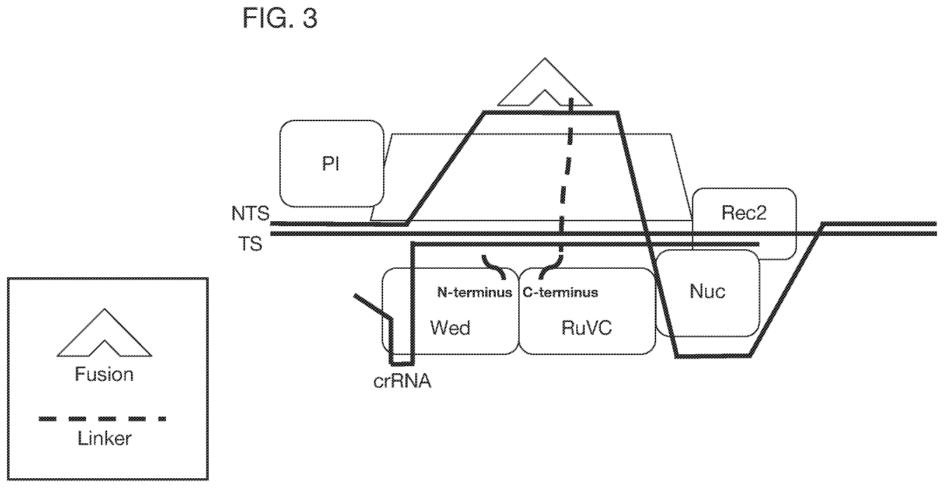

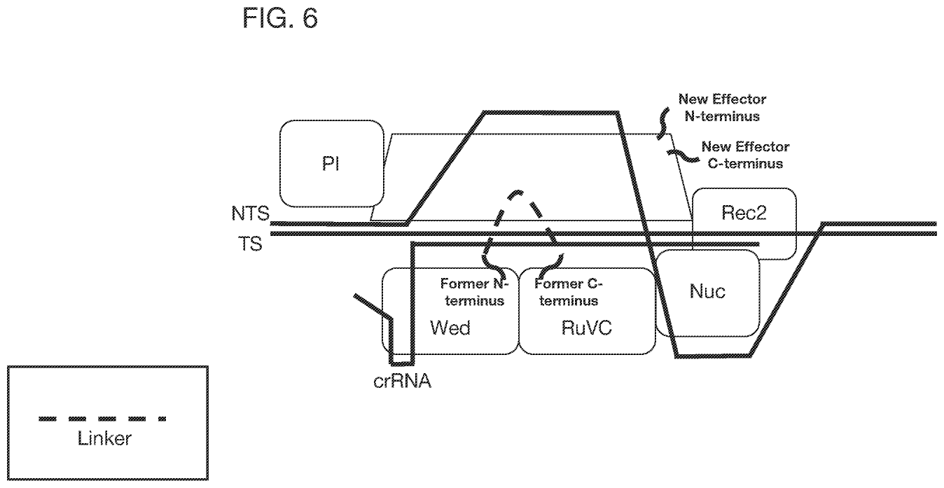

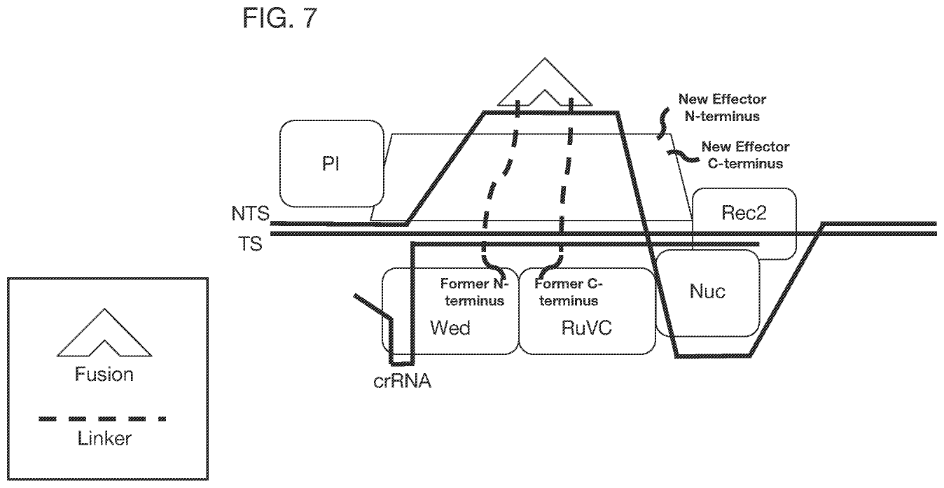

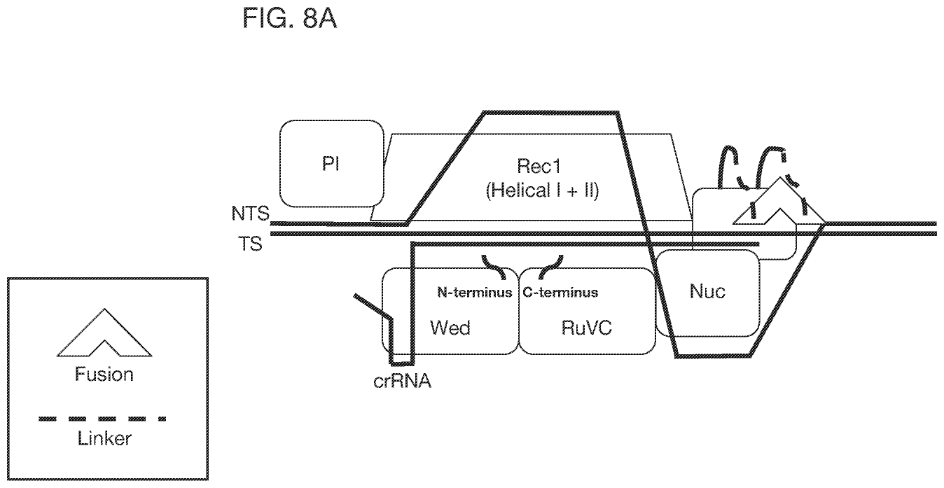

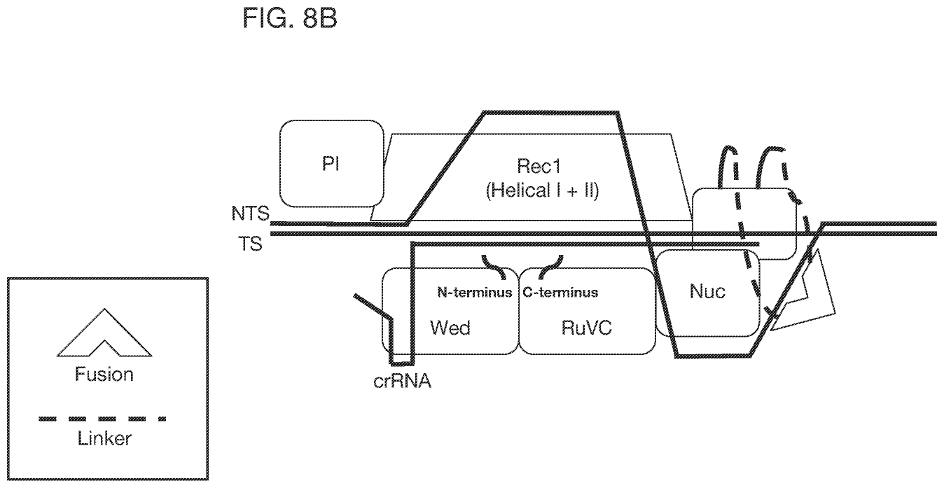

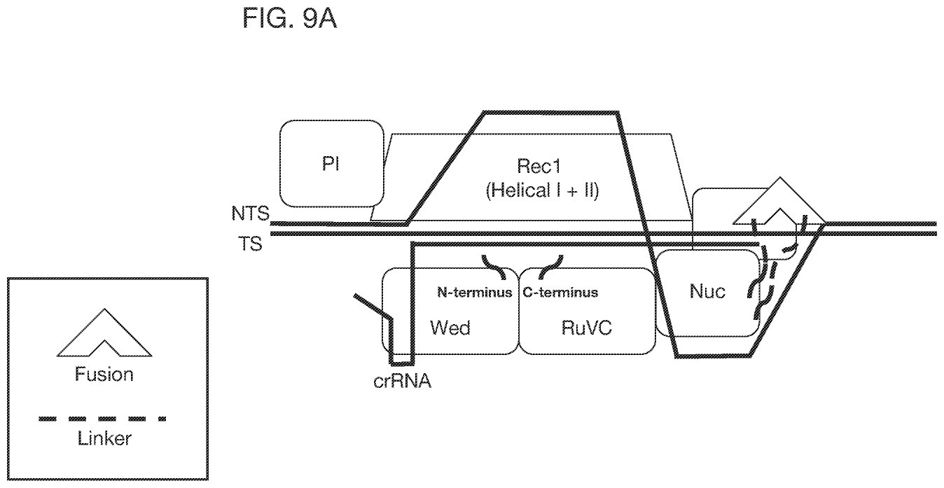

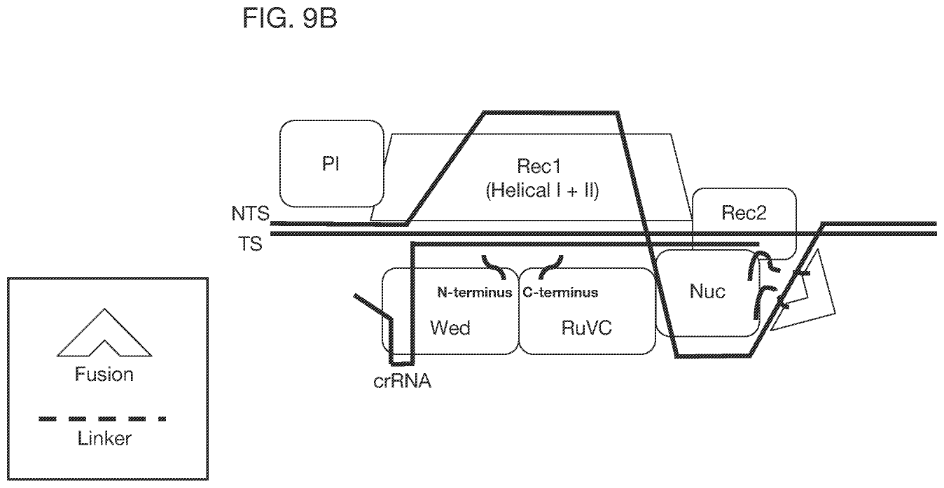

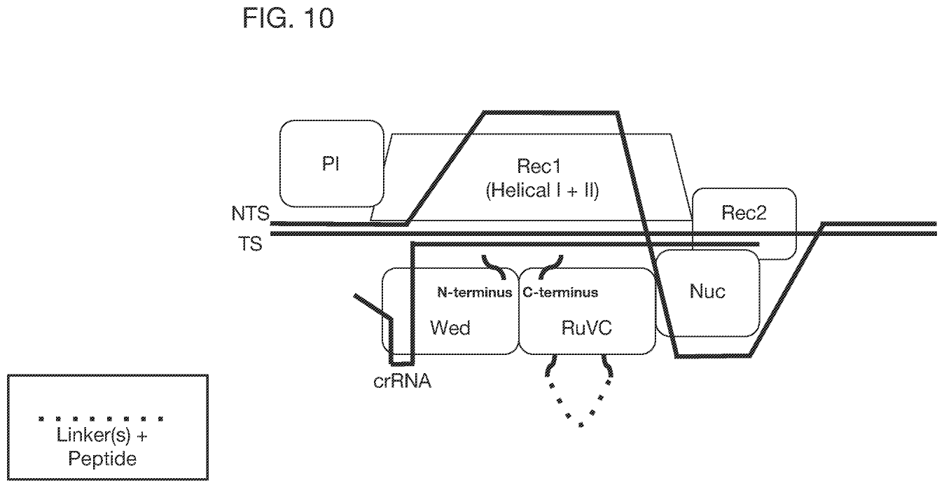

The present disclosure provides, e.g., fusion proteins including: i) one or more domains, wherein at least one of the domains includes a portion of a Cas12i2 domain and ii) a heterologous sequence, wherein the Cas12i2 fusion protein comes into contact with (e.g., associates with, recognizes, or binds) a target nucleic acid with an RNA guide. In some embodiments, the Cas12i2 fusion protein has enzymatic activity. In some embodiments, the enzymatic activity can be carried out by the Cas12i2 domain. In some embodiments, the heterologous sequence comprises a fusion domain (e.g., a domain having various activities, e.g., methylase activity, demethylase activity, transcription activation activity, transcription repression activity, transcription release factor activity, histone modification activity, RNA cleavage activity, DNA cleavage activity, nucleic acid binding activity, and switch activity (e.g., light inducible)). In some embodiments, the Cas12i2 fusion protein comprises a domain architecture shown, for example, in any of FIGS. 1-10.

In one aspect, the disclosure provides a Cas12i2 fusion protein comprising:
a) a first portion comprising amino acids 1-n of SEQ ID NO: 1, or a sequence having at least 80%, 85%, 90%, 95%, 96%, 97%, 98%, or 99% identity thereto;
b) a second portion comprising amino acids m-1054 of SEQ ID NO: 1, or a sequence having at least 80%, 85%, 90%, 95%, 96%, 97%, 98%, or 99% identity thereto; and
c) a heterologous sequence disposed between the first portion and the second portion,
wherein n and m are each independently a number between:
i) 342-358 (e.g., 342, 343, 344, 345, 346, 347, 348, 349, 350, 351, 352, 353, 354, 355, 356, 357, or 358);
ii) 373-378 (e.g., 373, 374, 375, 376, 377, or 378);
iii) 408-413 (e.g., 408, 409, 410, 411, 412, or 413);
iv) 677-685 (e.g., 677, 678, 679, 680, 681, 682, 683, 684, or 685);
v) 718-723 (e.g., 718, 719, 720, 721, 722, or 723);
vi) 771-782 (e.g., 771, 772, 773, 774, 775, 776, 777, 778, 779, 780, 781, or 782);
vii) 953-965 (e.g., 953, 954, 955, 956, 957, 958, 959, 960, 961, 962, 963, 964, or 965);
viii) 55-65 (e.g., 55, 56, 57, 58, 59, 60, 61, 62, 63, 64, or 65);
ix) 99-105 (e.g., 99, 100, 101, 102, 103, 104, or 105);
x) 112-120 (e.g., 112, 113, 114, 115, 116, 117, 118, 119, or 120);
xi) 195-206 (e.g., 195, 196, 197, 198, 199, 200, 201, 202, 203, 204, 205, or 206);
xii) 241-250 (e.g., 241, 242, 243, 244, 245, 246, 247, 248, 249, or 250);
xiii) 583-594 (e.g., 583, 584, 585, 586, 587, 588, 589, 590, 591, 592, 593, or 594);
xiv) 877-901 (e.g., 877, 878, 879, 880, 881, 882, 883, 884, 885, 886, 887, 888, 889, 890, 891, 892, 893, 894, 895, 896, 897, 898, 899, 900, or 901);
xv) 173-179 (e.g., 173, 174, 175, 176, 177, 178, or 179);
xvi) 216-221 (e.g., 216, 217, 218, 219, 220, or 221);
xvii) 265-272 (e.g., 265, 266, 267, 268, 269, 270, 271, or 272);
xviii) 456-468 (e.g., 456, 457, 458, 459, 460, 461, 462, 463, 464, 465, 466, 467, or 468);
xix) 476-482 (e.g., 476, 477, 478, 479, 480, 481, or 482);
xx) 498-513 (e.g., 498, 499, 500, 501, 502, 503, 504, 505, 506, 507, 508, 509, 510, 511, 512, or 513);
xxi) 614-625 (e.g., 614, 615, 616, 617, 618, 619, 620, 621, 622, 623, 624, or 625);
xxii) 977-982 (e.g., 977, 978, 979, 980, 981, or 982); or
xxiii) 1007-1012 (e.g., 1007, 1008, 1009, 1010, 1011, or 1012).

In some embodiments, n<m. In some embodiments, m=n+1.

### Exemplary Cas12i2 Fusion Proteins Having a Heterologous Sequence at the PAM Distal Region of Amino Acids S342-L358

In some embodiments of any Cas12i2 fusion protein described herein, a) n is 342 and m is 343, or b) n is 347 and m is 348. In some embodiments, the first portion comprises at least 273, 280, 290, 300, 310, 320, 330, 340, 341, or 342 amino acids. In certain embodiments, the second portion comprises at least 570, 580, 590, 600, 610, 620, 630, 640, 650, 660, 670, 680, 690, 700, 710, 711, or 712 amino acids. In certain embodiments, the C-terminal amino acid(s) of the first portion comprise FDS, DS, or S. In some embodiments, the N-terminal amino acid(s) of the second portion comprise EFS, EF, or E. In some embodiments, the heterologous moiety is situated between any two adjacent amino acids of SEFFSGEETYTICVHHL (SEQ ID NO: 2), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, 5 and 6, 6 and 7, 7 and 8, 8 and 9, 9 and 10, 10 and 11, 11 and 12, 12 and 13, or 13 and 14, of SEQ ID NO: 2. In certain embodiments, one or more amino acids of SEQ ID NO: 2 are absent from the Cas12i2 fusion protein. In other embodiments, 1, 2, 3, 4, 5, 6, 7, 8, 9, 10, 11, 12, or 13 sequential amino acids of SEQ ID NO: 2 that are N-terminal to the heterologous sequence are absent from the Cas12i2 fusion protein. In certain embodiments, 1, 2, 3, 4, 5, 6, 7, 8, 9, 10, 11, 12, or 13 sequential amino acids of SEQ ID NO: 2 that are C-terminal to the heterologous sequence are absent from the Cas12i2 fusion protein.

### Exemplary Cas12i2 Fusion Proteins Having a Heterologous Sequence at the PAM Distal Region of Amino Acids D373-E378

In certain embodiments, n is 374 and m is 375. In some embodiments, the first portion comprises at least 300, 310, 320, 330, 340, 350, 360, 370, 373, 374, 375, 376, or 377 amino acids. In certain embodiments, the second portion comprises at least 544, 560, 570, 580, 590, 600, 610, 620, 630, 640, 650, 660, 670, or 680 amino acids. In some embodiments, the C-terminal amino acid(s) of the first portion comprise DDP, DP, or P. In some embodiments, the N-terminal amino acid(s) of the second portion comprise ADP, AD, or A. In certain embodiments, the heterologous moiety is situated between any two adjacent amino acids of DPADPE (SEQ ID NO: 3), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, or 5 and 6 of SEQ ID NO: (3). In some embodiments, one or more amino acids of SEQ ID NO: 3 are absent from the Cas12i2 fusion protein. In some embodiments, 1, 2, 3, 4, 5, or 6 sequential amino acids of SEQ ID NO: 3 that are N-terminal to the heterologous sequence are absent from the Cas12i2 fusion protein. In some embodiments, 1, 2, 3, 4, 5, or 6 sequential amino acids of SEQ ID NO: 3 that are C-terminal to the heterologous sequence are absent from the Cas12i2 fusion protein.

### Exemplary Cas12i2 Fusion Proteins Having a Heterologous Sequence at the PAM Distal Region of Amino Acids R408-A413

In some embodiments of the fusion Cas12i2 proteins described herein, a) n is 409 and m is 410 or b) n is 410 and m is 411. In certain embodiments, the first portion comprises at least 328 330, 340, 350, 360, 370, 380, 390, 400, 405, 406, 407, 408, 409, or 410 amino acids. In some embodiments, the second portion comprises at least 516, 520, 530, 540, 550, 560, 570, 580, 590, 600, 610, 620, 630, 640, 641, 642, 643, 644, or 645 amino acids. In certain embodiments, the C-terminal amino acid(s) of the first portion comprise IRQE, RQ, Q, or E. In some embodiments, the N-terminal amino acid(s) of the second portion comprise ECS, EC, E, or C. In some embodiments, the heterologous moiety is situated between any two adjacent amino acids of RQECSA (SEQ ID NO: 4), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, or 5 and 6 of SEQ ID NO: 4. In some embodiments, one or more amino acids of SEQ ID NO: 4 are absent from the Cas12i2 fusion protein. In some embodiments, 1, 2, 3, 4, 5, or 6 sequential amino acids of SEQ ID NO: 4 that are N-terminal to the heterologous sequence are absent from the Cas12i2 fusion protein. In certain embodiments, 1, 2, 3, 4, 5, or 6 sequential amino acids of SEQ ID NO: 4 that are C-terminal to the heterologous sequence are absent from the Cas12i2 fusion protein.

### Exemplary Cas12i2 Fusion Proteins Having a Heterologous Sequence at the PAM Distal Region of Amino Acids K677-V685

In some embodiments, n is 682 and m is 683. In some embodiments, the first portion comprises at least 546, 550, 560, 570, 580, 590, 600, 610, 620, 630, 640, 650, 660, 670, 680, 681, or 682 amino acids. In certain embodiments, the second portion comprises at least 298, 300, 310, 320, 330, 340, 350, 360, 370, 371, or 372 amino acids. In some embodiments, the C-terminal amino acid(s) of the first portion comprise KKK, KK, or K. In some embodiments, the N-terminal amino acid(s) of the second portion comprise EIV, EI, or E. In certain embodiments, the heterologous moiety is situated between any two adjacent amino acids of KKNKKKEIV (SEQ ID NO: 5), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, 5 and 6, 7 and 8, or 8 and 9 of SEQ ID NO: 5. In some embodiments, one or more amino acids of SEQ ID NO: 5 are absent from the Cas12i2 fusion protein. In some embodiments, 1, 2, 3, 4, 5, 6, 7, 8, or 9 sequential amino acids of SEQ ID NO: 5 that are N-terminal to the heterologous sequence are absent from the Cas12i2 fusion protein. In some embodiments, 1, 2, 3, 4, 5, 6, 7, 8, or 9 sequential amino acids of SEQ ID NO: 5 that are C-terminal to the heterologous sequence are absent from the Cas12i2 fusion protein.

Exemplary Cas12i2 Fusion Proteins Having a Heterologous Sequence at the PAM Distal Region of Amino Acids V718-L723 In some embodiments, n is 721 and m is 722. In some embodiments, the first portion comprises at least 577, 580, 590, 600, 610, 620, 630, 640, 650, 660, 670, 680, 690, 700, 710, 720, or 721 amino acids. In certain embodiments, the second portion comprises at least 266, 270, 280, 290, 300, 310, 320, 330, 331, 332, or 333 amino acids. In some embodiments, the C-terminal amino acid(s) of the first portion comprise RGK, GK, or K. In some embodiments, the N-terminal amino acid(s) of the second portion comprise SLV, SL, or S. In certain embodiments, the heterologous moiety is situated between any two adjacent amino acids of VRGKSL (SEQ ID NO: 6), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, or 5 and 6 of SEQ ID NO: 6. In some embodiments, one or more amino acids of SEQ ID NO: 6 are absent from the Cas12i2 fusion protein. In some embodiments, 1, 2, 3, 4, 5, or 6 sequential amino acids of SEQ ID NO: 6 that are N-terminal to the heterologous sequence are absent from the Cas12i2 fusion protein. In certain embodiments, 1, 2, 3, 4, 5, or 6 sequential amino acids of SEQ ID NO: 6 that are C-terminal to the heterologous sequence are absent from the Cas12i2 fusion protein.

### Exemplary Cas12i2 Fusion Proteins Having a Heterologous Sequence at the PAM Distal Region of Amino Acids A771-D782

In some embodiments, n is 778 and m is 779. In certain embodiments, the first portion comprises at least 622, 630, 640, 650, 660, 670, 680, 690, 700, 710, 720, 730, 740, 750, 760, 770, 775, 776, 777 or 778 amino acids. In certain embodiments, the second portion comprises at least 221, 225, 230, 240, 250, 260, 270, 275, or 276 amino acids. In some embodiments, the C-terminal amino acid(s) of the first portion comprise KNN, NN, or N. In certain embodiments, the N-terminal amino acid(s) of the second portion comprise PIS, PI, or P. In some embodiments, the heterologous moiety is situated between any two adjacent amino acids of ALNASKNNPISD (SEQ ID NO: 7), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, 5 and 6, 6 and 7, 7 and 8, 8 and 9, 9 and 10, 10 and 11, or 11 and 12 of SEQ ID NO: 7. In some embodiments, one or more amino acids of SEQ ID NO: Xe are absent from the Cas12i2 fusion protein. In certain embodiments, 1, 2, 3, 4, 5, 6, 7, 8, 9, 10, 11, or 12 sequential amino acids of SEQ ID NO: 7 that are N-terminal to the heterologous sequence are absent from the Cas12i2 fusion protein. In some embodiments, 1, 2, 3, 4, 5, 6, 7, 8, 9, 10, 11, or 12 sequential amino acids of SEQ ID NO: 7 that are C-terminal to the heterologous sequence are absent from the Cas12i2 fusion protein.

### Exemplary Cas12i2 Fusion Proteins Having a Heterologous Sequence at the PAM Distal Region of Amino Acids L953-C965

In some embodiments, n is 960 and m is 961. In certain embodiments, the first portion comprises at least 768, 770, 780, 790, 800, 810, 820, 830, 840, 850, 860, 870, 880, 890, 900, 910, 920, 930, 940, 950, or 960 amino acids. In certain embodiments, the second portion comprises at least 75, 80, 85, 90, 91, 92, 93, or 94 amino acids. In some embodiments, the C-terminal amino acid(s) of the first portion comprise DRK, RK, or K. In certain embodiments, the N-terminal amino acid(s) of the second portion comprise SNI, SN, or S. In some embodiments, the heterologous moiety is situated between any two adjacent amino acids of LKWRSDRKSNIPC (SEQ ID NO: 8), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, 5 and 6, 6 and 7, 7 and 8, 8 and 9, 9 and 10, 10 and 11, 11 and 12, or 12 and 13 of SEQ ID NO: 8. In certain embodiments, one or more amino acids of SEQ ID NO: 8 are absent from the Cas12i2 fusion protein. In some embodiments, 1, 2, 3, 4, 5, 6, 7, 8, 9, 10, 11, 12, or 13 sequential amino acids of SEQ ID NO: 8 that are N-terminal to the heterologous sequence are absent from the Cas12i2 fusion protein. In certain embodiments, 1, 2, 3, 4, 5, 6, 7, 8, 9, 10, 11, 12, or 13 sequential amino acids of SEQ ID NO: 8 that are C-terminal to the heterologous sequence are absent from the Cas12i2 fusion protein.

### Exemplary Cas12i2 Fusion Proteins Having a Heterologous Sequence at the PAM Proximal Region of Amino Acids S55-65

In some embodiments of the Cas12i2 fusion protein described herein, a) n is 61 and m is 62, or b) n is 62 and m is 63. In some embodiments, the first portion comprises at least 49, 50, 51, 52, 53, 54, 55, 56, 57, 58, 59, 60, or 61 amino acids. In certain embodiments, the second portion comprises at least 795, 800, 810, 820, 830, 840, 850, 860, 870, 880, 890, 900, 910, 920, 930, 940, 950, 960, 970, 980, 990, or 991 amino acids. In some embodiments, the C-terminal amino acid(s) of the first portion comprise EKQ, KQ, or Q. In certain embodiments, the N-terminal amino acid(s) of the second portion comprise QQD, QQ, or Q. In some embodiments, the heterologous moiety is situated between any two adjacent amino acids of STEQEKQQQDI (SEQ ID NO: 9), e.g., between positions 1 and 2, 2 and 3, 3 and 4, 4 and 5, 5 and 6, 6 and 7, 7 and 8, or 8 and 9 of SEQ ID NO: 9. In certain embodiments, one or more amino acids of SEQ ID NO: 9 are absent from the Cas12i2 fusion protein. In some embodiments, 1, 2, 3, 4, 5, 6, 7, 8, or 9 amino acids of SEQ ID NO: 9 that are N-terminal to the heterologous sequence are absent from the Cas12i2 fusion protein. In some embodiments, 1, 2, 3, 4, 5, 6, 7, 8, or 9 sequential amino acids of SEQ ID NO: 9 that are C-terminal to the heterologous sequence are absent from the Cas12i2 fusion protein.

### Exemplary Cas12i2 Fusion Proteins Having a Heterologous Sequence at the PAM Proximal Region of Amino Acids Y99-D105

In certain embodiments of the Cas12i2 fusion protein described herein, a) n is 101 and m is 102, or b) n is 102 and m is 103. In certain embodiments, the first portion comprises at least 81, 90, 100, or 101 amino acids. In certain embodiments, the second portion comprises at least 762, 770, 780, 790, 800, 810, 820, 830, 840, 850, 860, 870, 880, 890, 900, 910, 920, 930, 940, 950, 951, 952, or 953 amino acids. In some embodiments, the C-terminal amino acid(s) of the first portion comprise YGGT, YGG, GG, G, or T. In some embodiments, the N-terminal amino acid(s) of the second portion comprise TAS, TA, AS, T, or A. In some embodiments, the heterologous moiety is situated between any two adjacent amino acids of YGGTASD (SEQ ID NO: 10), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, 5 and 6, or 6 and 7 of SEQ ID NO: 10. In some embodiments, one or more amino acids of SEQ ID NO: 10 are absent from the Cas12i2 fusion protein. In certain embodiments, 1, 2, 3, 4, 5, 6, or 7 sequential amino acids of SEQ ID NO: 10 that are N-terminal to the heterologous sequence are absent from the Cas12i2 fusion protein. In some embodiments, 1, 2, 3, 4, 5, 6, or 7 sequential amino acids of SEQ ID NO: 10 that are C-terminal to the heterologous sequence are absent from the Cas12i2 fusion protein.

### Exemplary Cas12i2 Fusion Proteins Having a Heterologous Sequence at the PAM Proximal Region of Amino Acids S112-Y120

In some embodiments, n is 116 and m is 117. In certain embodiments, the first portion comprises at least 81, 90, 100, or 101 amino acids. In some embodiments, the second portion comprises at least 762, 780, 790, 800, 810, 820, 830, 840, 850, 860, 870, 880, 890, 900, 910, 920, 930, 940, 950, 951, 952, or 953 amino acids. In other embodiments, the C-terminal amino acid(s) of the first portion comprise SIG, IG, or G. In certain embodiments, the N-terminal amino acid(s) of the second portion comprise ESY, ES, or E. In other embodiments, the heterologous moiety is situated between any two adjacent amino acids of SASIGESYY (SEQ ID NO: 11), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, 5 and 6, 6 and 7, 7 and 8, or 8 and 9 of SEQ ID NO: 11. In some embodiments, one or more amino acids of SEQ ID NO: 11 are absent from the Cas12i2 fusion protein. In other embodiments, 1, 2, 3, 4, 5, 6, 7, 8, or 9 sequential amino acids of SEQ ID NO: 11 that are N-terminal to the heterologous sequence are absent from the Cas12i2 fusion protein. In certain embodiments, 1, 2, 3, 4, 5, 6, 7, 8, or 9 sequential amino acids of SEQ ID NO: 11 that are C-terminal to the heterologous sequence are absent from the Cas12i2 fusion protein.

### Exemplary Cas12i2 Fusion Proteins Having a Heterologous Sequence at the PAM Proximal Region of Amino Acids S195-P206

In some embodiments, n is 199 and m is 200. In other embodiments, the first portion comprises at least 160, 170, 180, 190, 195, 196, 197, 198, or 199 amino acids. In certain embodiments, the second portion comprises at least 684, 690, 700, 710, 720, 730, 740, 750, 760, 780, 790, 800, 810, 820, 830, 840, 850, or 855 amino acids. In some embodiments, the C-terminal amino acid(s) of the first portion comprise LKE, KE, or E. In some embodiments, the N-terminal amino acid(s) of the second portion comprise IPK, IP, or I. In some embodiments, the heterologous moiety is situated between any two adjacent amino acids of SNLKEIPKNVAP (SEQ ID NO: 12), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, 5 and 6, 6 and 7, 7 and 8, 8 and 9, 9 and 10, 10 and 11, or 11 and 12 of SEQ ID NO: 12. In some embodiments, one or more amino acids of SEQ ID NO: 12 are absent from the Cas12i2 fusion protein. In other embodiments, 1, 2, 3, 4, 5, 6, 7, 8, 9, 10, 11, or 12 sequential amino acids of SEQ ID NO: 12 that are N-terminal to the heterologous sequence are absent from the Cas12i2 fusion protein. In other embodiments, 1, 2, 3, 4, 5, 6, 7, 8, 9, 10, 11, or 12 sequential amino acids of SEQ ID NO: 12 that are C-terminal to the heterologous sequence are absent from the Cas12i2 fusion protein.

### Exemplary Cas12i2 Fusion Proteins Having a Heterologous Sequence at the PAM Proximal Region of Amino Acids K241-L250

In some embodiments, n is 246 and m is 247. In other embodiments, the first portion comprises at least 197, 200, 210, 220, 230, 240, 245, or 246 amino acids. In certain embodiments, the second portion comprises at least 646, 650, 670, 680, 690, 700, 710, 720, 730, 740, 750, 760, 780, 790, 800, 805, 806, 807, or 808 amino acids. In yet another embodiment, the C-terminal amino acid(s) of the first portion comprise GQK, QK, or K. In certain embodiments, the N-terminal amino acid(s) of the second portion comprise EFD, EF, or E. In some embodiments, the heterologous moiety is situated between any two adjacent amino acids of KDGQKEFDL (SEQ ID NO: 13), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, 5 and 6, 6 and 7, 7 and 8, or 8 and 9 of SEQ ID NO: 13. In some embodiments, one or more amino acids of SEQ ID NO: 13 are absent from the Cas12i2 fusion protein. In certain embodiments, 1, 2, 3, 4, 5, 6, 7, 8, or 9 sequential amino acids of SEQ ID NO: 13 that are N-terminal to the heterologous sequence are absent from the Cas12i2 fusion protein. In certain embodiments, 1, 2, 3, 4, 5, 6, 7, 8, or 9 sequential amino acids of SEQ ID NO: 13 that are C-terminal to the heterologous sequence are absent from the Cas12i2 fusion protein.

### Exemplary Cas12i2 Fusion Proteins Having a Heterologous Sequence at the PAM Proximal Region of Amino Acids G583-R594

In some embodiments of the Cas12i2 fusion protein described herein, a) n is 587 and m is 588, or b) n is 590 and m is 591. In other embodiments, the first portion comprises at least 470, 472, 480, 490, 500, 510, 520, 530, 540, 550, 560, 570, 580, 585, 587, or 590 amino acids. In certain embodiments, the second portion comprises at least 371, 374, 380, 390, 400, 410, 420, 430, 440, 450, 460, 464, or 467 amino acids. In some embodiments, the C-terminal amino acid(s) of the first portion comprise: a) QKG, KG, or G; or b) TLQ, LQ, or Q. In some embodiments, the N-terminal amino acid(s) of the second portion comprise: a) TLQ, TL, or T; or b) IGD, IG, or I. In some embodiments, the heterologous moiety is situated between any two adjacent amino acids of GRQKGTLQIGDR (SEQ ID NO: 14), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, 5 and 6, 6 and 7, 7 and 8, 8 and 9, 9 and 10, 10 and 11, or 11 and 12 of SEQ ID NO: 14. In certain embodiments, one or more amino acids of SEQ ID NO: 14 are absent from the Cas12i2 fusion protein. In some embodiments, 1, 2, 3, 4, 5, 6, 7, 8, 9, 10, 11, or 12 sequential amino acids of SEQ ID NO: 14 that are N-terminal to the heterologous sequence are absent from the Cas12i2 fusion protein. In some embodiments, 1, 2, 3, 4, 5, 6, 7, 8, 9, 10, 11, or 12 sequential amino acids of SEQ ID NO: 14 that are C-terminal to the heterologous sequence are absent from the Cas12i2 fusion protein.

### Exemplary Cas12i2 Fusion Proteins Having a Heterologous Sequence at the PAM Proximal Region of Amino Acids C877-W901

In some embodiments of the Cas12i2 fusion protein described herein, a) n is 893 and m is 894, or b) n is 894 and m is 895. In other embodiments, the first portion comprises at least 715, 716, 720, 730, 740, 750, 760, 770, 780, 790, 800, 810, 820, 830, 840, 850, 860, 870, 880, 890, 891, 892, 893, or 894 amino acids. In some embodiments, the second portion comprises at least 128, 129, 130, 140, 150, 160, or 161 amino acids. In certain embodiments, the C-terminal amino acid(s) of the first portion comprise: a) RNP, NP, or P; or b) NPD, PD, or D. In some embodiments, the N-terminal amino acid(s) of the second portion comprise: a) DKA, DK, or D; or b) KAM, KA, or K. In some embodiments, the heterologous moiety is situated between any two adjacent amino acids of CGSLYTSHQDPLVHRNPDKAMKCRW (SEQ ID NO: 15), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, 5 and 6, 6 and 7, 7 and 8, 8 and 9, 9 and 10, 10 and 11, 11 and 12, 12 and 13, 13 and 14, 14 and 15, 15 and 16, 16 and 17, 17 and 18, 18 and 19, 19 and 20, 20 and 21, 21 and 22, 22 and 23, 23 and 24, 24 and 25, of SEQ ID NO: 15. In other embodiments, one or more amino acids of SEQ ID NO: 15 are absent from the Cas12i2 fusion protein. In some embodiments, 1, 2, 3, 4, 5, 6, 7, 8, 9, 10, 11, 12, 13, 14, 15, 16, 17, 18, 19, 20, 21, 22, 23, or 24 sequential amino acids of SEQ ID NO: 15 that are N-terminal to the heterologous sequence are absent from the Cas12i2 fusion protein. In other embodiments, 1, 2, 3, 4, 5, 6, 7, 8, 9, 10, 11, 12, 13, 14, 15, 16, 17, 18, 19, 20, 21, 22, 23, or 24 sequential amino acids of SEQ ID NO: 15 that are C-terminal to the heterologous sequence are absent from the Cas12i2 fusion protein.

### Exemplary Cas12i2 Fusion Proteins Comprising a Heterologous Sequence Such as an NLS at Amino Acid Residues G173-D179

In some embodiments, n is 175 and m is 176. In certain embodiments, the heterologous sequence comprises a localization sequence, e.g., a nuclear localization sequence (NLS).

In certain embodiments, the heterologous sequence comprises an NLS, and n and m are each independently a number between:
iii) 408-413 (e.g., 408, 409, 410, 411, 412, or 413);
xv) 173-179 (e.g., 173, 174, 175, 176, 177, 178, or 179);
xvi) 216-221 (e.g., 216, 217, 218, 219, 220, or 221);
xvii) 265-272 (e.g., 265, 266, 267, 268, 269, 270, 271, or 272);
xix) 456-468 (e.g., 456, 457, 458, 459, 460, 461, 462, 463, 464, 465, 466, 467, or 468);
xx) 476-482 (e.g., 476, 477, 478, 479, 480, 481, or 482);
xxi) 498-513 (e.g., 498, 499, 500, 501, 502, 503, 504, 505, 506, 507, 508, 509, 510, 511, 512, or 513);
xxii) 614-625 (e.g., 614, 615, 616, 617, 618, 619, 620, 621, 622, 623, 624, or 625);
xxiii) 977-982 (e.g., 977, 978, 979, 980, 981, or 982); or
xxiv) 1007-1012 (e.g., 1007, 1008, 1009, 1010, 1011, or 1012), wherein m is greater than n.

In some embodiments, the first portion comprises at least 140, 145, 150, 155, 160, 165, 170, or 175 amino acids. In some embodiments, the second portion comprises at least 703, 710, 720, 730, 740, 750, 760, 770, 780, 790, 800, 810, 820, 830, 840, 850, 860, 870, 875, 876, 877, or 878 amino acids. In some embodiments, the C-terminal amino acid(s) of the first portion comprise GTG, TG, or G. In certain embodiments, the N-terminal amino acid(s) of the second portion comprise EKE, EK, or E. In some embodiments, the heterologous moiety is situated between any two adjacent amino acid residues of GTGEKED (SEQ ID NO: 16), e.g., between positions 1 and 2, 2 and 3, 3 and 4, or 4 and 5 of SEQ ID NO: 17. In some embodiments, one or more amino acids of SEQ ID NO: 16 are absent from the Cas12i2 fusion protein. In some embodiments, 1, 2, 3, 4, or 5 amino acids of SEQ ID NO: 16 that are N-terminal to the heterologous sequence are absent from the Cas12i2 fusion protein. In certain embodiments, 1, 2, 3, 4, or 5 sequential amino acids of SEQ ID NO: 16 that are C-terminal to the heterologous sequence are absent from the Cas12i2 fusion protein.

### Exemplary Cas12i2 Fusion Proteins Comprising a Heterologous Sequence Such as an NLS at Amino Acid Residues K216-T221

In some embodiments of the Cas12i2 fusion proteins described herein, a) n is 218 and m is 219; or b) n is 219 and m is 220. In certain embodiments, the first portion comprises at least 175, 176, 180, 190, 200, 210, 218, or 219 amino acids. In some embodiments, the second portion comprises at least 668, 669, 670, 680, 690, 700, 710, 720, 730, 740, 750, 760, 770, 780, 790, 800, 810, 820, 830, 835, or 836 amino acids. In some embodiments, the C-terminal amino acid(s) of the first portion comprise: a) KAT, AT, or T; or b) ATK, TK, or K. In certain embodiments, N-terminal amino acid(s) of the second portion comprise: a) KET, KE, or K; or b) ETF, ET, or E. In some embodiments, the heterologous moiety is situated between any two adjacent amino acids of KATKET (SEQ ID NO: 17), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, 5 and 6, or 6 and 7 of SEQ ID NO: 17. In certain embodiments, one or more amino acids of SEQ ID NO: 17 are absent from the Cas12i2 fusion protein. In certain embodiments, 1, 2, 3, 4, 5, 6, or 7 sequential amino acids of SEQ ID NO: 17 that are N-terminal to the heterologous sequence are absent from the Cas12i2 fusion protein. In some embodiments, 1, 2, 3, 4, 5, 6, or 7 sequential amino acids of SEQ ID NO: 17 that are C-terminal to the heterologous sequence are absent from the Cas12i2 fusion protein.

### Exemplary Cas12i2 Fusion Proteins Comprising a Heterologous Sequence Such as an NLS at Amino Acid Residues S265-C272

In certain embodiments, n is 266 and m is 267. In some embodiments, the first portion comprises at least 213, 220, 230, 240, 250, 260, 265, or 266 amino acids. In some embodiments, the second portion comprises at least 630, 640, 650, 660, 670, 680, 690, 700, 710, 720, 730, 740, 750, 760, 770, 780, or 788 amino acids. In certain embodiments, the C-terminal amino acid(s) of the first portion comprise KSK, SK, or K. In other embodiments, the N-terminal amino acid(s) of the second portion comprise ERD, ER, or E. In certain embodiments, the heterologous moiety is situated between any two adjacent amino acids of SKERDWCC (SEQ ID NO: 18), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, 5 and 6, 6 and 7, or 7 and 8 of SEQ ID NO: 18. In other embodiments, one or more amino acids of SEQ ID NO: 18 are absent from the Cas12i2 fusion protein. In some embodiments, 1, 2, 3, 4, 5, 6, 7, or 8 sequential amino acids of SEQ ID NO: 18 that are N-terminal to the heterologous sequence are absent from the Cas12i2 fusion protein. In certain embodiments, 1, 2, 3, 4, 5, 6, 7, or 8 sequential amino acids of SEQ ID NO: 18 that are C-terminal to the heterologous sequence are absent from the Cas12i2 fusion protein.

### Exemplary Cas12i2 Fusion Proteins Comprising a Heterologous Sequence Such as an NLS at Amino Acid Residues R408-A413

In some embodiments of the Cas12i2 fusion protein described herein, a) n is 409 and m is 410, or b) n is 410 and m is 411. In certain embodiments, the first portion comprises at least 328 330, 340, 350, 360, 370, 380, 390, 400, 405, 406, 407, 408, 409, or 410 amino acids. In some embodiments, the second portion comprises at least 516, 520, 530, 540, 550, 560, 570, 580, 590, 600, 610, 620, 630, 640, 641, 642, 643, 644, or 645 amino acids. In some embodiments, the C-terminal amino acid(s) of the first portion comprise IRQE, RQ, Q, or E. In some embodiments, the N-terminal amino acid(s) of the second portion comprise ECS, EC, E, or C. In certain embodiments, the heterologous moiety is situated between any two adjacent amino acids of RQECSA (SEQ ID NO: 19), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, or 5 and 6 of SEQ ID NO: 19. In some embodiments, one or more amino acids of SEQ ID NO: 19 are absent from the Cas12i2 fusion protein. In certain embodiments, 1, 2, 3, 4, 5, or 6 sequential amino acids of SEQ ID NO: 19 that are N-terminal to the heterologous sequence are absent from the Cas12i2 fusion protein. In some embodiments, 1, 2, 3, 4, 5, or 6 sequential amino acids of SEQ ID NO: 19 that are C-terminal to the heterologous sequence are absent from the Cas12i2 fusion protein.

### Exemplary Cas12i2 Fusion Proteins Comprising a Heterologous Sequence Such as an NLS at Amino Acid Residues A456-R468

In some embodiments, n is 462 and m is 463. In other embodiments, the first portion comprises at least 370, 380, 390, 400, 410, 420, 430, 440, 450, 460, 461, or 462 amino acids. In some embodiments, the second portion comprises at least 474, 480, 490, 500, 510, 520, 530, 540, 550, 560, 570, 580, 590, 591, or 592 amino acids. In some embodiments, the C-terminal amino acid(s) of the first portion comprise DRP, RP, or P. In some embodiments, the N-terminal amino acid(s) of the second portion comprise NSL, NS, or S. In some embodiments, the heterologous moiety is situated between any two adjacent amino acids of AQRNDRPNSLDLR (SEQ ID NO: 20), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, 5 and 6, 6 and 7, 7 and 8, 8 and 9, 9 and 10, 10 and 11, 11 and 12, or 12 and 13 of SEQ ID NO: 20. In other embodiments, one or more amino acids of SEQ ID NO: 20 are absent from the Cas12i2 fusion protein. In some embodiments, 1, 2, 3, 4, 5, 6, 7, 8, 9, 10, 11, 12, or 13 sequential amino acids of SEQ ID NO: 20 that are N-terminal to the heterologous sequence are absent from the Cas12i2 fusion protein. In some embodiments, 1, 2, 3, 4, 5, 6, 7, 8, 9, 10, 11, 12, or 13 sequential amino acids of SEQ ID NO: 20 that are C-terminal to the heterologous sequence are absent from the Cas12i2 fusion protein.

### Exemplary Cas12i2 Fusion Proteins Comprising a Heterologous Sequence Such as an NLS at Amino Acid Residues H476-W482

In certain embodiments, n is 478 and m is 479. In some embodiments, the first portion comprises at least 383, 390, 400, 410, 420, 430, 440, 450, 460, 470, 475, or 478 amino acids. In some embodiments, the second portion comprises at least 461, 470, 480, 490, 500, 510, 520, 530, 540, 550, 560, 570, or 578 amino acids. In other embodiments, the C-terminal amino acid(s) of the first portion comprise RHP, HP, or P. In certain embodiments, the N-terminal amino acid(s) of the second portion comprise DGR, DG, or D. In certain embodiments, the heterologous moiety is situated between any two adjacent amino acids of HPDGRW (SEQ ID NO: 21), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, or 5 and 6 of SEQ ID NO: 21. In other embodiments, one or more amino acids of SEQ ID NO: 21 are absent from the Cas12i2 fusion protein. In some embodiments, 1, 2, 3, 4, 5, or 6 sequential amino acids of SEQ ID NO: 21 that are N-terminal to the heterologous sequence are absent from the Cas12i2 fusion protein. In certain embodiments, 1, 2, 3, 4, 5, or 6 sequential amino acids of SEQ ID NO: 21 that are C-terminal to the heterologous sequence are absent from the Cas12i2 fusion protein.

Exemplary Cas12i2 Fusion Proteins Comprising a Heterologous Sequence Such as an NLS at Amino Acid Residues I498-T513 In some embodiments of any Cas12i2 fusion protein described herein, a) n is 504 and m is 505; or b) n is 505 and m is 506. In other embodiments, the first portion comprises at least 404, 410, 420, 430, 440, 450, 460, 470, 480, 490, 500, 504, or 505 amino acids. In certain embodiments, the second portion comprises at least 439, 440, 450, 460, 470, 480, 490, 500, 510, 520, 530, 540, 549, or 550 amino acids. In some embodiments, the C-terminal amino acid(s) of the first portion comprise: a) GNS, NS, or S; or b) NSP, SP, or P. In some embodiments, the N-terminal amino acid(s) of the second portion comprise: a) PVD, PV, or P; or b) VDT, VD, or V. In certain embodiments, the heterologous moiety is situated between any two adjacent amino acids of IYAAGNSPVDTCQFRT (SEQ ID NO: 22), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, 5 and 6, 6 and 7, 7 and 8, 8 and 9, 9 and 10, 10 and 11, 11 and 12, 12 and 13, 13 and 14, 14 and 15, or 15 and 16 of SEQ ID NO: 22. In some embodiments, one or more amino acids of SEQ ID NO: 22 are absent from the Cas12i2 fusion protein. In some embodiments, 1, 2, 3, 4, 5, 6, 7, 8, 9, 10, 11, 12, 13, 14, 15, or 16 sequential amino acids of SEQ ID NO: 22 that are N-terminal to the heterologous sequence are absent from the Cas12i2 fusion protein. In some embodiments, 1, 2, 3, 4, 5, 6, 7, 8, 9, 10, 11, 12, 13, 14, 15, or 16 sequential amino acids of SEQ ID NO: 22 that are C-terminal to the heterologous sequence are absent from the Cas12i2 fusion protein.

### Exemplary Cas12i2 Fusion Proteins Comprising a Heterologous Sequence Such as an NLS at Amino Acid Residues V614-C625

In some embodiments, n is 614 and m is 615. In some embodiments, the first portion comprises at least 492, 500, 510, 520, 530, 540, 550, 560, 570, 580, 590, 600, 610, or 614 amino acids. In certain embodiments, the second portion comprises at least 352, 360, 370, 380, 390, 400, 410, 420, 430, or 440 amino acids. In some embodiments, the C-terminal amino acid(s) of the first portion comprise EVV, VV, or V. In certain embodiments, the N-terminal amino acid(s) of the second portion comprise KEG, KE, or K. In some embodiments, the heterologous moiety is situated between any two adjacent amino acids of VKEGQYHKELGC (SEQ ID NO: 23), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, 5 and 6, 6 and 7, 7 and 8, 8 and 9, 9 and 10, 10 and 11, or 11 and 12 of SEQ ID NO: 23. In certain embodiments, one or more amino acids of SEQ ID NO: 23 are absent from the Cas12i2 fusion protein. In some embodiments, 1, 2, 3, 4, 5, 6, 7, 8, 9, 10, 11, or 12 sequential amino acids of SEQ ID NO: 23 that are N-terminal to the heterologous sequence are absent from the Cas12i2 fusion protein. In certain embodiments, 1, 2, 3, 4, 5, 6, 7, 8, 9, 10, 11, or 12 sequential amino acids of SEQ ID NO: 23 that are C-terminal to the heterologous sequence are absent from the Cas12i2 fusion protein.

### Exemplary Cas12i2 Fusion Proteins Comprising a Heterologous Sequence Such as an NLS at Amino Acid Residues G977-V982

In some embodiments, n is 977 and m is 978. In some embodiments, the first portion comprises at least 782, 790, 800, 810, 820, 830, 840, 850, 860, 870, 880, 890, 900, 910, 920, 930, 940, 950, 960, 970, or 977 amino acids. In some embodiments, the second portion comprises at least 352, 360, 370, 380, 390, 400, 410, 420, 430, or 440 amino acids. In certain embodiments, the C-terminal amino acid(s) of the first portion comprise KLG, LG, or G. In some embodiments, the N-terminal amino acid(s) of the second portion comprise NKE, NK, or N. In certain embodiments, the heterologous moiety is situated between any two adjacent amino acids of GNKEAV (SEQ ID NO: 24), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, and 5 and 6 of SEQ ID NO: 24. In some embodiments, one or more amino acids of SEQ ID NO: 24 are absent from the Cas12i2 fusion protein. In certain embodiments, 1, 2, 3, 4, 5, or 6 sequential amino acids of SEQ ID NO: 24 that are N-terminal to the heterologous sequence are absent from the Cas12i2 fusion protein. In some embodiments, 1, 2, 3, 4, 5, or 6 sequential amino acids of SEQ ID NO: 24 that are C-terminal to the heterologous sequence are absent from the Cas12i2 fusion protein.

### Exemplary Cas12i2 Fusion Proteins Comprising a Heterologous Sequence Such as an NLS at Amino Acid Residues V1007-Q1012

In some embodiments, n is 1007 and m is 1008. In certain embodiments, the first portion comprises at least 806, 810, 820, 830, 840, 850, 860, 870, 880, 890, 900, 910, 920, 930, 940, 950, 960, 970, 980, 990, 1000, or 1007 amino acids. In certain embodiments, the second portion comprises at least 38, 39, 40, 41, 42, 43, 44, 45, 46, or 47 amino acids. In some embodiments, the C-terminal amino acid(s) of the first portion comprise SIV, IV, or V. In some embodiments, the N-terminal amino acid(s) of the second portion comprise FDW, FD, or F. In some embodiments, the heterologous moiety is situated between any two adjacent amino acids of VFDQKQ (SEQ ID NO: 25), or an amino acid sequence having at least 80% (e.g., at least 80%, 85%, 90%, 95%, 96%, 97%, 98%, or 99%) identity thereto, e.g., between positions 1 and 2, 2 and 3, 3 and 4, 4 and 5, and 5 and 6 of SEQ ID NO: 25. In some embodiments, one or more amino acids of SEQ ID NO: 25 are absent from the Cas12i2 fusion protein. In certain embodiments, 1, 2, 3, 4, 5, or 6 sequential amino acids of SEQ ID NO: 25 that are N-terminal to the heterologous sequence are absent from the Cas12i2 fusion protein. In some embodiments, 1, 2, 3, 4, 5, or 6 sequential amino acids of SEQ ID NO: 25 that are C-terminal to the heterologous sequence are absent from the Cas12i2 fusion protein.

In some embodiments, the heterologous sequence comprises a fusion domain (e.g., a base editing domain, a ssDNA binding domain, an NLS, or a poly-basic domain). For instance, a Cas12i2 fusion protein of this disclosure may comprise a nuclear localization sequence (NLS) such as an SV40 (simian virus 40) NLS, c-Myc NLS, or other suitable monopartite NLS. The NLS may be fused to the N-terminus and/or C-terminus of the Cas12i2 polypeptide, and may be fused singly (i.e., a single NLS) or concatenated (e.g., a chain of 2, 3, 4, etc. NLS).

In some embodiments, at least one Nuclear Export Signal (NES) is attached to a nucleic acid sequences encoding the Cas12i2 fusion protein. In some embodiments, a C-terminal and/or N-terminal NLS or NES is attached for optimal expression and nuclear targeting in eukaryotic cells, e.g., human cells.

In certain embodiments, the heterologous sequence comprises at least one linker sequence. In some embodiments, the heterologous sequence comprises a first linker (e.g., a first peptide linker) and a second linker (e.g., a second peptide linker). In some embodiments, the first linker and the second linker each independently comprise between 3 and 60 amino acid residues (e.g., 5, 10, 15, 20, 25, 30, 35, 40, 50, 55, or 60, between 3-10, between 10-20, between 20-30, between 30-40, between 40-50, or between 50-60). In some embodiments, the first linker and the second linker each independently comprise one or more Gly residues and/or one or more Ser residues. In other embodiments, the first linker and the second peptide linker each independently comprise (GSG)ₓ, (GGGS)ₓ, or (GSSG)ₓ, wherein x is an integer between 0 and 15 (e.g., 0, 1, 2, 3, 4, 5, 6, 7, 8, 9, 10, 11, 12, 13, 14, or 15). In some embodiments, the first linker is N-terminal of the fusion domain and the second linker is C-terminal of the fusion domain. In certain embodiments, the first linker and the second linker are the same. In some embodiments, the first linker and the second linker are different.

### Exemplary Cas12i2 Fusion Proteins Comprising an Insertion in the Wed Domain, Rec1 Domain, or Nuc Domain

In some embodiments, a Cas12i2 protein comprises a heterologous sequence (e.g., an insertion) within the Wed domain, the Rec1 domain, or the Nuc domain. In some embodiments, the insertion occurs at the interface of the Wed domain and the Rec1 domain.

In some embodiments, n is 430 and m is 431. In some embodiments, n is 431 and m is 432. In some embodiments, n is 432 and m is 433. In some embodiments, n is 433 and m is 434. In some embodiments, n is 434 and m is 435. In some embodiments, n is 435 and m is 436. In some embodiments, n is 436 and m is 437. In some embodiments, n is 437 and m is 438. In some embodiments, n is 438 and m is 439. In some embodiments, n is 440 and m is 441. In some embodiments, n is 441 and m is 442. In some embodiments, n is 442 and m is 443. In some embodiments, n is 443 and m is 444. In some embodiments, n is 444 and m is 445. In some embodiments, n is 445 and m is 446. In some embodiments, n is 446 and m is 447. In some embodiments, n is 447 and m is 448. In some embodiments, n is 448 and m is 449. In some embodiments, n is 449 and m is 450. In some embodiments, n is 920 and m is 921. In some embodiments, n is 921 and m is 922. In some embodiments, n is 922 and m is 923. In some embodiments, n is 923 and m is 924. In some embodiments, n is 924 and m is 925. In some embodiments, n is 925 and m is 926. In some embodiments, n is 926 and m is 927. In some embodiments, n is 927 and m is 928. In some embodiments, n is 928 and m is 929. In some embodiments, n is 929 and m is 930. In some embodiments, n is 930 and m is 931. In some embodiments, n is 931 and m is 932. In some embodiments, n is 932 and m is 933. In some embodiments, n is 933 and m is 934. In some embodiments, n is 934 and m is 935. In some embodiments, n is 935 and m is 936. In some embodiments, n is 936 and m is 937. In some embodiments, n is 937 and m is 938. In some embodiments, n is 938 and m is 939. In some embodiments, n is 939 and m is 940.

In some embodiments, the insertion is one residue to about 10 residues in length (e.g., 1, 2, 3, 4, 5, 6, 7, 8, 9, or 10 residues). In some embodiments, the insertion comprises one or more of a glycine, serine, aspartate, or asparagine residue. In some embodiments, the insertion comprises a one-residue insertion (e.g., one glycine, one serine, one aspartate, or one asparagine). In some embodiments, the insertion comprises a two-residue insertion (e.g., two glycines, two serines, two aspartates, or two asparagines). In some embodiments, the insertion comprises a two-residue insertion comprising at least one glycine. In some embodiments, the insertion comprises a three-residue insertion (e.g., three glycines, three serines, three aspartates, or three asparagines). In some embodiments, the insertion comprises a three-residue insertion comprising at least one glycine. In some embodiments, the insertion comprises a four-residue insertion (e.g., four glycines, four serines, four aspartates, or four asparagines). In some embodiments, the insertion comprises a four-residue insertion comprising at least one glycine. In some embodiments, the insertion comprises a five-residue insertion (e.g., five glycines, five serines, five aspartates, or five asparagines). In some embodiments, the insertion comprises a five-residue insertion comprising at least one glycine.

In some embodiments, a Cas12i2 protein has a glycine-glycine insertion in the Wed domain or the Rec domain. In some embodiments, n is 440, m is 441, and the heterologous sequence is a glycine-glycine insertion. In some embodiments, n is 440, m is 441, and the heterologous sequence is a serine-serine insertion. In some embodiments, n is 440, m is 441, and the heterologous sequence is an aspartate-aspartate insertion. In some embodiments, n is 440, m is 441, and the heterologous sequence is an asparagine-asparagine insertion. In some embodiments, n is 440, m is 441, and the heterologous sequence is a glycine-serine insertion. In some embodiments, n is 440, m is 441, and the heterologous sequence is a glycine-aspartate insertion. In some embodiments, n is 440, m is 441, and the heterologous sequence is a glycine-asparagine insertion. In some embodiments, n is 440, m is 441, and the heterologous sequence is a serine-glycine insertion. In some embodiments, n is 440, m is 441, and the heterologous sequence is an aspartate-glycine insertion. In some embodiments, n is 440, m is 441, and the heterologous sequence is an asparagine-glycine insertion.

In some embodiments, a Cas12i2 protein has a glycine-glycine insertion in the Nuc domain. In some embodiments, n is 927, m is 928, and the heterologous sequence is a glycine-glycine insertion. In some embodiments, n is 927, m is 928, and the heterologous sequence is a serine-serine insertion. In some embodiments, n is 927, m is 928, and the heterologous sequence is an aspartate-aspartate insertion. In some embodiments, n is 927, m is 928, and the heterologous sequence is an asparagine-asparagine insertion. In some embodiments, n is 927, m is 928, and the heterologous sequence is a glycine-serine insertion. In some embodiments, n is 927, m is 928, and the heterologous sequence is a glycine-aspartate insertion. In some embodiments, n is 927, m is 928, and the heterologous sequence is a glycine-asparagine insertion. In some embodiments, n is 927, m is 928, and the heterologous sequence is a serine-glycine insertion. In some embodiments, n is 927, m is 928, and the heterologous sequence is an aspartate-glycine insertion. In some embodiments, n is 927, m is 928, and the heterologous sequence is an asparagine-glycine insertion.

Fusion Proteins with Dimerization Domains

### PAM Proximal Dimerization

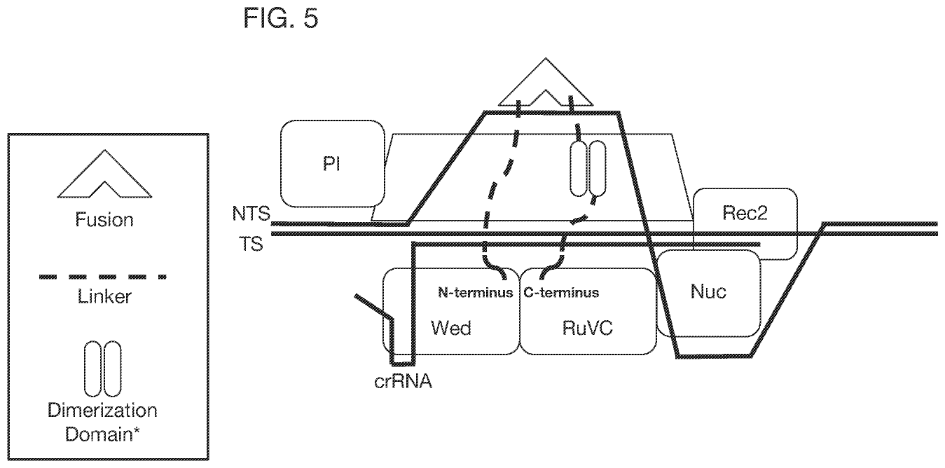

In another aspect, the disclosure provides a Cas12i2 fusion protein (see, e.g., FIG. 5) comprising:
a) a Cas12i2 domain comprising an amino acid sequence of SEQ ID NO: 1, or a sequence having at least 80%, 85%, 90%, 95%, 96%, 97%, 98%, or 99% identity thereto;
b) a first heterologous sequence disposed N-terminal of the Cas12i2 domain;
c) a second heterologous sequence disposed C-terminal of the Cas12i2 domain,
wherein the first heterologous sequence comprises a dimerization domain, the second heterologous sequence comprises a dimerization domain, or the first heterologous sequence comprises a first dimerization domain and the second heterologous sequences comprises a second, compatible dimerization domain.

In certain embodiments, the first heterologous sequence further comprises a fusion domain. In some embodiments, the fusion domain is disposed between the Cas12i2 domain and the dimerization domain. In some embodiments, the first heterologous sequence comprises (i) a first dimerization domain and (ii) a fusion domain, wherein the fusion domain is disposed between the first dimerization domain and the Cas12i2 domain. In some embodiments, the second heterologous sequence comprises a second, compatible dimerization domain. In some embodiments, the Cas12i2 domain is linked to the first heterologous sequence by a first linker (e.g., a first peptide linker). In some embodiments, the Cas12i2 domain is linked to the second heterologous sequence by a second linker (e.g., a second peptide linker). In certain embodiments, the fusion domain is linked to the first dimerization domain by a third linker (e.g., a third peptide linker). In other embodiments, the first linker, the second linker, or the third linker each independently comprise between 4 and 60 amino acid residues. In certain embodiments, the first linker, the second linker, or the third linker each independently comprise a combination of Gly residues and Ser residues. In some embodiments, the first linker, the second linker, or the third linker each independently comprise an amino acid sequence comprising (GSG)ₓ, (GGGS)ₓ, or (GSSG)ₓ, wherein X is an integer between 0 and 15 (e.g., 0, 1, 2, 3, 4, 5, 6, 7, 8, 9, 10, 11, 12, 13, 14, or 15).

### N-Terminal and C-Terminal Split Fusion Domains

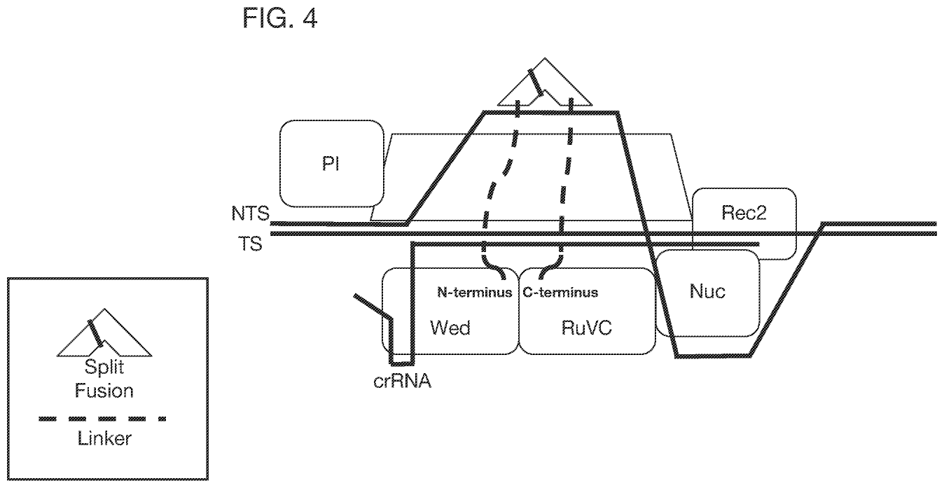

In another aspect, the disclosure features a Cas12i2 fusion protein (see, e.g., FIG. 4) comprising:
a) a Cas12i2 domain comprising an amino acid sequence of SEQ ID NO: 1, or a sequence having at least 80%, 85%, 90%, 95%, 96%, 97%, 98%, or 99% identity thereto;
b) a first heterologous sequence disposed N-terminal of the Cas12i2 domain, wherein the first heterologous sequence comprises a first portion of a split fusion domain;
c) a second heterologous sequence disposed C-terminal of the Cas12i2 domain, wherein the second heterologous sequence comprises a second portion of a split fusion domain, wherein the second portion of the split fusion domain can bind the first portion of the split fusion domain.

In some embodiments, the first portion of a split fusion domain is linked to the Cas12i2 domain by a first linker (e.g., a first peptide linker). In some embodiments, the second portion of a split fusion domain is linked to the Cas12i2 domain by a second linker (e.g., a second peptide linker). In certain embodiments, the first linker and the second linker each independently comprise between 4 and 60 amino acid residues. In certain embodiments, the first linker and the second linker each independently comprise a combination of Gly and Ser residues. In certain embodiments, the first linker and the second peptide linker each independently comprise (GSG)ₓ, (GGGS)ₓ or (GSSG)ₓ, wherein X is an integer between 0 and 15 (e.g., 0, 1, 2, 3, 4, 5, 6, 7, 8, 9, 10, 11, 12, 13, 14, or 15). Examples of split fusion domains that may be used are beta-lactamase, dihydrofolate reductase (DHFR), focal adhesion kinase (FAK), green fluorescent protein GFP), enhanced GFP (EGFP), horseradish peroxidase, infrared fluorescent protein IFP1.4, LacZ, luciferase (e.g., recombinase enhanced bimolecular luciferase (ReBiL), Gaussia princeps luciferase, NanoLuc, and NanoBIT), Tobacco etch virus protease (TEV), and ubiquitin.

### Circular Permutation Cas12i2 Fusion Proteins

In another aspect, the disclosure provides an engineered, non-naturally occurring Cas12i2 protein comprising:
a) a first portion comprising an amino acid sequence of an N-terminal portion of SEQ ID NO: 1 or any one of SEQ ID NOs: 39-43, or a sequence having at least 80%, 85%, 90%, 95%, 96%, 97%, 98%, or 99% identity thereto; and
b) a second portion comprising an amino acid sequence of a C-terminal portion of SEQ ID NO: 1, or a sequence having at least 80%, 85%, 90%, 95%, 96%, 97%, 98%, or 99% identity thereto,
wherein the second portion is N-terminal of the first portion,
wherein the first portion and second portion together bind to an RNA guide comprising a direct repeat sequence and a spacer sequence. In some embodiments, the circularly permuted Cas12i2 protein is capable of specifically binding a target nucleic acid complementary to the spacer sequence.

In certain embodiments, the first portion and the second portion are linked by a heterologous sequence. In some embodiments, the heterologous sequence comprises one or more of:
a) a first linker (e.g., a first peptide linker);
b) a second linker (e.g., a second peptide linker); and
c) a fusion domain.

In some embodiments, the heterologous sequence comprises each of a first linker (e.g., a first peptide linker), a second linker (e.g., a second peptide linker), and a fusion domain, wherein the fusion domain is disposed between the first linker and the second linker. In certain embodiments, the first linker and the second linker, when present, comprise between 3 and 60 amino acid residues. In some embodiments, the first linker and the second linker each independently comprise the amino acid sequence (GSS)ₓ, (GSG)ₓ, (GGGS)ₓ, or (GSSG)ₓ, wherein X is an integer between 0 and 15 (e.g., 0, 1, 2, 3, 4, 5, 6, 7, 8, 9, 10, 11, 12, 13, 14, or 15).

In some embodiments, the C-terminal most amino acid of the first portion is any amino acid residue of SEQ ID NO: 1 or any one of SEQ ID NOs: 39-43 chosen from residues:
a) 342-358 (e.g., residue 342, 343, 344, 345, 346, 347, 348, 349, 350, 351, 352, 353, 354, 355, 356, 357, or 358);
b) 373-378 (e.g., residue 373, 374, 375, 376, 377, or 378);
c) 408-413 (e.g., residue 408, 409, 410, 411, 412, or 413);
d) 677-685 (e.g., residue 677, 678, 679, 680, 681, 682, 683, 684, or 685);
e) 718-723 (e.g., residue 718, 719, 720, 721, 722, or 723);
f) 771-782 (e.g., residue 771, 772, 773, 774, 775, 776, 777, 778, 779, 780, 781, or 782);
g) 953-965 (e.g., residue 953, 954, 955, 956, 957, 958, 959, 960, 961, 962, 963, 964, or 965);
h) 55-65 (e.g., residue 55, 56, 57, 58, 59, 60, 61, 62, 63, 64, or 65);
i) 99-105 (e.g., residue 99, 100, 101, 102, 103, 104, or 105);
j) 112-120 (e.g., residue 112, 113, 114, 115, 116, 117, 118, 119, or 120);
k) 195-206 (e.g., residue 195, 196, 197, 198, 199, 200, 201, 202, 203, 204, 205, or 206);
l) 241-250 (e.g., residue 241, 242, 243, 244, 245, 246, 247, 248, 249, or 250);
m) 583-594 (e.g., residue 583, 584, 585, 586, 587, 588, 589, 590, 591, 592, 593, or 594);
n) 877-901 (e.g., residue 877, 878, 879, 880, 881, 882, 883, 884, 885, 886, 887, 888, 889, 890, 891, 892, 893, 894, 895, 896, 897, 898, 899, 900, or 901);
o) 173-179 (e.g., residue 173, 174, 175, 176, 177, 178, or 179);
p) 216-221 (e.g., residue 216, 217, 218, 219, 220, or 221);
q) 265-272 (e.g., residue 265, 266, 267, 268, 269, 270, 271, or 272);
r) 456-468 (e.g., residue 456, 457, 458, 459, 460, 461, 462, 463, 464, 465, 466, 467, or 468);
s) 476-482 (e.g., residue 476, 477, 478, 479, 480, 481, or 482);
t) 498-513 (e.g., residue 498, 499, 500, 501, 502, 503, 504, 505, 506, 507, 508, 509, 510, 511, 512, or 513);
u) 614-625 (e.g., residue 614, 615, 616, 617, 618, 619, 620, 621, 622, 623, 624, or 625);
v) 977-982 (e.g., residue 977, 978, 979, 980, 981, or 982);
w) 1007-1012 (e.g., residue 1007, 1008, 1009, 1010, 1011, or 1012);
x) 386-397 (e.g., residue 386, 387, 388, 389, 390, 391, 392, 393, 394, 395, 396, or 397); or
y) 831-844 (e.g., residue 831, 832, 833, 834, 835, 836, 837, 838, 839, 840, 841, 842, 843, or 844).

In some embodiments, the N-terminal most amino acid of the second portion is any amino acid residue of SEQ ID NO: 1 or any one of SEQ ID NOs: 39-43 chosen from residues:
a) 342-358 (e.g., residue 342, 343, 344, 345, 346, 347, 348, 349, 350, 351, 352, 353, 354, 355, 356, 357, or 358);
b) 373-378 (e.g., residue 373, 374, 375, 376, 377, or 378);
c) 408-413 (e.g., residue 408, 409, 410, 411, 412, or 413);
d) 677-685 (e.g., residue 677, 678, 679, 680, 681, 682, 683, 684, or 685);
e) 718-723 (e.g., residue 718, 719, 720, 721, 722, or 723);
f) 771-782 (e.g., residue 771, 772, 773, 774, 775, 776, 777, 778, 779, 780, 781, or 782);
g) 953-965 (e.g., residue 953, 954, 955, 956, 957, 958, 959, 960, 961, 962, 963, 964, or 965);
h) 55-65 (e.g., residue 55, 56, 57, 58, 59, 60, 61, 62, 63, 64, or 65);
i) 99-105 (e.g., residue 99, 100, 101, 102, 103, 104, or 105);
j) 112-120 (e.g., residue 112, 113, 114, 115, 116, 117, 118, 119, or 120);
k) 195-206 (e.g., residue 195, 196, 197, 198, 199, 200, 201, 202, 203, 204, 205, or 206);
l) 241-250 (e.g., residue 241, 242, 243, 244, 245, 246, 247, 248, 249, or 250);
m) 583-594 (e.g., residue 583, 584, 585, 586, 587, 588, 589, 590, 591, 592, 593, or 594);
n) 877-901 (e.g., residue 877, 878, 879, 880, 881, 882, 883, 884, 885, 886, 887, 888, 889, 890, 891, 892, 893, 894, 895, 896, 897, 898, 899, 900, or 901);
o) 173-179 (e.g., residue 173, 174, 175, 176, 177, 178, or 179);
p) 216-221 (e.g., residue 216, 217, 218, 219, 220, or 221);
q) 265-272 (e.g., residue 265, 266, 267, 268, 269, 270, 271, or 272);
r) 456-468 (e.g., residue 456, 457, 458, 459, 460, 461, 462, 463, 464, 465, 466, 467, or 468);
s) 476-482 (e.g., residue 476, 477, 478, 479, 480, 481, or 482);
t) 498-513 (e.g., residue 498, 499, 500, 501, 502, 503, 504, 505, 506, 507, 508, 509, 510, 511, 512, or 513);
u) 614-625 (e.g., residue 614, 615, 616, 617, 618, 619, 620, 621, 622, 623, 624, or 625);
v) 977-982 (e.g., residue 977, 978, 979, 980, 981, or 982);
w) 1007-1012 (e.g., residue 1007, 1008, 1009, 1010, 1011, or 1012);
x) 386-397 (e.g., residue 386, 387, 388, 389, 390, 391, 392, 393, 394, 395, 396, or 397); or
y) 831-844 (e.g., residue 831, 832, 833, 834, 835, 836, 837, 838, 839, 840, 841, 842, 843, or 844).

In any of the embodiments described herein, the circularly permuted Cas12i2 protein further comprises a second heterologous sequence at its N-terminus. In some embodiments, the circularly permuted Cas12i2 protein further comprises an additional heterologous sequence at its C-terminus. In some embodiments, the second heterologous sequence and/or the additional heterologous sequence a chosen from a purification tag, a stability tag, or a restriction endonuclease or restriction endonuclease domain.

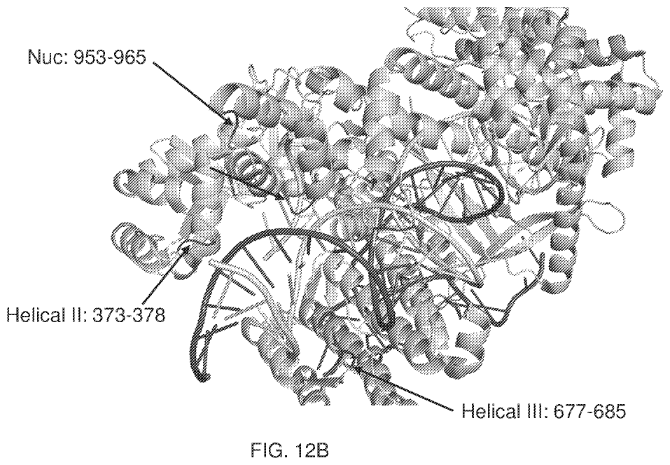

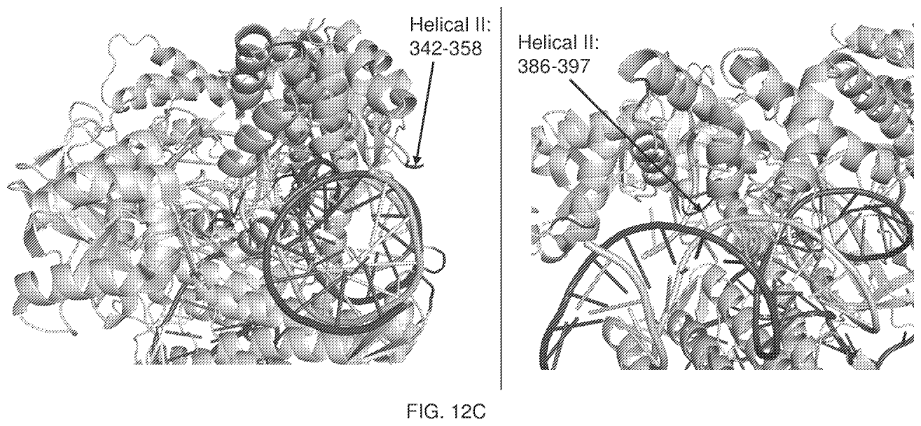

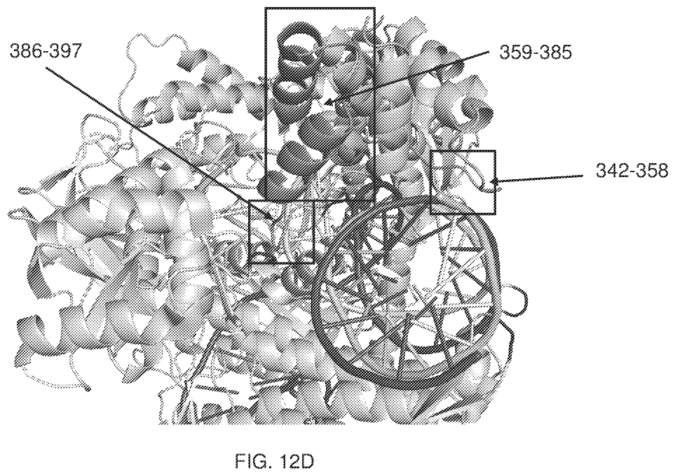

In some embodiments, a circularly permutated Cas12i2 protein comprises a) a first portion comprising an amino acid sequence of an N-terminal portion of SEQ ID NO: 1 or any one of SEQ ID NOs: 39-43, or a sequence having at least 80%, 85%, 90%, 95%, 96%, 97%, 98%, or 99% identity thereto; and b) a second portion comprising an amino acid sequence of a C-terminal portion of SEQ ID NO: 1 or any one of SEQ ID NOs: 39-43, or a sequence having at least 80%, 85%, 90%, 95%, 96%, 97%, 98%, or 99% identity thereto, wherein the second portion is N-terminal of the first portion, and wherein the C-terminal most amino acid of the first portion is an amino acid residue of a flexible loop within the Helical II, Helical III, Nuc, or RuvC II domain. In some embodiments, the flexible loop is in proximity to or in contact with target DNA, such as a loop depicted in FIG. 12A-D.

In some embodiments, a circularly permutated Cas12i2 protein comprises a) a first portion comprising an amino acid sequence of an N-terminal portion of SEQ ID NO: 1 or any one of SEQ ID NOs: 39-43, or a sequence having at least 80%, 85%, 90%, 95%, 96%, 97%, 98%, or 99% identity thereto; and b) a second portion comprising an amino acid sequence of a C-terminal portion of SEQ ID NO: 1 or any one of SEQ ID NOs: 39-43, or a sequence having at least 80%, 85%, 90%, 95%, 96%, 97%, 98%, or 99% identity thereto, wherein the second portion is N-terminal of the first portion, and wherein the N-terminal most amino acid of the second portion is an amino acid residue of a flexible loop within the Helical II, Helical III, Nuc, or RuvC II domain. In some embodiments, the flexible loop is in proximity to or in contact with target DNA, such as a loop depicted in FIG. 12A-D.

In some embodiments, a circularly permutated Cas12i2 protein comprises a) a first portion comprising an amino acid sequence of an N-terminal portion of SEQ ID NO: 1 or any one of SEQ ID NOs: 39-43, or a sequence having at least 80%, 85%, 90%, 95%, 96%, 97%, 98%, or 99% identity thereto; and b) a second portion comprising an amino acid sequence of a C-terminal portion of SEQ ID NO: 1 or any one of SEQ ID NOs: 39-43, or a sequence having at least 80%, 85%, 90%, 95%, 96%, 97%, 98%, or 99% identity thereto, wherein the second portion is N-terminal of the first portion, and wherein the C-terminal most amino acid of the first portion is any amino acid residue of SEQ ID NO: 1 or any one of SEQ ID NOs: 39-43 chosen from residues a) 342-358 (e.g., residue 342, 343, 344, 345, 346, 347, 348, 349, 350, 351, 352, 353, 354, 355, 356, 357, or 358); b) 373-378 (e.g., residue 373, 374, 375, 376, 377, or 378); c) 386-397 (e.g., residue 386, 387, 388, 389, 390, 391, 392, 393, 394, 395, 396, or 397); d) 677-685 (e.g., residue 677, 678, 679, 680, 681, 682, 683, 684, or 685); e) 771-782 (e.g., residue 771, 772, 773, 774, 775, 776, 777, 778, 779, 780, 781, or 782); f) 831-844 (e.g., residue 831, 832, 833, 834, 835, 836, 837, 838, 839, 840, 841, 842, 843, or 844), g) 953-965 (e.g., residue 953, 954, 955, 956, 957, 958, 959, 960, 961, 962, 963, 964, or 965) h) 55-65 (e.g., residue 55, 56, 57, 58, 59, 60, 61, 62, 63, 64, or 65); i) 99-105 (e.g., residue 99, 100, 101, 102, 103, 104, or 105); j) 112-120 (e.g., residue 112, 113, 114, 115, 116, 117, 118, 119, or 120); k) 195-206 (e.g., residue 195, 196, 197, 198, 199, 200, 201, 202, 203, 204, 205, or 206); 1) 241-250 (e.g., residue 241, 242, 243, 244, 245, 246, 247, 248, 249, or 250); or n) 877-901 (e.g., residue 877, 878, 879, 880, 881, 882, 883, 884, 885, 886, 887, 888, 889, 890, 891, 892, 893, 894, 895, 896, 897, 898, 899, 900, or 901). The positions of the residues are indicated in FIG. 12A-D.

In some embodiments, a circularly permutated Cas12i2 protein comprises a) a first portion comprising an amino acid sequence of an N-terminal portion of SEQ ID NO: 1 or any one of SEQ ID NOs: 39-43, or a sequence having at least 80%, 85%, 90%, 95%, 96%, 97%, 98%, or 99% identity thereto; and b) a second portion comprising an amino acid sequence of a C-terminal portion of SEQ ID NO: 1 or any one of SEQ ID NOs: 39-43, or a sequence having at least 80%, 85%, 90%, 95%, 96%, 97%, 98%, or 99% identity thereto, wherein the second portion is N-terminal of the first portion, and wherein the N-terminal most amino acid of the second portion is any amino acid residue of SEQ ID NO: 1 or any one of SEQ ID NOs: 39-43 chosen from residues a) 342-358 (e.g., residue 342, 343, 344, 345, 346, 347, 348, 349, 350, 351, 352, 353, 354, 355, 356, 357, or 358); b) 373-378 (e.g., residue 373, 374, 375, 376, 377, or 378); c) 386-397 (e.g., residue 386, 387, 388, 389, 390, 391, 392, 393, 394, 395, 396, or 397); d) 677-685 (e.g., residue 677, 678, 679, 680, 681, 682, 683, 684, or 685); e) 771-782 (e.g., residue 771, 772, 773, 774, 775, 776, 777, 778, 779, 780, 781, or 782); f) 831-844 (e.g., residue 831, 832, 833, 834, 835, 836, 837, 838, 839, 840, 841, 842, 843, or 844), or g) 953-965 (e.g., residue 953, 954, 955, 956, 957, 958, 959, 960, 961, 962, 963, 964, or 965). The positions of the residues are indicated in FIG. 12A-D.

In some embodiments, a circularly permutated Cas12i2 protein comprises a) a first portion comprising an amino acid sequence of an N-terminal portion of SEQ ID NO: 1 or any one of SEQ ID NOs: 39-43, or a sequence having at least 80%, 85%, 90%, 95%, 96%, 97%, 98%, or 99% identity thereto; and b) a second portion comprising an amino acid sequence of a C-terminal portion of SEQ ID NO: 1 or any one of SEQ ID NOs: 39-43, or a sequence having at least 80%, 85%, 90%, 95%, 96%, 97%, 98%, or 99% identity thereto, wherein the second portion is N-terminal of the first portion, and wherein the C-terminal most amino acid of the first portion is any amino acid residue of SEQ ID NO: 1 or any one of SEQ ID NOs: 39-43 chosen from residues c) 408-413 (e.g., residue 408, 409, 410, 411, 412, or 413); d) 677-685 (e.g., residue 677, 678, 679, 680, 681, 682, 683, 684, or 685); h) 55-65 (e.g., residue 55, 56, 57, 58, 59, 60, 61, 62, 63, 64, or 65); i) 99-105 (e.g., residue 99, 100, 101, 102, 103, 104, or 105); j) 112-120 (e.g., residue 112, 113, 114, 115, 116, 117, 118, 119, or 120); k) 195-206 (e.g., residue 195, 196, 197, 198, 199, 200, 201, 202, 203, 204, 205, or 206); 1) 241-250 (e.g., residue 241, 242, 243, 244, 245, 246, 247, 248, 249, or 250); or n) 877-901 (e.g., residue 877, 878, 879, 880, 881, 882, 883, 884, 885, 886, 887, 888, 889, 890, 891, 892, 893, 894, 895, 896, 897, 898, 899, 900, or 901).

In some embodiments, a circularly permutated Cas12i2 protein comprises a) a first portion comprising an amino acid sequence of an N-terminal portion of SEQ ID NO: 1 or any one of SEQ ID NOs: 39-43, or a sequence having at least 80%, 85%, 90%, 95%, 96%, 97%, 98%, or 99% identity thereto; and b) a second portion comprising an amino acid sequence of a C-terminal portion of SEQ ID NO: 1 or any one of SEQ ID NOs: 39-43, or a sequence having at least 80%, 85%, 90%, 95%, 96%, 97%, 98%, or 99% identity thereto, wherein the second portion is N-terminal of the first portion, and wherein the N-terminal most amino acid of the second portion is any amino acid residue of SEQ ID NO: 1 or any one of SEQ ID NOs: 39-43 chosen from residues c) 408-413 (e.g., residue 408, 409, 410, 411, 412, or 413); d) 677-685 (e.g., residue 677, 678, 679, 680, 681, 682, 683, 684, or 685); h) 55-65 (e.g., residue 55, 56, 57, 58, 59, 60, 61, 62, 63, 64, or 65); i) 99-105 (e.g., residue 99, 100, 101, 102, 103, 104, or 105); j) 112-120 (e.g., residue 112, 113, 114, 115, 116, 117, 118, 119, or 120); k) 195-206 (e.g., residue 195, 196, 197, 198, 199, 200, 201, 202, 203, 204, 205, or 206); 1) 241-250 (e.g., residue 241, 242, 243, 244, 245, 246, 247, 248, 249, or 250); or n) 877-901 (e.g., residue 877, 878, 879, 880, 881, 882, 883, 884, 885, 886, 887, 888, 889, 890, 891, 892, 893, 894, 895, 896, 897, 898, 899, 900, or 901).

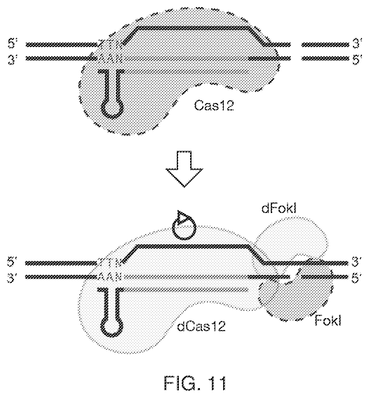

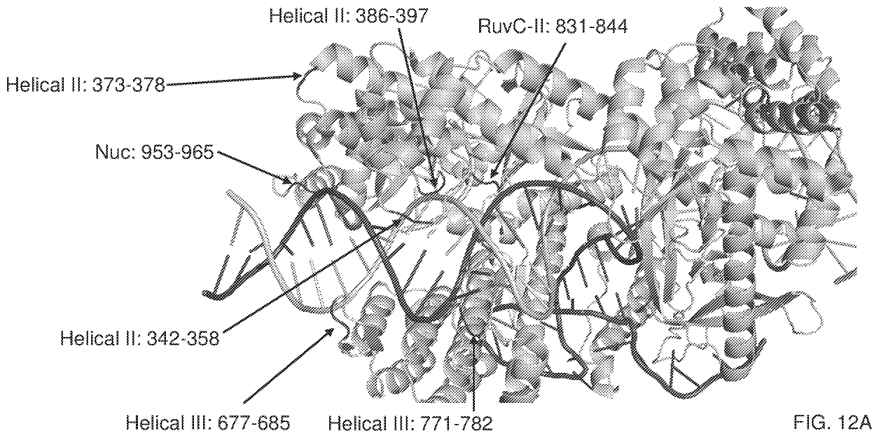

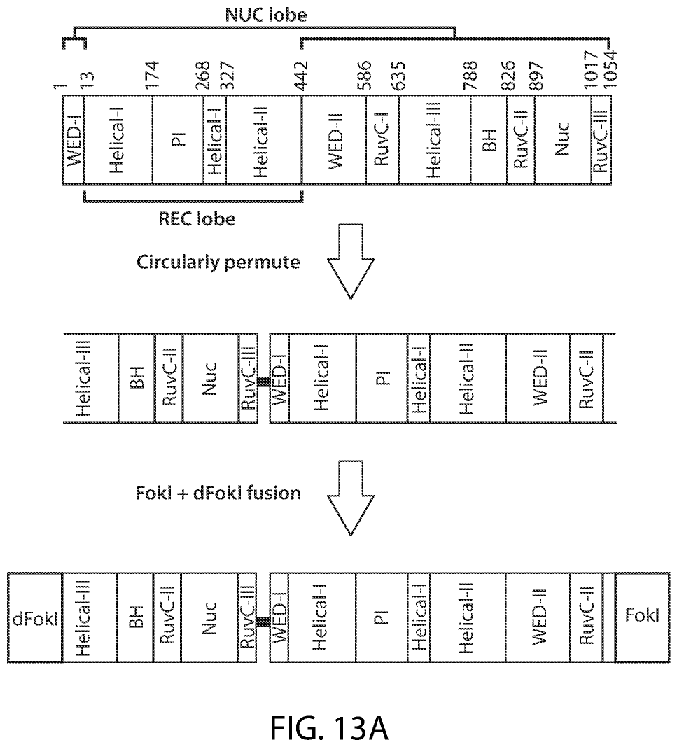

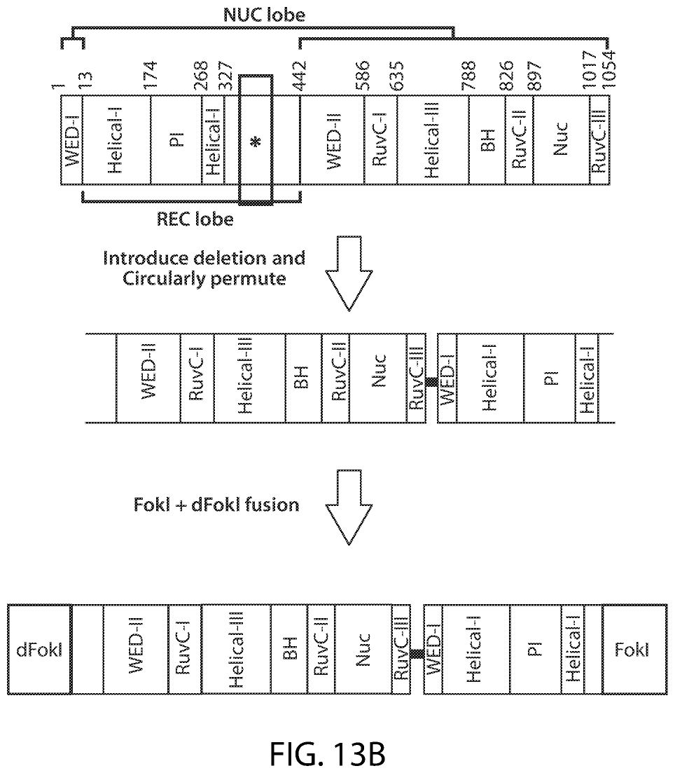

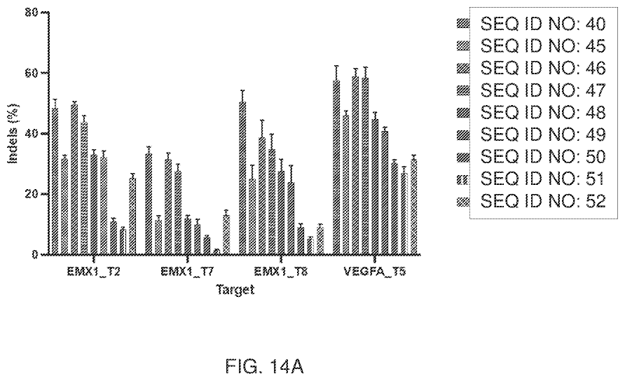

In some embodiments, the N-terminus of a circularly permutated Cas12i2 protein comprises at least one fusion domain. In some embodiments, the fusion domain comprises an NLS. In some embodiments, the circularly permuted Cas12i2 protein comprises an NLS at its N-terminus and/or C-terminus. In some embodiments, the circularly permuted Cas12i2 protein comprises an NLS at its N-terminus. In some embodiments, the circularly permuted Cas12i2 protein comprises an NLS at its C-terminus. In some embodiments, the NLS comprises an amino acid sequence of any one of SEQ ID NOs: 61-65, or a sequence having at least 80%, 85%, 90%, 95%, 96%, 97%, 98%, or 99% identity thereto. In some embodiments, the fusion domain is a FokI nuclease domain. See e.g., Ramirez et al., Nucleic Acids Res. 40(12): 5560-8 (2012) and Guilinger et al., Nature Biotechnology 32: 577-82 (2014). In some embodiments, the FokI nuclease domain is a catalytically active FokI nuclease domain. In some embodiments, the FokI nuclease domain is a dead (e.g., a catalytically inactive) FokI nuclease domain. In some embodiments, the circularly permuted Cas12i2 protein comprises a FokI nuclease domain at its N-terminus (e.g., a catalytically active FokI nuclease domain or a catalytically inactive FokI nuclease domain). In some embodiments, the circularly permuted Cas12i2 protein comprises a FokI nuclease domain at its C-terminus (e.g., a catalytically active FokI nuclease domain or a catalytically inactive FokI nuclease domain). In some embodiments, the circularly permuted Cas12i2 protein comprises a FokI nuclease domain at its N-terminus and at its C-terminus. In some embodiments, the circularly permuted Cas12i2 protein comprises a catalytically active FokI nuclease domain at its N-terminus and a catalytically active FokI nuclease domain at its C-terminus. In some embodiments, the circularly permuted Cas12i2 protein comprises a catalytically active FokI nuclease domain at its N-terminus and a catalytically inactive FokI nuclease domain at its C-terminus. In some embodiments, the circularly permuted Cas12i2 protein comprises a catalytically inactive FokI nuclease domain at its N-terminus and a catalytically active FokI nuclease domain at its C-terminus. In some embodiments, the circularly permuted Cas12i2 protein comprises a catalytically inactive FokI nuclease domain at its N-terminus and a catalytically inactive FokI nuclease domain at its C-terminus. In some embodiments wherein a circularly permuted Cas12i2 protein comprises a FokI nuclease domain at its N-terminus and at its C-terminus, the FokI nuclease domains form a dimer (e.g., a homodimer or a heterodimer). See, e.g., FIG. 11, FIG. 13A, and FIG. 13B.

In some embodiments, the FokI nuclease domain further comprises an additional fusion domain. In some embodiments, the FokI nuclease domain is a catalytically active FokI nuclease domain, and the additional fusion domain is a protein or a peptide. In some embodiments, the FokI nuclease domain is a catalytically inactive FokI nuclease domain and the additional fusion domain is a protein or a peptide. In some embodiments, the protein is a polymerase.

In some embodiments, the N-terminal residue of a circularly permuted Cas12i2 protein comprises an amino acid residue “x” of SEQ ID NO: 1 or any one of SEQ ID NOs: 39-43 (or a sequence having at least 80%, 85%, 90%, 95%, 96%, 97%, 98%, or 99% identity thereto), wherein x is chosen from residues 55-65 (e.g., residue 55, 56, 57, 58, 59, 60, 61, 62, 63, 64, or 65). In some embodiments, the C-terminal residue of the circularly permuted Cas12i2 protein comprises an amino acid residue “y” of SEQ ID NO: 1 or any one of SEQ ID NOs: 39-43 (or a sequence having at least 80%, 85%, 90%, 95%, 96%, 97%, 98%, or 99% identity thereto), wherein y is x−1. For example, in some embodiments, the N-terminal residue of a circularly permutated Cas12i2 protein comprises residue 61 corresponding to SEQ ID NO: 40, and the C-terminal residue of a circularly permutated Cas12i2 protein comprises residue 60 corresponding to SEQ ID NO: 40. In some embodiments, the circularly permuted Cas12i2 protein comprises an amino acid sequence of SEQ ID NO: 47, or a sequence having at least 80%, 85%, 90%, 95%, 96%, 97%, 98%, or 99% identity thereto. In some embodiments, residue “x” and/or residue “y” is linked to a fusion domain. In some embodiments, the fusion domain comprises an NLS.

In some embodiments, the N-terminal residue of a circularly permuted Cas12i2 protein comprises an amino acid residue “x” of SEQ ID NO: 1 or any one of SEQ ID NOs: 39-43 (or a sequence having at least 80%, 85%, 90%, 95%, 96%, 97%, 98%, or 99% identity thereto), wherein x is chosen from residues 99-105 (e.g., residue 99, 100, 101, 102, 103, 104, or 105). In some embodiments, the C-terminal residue of the circularly permuted Cas12i2 protein comprises an amino acid residue “y” of SEQ ID NO: 1 or any one of SEQ ID NOs: 39-43 (or a sequence having at least 80%, 85%, 90%, 95%, 96%, 97%, 98%, or 99% identity thereto), wherein y is x−1. For example, in some embodiments, the N-terminal residue of a circularly permutated Cas12i2 protein comprises residue 102 corresponding to SEQ ID NO: 40, and the C-terminal residue of a circularly permutated Cas12i2 protein comprises residue 101 corresponding to SEQ ID NO: 40. In some embodiments, the circularly permuted Cas12i2 protein comprises an amino acid sequence of SEQ ID NO: 48, or a sequence having at least 80%, 85%, 90%, 95%, 96%, 97%, 98%, or 99% identity thereto. In some embodiments, residue “x” and/or residue “y” is linked to a fusion domain. In some embodiments, the fusion domain comprises an NLS.

In some embodiments, the N-terminal residue of a circularly permuted Cas12i2 protein comprises an amino acid residue “x” of SEQ ID NO: 1 or any one of SEQ ID NOs: 39-43 (or a sequence having at least 80%, 85%, 90%, 95%, 96%, 97%, 98%, or 99% identity thereto), wherein x is chosen from residues 112-120 (e.g., residue 112, 113, 114, 115, 116, 117, 118, 119, or 120). In some embodiments, the C-terminal residue of the circularly permuted Cas12i2 protein comprises an amino acid residue “y” of SEQ ID NO: 1 or any one of SEQ ID NOs: 39-43 (or a sequence having at least 80%, 85%, 90%, 95%, 96%, 97%, 98%, or 99% identity thereto), wherein y is x−1. For example, in some embodiments, the N-terminal residue of a circularly permutated Cas12i2 protein comprises residue 117 corresponding to SEQ ID NO: 40, and the C-terminal residue of a circularly permutated Cas12i2 protein comprises residue 116 corresponding to SEQ ID NO: 40. In some embodiments, the circularly permuted Cas12i2 protein comprises an amino acid sequence of SEQ ID NO: 49, or a sequence having at least 80%, 85%, 90%, 95%, 96%, 97%, 98%, or 99% identity thereto. In some embodiments, residue “x” and/or residue “y” is linked to a fusion domain. In some embodiments, the fusion domain comprises an NLS.

In some embodiments, the N-terminal residue of a circularly permuted Cas12i2 protein comprises an amino acid residue “x” of SEQ ID NO: 1 or any one of SEQ ID NOs: 39-43 (or a sequence having at least 80%, 85%, 90%, 95%, 96%, 97%, 98%, or 99% identity thereto), wherein x is chosen from residues 195-206 (e.g., residue 195, 196, 197, 198, 199, 200, 201, 202, 203, 204, 205, or 206). In some embodiments, the C-terminal residue of the circularly permuted Cas12i2 protein comprises an amino acid residue “y” of SEQ ID NO: 1 or any one of SEQ ID NOs: 39-43 (or a sequence having at least 80%, 85%, 90%, 95%, 96%, 97%, 98%, or 99% identity thereto), wherein y is x−1. For example, in some embodiments, the N-terminal residue of a circularly permutated Cas12i2 protein comprises residue 200 corresponding to SEQ ID NO: 40, and the C-terminal residue of a circularly permutated Cas12i2 protein comprises residue 199 corresponding to SEQ ID NO: 40. In some embodiments, the circularly permuted Cas12i2 protein comprises an amino acid sequence of SEQ ID NO: 50, or a sequence having at least 80%, 85%, 90%, 95%, 96%, 97%, 98%, or 99% identity thereto. In some embodiments, residue “x” and/or residue “y” is linked to a fusion domain. In some embodiments, the fusion domain comprises an NLS.

In some embodiments, the N-terminal residue of a circularly permuted Cas12i2 protein comprises an amino acid residue “x” of SEQ ID NO: 1 or any one of SEQ ID NOs: 39-43 (or a sequence having at least 80%, 85%, 90%, 95%, 96%, 97%, 98%, or 99% identity thereto), wherein x is chosen from residues 241-250 (e.g., residue 241, 242, 243, 244, 245, 246, 247, 248, 249, or 250). In some embodiments, the C-terminal residue of the circularly permuted Cas12i2 protein comprises an amino acid residue “y” of SEQ ID NO: 1 or any one of SEQ ID NOs: 39-43 (or a sequence having at least 80%, 85%, 90%, 95%, 96%, 97%, 98%, or 99% identity thereto), wherein y is x−1. For example, in some embodiments, the N-terminal residue of a circularly permutated Cas12i2 protein comprises residue 247 corresponding to SEQ ID NO: 40, and the C-terminal residue of a circularly permutated Cas12i2 protein comprises residue 246 corresponding to SEQ ID NO: 40. In some embodiments, the circularly permuted Cas12i2 protein comprises an amino acid sequence of SEQ ID NO: 51, or a sequence having at least 80%, 85%, 90%, 95%, 96%, 97%, 98%, or 99% identity thereto. In some embodiments, residue “x” and/or residue “y” is linked to a fusion domain. In some embodiments, the fusion domain comprises an NLS.

In some embodiments, the N-terminal residue of a circularly permuted Cas12i2 protein comprises an amino acid residue “x” of SEQ ID NO: 1 or any one of SEQ ID NOs: 39-43 (or a sequence having at least 80%, 85%, 90%, 95%, 96%, 97%, 98%, or 99% identity thereto), wherein x is chosen from residues 342-358 (e.g., residue 342, 343, 344, 345, 346, 347, 348, 349, 350, 351, 352, 353, 354, 355, 356, 357, or 358). In some embodiments, the C-terminal residue of the circularly permuted Cas12i2 protein comprises an amino acid residue “y” of SEQ ID NO: 1 or any one of SEQ ID NOs: 39-43 (or a sequence having at least 80%, 85%, 90%, 95%, 96%, 97%, 98%, or 99% identity thereto), wherein y is x−1. For example, in some embodiments, the N-terminal residue of a circularly permutated Cas12i2 protein comprises residue 343 corresponding to SEQ ID NO: 1 or any one of SEQ ID NOs: 39-43, and the C-terminal residue of a circularly permutated Cas12i2 protein comprises residue 342. In some embodiments, residue “x” and/or residue “y” is linked to a fusion domain. In some embodiments, the fusion domain is a FokI nuclease domain (e.g., a catalytically active FokI nuclease domain or a catalytically inactive FokI nuclease domain).

In some embodiments, the N-terminal residue of a circularly permuted Cas12i2 protein comprises an amino acid residue “x” of SEQ ID NO: 1 or any one of SEQ ID NOs: 39-43 (or a sequence having at least 80%, 85%, 90%, 95%, 96%, 97%, 98%, or 99% identity thereto), wherein x is chosen from residues 373-378 (e.g., residue 373, 374, 375, 376, 377, or 378). In some embodiments, the C-terminal residue of the circularly permuted Cas12i2 protein comprises an amino acid residue “y” of SEQ ID NO: 1 or any one of SEQ ID NOs: 39-43 (or a sequence having at least 80%, 85%, 90%, 95%, 96%, 97%, 98%, or 99% identity thereto), wherein y is x−1. For example, in some embodiments, the N-terminal residue of a circularly permutated Cas12i2 protein comprises residue 374 corresponding to SEQ ID NO: 1 or any one of SEQ ID NOs: 39-43, and the C-terminal residue of a circularly permutated Cas12i2 protein comprises residue 373. In some embodiments, residue “x” and/or residue “y” is linked to a fusion domain. In some embodiments, the fusion domain is a FokI nuclease domain (e.g., a catalytically active FokI nuclease domain or a catalytically inactive FokI nuclease domain).

In some embodiments, the N-terminal residue of a circularly permuted Cas12i2 protein comprises an amino acid residue “x” of SEQ ID NO: 1 or any one of SEQ ID NOs: 39-43 (or a sequence having at least 80%, 85%, 90%, 95%, 96%, 97%, 98%, or 99% identity thereto), wherein x is chosen from residues 386-397 (e.g., residue 386, 387, 388, 389, 390, 391, 392, 393, 394, 395, 396, or 397). In some embodiments, the C-terminal residue of the circularly permuted Cas12i2 protein comprises an amino acid residue “y” of SEQ ID NO: 1 or any one of SEQ ID NOs: 39-43 (or a sequence having at least 80%, 85%, 90%, 95%, 96%, 97%, 98%, or 99% identity thereto), wherein y is x−1. For example, in some embodiments, the N-terminal residue of a circularly permutated Cas12i2 protein comprises residue 387 corresponding to SEQ ID NO: 1, and the C-terminal residue of a circularly permutated Cas12i2 protein comprises residue 386. In some embodiments, residue “x” and/or residue “y” is linked to a fusion domain. In some embodiments, the fusion domain is a FokI nuclease domain (e.g., a catalytically active FokI nuclease domain or a catalytically inactive FokI nuclease domain).

In some embodiments, the N-terminal residue of a circularly permuted Cas12i2 protein comprises an amino acid residue “x” of SEQ ID NO: 1 or any one of SEQ ID NOs: 39-43 (or a sequence having at least 80%, 85%, 90%, 95%, 96%, 97%, 98%, or 99% identity thereto), wherein x is chosen from residues 408-413 (e.g., residue 408, 409, 410, 411, 412, or 413). In some embodiments, the C-terminal residue of the circularly permuted Cas12i2 protein comprises an amino acid residue “y” of SEQ ID NO: 1 or any one of SEQ ID NOs: 39-43 (or a sequence having at least 80%, 85%, 90%, 95%, 96%, 97%, 98%, or 99% identity thereto), wherein y is x−1. For example, in some embodiments, the N-terminal residue of a circularly permutated Cas12i2 protein comprises residue 410 corresponding to SEQ ID NO: 40, and the C-terminal residue of a circularly permutated Cas12i2 protein comprises residue 409 corresponding to SEQ ID NO: 40. In some embodiments, the circularly permuted Cas12i2 protein comprises an amino acid sequence of SEQ ID NO: 45, or a sequence having at least 80%, 85%, 90%, 95%, 96%, 97%, 98%, or 99% identity thereto. In some embodiments, residue “x” and/or residue “y” is linked to a fusion domain. In some embodiments, the fusion domain comprises an NLS.

In some embodiments, the N-terminal residue of a circularly permuted Cas12i2 protein comprises an amino acid residue “x” of SEQ ID NO: 1 or any one of SEQ ID NOs: 39-43 (or a sequence having at least 80%, 85%, 90%, 95%, 96%, 97%, 98%, or 99% identity thereto), wherein x is chosen from residues 677-685 (e.g., residue 677, 678, 679, 680, 681, 682, 683, 684, or 685). In some embodiments, the C-terminal residue of the circularly permuted Cas12i2 protein comprises an amino acid residue “y” of SEQ ID NO: 1 or any one of SEQ ID NOs: 39-43 (or a sequence having at least 80%, 85%, 90%, 95%, 96%, 97%, 98%, or 99% identity thereto), wherein y is x−1. For example, in some embodiments, the N-terminal residue of a circularly permutated Cas12i2 protein comprises residue 678 corresponding to SEQ ID NO: 1, and the C-terminal residue of a circularly permutated Cas12i2 protein comprises residue 677. In some embodiments, the N-terminal residue of a circularly permutated Cas12i2 protein comprises residue 681 corresponding to SEQ ID NO: 40, and the C-terminal residue of a circularly permutated Cas12i2 protein comprises residue 680 corresponding to SEQ ID NO: 40. In some embodiments, the circularly permuted Cas12i2 protein comprises an amino acid sequence of SEQ ID NO: 46, or a sequence having at least 80%, 85%, 90%, 95%, 96%, 97%, 98%, or 99% identity thereto. In some embodiments, residue “x” and/or residue “y” is linked to a fusion domain. In some embodiments, the fusion domain comprises an NLS. In some embodiments, the fusion domain is a FokI nuclease domain (e.g., a catalytically active FokI nuclease domain or a catalytically inactive FokI nuclease domain).

In some embodiments, the N-terminal residue of a circularly permuted Cas12i2 protein comprises an amino acid residue “x” of SEQ ID NO: 1 or any one of SEQ ID NOs: 39-43 (or a sequence having at least 80%, 85%, 90%, 95%, 96%, 97%, 98%, or 99% identity thereto), wherein x is chosen from residues 771-782 (e.g., residue 771, 772, 773, 774, 775, 776, 777, 778, 779, 780, 781, or 782). In some embodiments, the C-terminal residue of the circularly permuted Cas12i2 protein comprises an amino acid residue “y” of SEQ ID NO: 1 or any one of SEQ ID NOs: 39-43 (or a sequence having at least 80%, 85%, 90%, 95%, 96%, 97%, 98%, or 99% identity thereto), wherein y is x−1. For example, in some embodiments, the N-terminal residue of a circularly permutated Cas12i2 protein comprises residue 772 corresponding to SEQ ID NO: 1, and the C-terminal residue of a circularly permutated Cas12i2 protein comprises residue 771. In some embodiments, residue “x” and/or residue “y” is linked to a fusion domain. In some embodiments, the fusion domain is a FokI nuclease domain (e.g., a catalytically active FokI nuclease domain or a catalytically inactive FokI nuclease domain).

In some embodiments, the N-terminal residue of a circularly permuted Cas12i2 protein comprises an amino acid residue “x” of SEQ ID NO: 1 or any one of SEQ ID NOs: 39-43 (or a sequence having at least 80%, 85%, 90%, 95%, 96%, 97%, 98%, or 99% identity thereto), wherein x is chosen from residues 831-844 (e.g., residue 831, 832, 833, 834, 835, 836, 837, 838, 839, 840, 841, 842, 843, or 844). In some embodiments, the C-terminal residue of the circularly permuted Cas12i2 protein comprises an amino acid residue “y” of SEQ ID NO: 1 or any one of SEQ ID NOs: 39-43 (or a sequence having at least 80%, 85%, 90%, 95%, 96%, 97%, 98%, or 99% identity thereto), wherein y is x−1. For example, in some embodiments, the N-terminal residue of a circularly permutated Cas12i2 protein comprises residue 832 corresponding to SEQ ID NO: 1, and the C-terminal residue of a circularly permutated Cas12i2 protein comprises residue 831. In some embodiments, residue “x” and/or residue “y” is linked to a fusion domain. In some embodiments, the fusion domain is a FokI nuclease domain (e.g., a catalytically active FokI nuclease domain or a catalytically inactive FokI nuclease domain).

In some embodiments, the N-terminal residue of a circularly permuted Cas12i2 protein comprises an amino acid residue “x” of SEQ ID NO: 1 or any one of SEQ ID NOs: 39-43 (or a sequence having at least 80%, 85%, 90%, 95%, 96%, 97%, 98%, or 99% identity thereto), wherein x is chosen from residues 877-901 (e.g., residue 877, 878, 879, 880, 881, 882, 883, 884, 885, 886, 887, 888, 889, 890, 891, 892, 893, 894, 895, 896, 897, 898, 899, 900, or 901). In some embodiments, the C-terminal residue of the circularly permuted Cas12i2 protein comprises an amino acid residue “y” of SEQ ID NO: 1 or any one of SEQ ID NOs: 39-43 (or a sequence having at least 80%, 85%, 90%, 95%, 96%, 97%, 98%, or 99% identity thereto), wherein y is x−1. For example, in some embodiments, the N-terminal residue of a circularly permutated Cas12i2 protein comprises residue 893 corresponding to SEQ ID NO: 40, and the C-terminal residue of a circularly permutated Cas12i2 protein comprises residue 892 corresponding to SEQ ID NO: 40. In some embodiments, the circularly permuted Cas12i2 protein comprises an amino acid sequence of SEQ ID NO: 52, or a sequence having at least 80%, 85%, 90%, 95%, 96%, 97%, 98%, or 99% identity thereto. In some embodiments, residue “x” and/or residue “y” is linked to a fusion domain. In some embodiments, the fusion domain comprises an NLS.

In some embodiments, the N-terminal residue of a circularly permuted Cas12i2 protein comprises an amino acid residue “x” of SEQ ID NO: 1 or any one of SEQ ID NOs: 39-43 (or a sequence having at least 80%, 85%, 90%, 95%, 96%, 97%, 98%, or 99% identity thereto), wherein x is chosen from residues 953-965 (e.g., residue 953, 954, 955, 956, 957, 958, 959, 960, 961, 962, 963, 964, or 965). In some embodiments, the C-terminal residue of the circularly permuted Cas12i2 protein comprises an amino acid residue “y” of SEQ ID NO: 1 or any one of SEQ ID NOs: 39-43 (or a sequence having at least 80%, 85%, 90%, 95%, 96%, 97%, 98%, or 99% identity thereto), wherein y is x−1. For example, in some embodiments, the N-terminal residue of a circularly permutated Cas12i2 protein comprises residue 954 corresponding to SEQ ID NO: 1, and the C-terminal residue of a circularly permutated Cas12i2 protein comprises residue 953. In some embodiments, residue “x” and/or residue “y” is linked to a fusion domain. In some embodiments, the fusion domain is a FokI nuclease domain (e.g., a catalytically active FokI nuclease domain or a catalytically inactive FokI nuclease domain).

In some embodiments, a circularly permuted Cas12i2 protein is truncated relative to a Cas12i2 protein of SEQ ID NO: 1 or any one of SEQ ID NOs: 39-43. In some embodiments, a circularly permuted Cas12i2 protein has a modified Helical II domain relative to the Cas12i2 protein of SEQ ID NO: 1 or any one of SEQ ID NOs: 39-43. For example, in some embodiments, the circularly permuted Cas12i2 protein comprises substitutions or deletions in the Helical II domain relative to the sequence of SEQ ID NO: 1 or any one of SEQ ID NOs: 39-43. In some embodiments, a circularly permuted Cas12i2 protein comprises a truncated Helical II domain. For example, in some embodiments, the circularly permuted Cas12i2 protein does not comprise one or more flexible loops or alpha helices of the Helical II domain. For example, in some embodiments, the circularly permuted Cas12i2 protein does not comprise the loop of residues 342-358 (or 343-357), the loop of residues 386-397 (or 387-396), or the alpha helices of residues 359-385 (or 358-386).

In some embodiments, the N-terminal residue of a circularly permuted Cas12i2 protein comprises an amino acid residue of SEQ ID NO: 1 or any one of SEQ ID NOs: 39-43 (or a sequence having at least 80%, 85%, 90%, 95%, 96%, 97%, 98%, or 99% identity thereto) chosen from residues 386-397 (e.g., residue 386, 387, 388, 389, 390, 391, 392, 393, 394, 395, 396, or 397); d) 677-685 (e.g., residue 677, 678, 679, 680, 681, 682, 683, 684, or 685). In some embodiments, the C-terminal residue of the circularly permuted Cas12i2 protein comprises an amino acid residue of SEQ ID NO: 1 or any one of SEQ ID NOs: 39-43 (or a sequence having at least 80%, 85%, 90%, 95%, 96%, 97%, 98%, or 99% identity thereto) chosen from residues 342-358 (e.g., residue 342, 343, 344, 345, 346, 347, 348, 349, 350, 351, 352, 353, 354, 355, 356, 357, or 358). In some embodiments, the C-terminal residue of the circularly permuted Cas12i2 protein comprises an amino acid residue of SEQ ID NO: 1 or any one of SEQ ID NOs: 39-43 (or a sequence having at least 80%, 85%, 90%, 95%, 96%, 97%, 98%, or 99% identity thereto) chosen from residues 330-342 (e.g., residue 330, 331, 332, 333, 334, 335, 336, 337, 338, 339, 340, 341, or 342). In some embodiments, the N-terminal residue and/or C-terminal residue further comprises a fusion domain. In some embodiments, the fusion domain is a FokI nuclease domain (e.g., a catalytically active FokI nuclease domain or a catalytically inactive FokI nuclease domain). In some embodiments, the fusion domain comprises an NLS.

In certain embodiments, the circularly permuted Cas12i2 protein comprises an additional heterologous sequence disposed between a first amino acid residue “n” and a second amino acid residue “m” of the circularly permuted Cas12i2 protein, wherein n and m are each independently an amino acid residue of SEQ ID NO: 1 or any one of SEQ ID NOs: 39-43. In some embodiments, n and m are each independently a number between:
i) 342-358 (e.g., 342, 343, 344, 345, 346, 347, 348, 349, 350, 351, 352, 353, 354, 355, 356, 357, or 358);
ii) 373-378 (e.g., 373, 374, 375, 376, 377, or 378);
iii) 408-413 (e.g., 408, 409, 410, 411, 412, or 413);
iv) 677-685 (e.g., 677, 678, 679, 680, 681, 682, 683, 684, or 685);
v) 718-723 (e.g., 718, 719, 720, 721, 722, or 723);
vi) 771-782 (e.g., 771, 772, 773, 774, 775, 776, 777, 778, 779, 780, 781, or 782);
vii) 953-965 (e.g., 953, 954, 955, 956, 957, 958, 959, 960, 961, 962, 963, 964, or 965);
viii) 55-65 (e.g., 55, 56, 57, 58, 59, 60, 61, 62, 63, 64, or 65);
ix) 99-105 (e.g., 99, 100, 101, 102, 103, 104, or 105);
x) 112-120 (e.g., 112, 113, 114, 115, 116, 117, 118, 119, or 120);
xi) 195-206 (e.g., 195, 196, 197, 198, 199, 200, 201, 202, 203, 204, 205, or 206);
xii) 241-250 (e.g., 241, 242, 243, 244, 245, 246, 247, 248, 249, or 250);
xiii) 583-594 (e.g., 583, 584, 585, 586, 587, 588, 589, 590, 591, 592, 593, or 594);
xiv) 877-901 (e.g., 877, 878, 879, 880, 881, 882, 883, 884, 885, 886, 887, 888, 889, 890, 891, 892, 893, 894, 895, 896, 897, 898, 899, 900, or 901);
xv) 173-179 (e.g., 173, 174, 175, 176, 177, 178, or 179);
xvi) 216-221 (e.g., 216, 217, 218, 219, 220, or 221);
xvii) 265-272 (e.g., 265, 266, 267, 268, 269, 270, 271, or 272);
xviii) 456-468 (e.g., 456, 457, 458, 459, 460, 461, 462, 463, 464, 465, 466, 467, or 468);
xix) 476-482 (e.g., 476, 477, 478, 479, 480, 481, or 482);
xx) 498-513 (e.g., 498, 499, 500, 501, 502, 503, 504, 505, 506, 507, 508, 509, 510, 511, 512, or 513);
xxi) 614-625 (e.g., 614, 615, 616, 617, 618, 619, 620, 621, 622, 623, 624, or 625);
xxii) 977-982 (e.g., 977, 978, 979, 980, 981, or 982); or
xxiii) 1007-1012 (e.g., 1007, 1008, 1009, 1010, 1011, or 1012).

In some embodiments, n<m. In some embodiments, m=n+1.

In certain embodiments, the N-terminal Met residue of any of SEQ ID NO: 1 or any one of SEQ ID NOs: 39-43 is absent. In some embodiments, the N-terminal residue of a circularly permuted Cas12i2 protein is a Met residue. In some embodiments, the Met residue is added to the N-terminus of any one of the circularly permuted Cas12i2 proteins described herein.

In some embodiments, the circularly permuted Cas12i2 protein is capable of binding an RNA guide comprising a direct repeat sequence and a spacer sequence, wherein the spacer sequence is capable of hybridizing to a target nucleic acid.

In any of the aspects described herein, the circularly permuted Cas12i2 protein comprises a catalytic residue (e.g., D599, E833, and D1019). In certain embodiments, the circularly permuted Cas12i2 protein comprises a mutation (e.g., an alanine mutation) at any one of amino acid residue D599, E833, or D1019 of SEQ ID NO: 1. In certain embodiments, the circularly permuted Cas12i2 protein is a dead Cas12i2 protein (e.g., a catalytically inactive Cas12i2 protein).

In some embodiments, a circularly permuted Cas12i2 protein described herein comprises nickase activity. In some embodiments, a circularly permuted Cas12i2 protein described herein nicks the target strand of a target nucleic acid. In some embodiments, a circularly permuted Cas12i2 protein described herein nicks the non-target strand of a target nucleic acid. In some embodiments, a circularly permuted Cas12i2 protein described herein nicks a target sequence adjacent to a Cas12i2 PAM sequence (e.g., a 5′-NTTN-3′ sequence). See, e.g., FIG. 11.

In some embodiments, the heterologous sequence comprises a peptide tag, a fluorescent protein, a base-editing domain, a DNA methylation domain, a histone residue modification domain, a localization factor (e.g., an NLS), a transcription modification factor, a light-gated control factor, a chemically inducible factor, a chromatin visualization factor, or a restriction endonuclease. In some embodiments, the heterologous sequence is about 5-10, 10-20, 20-30, 30-40, 40-50, 50-100, 100-200, 200-300, 300-400, 400-500, 500-600, 600-700, 700-800, 800-900, 900-1100, 1100-1400, 1400-1600, 1600-1800, or 1800-2000 amino acids in length.

### Fusion Domains

This application related to Cas12i2 fusion proteins comprising heterologous sequences as described herein. In some instances, the heterologous sequence comprises a fusion domain (e.g., a base editing domain, a ssDNA binding domain, an NLS domain, a poly-basic domain, or a nuclease domain). In some embodiments, the fusion domain can have various activities, e.g., methylase activity, demethylase activity, transcription activation activity, transcription repression activity, transcription release factor activity, histone modification activity, RNA cleavage activity, DNA cleavage activity, nucleic acid binding activity, ligase activity (e.g., an EC 6.1, 6.2, 6.3, 6.4, 6.5, or 6.6 ligase), transcriptase activity, reverse transcriptase activity, and switch activity (e.g., light inducible). In some embodiments, the fusion domain is chosen from peptide tag, a fluorescent protein, a base-editing domain, a DNA methylation domain, a histone residue modification domain, a localization factor (e.g., an NLS), a transcription modification factor, a ligase a light-gated control factor, a chemically inducible factor, or a chromatin visualization factor. In some embodiments, the fusion domains are chosen from Krüppel associated box (KRAB), VP64, VP16, Fok1, P65, HSF1, MyoD1, Geminin, Streptavidin, an asialoglycoprotein receptor ligand, and biotin-APEX, or biologically active portions thereof. In some embodiments, the fusion domain is selected from a restriction endonuclease, a CRISPR nuclease, or a domain thereof. The restriction endonuclease can be any restriction endonuclease known in the art (see, e.g., https://www.neb.com/tools-and-resources/selection-charts/alphabetized-list-of-recognition-specificities). In some embodiments, the restriction endonuclease is FokI or the nuclease domain thereof. The CRISPR nuclease can be any CRISPR nuclease known in the art, e.g., a class I or class II enzyme. The CRISPR nuclease can be a type I, type II, type III, type IV, type V, or type VI CRISPR nuclease. In some embodiments, the CRISPR nuclease is any CRISPR nuclease having a RuvC domain or split RuvC domain such that a Cas12i2 fusion protein comprises two or more RuvC domains or two or more split RuvC domains. The CRISPR nuclease can be a Cas9, Cas12, or Cas13 ortholog. The CRISPR nuclease can be a Cpf1 (Cas12a), C2c1 (Cas12b), Cas12c, Cas12d, Cas12e, Cas12g, Cas12h, Cas12i (e.g., Cas12i1 or Cas12i2), or Cas12j (also known as CasPhi). In some embodiments, the fusion domain is a splint ligase. In some embodiments, the fusion domains are chosen from a protein comprising a DNA binding domain (e.g., a helix-turn-helix motif (Aravind et al., FEMS Microbiology 29(2): 231-262, 2005), a zinc finger domain, a leucine zipper domain, a winged helix domain, a winged helix-turn-helix domain, a basic helix-loop-helix domain, an HMG-Box domain, a Wor3 domain, an OB-fold domain (Flynn and Zou Crit. Rev. Biochem. Mol. Biol. 45(4): 266-275, 2010), an immunoglobulin fold domain, a B3 domain, or a Tal effector domain), or a biologically active portion thereof. In some embodiments, the fusion domain comprises a multimerized fusion domain comprising two or more copies of any fusion domain described herein, optionally linked by a linker. The positioning of the one or more functional domains on the inactivated CRISPR nuclease is one that allows for correct spatial orientation for the fusion domain to affect the target with the attributed functional effect.

### Base Editing Domains

In some embodiments, Cas12i2 fusion proteins described herein comprise a fusion domain comprising a base editor that enable the Cas12i2 fusion proteins to edit a single nucleic acid base. In some instances, the fusion domain comprises a polypeptide that is capable of making a modification to a base (e.g., A, T, C, G, or U) within a nucleic acid sequence (e.g., DNA or RNA). In some instances, the base editing domain is capable of deamidating a base within a nucleic acid. In some instances, the base editing domain is capable of deamidating a base within a DNA molecule. In some instances, the base editing domain is capable of deamidating cytosine (C) in DNA. In some embodiments, the base editing domain is capable of deamidating a thymine (T) in DNA.

### Methylation and Demethylation Domains

In some embodiments, the fusion domain is capable of methylating a base within a nucleic acid. In some instances, the fusion domain is capable of methylating cytosine (C) in DNA. In some embodiments, the fusion domain is capable of methylating adenine (A) in DNA. In some embodiments, the fusion domain is capable of methylating uracil (U) in RNA.

In some embodiments, the fusion domain is capable of demethylating a base within a nucleic acid. In some embodiments, the fusion domain is capable of demethylating a thymine (T) in DNA. In some embodiments, the fusion domain is capable of demethylating guanine (G) in DNA.

Examples of fusion domains are methylase (e.g., an M6a (EC 2.1.1.72), M4c (EC 2.1.1.113), M5c (EC 2.1.1.37), RNA methyltransferase (NSUN1, NSUN2, NSUN3, NSUN4, NSUN5, NSUN6, NSUN7, TRDMT1 (previously DNMT2)), and DNA methyltransferase (DNMT1, DNMT3 (3a, 3b, 3c, 3L)).

### NLS Fusion Domains

In some embodiments, Cas12i2 fusion protein comprises a nuclear localization sequence (also known as a nuclear localization signal) that promotes translocation through the nuclear envelope via nuclear pore complexes. The nuclear pore complex is composed of nucleoporins. Nucleoporins interact with transport molecules known as karyopherins. Karyopherins bind to proteins containing a nuclear localization sequence and transport the protein across the nuclear pore complex. In some embodiments, a nuclear localization sequence consists of one or more short (e.g., <50 amino-acid residues) sequence of basic amino acids. In some embodiments, a nuclear localization sequence consists of one or more short (e.g., <50 amino-acid residues) sequence of lysines or arginines. In some embodiments, the nuclear localization sequence is monopartite or bipartite. In some embodiments, the nuclear localization sequence is a nucleoplasmin NLS (npNLS).

In some embodiments, the NLS comprises: KRPAATKKAGQAKKKK (SEQ ID NO: 61), MKRTADGSEFESPKKKRKV (SEQ ID NO: 62), MKRTADGSEFESPKKKRKVE (SEQ ID NO: 63), KRTADGSEFESPKKKRKV (SEQ ID NO: 64), or KRTADGSEFESPKKKRKVE (SEQ ID NO: 65). In some embodiments, the NLS comprises an amino acid sequence of any one of SEQ ID NOs: 61-65, or an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) identity thereto. In some embodiments, a linker (e.g., a polypeptide linker) is disposed between the Cas12i2 domain and the NLS. In some embodiments, the polypeptide linker comprises a glycine and/or serine residue (e.g., a GS linker). For example, in some embodiments, the Cas12i2 fusion proteins of SEQ ID NO: 68 and SEQ ID NO: 73 comprise the NLS of SEQ ID NO: 65, and the Cas12i2 fusion proteins of SEQ ID NO: 69 and SEQ ID NO: 74 comprise the NLS of SEQ ID NO: 64. In some embodiments, a Cas12i2 fusion protein comprises at least 80% (81%, 82%, 83%, 84%, 85%, 86%, 87%, 88%, 89%, 90%, 91%, 92%, 93%, 94%, 95%, 96%, 97%, 98%, 99%, or 100%) identity to SEQ ID NO: 68, SEQ ID NO: 69, SEQ ID NO: 73, or SEQ ID NO: 74.

In some embodiments, the nuclear localization sequence is disposed in the middle of the Cas12i2 fusion protein and is exposed on the fusion protein surface. In some embodiments, a nuclear localization sequence is recognized by a karyopherin. In some embodiment the nuclear localization sequence interacts with one or more karyopherin. In some embodiments, the karyopherin recognizes a nuclear localization sequence as it emerges from a ribosome. In some embodiments, the karyopherin recognizes a nuclear localization sequence on a fully translated protein.

In some embodiments, the nuclear localization sequence is defined as the nuclear localization sequence from the proteins listed in Table 6 of US 2015-0246139, which is incorporated by reference herein.

In some embodiments, the nuclear localization sequence is included in a heterologous sequence. In certain embodiments, the heterologous sequence comprising an NLS is located between a first portion comprising amino acids 1-n of SEQ ID NO: 1, or a sequence having at least 80%, 85%, 90%, 95%, 96%, 97%, 98%, or 99% identity thereto, and a second portion comprising amino acids m-1054 of SEQ ID NO: 1, or a sequence having at least 80%, 85%, 90%, 95%, 96%, 97%, 98%, or 99% identity thereto, wherein n and m are each independently a number between:
i) 342-358 (e.g., 342, 343, 344, 345, 346, 347, 348, 349, 350, 351, 352, 353, 354, 355, 356, 357, or 358);
ii) 373-378 (e.g., 373, 374, 375, 376, 377, or 378);
iii) 408-413 (e.g., 408, 409, 410, 411, 412, or 413);
iv) 677-685 (e.g., 677, 678, 679, 680, 681, 682, 683, 684, or 685);
v) 718-723 (e.g., 718, 719, 720, 721, 722, or 723);
vi) 771-782 (e.g., 771, 772, 773, 774, 775, 776, 777, 778, 779, 780, 781, or 782);
vii) 953-965 (e.g., 953, 954, 955, 956, 957, 958, 959, 960, 961, 962, 963, 964, or 965);
viii) 55-65 (e.g., 55, 56, 57, 58, 59, 60, 61, 62, 63, 64, or 65);
ix) 99-105 (e.g., 99, 100, 101, 102, 103, 104, or 105);
x) 112-120 (e.g., 112, 113, 114, 115, 116, 117, 118, 119, or 120);
xi) 195-206 (e.g., 195, 196, 197, 198, 199, 200, 201, 202, 203, 204, 205, or 206);
xii) 241-250 (e.g., 241, 242, 243, 244, 245, 246, 247, 248, 249, or 250);
xiii) 583-594 (e.g., 583, 584, 585, 586, 587, 588, 589, 590, 591, 592, 593, or 594);
xiv) 877-901 (e.g., 877, 878, 879, 880, 881, 882, 883, 884, 885, 886, 887, 888, 889, 890, 891, 892, 893, 894, 895, 896, 897, 898, 899, 900, or 901);
xv) 173-179 (e.g., 173, 174, 175, 176, 177, 178, or 179);
xvi) 216-221 (e.g., 216, 217, 218, 219, 220, or 221);
xvii) 265-272 (e.g., 265, 266, 267, 268, 269, 270, 271, or 272);
xviii) 456-468 (e.g., 456, 457, 458, 459, 460, 461, 462, 463, 464, 465, 466, 467, or 468);
xix) 476-482 (e.g., 476, 477, 478, 479, 480, 481, or 482);
xx) 498-513 (e.g., 498, 499, 500, 501, 502, 503, 504, 505, 506, 507, 508, 509, 510, 511, 512, or 513);
xxi) 614-625 (e.g., 614, 615, 616, 617, 618, 619, 620, 621, 622, 623, 624, or 625);
xxii) 977-982 (e.g., 977, 978, 979, 980, 981, or 982); or
xxiii) 1007-1012 (e.g., 1007, 1008, 1009, 1010, 1011, or 1012), wherein m is greater than n.

In some embodiments, the heterologous sequence comprises an NLS. In certain embodiments, the heterologous sequence comprising an NLS is located at the N-terminus and/or C-terminus of a circularly permuted Cas12i2 protein. In certain embodiments, the heterologous sequence comprising an NLS is located at the N-terminus of a circularly permuted Cas12i2 protein. In certain embodiments, the heterologous sequence comprising an NLS is located at the C-terminus of a circularly permuted Cas12i2 protein.

### Split Fusion Domain

In some embodiments, the Cas12i2 fusion protein comprises a split fusion domain. Typically, a split fusion domain is a domain wherein a reference protein is split into two parts, which together substantially comprises a functioning fusion domain. A split can be done in any way that the function of the fusion domain(s) is unaffected. In some embodiments, the split is substantially proportional (e.g., a first split fusion portion and a second split fusion portion are substantially equal in amino acid length). In some embodiments, one portion of the split fusion domain has a greater number of amino acid residues than a second portion of the split fusion protein. In some embodiments, a split fusion domain is chosen from beta-lactamase, dihydrofolate reductase (DHFR), focal adhesion kinase (FAK), green fluorescent protein GFP), enhanced GFP (EGFP), horseradish peroxidase, infrared fluorescent protein IFP1.4, LacZ, luciferase (e.g., recombinase enhanced bimolecular luciferase (ReBiL), Gaussia princeps luciferase, NanoLuc, and NanoBIT), Tobacco etch virus protease (TEV), and ubiquitin.

### Dimerization Domain

In some embodiments, the Cas12i2 fusion protein comprises a dimerization domain. Typically, a dimerization domain is a polypeptide domain capable of specifically binding a separate, and compatible, polypeptide domain (e.g., a second compatible dimerization domain). In some embodiments, the dimer is formed by a non-covalent bond between the first dimerization domain and the second compatible dimerization domain. In some embodiments, the first dimerization domain and the second compatible dimerization domain are identical (e.g., a homodimer). In some embodiments, the first dimerization domain and the second dimerization domain are not identical (e.g., a heterodimer). In some embodiments, a dimerization domain is a leucine zipper. In some instances, the dimerization domain is a chemically inducible dimerization domain (e.g., a rapamycin sensitive dimerization domain) that can be regulated by the presence of a small molecule. In some embodiments, the dimerization domain is a light inducible dimerization domain (e.g., a far-red light inducible) that can be regulated by light exposure.

### Cas12i2 Domain

In some embodiments, the Cas12i2 fusion protein of the present invention includes a Cas12i2 domain described herein.

A nucleic acid sequence encoding a Cas12i2 domain described herein may be substantially identical to a reference nucleic acid sequence if the nucleic acid encoding the Cas12i2 domain comprises a sequence having least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the two nucleic acid molecules hybridize to each other under stringent conditions (e.g., within a range of medium to high stringency).

In some embodiments, a Cas12i2 domain described herein is encoded by a nucleic acid sequence having at least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a reference nucleic acid sequence.

A nuclease described herein may substantially identical to a reference polypeptide if the nuclease comprises an amino acid sequence having at least about 60%, least about 65%, least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the amino acid sequence of the reference polypeptide. The percent identity between two such polypeptides can be determined manually by inspection of the two optimally aligned polypeptide sequences or by using software programs or algorithms (e.g., BLAST, ALIGN, CLUSTAL) using standard parameters. One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative amino acid substitution or one or more conservative amino acid substitutions.

In some embodiments, a Cas12i2 domain of the present invention comprises a polypeptide sequence having 50, 60, 65, 70, 75, 80, 85, 90, 91, 92, 93, 94, 95, 96, 97, 98, 99 or 100% identity to any one of SEQ ID NOs: 1 and 39-43. In some embodiments, a Cas12i2 domain of the present invention comprises a polypeptide sequence having greater than 50, 60, 65, 70, 75, 80, 85, 90, 91, 92, 93, 94, 95, 96, 97, 98, 99 or 100% identity to any one of SEQ ID NO: 1 and SEQ ID NOs: 39-43.

TABLE 1
Amino acid sequences of Cas12i2 polypeptides
of SEQ ID NO: 1 and SEQ ID NOs: 39-43.

| SEQ | |
|---|---|
| ID | |
| NO | Amino Acid Sequence |
| 1 | MSSAIKSYKSVLRPNERKNQLLKSTIQCLE |
| | DGSAFFFKMLQGLFGGITPEIVRESTEQEK |
| | QQQDIALWCAVNWFRPVSQDSLTHTIASDN |
| | LVEKFEEYYGGTASDAIKQYFSASIGESYY |
| | WNDCRQQYYDLCRELGVEVSDLTHDLEILC |
| | REKCLAVATESNQNNSIISVLFGTGEKEDR |
| | SVKLRITKKILEAISNLKEIPKNVAPIQEI |
| | ILNVAKATKETFRQVYAGNLGAPSTLEKFI |
| | AKDGQKEFDLKKLQTDLKKVIRGKSKERDW |
| | CCQEELRSYVEQNTIQYDLWAWGEMFNKAH |
| | TALKIKSTRNYNFAKQRLEQFKEIQSLNNL |
| | LVVKKLNDFFDSEFFSGEETYTICVHHLGG |
| | KDLSKLYKAWEDDPADPENAIVVLCDDLKN |
| | NFKKEPIRNILRYIFTIRQECSAQDILAAA |
| | KYNQQLDRYKSQKANPSVLGNQGFTWTNAV |
| | ILPEKAQRNDRPNSLDLRIWLYLKLRHPDG |
| | RWKKHHIPFYDTRFFQEIYAAGNSPVDTCQ |
| | FRIPRFGYHLPKLIDQTAIRVNKKHVKAAK |
| | TEARIRLAIQQGTLPVSNLKITEISATINS |
| | KGQVRIPVKFDVGRQKGTLQIGDRFCGYDQ |
| | NQTASHAYSLWEVVKEGQYHKELGCFVRFI |
| | SSGDIVSITENRGNQFDQLSYEGLAYPQYA |
| | DWRKKASKFVSLWQITKKNKKKEIVTVEAK |
| | EKFDAICKYQPRLYKENKEYAYLLRDIVRG |
| | KSLVELQQIRQEIFRFIEQDCGVTRLGSLS |
| | LSTLETVKAVKGIIYSYFSTALNASKNNPI |
| | SDEQRKEFDPELFALLEKLELIRTRKKKQK |
| | VERIANSLIQTCLENNIKFIRGEGDLSTIN |
| | NATKKKANSRSMDWLARGVENKIRQLAPMH |
| | NITLFGCGSLYTSHQDPLVHRNPDKAMKCR |
| | WAAIPVKDIGDWVLRKLSQNLRAKNIGTGE |
| | YYHQGVKEFLSHYELQDLEEELLKWRSDRK |
| | SNIPCWVLQNRLAEKLGNKEAVVYIPVRGG |
| | RIYFATHKVATGAVSIVFDQKQVWVCNADH |
| | VAAANIALTVKGIGEQSSDEENPDGSRIKL |
| | QLTS |
| 39 | MSSAIKSYKSVLRPNERKNQLKSTIQCLED |
| | GSAFFFKMLQGLFGGITPEIVRESTEQEKQ |
| | QQDIALWCAVNWFRPVSQDSLTHTIASDNL |
| | VEKFEEYYGGTASDAIKQYFSASIGESYYW |
| | NDCRQQYYDLCRELGVEVSDLTHDLEILCR |
| | EKCLAVATESNQNNSIISVLFGTGEKEDRS |
| | VKLRITKKILEAISNLKEIPKNVAPIQEII |
| | LNVAKATKETFRQVYAGNLGAPSTLEKFIA |
| | KDGQKEFDLKKLQTDLKKVIRGKSKERDWC |
| | CQEELRSYVEQNTIQYDLWAWGEMFNKAHT |
| | ALKIKSTRNYNFAKQRLEQFKEIQSLNNLL |
| | VVKKLNDFFDSEFFSGEETYTICVHHLGGK |
| | DLSKLYKAWEDDPADPENAIVVLCDDLKNN |
| | FKKEPIRNILRYIFTIRQECSAQDILAAAK |
| | YNQQLDRYKSQKANPSVLGNQGFTWTNAVI |
| | LPEKAQRNDRPNSLDLRIWLYLKLRHPDGR |
| | WKKHHIPFYDTRFFQEIYAAGNSPVDTCQF |
| | RTPRFGYHLPKLTDQTAIRVNKKHVKAAKT |
| | EARIRLAIQQGTLPVSNLKITEISATINSK |
| | GQVRIPVKFRVGRQKGTLQIGDRFCGYDQN |
| | QTASHAYSLWEVVKEGQYHKELGCFVRFIS |
| | SGDIVSITENRGNQFDQLSYEGLAYPQYAD |
| | WRKKASKFVSLWQITKKNKKKEIVTVEAKE |
| | KFDAICKYQPRLYKENKEYAYLLRDIVRGK |
| | SLVELQQIRQEIFRFIEQDCGVTRLGSLSL |
| | STLETVKAVKGIIYSYFSTALNASKNNPIS |
| | DEQRKEFDPELFALLEKLELIRTRKKKQKV |
| | ERIANSLIQTCLENNIKFIRGEGDLSTTNN |
| | ATKKKANSRSMDWLARGVFNKIRQLAPMHN |
| | ITLFGCGSLYTSHQDPLVHRNPDKAMKCRW |
| | AAIPVKDIGRWVLRKLSQNLRAKNRGTGEY |
| | YHQGVKEFLSHYELQDLEEELLKWRSDRKS |
| | NIPCWVLQNRLAEKLGNKEAVVYIPVRGGR |
| | IYFATHKVATGAVSIVFDQKQVWVCNADHV |
| | AAANIALTGKGIGEQSSDEENPDGSRIKLQ |
| | LTS |
| 40 | MSSAIKSYKSVLRPNERKNQLLKSTIQCLE |
| | DGSAFFFKMLQGLEGGITPEIVRESTEQEK |
| | QQQDIALWCAVNWFRPVSQDSLTHTIASDN |
| | LVEKFEEYYGGTASDAIKQYFSASIGESYY |
| | WNDCRQQYYDLCRELGVEVSDLTHDLEILC |
| | REKCLAVATESNQNNSIISVLFGTGEKEDR |
| | SVKLRITKKILEAISNLKEIPKNVAPIQEI |
| | ILNVAKATKETFRQVYAGNLGAPSTLEKFI |
| | AKDGQKEFDLKKLQTDLKKVIRGKSKERDW |
| | CCQEELRSYVEQNTIQYDLWAWGEMFNKAH |
| | TALKIKSTRNYNFAKQRLEQFKEIQSLNNL |
| | LVVKKLNDFFDSEFFSGEETYTICVHHLGG |
| | KDLSKLYKAWEDDPADPENAIVVLCDDLKN |
| | NFKKEPIRNILRYIFTIRQECSAQDILAAA |
| | KYNQQLDRYKSQKANPSVLGNQGFTWINAV |
| | ILPEKAQRNDRPNSLDLRIWLYLKLRHPDG |
| | RWKKHHIPFYDTRFFQEIYAAGNSPVDTCQ |
| | FRTPRFGYHLPKLIDQTAIRVNKKHVKAAK |
| | TEARIRLAIQQGTLPVSNLKITEISATINS |
| | KGQVRIPVKFRVGRQKGTLQIGDRFCGYDQ |
| | NQTASHAYSLWEVVKEGQYHKELGCFVRFI |
| | SSGDIVSITENRGNQFDQLSYEGLAYPQYA |
| | DWRKKASKFVSLWQITKKNKKKEIVIVEAK |
| | EKFDAICKYQPRLYKENKEYAYLLRDIVRG |
| | KSLVELQQIRQEIFRFIEQDCGVTRLGSLS |
| | LSTLETVKAVKGIIYSYFSTALNASKNNPI |
| | SDEQRKEFDPELFALLEKLELIRTRKKKQK |
| | VERIANSLIQTCLENNIKFIRGEGDLSTIN |
| | NATKKKANSRSMDWLARGVENKIRQLAPMH |
| | NITLFGCGSLYTSHQDPLVHRNPDKAMKCR |
| | WAAIPVKDIGDWVLRKLSQNLRAKNRGIGE |
| | YYHQGVKEFLSHYELQDLEEELLKWRSDRK |
| | SNIPCWVLQNRLAEKLGNKEAVVYIPVRGG |
| | RIYFATHKVATGAVSIVFDQKQVWVCNADH |
| | VAAANIALTGKGIGEQSSDEENPDGSRIKL |
| | QLTS |
| 41 | MSSAIKSYKSVLRPNERKNQLLKSTIQCLE |
| | DGSAFFFKMLQGLFGGITPEIVRESTEQEK |
| | QQQDIALWCAVNWFRPVSQDSLTHTIASDN |
| | LVEKFEEYYGGTASDAIKQYFSASIGESYY |
| | WNDCRQQYYDLCRELGVEVSDLTHDLEILC |
| | REKCLAVATESNQNNSIISVLFGTGEKEDR |
| | SVKLRITKKILEAISNLKEIPKNVAPIQEI |
| | ILNVAKATKETFRQVYAGNLGAPSTLEKFI |
| | AKDGQKEFDLKKLQTDLKKVIRGKSKERDW |
| | CCQEELRSYVEQNTIQYDLWAWGEMFNKAH |
| | TALKIKSTRNYNFAKQRLEQFKEIQSLNNL |
| | LVVKKLNDFFDSEFFSGEETYTICVHHLGG |
| | KDLSKLYKAWEDDPADPENAIVVLCDDLKN |
| | NFKKEPIRNILRYIFTIRQECSAQDILAAA |
| | KYNQQLDRYKSQKANPSVLGNQGFTWINAV |
| | ILPEKAQRNDRPNSLDLRIWLYLKLRHPDG |
| | RWKKHHIPFYDTRFFQEIYAAGNSPVDTCQ |
| | FRTPRFGYHLPKLIDQTAIRVNKKHVKAAK |
| | TEARIRLAIQQGTLPVSNLKITEISATINS |
| | KGQVRIPVKFRVGRQKGTLQIGDRFCGYDQ |
| | NQTASHAYSLWEVVKEGQYHKELGCFVRFI |
| | SSGDIVSITENRGNQFDQLSYEGLAYPQYA |
| | DWRKKASKFVSLWQITKKNKKKEIVTVEAK |
| | EKFDAICKYQPRLYKENKEYAYLLRDIVRG |
| | KSLVELQQIRQEIFRFIEQDCGVTRLGSLS |
| | LSTLETVKAVKGIIYSYFSTALNASKNNPI |
| | SDEQRKEFDPELFALLEKLELIRTRKKKQK |
| | VERIANSLIQTCLENNIKFIRGEGDLSTTN |
| | NATKKKANSRSMDWLARGVENKIRQLAPMH |
| | NITLFGCGSLYTSHQDPLVHRNPDKAMKCR |
| | WAAIPVKDIGDWVLRKLSQNLRAKNRGTGE |
| | YYHQGVKEFLSHYELQDLEEELLKWRSDRK |
| | SNIPCWVLQNRLAEKLGNKEAVVYIPVRGG |
| | RIYFATHKVATGAVSIVFDQKQVWVCNADH |
| | VAAANIALTGKGIGEQSSDEENPDGGRIKL |
| | QLTS |
| 42 | MSSAIKSYKSVLRPNERKNQLLKSTIQCLE |
| | DGSAFFFKMLQGLFGGITPEIVRESTEQEK |
| | QQQDIALWCAVNWFRPVSQDSLTHTIASDN |
| | LVEKFEEYYGGTASDAIKQYFSASIGESYY |
| | WNDCRQQYYDLCRELGVEVSDLTHDLEILC |
| | REKCLAVATESNQNNSIISVLFGTGEKEDR |
| | SVKLRITKKILEAISNLKEIPKNVAPIQEI |
| | ILNVAKATKETFRQVYAGNLGAPSTLEKFI |
| | AKDGQKEFDLKKLQTDLKKVIRGKSKERDW |
| | CCQEELRSYVEQNTIQYDLWAWGEMFNKAH |
| | TALKIKSTRNYNFAKQRLEQFKEIQSLNNL |
| | LVVKKLNDFFDSEFFSGEETYTICVHHLGG |
| | KDLSKLYKAWEDDPADPENAIVVLCDDLKN |
| | NFKKEPIRNILRYIFTIRQECSAQDILAAA |
| | KYNQQLDRYKSQKANPSVLGNQGFTWINAV |
| | ILPEKAQRNDRPNSLDLRIWLYLKLRHPDG |
| | RWKKHHIPFYDTRFFQEIYAAGNSPVDTCQ |
| | FRTPRFGYHLPKLTDQTAIRVNKKHVKAAK |
| | TEARIRLAIQQGTLPVSNLKITEISATINS |
| | KGQVRIPVKFRVGRQKGTLQIGDRFCGYDQ |
| | NQTASHAYSLWEVVKEGQYHKELRCRVRFI |
| | SSGDIVSITENRGNQFDQLSYEGLAYPQYA |
| | DWRKKASKFVSLWQITKKNKKKEIVTVEAK |
| | EKFDAICKYQPRLYKENKEYAYLLRDIVRG |
| | KSLVELQQIRQEIFRFIEQDCGVTRLGSLS |
| | LSTLETVKAVKGIIYSYFSTALNASKNNPI |
| | SDEQRKEFDPELFALLEKLELIRTRKKKQK |
| | VERIANSLIQTCLENNIKFIRGEGDLSTIN |
| | NATKKKANSRSMDWLARGVFNKIRQLAPMH |
| | NITLFGCGSLYTSHQDPLVHRNPDKAMKCR |
| | WAAIPVKDIGDWVLRKLSQNLRAKNRGTGE |
| | YYHQGVKEFLSHYELQDLEEELLKWRSDRK |
| | SNIPCWVLQNRLAEKLGNKEAVVYIPVRGG |
| | RIYFATHKVATGAVSIVFDQKQVWVCNADH |
| | VAAANIALIGKGIGRQSSDEENPDGGRIKL |
| | QLTS |
| 43 | MSSAIKSYKSVLRPNERKNQLKSTIQCLED |
| | GSAFFFKMLQGLEGGITPEIVRESTEQEKQ |
| | QQDIALWCAVNWFRPVSQDSLTHTIASDNL |
| | VEKFEEYYGGTASDAIKQYFSASIGESYYW |
| | NDCRQQYYDLCRELGVEVSDLTHDLEILCR |
| | EKCLAVATESNQNNSIISVLFGTGEKEDRS |
| | VKLRITKKILEAISNLKEIPKNVAPIQEII |
| | LNVAKATKETFRQVYAGNLGAPSTLEKFIA |
| | KDGQKEFDLKKLQTDLKKVIRGKSKERDWC |
| | CQEELRSYVEQNTIQYDLWAWGEMFNKAHT |
| | ALKIKSTRNYNFAKQRLEQFKEIQSLNNLL |
| | VVKKLNDFFDSEFFSGEETYTICVHHLGGK |
| | DLSKLYKAWEDDPADPENAIVVLCDDLKNN |
| | FKKEPIRNILRYIFTIRQECSAQDILAAAK |
| | YNQQLDRYKSQKANPSVLGNQGFTWINAVI |
| | LPEKAQRNDRPNSLDLRIWLYLKLRHPDGR |
| | WKKHHIPFYDTRFFQEIYAAGNSPVDTCQF |
| | RIPRFGYHLPKLIDQTAIRVNKKHVKAAKT |
| | EARIRLAIQQGTLPVSNLKITEISATINSK |
| | GQVRIPVKFRVGRQKGTLQIGDRFCGYDQN |
| | QTASHAYSLWEVVKEGQYHKELGCFVRFIS |
| | SGDIVSITENRGNQFDQLSYEGLAYPQYAD |
| | WRKKASKFVSLWQITKKNKKKEIVTVEAKE |
| | KFDAICKYQPRLYKENKEYAYLLRDIVRGK |
| | SLVELQQIRQEIFRFIEQDCGVTRLGSLSL |
| | STLETVKAVKGIIYSYFSTALNASKNNPIS |
| | DEQRKEFDPELFALLEKLELIRTRKKKQKV |
| | ERIANSLIQTCLENNIKFIRGEGDLSTINN |
| | ATKKKANSRSMDWLARGVENKIRQLAPMHN |
| | ITLFGCGSLYTSHQDPLVHRNPDKAMKCRW |
| | AAIPVKDIGRWVLRKLSQNLRAKNRGTGEY |
| | YHQGVKEFLSHYELQDLEEELLKWRSDRKS |
| | NIPCWVLQNRLAEKLGNKEAVVYIPVRGGR |
| | IYFATHKVATGAVSIVFDQKQVWVCNADHV |
| | AAANIALTGKGIGEQSSDEENPDGSRIKLQ |
| | LTS |

In some embodiments, a nuclease of the present invention is a Cas12i2 domain having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity to the amino acid sequence of any one of SEQ ID NO: 1 and SEQ ID NOs: 39-43. Homology or identity can be determined by amino acid sequence alignment, e.g., using a program such as BLAST, ALIGN, or CLUSTAL, as described herein. In some embodiments, a Cas12i2 domain having a specified degree of amino acid sequence identity to one or more reference polypeptides retains one or more characteristics, e.g., nuclease activity and/or DNA binding activity, as the one or more reference polypeptides.

Also provided is a Cas12i2 domain of the present invention having enzymatic activity, e.g., nuclease activity, and comprising an amino acid sequence which differs from the amino acid sequences of any one of any one of SEQ ID NO: 1 and SEQ ID NOs: 39-43 by no more than 50, no more than 40, no more than 35, no more than 30, no more than 25, no more than 20, no more than 19, no more than 18, no more than 17, no more than 16, no more than 15, no more than 14, no more than 13, no more than 12, no more than 11, no more than 10, no more than 9, no more than 8, no more than 7, no more than 6, no more than 5, no more than 4, no more than 3, no more than 2, or no more than 1 amino acid residue(s), when aligned using any of the previously described alignment methods.

In some embodiments, a Cas12i2 domain of the present invention comprises a RuvC domain. In some embodiments, a Cas12i2 domain of the present invention comprises a split RuvC domain or two or more partial RuvC domains. For example, a Cas12i2 domain comprises RuvC motifs that are not contiguous with respect to the primary amino acid sequence of the Cas12i2 domain but form a RuvC domain once the protein folds. In some embodiments, the catalytic residue of a RuvC motif is a glutamic acid residue and/or an aspartic acid residue. For example, in some embodiments, the nuclease of SEQ ID NO: 1 comprises one or more of the following catalytic residues: D599, E833, and D1019.

In some embodiments, the invention includes an isolated, recombinant, substantially pure, or non-naturally occurring Cas12i2 fusion protein comprising a Cas12i2 domain comprising a RuvC domain, wherein the Cas12i2 domain has enzymatic activity, e.g., nuclease activity, wherein the Cas12i2 domain comprises an amino acid sequence having at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any one of SEQ ID NO: 1 and SEQ ID NOs: 39-43.

### Biochemical Characteristics

In some embodiments, the biochemistry of a Cas12i2 fusion protein (e.g., a Cas12i2 domain of a Cas12i2 fusion protein) described herein is analyzed using one or more assays. In some embodiments, the biochemical characteristics of a Cas12i2 fusion protein described herein are analyzed in vitro using a purified nuclease incubated with an RNA guide (e.g., a mature crRNA) and a target DNA molecule. In some embodiments, the biochemical characteristics of a Cas12i2 fusion protein described herein are analyzed in vitro using a fluorescence depletion assay. In some embodiments, the biochemical characteristics of a Cas12i2 fusion protein described herein are analyzed in mammalian cells, as described in Example 1.

Described herein are Cas12i2 fusion proteins, compositions, and methods relating to a Cas12i2 fusion protein of the present invention. The compositions and methods are based, in part, on the observation that cloned and expressed polypeptides of the present invention have nuclease activity.

In some embodiments, a Cas12i2 fusion protein and an RNA guide as described herein form a complex (e.g., an RNP). In some embodiments, the complex includes other components. In some embodiments, the complex is activated upon binding to a target nucleic acid, e.g., to a target strand of a target nucleic acid, that has complementarity to a spacer sequence in the RNA guide. In some embodiments, the target nucleic acid is a double-stranded DNA (dsDNA). In some embodiments, the target nucleic acid is a single-stranded DNA (ssDNA). In some embodiments, the target nucleic acid is a single-stranded RNA (ssRNA). In some embodiments, the target nucleic acid is a double-stranded RNA (dsRNA). In some embodiments, the sequence-specificity requires a complete match of the spacer sequence in the RNA guide to the target nucleic acid, e.g., to a target strand of the target nucleic acid. In other embodiments, the sequence specificity requires a partial (contiguous or non-contiguous) match of the spacer sequence in the RNA guide to the target nucleic acid, e.g., to a target strand of the target nucleic acid.

In some embodiments, the complex becomes activated upon binding to the target nucleic acid. In some embodiments, the activated complex exhibits “multiple turnover” activity, whereby upon acting on (e.g., cleaving) the target nucleic acid, the activated complex remains in an activated state. In some embodiments, the activated complex exhibits “single turnover” activity, whereby upon acting on the target nucleic acid, the complex reverts to an inactive state.

In some embodiments, a Cas12i2 fusion protein described herein comes into contact with a target nucleic acid at a sequence defined by the region of complementarity between the RNA guide and the target nucleic acid. In some embodiments, the PAM sequence of a Cas12i2 fusion protein described herein is located directly upstream of the target sequence of the target nucleic acid (e.g., directly 5′ of the target sequence). In some embodiments, the PAM sequence of a Cas12i2 fusion protein described herein is located directly 5′ of the target sequence on the non-spacer-complementary strand (e.g., non-target strand) of the target nucleic acid.

In some embodiments, a nuclease of the present invention targets a sequence adjacent to a PAM, wherein the PAM comprises a nucleotide sequence set forth as 5′-TTN-3′, 5′-TTH-3′, 5′-TTY-3′, or 5′-TTC-3′, wherein “N” is any nucleobase, “H” is A, C, or T, and “Y” is C or T. In some embodiments, a Cas12i2 fusion protein (e.g., a Cas12i2 domain) described herein cleaves ssDNA. In some embodiments, a Cas12i2 fusion protein described herein cleaves dsDNA. In some embodiments, a Cas12i2 fusion protein described herein is a nickase (e.g., the Cas12i2 domain cleaves one strand of a double-stranded target nucleic acid).

In some embodiments, a Cas12i2 fusion protein (e.g., the Cas12i2 domain or the fusion domain) of the present invention has enzymatic activity, e.g., nuclease activity, over a broad range of pH conditions. In some embodiments, the Cas12i2 fusion protein has enzymatic activity, e.g., nuclease activity, at a pH of from about 3.0 to about 12.0. In some embodiments, the Cas12i2 fusion protein has enzymatic activity at a pH of from about 4.0 to about 10.5. In some embodiments, the Cas12i2 fusion protein has enzymatic activity at a pH of from about 5.5 to about 8.5. In some embodiments, the Cas12i2 fusion protein has enzymatic activity at a pH of from about 6.0 to about 8.0. In some embodiments, the Cas12i2 fusion protein has enzymatic activity at a pH of about 7.0.

In some embodiments, a Cas12i2 fusion protein (e.g., the Cas12i2 domain or the fusion domain) of the present invention has enzymatic activity, e.g., nuclease activity, at a temperature range of from about 10° C. to about 100° C. In some embodiments, a Cas12i2 fusion protein of the present invention has enzymatic activity at a temperature range from about 20° C. to about 90° C. In some embodiments, a Cas12i2 fusion protein of the present invention has enzymatic activity at a temperature of about 20° C. to about 25° C. or at a temperature of about 37° C.

In some embodiments wherein a Cas12i2 fusion protein (e.g., the Cas12i2 domain or the fusion domain) of the present invention induces double-stranded breaks or single-stranded breaks in a target nucleic acid, (e.g. genomic DNA), the double-stranded break can stimulate cellular endogenous DNA-repair pathways, including Homology Directed Recombination (HDR), Non-Homologous End Joining (NHEJ), or Alternative Non-Homologues End-Joining (A-NHEJ). NHEJ can repair cleaved target nucleic acid without the need for a homologous template. This can result in deletion or insertion of one or more nucleotides at the target locus. HDR can occur with a homologous template, such as the donor DNA. The homologous template can comprise sequences that are homologous to sequences flanking the target nucleic acid cleavage site. In some cases, HDR can insert an exogenous polynucleotide sequence into the cleave target locus. The modifications of the target DNA due to NHEJ and/or HDR can lead to, for example, mutations, deletions, alterations, integrations, gene correction, gene replacement, gene tagging, transgene knock-in, gene disruption, and/or gene knock-outs.

In some embodiments, binding of a Cas12i2 fusion protein/RNA guide complex to a target locus in a cell recruits one or more endogenous cellular molecules or pathways other than DNA repair pathways to modify the target nucleic acid. In some embodiments, binding of a Cas12i2 fusion protein/RNA guide complex blocks access of one or more endogenous cellular molecules or pathways to the target nucleic acid, thereby modifying the target nucleic acid. For example, binding of a Cas12i2 fusion protein/RNA guide complex may block endogenous transcription or translation machinery to decrease the expression of the target nucleic acid.

### Variants

In some embodiments, the present invention includes variants of a Cas12i2 domain described herein. In some embodiments, a Cas12i2 domain described herein can be mutated at one or more amino acid residues to modify one or more functional activities. For example, in some embodiments, a Cas12i2 domain of the present invention is mutated at one or more amino acid residues to modify its nuclease activity (e.g., cleavage activity). For example, in some embodiments, a Cas12i2 domain may comprise one or more mutations that increase the ability of the Cas12i2 domain to cleave a target nucleic acid. In some embodiments, a Cas12i2 domain is mutated at one or more amino acid residues to modify its ability to functionally associate with an RNA guide. In some embodiments, a Cas12i2 domain is mutated at one or more amino acid residues to modify its ability to functionally associate with a target nucleic acid.

In some embodiments, a variant Cas12i2 domain has a conservative or non-conservative amino acid substitution, deletion or addition. In some embodiments, the variant Cas12i2 domain has a silent substitution, deletion or addition, or a conservative substitution, none of which alter the polypeptide activity of the present invention. Typical examples of the conservative substitution include substitution whereby one amino acid is exchanged for another, such as exchange among aliphatic amino acids Ala, Val, Leu and Ile, exchange between hydroxyl residues Ser and Thr, exchange between acidic residues Asp and Glu, substitution between amide residues Asn and Gln, exchange between basic residues Lys and Arg, and substitution between aromatic residues Phe and Tyr. In some embodiments, one or more residues of a Cas12i2 domain disclosed herein are mutated to an Arg residue. In some embodiments, one or more residues of a Cas12i2 domain disclosed herein are mutated to a Gly residue.

A variety of methods are known in the art that are suitable for generating modified polynucleotides that encode variant Cas12i2 domains of the invention, including, but not limited to, for example, site-saturation mutagenesis, scanning mutagenesis, insertional mutagenesis, deletion mutagenesis, random mutagenesis, site-directed mutagenesis, and directed-evolution, as well as various other recombinatorial approaches. Methods for making modified polynucleotides and proteins (e.g., nucleases) include DNA shuffling methodologies, methods based on non-homologous recombination of genes, such as ITCHY (See, Ostermeier et al., 7:2139-44 [1999]), SCRACHY (See, Lutz et al. 98:11248-53 [2001]), SHIPREC (See, Sieber et al., 19:456-60 [2001]), and NRR (See, Bittker et al., 20:1024-9 [2001]; Bittker et al., 101:7011-6 [2004]), and methods that rely on the use of oligonucleotides to insert random and targeted mutations, deletions and/or insertions (See, Ness et al., 20:1251-5 [2002]; Coco et al., 20:1246-50 [2002]; Zha et al., 4:34-9 [2003]; Glaser et al., 149:3903-13 [1992]).

In some embodiments, a Cas12i2 domain of the present invention comprises an alteration at one or more (e.g., several) amino acids, wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 162, 164, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, or more.

In some embodiments, a variant Cas12i2 domain comprises one or more of the amino acid substitutions listed in Table 2 relative to the sequence of SEQ ID NO: 1. In some embodiments, the variant Cas12i2 domain comprises at least one of a D581, G624, F626, D835, L836, P868, S879, D911, 1926, V1020, V1030, E1035, and S1046 substitution. In some embodiments, the variant Cas12i2 domain comprises at least one of a D581R, G624R, F626R, D835R, L836R, P868R, S879R, D911R, I926R, V1020R, V1030R, E1035R, and S1046R substitution. In some embodiments, the variant Cas12i2 domain comprises at least one of a D581G, G624G, F626G, D835G, L836G, P868G, S879G, D911G, I926G, V1020G, V1030G, and S1046G substitution. In some embodiments, the variant Cas12i2 domain comprises at least one of a D581R, G624R, F626R, D835R, L836R, P868R, S879R, D911R, I926R, V1020G, V1030G, E1035R, and S1046G substitution and at least one additional substitution listed in Table 2.

TABLE 2
Single Amino Acid Substitutions in Cas12i2 Domain Variants.

| | Wild-Type | |
|---|---|---|
| Position | Residue | Substitution(s) |
| 1 | M | |
| 2 | S | R, G, A, K, Q, N, H |
| 3 | S | R, G, A, K, Q, N, H |
| 4 | A | R, G, K, Q, N, H |
| 5 | I | R, G, A, K, Q, N, H, W, F, Y, M |
| 6 | K | R, G, A, Q, N, H |
| 7 | S | P, R, G, A, K, Q, N, H |
| 8 | Y | R, G, A, K, Q, N, H |
| 9 | K | R, G, A, Q, N, H |
| 10 | S | R, G, A, K, Q, N, H |
| 11 | V | R, K, G, A, Q, N, H |
| 12 | L | R, G, A, K, Q, N, H |
| 13 | R | A, G, K, Q, N, H |
| 14 | P | R, G, A, K, Q, N, H |
| 15 | N | R, D, G, A, K, Q, H |
| 16 | E | R, D, G, A, K, Q, N, H |
| 17 | R | G, A, K, Q, N, H |
| 18 | K | R, G, A, Q, N, H |
| 19 | N | R, G, A, K, Q, H |
| 20 | Q | R, K, G, A, N, H |
| 21 | L | R, M, G, A, K, Q, N, H |
| 22 | L | R, G, A, K, Q, N, H |
| 23 | K | R, N, G, A, Q, H |
| 24 | S | R, D, G, A, K, Q, N, H |
| 25 | T | R, G, A, K, Q, N, H |
| 26 | I | R, F, G, A, K, Q, N, H |
| 27 | Q | R, N, G, A, K, H |
| 28 | C | R, W, G, A, K, Q, N, H |
| 29 | L | R, G, A, K, Q, N, H |
| 30 | E | R, D, G, A, K, Q, N, H |
| 31 | D | R, G, A, K, Q, N, H |
| 32 | G | R, A, K, Q, N, H |
| 33 | S | R, G, A, K, Q, N, H |
| 34 | A | R, G, K, Q, N, H |
| 35 | F | R, V, G, A, K, Q, N, H |
| 36 | F | R, G, A, K, Q, N, H |
| 37 | F | R, G, A, K, Q, N, H |
| 38 | K | R, D, G, A, Q, N, H |
| 39 | M | R, G, A, K, Q, N, H |
| 40 | L | R, G, A, K, Q, N, H |
| 41 | Q | R, V, G, A, K, N, H |
| 42 | G | R, A, K, Q, N, H |
| 43 | L | R, G, A, K, Q, N, H |
| 44 | F | R, G, A, K, Q, N, H |
| 45 | G | R, A, K, Q, N, H |
| 46 | G | R, A, K, Q, N, H |
| 47 | I | R, G, A, K, Q, N, H |
| 48 | T | R, G, A, K, Q, N, H |
| 49 | P | R, H, G, A, K, Q, N |
| 50 | E | R, G, A, K, Q, N, H |
| 51 | I | R, G, A, K, Q, N, H |
| 52 | V | R, A, G, K, Q, N, H |
| 53 | R | G, A, K, Q, N, H |
| 54 | F | R, G, A, K, Q, N, H |
| 55 | S | R, G, A, K, Q, N, H |
| 56 | T | R, G, A, K, Q, N, H |
| 57 | E | R, G, A, K, Q, N, H |
| 58 | Q | R, G, A, K, N, H |
| 59 | E | R, G, A, K, Q, N, H |
| 60 | K | R, G, A, Q, N, H |
| 61 | Q | R, S, G, A, K, N, H |
| 62 | Q | R, K, G, A, K, N, H |
| 63 | Q | R, G, A, K, N, H |
| 64 | D | R, G, A, K, Q, N, H |
| 65 | I | R, G, A, K, Q, N, H |
| 66 | A | R, D, G, K, Q, N, H |
| 67 | L | R, G, A, K, Q, N, H |
| 68 | W | R, L, G, A, K, Q, N, H |
| 69 | C | R, G, A, K, Q, N, H |
| 70 | A | R, G, K, Q, N, H |
| 71 | V | R, I, G, A, K, Q, N, H |
| 72 | N | R, G, A, K, Q, H |
| 73 | W | R, G, A, K, Q, N, H |
| 74 | F | R, G, A, K, Q, N, H |
| 75 | R | G, A, K, Q, N, H |
| 76 | P | R, L, G, A, K, Q, N, H |
| 77 | V | R, G, A, K, Q, N, H |
| 78 | S | R, G, A, K, Q, N, H |
| 79 | Q | R, K, G, A, N, H |
| 80 | D | R, G, A, K, Q, N, H |
| 81 | S | R, G, A, K, Q, N, H |
| 82 | L | R, G, A, K, Q, N, H |
| 83 | T | R, G, A, K, Q, N, H |
| 84 | H | R, G, A, K, Q, N |
| 85 | T | R, G, A, K, Q, N, H |
| 86 | I | R, Q, G, A, K, N, H |
| 87 | A | R, T, G, K, Q, N, H |
| 88 | S | R, G, A, K, Q, N, H |
| 89 | D | R, E, G, A, K, Q, N, H |
| 90 | N | R, G, A, K, Q, H |
| 91 | L | R, G, A, K, Q, N, H |
| 92 | V | R, G, A, K, Q, N, H |
| 93 | E | R, G, A, K, Q, N, H |
| 94 | K | R, L, G, A, Q, N, H |
| 95 | F | R, G, A, K, Q, N, H |
| 96 | E | R, G, A, K, Q, N, H |
| 97 | E | R, G, A, K, Q, N, H |
| 98 | Y | R, G, A, K, Q, N, H |
| 99 | Y | R, S, G, A, K, Q, N, H |
| 100 | G | R, A, K, Q, N, H |
| 101 | G | R, H, A, K, Q, N |
| 102 | T | R, E, G, A, K, Q, N, H |
| 103 | A | R, P, G, K, Q, N, H |
| 104 | S | R, G, A, K, Q, N, H |
| 105 | D | R, S, G, A, K, Q, N, H |
| 106 | A | R, F, G, K, Q, N, H |
| 107 | I | R, G, A, K, Q, N, H |
| 108 | K | R, Q, G, A, N, H |
| 109 | Q | R, E, G, A, K, N, H |
| 110 | Y | R, G, A, K, Q, N, H |
| 111 | F | R, G, A, K, Q, N, H |
| 112 | S | R, G, A, K, Q, N, H |
| 113 | A | R, G, K, Q, N, H |
| 114 | S | R, N, G, A, K, Q, N, H |
| 115 | I | R, G, A, K, Q, N, H |
| 116 | G | R, D, A, K, Q, N, H |
| 117 | E | R, G, A, K, Q, N, H |
| 118 | S | R, K, G, A, K, Q, N, H |
| 119 | Y | R, G, A, K, Q, N, H |
| 120 | Y | R, G, A, K, Q, N, H |
| 121 | W | R, G, A, K, Q, N, H |
| 122 | N | R, V, G, A, K, Q, H |
| 123 | D | R, G, A, K, Q, N, H |
| 124 | C | R, G, A, K, Q, N, H |
| 125 | R | G, A, K, Q, N, H |
| 126 | Q | R, L, G, A, K, N, H |
| 127 | Q | R, G, A, K, N, H |
| 128 | Y | R, F, G, A, K, Q, N, H |
| 129 | Y | R, G, A, K, Q, N, H |
| 130 | D | R, G, A, K, Q, N, H |
| 131 | L | R, G, A, K, Q, N, H |
| 132 | C | R, A, G, K, Q, N, H |
| 133 | R | G, A, K, Q, N, H |
| 134 | E | R, N, G, A, K, Q, N, H |
| 135 | L | R, I, G, A, K, Q, N, H |
| 136 | G | R, A, K, Q, N, H |
| 137 | V | R, G, A, K, Q, N, H |
| 138 | E | R, S, G, A, K, Q, N, H |
| 139 | V | R, G, A, K, Q, N, H |
| 140 | S | R, G, A, K, Q, N, H |
| 141 | D | R, G, A, K, Q, N, H |
| 142 | L | R, G, A, K, Q, N, H |
| 143 | T | R, G, A, K, Q, N, H |
| 144 | H | R, G, A, K, Q, N |
| 145 | D | R, G, A, K, Q, N, H |
| 146 | L | R, A, G, K, Q, N, H |
| 147 | E | R, G, A, K, Q, N, H |
| 148 | I | R, T, G, A, K, Q, N, H |
| 149 | L | R, M, G, A, K, Q, N, H |
| 150 | C | R, G, A, K, Q, N, H |
| 151 | R | G, A, K, Q, N, H |
| 152 | E | R, G, A, K, Q, N, H |
| 153 | K | R, G, A, Q, N, H |
| 154 | C | R, L, G, A, K, Q, N, H |
| 155 | L | R, I, G, A, K, Q, N, H |
| 156 | A | R, P, G, K, Q, N, H |
| 157 | V | R, L, G, A, K, Q, N, H |
| 158 | A | R, G, K, Q, N, H |
| 159 | T | R, K, G, A, Q, N, H |
| 160 | E | R, D, G, A, K, Q, N, H, L |
| 161 | S | R, D, G, A, K, Q, N, H |
| 162 | N | R, F, G, A, K, Q, H |
| 163 | Q | R, N, G, A, K, H, M |
| 164 | N | R, A, G, K, Q, H, M, W, F |
| 165 | N | R, G, A, K, Q, H, M, P, F, Y, W |
| 166 | S | R, G, A, K, Q, N, H |
| 167 | I | R, G, A, K, Q, N, H |
| 168 | I | R, G, A, K, Q, N, H |
| 169 | S | R, G, A, K, Q, N, H |
| 170 | V | R, G, A, K, Q, N, H |
| 171 | L | R, G, A, K, Q, N, H |
| 172 | F | R, G, A, K, Q, N, H |
| 173 | G | R, A, K, Q, N, H, S |
| 174 | T | R, G, A, K, Q, N, H |
| 175 | G | R, A, K, Q, N, H |
| 176 | E | R, K, G, A, K, Q, N, H |
| 177 | K | R, G, A, Q, N, H |
| 178 | E | R, G, A, K, Q, N, H |
| 179 | D | R, G, A, K, Q, N, H |
| 180 | R | G, A, K, Q, N, H |
| 181 | S | R, G, A, K, Q, N, H |
| 182 | V | R, G, A, K, Q, N, H |
| 183 | K | R, G, A, Q, N, H |
| 184 | L | R, A, G, K, Q, N, H |
| 185 | R | G, A, K, Q, N, H |
| 186 | I | R, M, G, A, K, Q, N, H |
| 187 | T | R, L, G, A, K, Q, N, H |
| 188 | K | R, G, A, Q, N, H |
| 189 | K | R, G, A, Q, N, H |
| 190 | I | R, G, A, K, Q, N, H |
| 191 | L | R, S, G, A, K, Q, N, H |
| 192 | E | R, N, G, A, K, Q, H |
| 193 | A | R, G, K, Q, N, H |
| 194 | I | R, L, G, A, K, Q, N, H |
| 195 | S | R, G, A, K, Q, N, H |
| 196 | N | R, G, A, K, Q, H |
| 197 | L | R, G, A, K, Q, N, H |
| 198 | K | R, D, G, A, Q, N, H |
| 199 | E | R, K, G, A, Q, N, H |
| 200 | I | R, G, A, K, Q, N, H |
| 201 | P | R, G, A, K, Q, N, H |
| 202 | K | R, G, A, Q, N, H |
| 203 | N | R, T, G, A, K, Q, H |
| 204 | V | R, W, G, A, K, Q, N, H |
| 205 | A | R, E, G, K, Q, N, H |
| 206 | P | R, E, G, A, K, Q, N, H |
| 207 | I | R, Y, G, A, K, Q, N, H |
| 208 | Q | R, G, A, K, N, H |
| 209 | E | R, D, G, A, K, Q, N, H |
| 210 | I | R, L, G, A, K, Q, N, H |
| 211 | I | R, G, A, K, Q, N, H |
| 212 | L | R, G, A, K, Q, N, H |
| 213 | N | R, K, G, A, Q, H |
| 214 | V | R, G, A, K, Q, N, H |
| 215 | A | R, F, G, K, Q, N, H |
| 216 | K | R, G, A, Q, N, H |
| 217 | A | R, G, K, Q, N, H |
| 218 | T | R, G, A, K, Q, N, H |
| 219 | K | R, G, A, Q, N, H |
| 220 | E | R, G, A, K, Q, N, H |
| 221 | T | R, L, G, A, K, Q, N, H |
| 222 | F | R, K, G, A, Q, N, H |
| 223 | R | G, A, K, Q, N, H |
| 224 | Q | R, K, G, A, N, H |
| 225 | V | R, G, A, K, Q, N, H |
| 226 | Y | R, G, A, K, Q, N, H |
| 227 | A | R, G, K, Q, N, H |
| 228 | G | R, A, K, Q, N, H |
| 229 | N | R, S, G, A, K, Q, H |
| 230 | L | R, G, A, K, Q, N, H, S |
| 231 | G | R, A, K, Q, N, H |
| 232 | A | R, G, K, Q, N, H, S |
| 233 | P | R, G, A, K, Q, N, H, S |
| 234 | S | R, G, A, K, Q, N, H, P |
| 235 | T | R, S, G, A, K, Q, N, H |
| 236 | L | R, G, A, K, Q, N, H |
| 237 | E | R, V, G, A, K, Q, N, H, S |
| 238 | K | R, G, A, Q, N, H |
| 239 | F | R, D, G, A, K, Q, N, H |
| 240 | I | R, G, A, K, Q, N, H |
| 241 | A | R, K, G, Q, N, H |
| 242 | K | R, G, A, Q, N, H |
| 243 | D | R, G, A, K, Q, N, H |
| 244 | G | R, A, K, Q, N, H |
| 245 | Q | R, G, A, K, N, H |
| 246 | K | R, G, A, Q, N, H |
| 247 | E | R, G, A, K, Q, N, H |
| 248 | F | R, G, A, K, Q, N, H |
| 249 | D | R, G, A, K, Q, N, H |
| 250 | L | R, G, A, K, Q, N, H |
| 251 | K | R, G, A, Q, N, H |
| 252 | K | R, G, A, Q, N, H |
| 253 | L | R, G, A, K, Q, N, H |
| 254 | Q | R, I, G, A, K, N, H |
| 255 | T | R, G, A, K, Q, N, H |
| 256 | D | R, K, G, A, Q, N, H |
| 257 | L | R, F, G, A, K, Q, N, H |
| 258 | K | R, G, A, Q, N, H |
| 259 | K | R, G, A, Q, N, H |
| 260 | V | R, D, G, A, K, Q, N, H |
| 261 | I | R, A, G, K, Q, N, H |
| 262 | R | G, A, K, Q, N, H |
| 263 | G | R, K, A, K, Q, N, H |
| 264 | K | R, G, A, Q, N, H |
| 265 | S | R, G, A, K, Q, N, H |
| 266 | K | R, G, A, Q, N, H |
| 267 | E | R, G, A, K, Q, N, H |
| 268 | R | G, A, K, Q, N, H |
| 269 | D | R, G, A, K, Q, N, H |
| 270 | W | R, L, G, A, K, Q, N, H |
| 271 | C | R, P, G, A, K, Q, N, H |
| 272 | C | R, N, G, A, K, Q, H |
| 273 | Q | R, G, A, K, N, H |
| 274 | E | R, G, A, K, Q, N, H |
| 275 | E | R, K, G, A, Q, N, H |
| 276 | L | R, G, A, K, Q, N, H |
| 277 | R | K, G, A, Q, N, H |
| 278 | S | R, E, G, A, K, Q, N, H |
| 279 | Y | R, G, A, K, Q, N, H |
| 280 | V | R, I, G, A, K, Q, N, H |
| 281 | E | R, A, G, K, Q, N, H |
| 282 | Q | R, S, G, A, K, N, H |
| 283 | N | R, G, A, K, Q, H |
| 284 | T | R, I, G, A, K, Q, N, H |
| 285 | I | R, G, A, K, Q, N, H |
| 286 | Q | R, P, G, A, K, N, H |
| 287 | Y | R, F, G, A, K, Q, N, H |
| 288 | D | R, G, A, K, Q, N, H, S |
| 289 | L | R, Q, G, A, K, N, H, M |
| 290 | W | R, N, G, A, K, Q, H |
| 291 | A | R, S, G, K, Q, N, H |
| 292 | W | R, G, A, K, Q, N, H |
| 293 | G | R, S, A, K, Q, N, H |
| 294 | E | R, A, G, K, Q, N, H |
| 295 | M | R, G, A, K, Q, N, H |
| 296 | F | R, L, G, A, K, Q, N, H |
| 297 | N | R, G, A, K, Q, H |
| 298 | K | R, N, G, A, Q, H |
| 299 | A | R, G, K, Q, N, H |
| 300 | H | R, G, A, K, Q, N |
| 301 | T | R, G, A, K, Q, N, H |
| 302 | A | R, G, K, Q, N, H |
| 303 | L | R, I, G, A, K, Q, N, H |
| 304 | K | R, Q, G, A, N, H |
| 305 | I | R, S, G, A, K, Q, N, H |
| 306 | K | R, G, A, Q, N, H |
| 307 | S | R, N, G, A, K, Q, H |
| 308 | T | R, S, G, A, K, Q, N, H |
| 309 | R | G, A, K, Q, N, H |
| 310 | N | R, G, A, K, Q, H |
| 311 | Y | R, G, A, K, Q, N, H |
| 312 | N | R, L, G, A, K, Q, H |
| 313 | F | R, Y, G, A, K, Q, N, H |
| 314 | A | R, T, G, K, Q, N, H |
| 315 | K | R, G, A, Q, N, H |
| 316 | Q | R, E, G, A, K, N, H |
| 317 | R | K, G, A, Q, N, H |
| 318 | L | R, G, A, K, Q, N, H |
| 319 | E | R, G, A, K, Q, N, H |
| 320 | Q | R, G, A, K, N, H |
| 321 | F | R, G, A, K, Q, N, H |
| 322 | K | R, N, G, A, Q, H |
| 323 | E | R, G, A, K, Q, N, H |
| 324 | I | R, G, A, K, Q, N, H |
| 325 | Q | R, G, A, K, N, H |
| 326 | S | R, G, A, K, Q, N, H |
| 327 | L | R, G, A, K, Q, N, H, V |
| 328 | N | R, D, G, A, K, Q, H, S |
| 329 | N | R, G, A, K, Q, H, S |
| 330 | L | R, G, A, K, Q, N, H |
| 331 | L | R, G, A, K, Q, N, H |
| 332 | V | R, A, G, K, Q, N, H |
| 333 | V | R, G, A, K, Q, N, H |
| 334 | K | R, G, A, Q, N, H |
| 335 | K | R, I, G, A, Q, N, H |
| 336 | L | R, G, A, K, Q, N, H |
| 337 | N | R, G, A, K, Q, H |
| 338 | D | R, G, A, K, Q, N, H |
| 339 | F | R, G, A, K, Q, N, H |
| 340 | F | R, G, A, K, Q, N, H |
| 341 | D | R, G, A, K, Q, N, H |
| 342 | S | G, R, A, K, Q, N, H |
| 343 | E | R, G, A, K, Q, N, H |
| 344 | F | R, G, A, K, Q, N, H |
| 345 | F | R, G, A, K, Q, N, H |
| 346 | S | R, G, A, K, Q, N, H |
| 347 | G | R, A, K, Q, N, H |
| 348 | E | R, G, A, K, Q, N, H, S |
| 349 | E | R, N, G, A, K, Q, H, S |
| 350 | T | R, G, A, K, Q, N, H |
| 351 | Y | R, F, G, A, K, Q, N, H, S |
| 352 | T | R, V, G, A, K, Q, N, H |
| 353 | I | R, V, G, A, K, Q, N, H |
| 354 | C | R, G, A, K, Q, N, H |
| 355 | V | R, K, G, A, Q, N, H |
| 356 | H | R, G, A, K, Q, N |
| 357 | H | R, G, A, K, Q, N, S |
| 358 | L | R, G, A, K, Q, N, H |
| 359 | G | R, A, K, Q, N, H |
| 360 | G | R, A, K, Q, N, H |
| 361 | K | R, G, A, Q, N, H |
| 362 | D | R, G, A, K, Q, N, H |
| 363 | L | R, G, A, K, Q, N, H |
| 364 | S | R, G, A, K, Q, N, H |
| 365 | K | R, E, G, A, Q, N, H |
| 366 | L | R, G, A, K, Q, N, H |
| 367 | Y | R, F, G, A, K, Q, N, H |
| 368 | K | R, G, A, Q, N, H |
| 369 | A | R, I, G, K, Q, N, H |
| 370 | W | R, G, A, K, Q, N, H |
| 371 | E | R, G, A, K, Q, N, H |
| 372 | D | R, E, G, A, K, Q, N, H |
| 373 | D | R, L, G, A, K, Q, N, H |
| 374 | P | R, G, A, K, Q, N, H |
| 375 | A | R, G, K, Q, N, H |
| 376 | D | R, G, A, K, Q, N, H |
| 377 | P | R, M, G, A, K, Q, N, H |
| 378 | E | R, D, G, A, K, Q, N, H |
| 379 | N | R, G, A, K, Q, H |
| 380 | A | R, G, K, Q, N, H |
| 381 | I | R, G, A, K, Q, N, H |
| 382 | V | R, G, A, K, Q, N, H |
| 383 | V | R, G, A, K, Q, N, H, D, E |
| 384 | L | R, Y, G, A, K, Q, N, H |
| 385 | C | R, G, A, K, Q, N, H |
| 386 | D | R, G, A, K, Q, N, H, E, D |
| 387 | D | R, G, A, K, Q, N, H, E |
| 388 | L | R, C, G, A, K, Q, N, H |
| 389 | K | R, G, A, Q, N, H |
| 390 | N | R, D, G, A, K, Q, H |
| 391 | N | R, K, G, A, Q, H |
| 392 | F | R, G, A, K, Q, N, H |
| 393 | K | R, S, G, A, Q, N, H |
| 394 | K | R, G, A, Q, N, H |
| 395 | E | R, G, A, K, Q, N, H, S |
| 396 | P | R, G, A, K, Q, N, H |
| 397 | I | R, G, A, K, Q, N, H, V |
| 398 | R | G, A, K, Q, N, H |
| 399 | N | R, G, A, K, Q, H, S |
| 400 | I | R, L, G, A, K, Q, N, H |
| 401 | L | R, G, A, K, Q, N, H |
| 402 | R | G, A, K, Q, N, H, S |
| 403 | Y | R, G, A, K, Q, N, H |
| 404 | I | R, G, A, K, Q, N, H |
| 405 | F | R, Y, G, A, K, Q, N, H |
| 406 | T | R, G, A, K, Q, N, H |
| 407 | I | R, Y, G, A, K, Q, N, H |
| 408 | R | G, A, K, Q, N, H |
| 409 | Q | R, D, G, A, K, N, H |
| 410 | E | R, K, G, A, Q, N, H |
| 411 | C | R, I, G, A, K, Q, N, H |
| 412 | S | R, G, A, K, Q, N, H |
| 413 | A | R, G, K, Q, N, H |
| 414 | Q | R, K, G, A, N, H |
| 415 | D | R, G, A, K, Q, N, H |
| 416 | I | R, F, G, A, K, Q, N, H |
| 417 | L | R, G, A, K, Q, N, H |
| 418 | A | R, G, K, Q, N, H |
| 419 | A | R, D, G, K, Q, N, H |
| 420 | A | R, G, K, Q, N, H |
| 421 | K | R, G, A, Q, N, H |
| 422 | Y | R, G, A, K, Q, N, H |
| 423 | N | R, G, A, K, Q, H |
| 424 | Q | R, G, A, K, N, H |
| 425 | Q | R, L, G, A, K, N, H |
| 426 | L | R, G, A, K, Q, N, H |
| 427 | D | R, E, G, A, K, Q, N, H |
| 428 | R | K, G, A, Q, N, H |
| 429 | Y | R, N, G, A, K, Q, H |
| 430 | K | R, G, A, Q, N, H |
| 431 | S | R, G, A, K, Q, N, H |
| 432 | Q | R, K, G, A, K, N, H |
| 433 | K | R, G, A, Q, N, H, S |
| 434 | A | R, I, G, K, Q, N, H |
| 435 | N | R, H, G, A, K, Q |
| 436 | P | R, G, A, K, Q, N, H |
| 437 | S | R, T, G, A, K, Q, N, H |
| 438 | V | R, G, A, K, Q, N, H |
| 439 | L | R, G, A, K, Q, N, H |
| 440 | G | R, A, K, Q, N, H |
| 441 | N | R, G, A, K, Q, H |
| 442 | Q | R, T, G, A, K, N, H |
| 443 | G | R, A, K, Q, N, H |
| 444 | F | R, G, A, K, Q, N, H |
| 445 | T | R, N, G, A, K, Q, H |
| 446 | W | R, G, A, K, Q, N, H |
| 447 | T | R, G, A, K, Q, N, H, S, M |
| 448 | N | R, G, A, K, Q, H |
| 449 | A | R, S, G, K, Q, N, H |
| 450 | V | R, T, G, A, K, Q, N, H |
| 451 | I | R, G, A, K, Q, N, H |
| 452 | L | R, T, G, A, K, Q, N, H |
| 453 | P | R, G, A, K, Q, N, H |
| 454 | E | R, P, G, A, K, Q, N, H |
| 455 | K | R, N, G, A, Q, H |
| 456 | A | R, G, K, Q, N, H |
| 457 | Q | R, G, A, K, N, H |
| 458 | R | V, G, A, K, Q, N, H |
| 459 | N | R, K, G, A, Q, H |
| 460 | D | R, G, A, K, Q, N, H |
| 461 | R | G, A, K, Q, N, H |
| 462 | P | R, G, A, K, Q, N, H |
| 463 | N | R, A, G, K, Q, H |
| 464 | S | R, G, A, K, Q, N, H |
| 465 | L | R, S, G, A, K, Q, N, H |
| 466 | D | R, G, A, K, Q, N, H |
| 467 | L | R, G, A, K, Q, N, H |
| 468 | R | M, G, A, K, Q, N, H |
| 469 | I | R, G, A, K, Q, N, H |
| 470 | W | R, G, A, K, Q, N, H |
| 471 | L | R, V, G, A, K, Q, N, H |
| 472 | Y | R, T, G, A, K, Q, N, H |
| 473 | L | R, M, G, A, K, Q, N, H |
| 474 | K | R, T, G, A, Q, N, H |
| 475 | L | R, V, G, A, K, Q, N, H |
| 476 | R | G, A, K, Q, N, H |
| 477 | H | R, G, A, K, Q, N |
| 478 | P | R, D, G, A, K, Q, N, H |
| 479 | D | R, N, G, A, K, Q, H |
| 480 | G | R, A, K, Q, N, H |
| 481 | R | G, A, K, Q, N, H |
| 482 | W | R, G, A, K, Q, N, H |
| 483 | K | R, G, A, Q, N, H |
| 484 | K | R, G, A, Q, N, H |
| 485 | H | R, G, A, K, Q, N |
| 486 | H | R, G, A, K, Q, N |
| 487 | I | R, G, A, K, Q, N, H |
| 488 | P | R, G, A, K, Q, N, H |
| 489 | F | R, G, A, K, Q, N, H |
| 490 | Y | R, H, G, A, K, Q, N |
| 491 | D | R, N, G, A, K, Q, N, H |
| 492 | T | R, S, G, A, K, Q, N, H |
| 493 | R | G, A, K, Q, N, H |
| 494 | F | R, Y, G, A, K, Q, N, H |
| 495 | F | R, Y, G, A, K, Q, N, H |
| 496 | Q | R, E, G, A, K, N, H, S |
| 497 | E | R, G, A, K, Q, N, H |
| 498 | I | R, G, A, K, Q, N, H |
| 499 | Y | R, G, A, K, Q, N, H |
| 500 | A | R, G, K, Q, N, H |
| 501 | A | R, Y, G, K, Q, N, H |
| 502 | G | R, A, K, Q, N, H |
| 503 | N | R, G, A, K, Q, H |
| 504 | S | R, G, G, A, K, Q, N, H |
| 505 | P | R, L, G, A, K, Q, N, H |
| 506 | V | R, P, G, A, K, Q, N, H |
| 507 | D | R, T, G, A, K, Q, N, H |
| 508 | T | R, G, A, K, Q, N, H |
| 509 | C | R, G, A, K, Q, N, H |
| 510 | Q | R, G, A, K, N, H |
| 511 | F | R, P, G, A, K, Q, N, H |
| 512 | R | G, A, K, Q, N, H |
| 513 | T | R, G, A, K, Q, N, H |
| 514 | P | R, K, G, A, K, Q, N, H |
| 515 | R | G, A, K, Q, N, H |
| 516 | F | R, G, A, K, Q, N, H |
| 517 | G | R, A, K, Q, N, H |
| 518 | Y | R, G, A, K, Q, N, H |
| 519 | H | R, G, A, K, Q, N |
| 520 | L | R, G, A, K, Q, N, H |
| 521 | P | R, G, A, K, Q, N, H |
| 522 | K | R, G, A, Q, N, H |
| 523 | L | R, I, G, A, K, Q, N, H |
| 524 | T | R, S, G, A, K, Q, N, H |
| 525 | D | R, G, A, K, Q, N, H |
| 526 | Q | R, G, A, K, N, H |
| 527 | T | R, G, A, K, Q, N, H |
| 528 | A | R, G, K, Q, N, H |
| 529 | I | R, G, A, K, Q, N, H |
| 530 | R | G, A, K, Q, N, H |
| 531 | V | R, L, G, A, K, Q, N, H |
| 532 | N | R, K, G, A, Q, H |
| 533 | K | R, G, A, Q, N, H |
| 534 | K | R, G, A, Q, N, H |
| 535 | H | R, G, A, K, Q, N |
| 536 | V | R, G, A, K, Q, N, H |
| 537 | K | R, G, A, Q, N, H |
| 538 | A | R, G, K, Q, N, H |
| 539 | A | R, G, K, Q, N, H |
| 540 | K | R, G, A, Q, N, H |
| 541 | T | R, G, A, K, Q, N, H, Y |
| 542 | E | R, G, A, K, Q, N, H |
| 543 | A | R, G, K, Q, N, H |
| 544 | R | G, A, K, Q, N, H |
| 545 | I | R, G, A, K, Q, N, H |
| 546 | R | G, A, K, Q, N, H |
| 547 | L | R, G, A, K, Q, N, H |
| 548 | A | R, N, G, K, Q, N, H |
| 549 | I | R, G, A, K, Q, N, H |
| 550 | Q | R, G, A, K, N, H |
| 551 | Q | R, G, A, K, N, H |
| 552 | G | R, N, A, K, Q, N, H |
| 553 | T | R, V, G, A, K, Q, N, H |
| 554 | L | R, A, G, A, K, Q, N, H |
| 555 | P | R, G, A, K, Q, N, H |
| 556 | V | R, G, A, K, Q, N, H |
| 557 | S | R, G, A, K, Q, N, H |
| 558 | N | R, G, A, K, Q, H |
| 559 | L | R, F, G, A, K, Q, N, H |
| 560 | K | R, D, G, A, Q, N, H |
| 561 | I | R, G, A, K, Q, N, H |
| 562 | T | R, G, A, K, Q, N, H |
| 563 | E | R, G, A, K, Q, N, H |
| 564 | I | R, T, G, A, K, Q, N, H |
| 565 | S | R, N, G, A, K, Q, H |
| 566 | A | R, F, G, K, Q, N, H |
| 567 | T | R, G, A, K, Q, N, H |
| 568 | I | R, V, G, A, K, Q, N, H |
| 569 | N | R, G, A, K, Q, H |
| 570 | S | R, G, A, K, Q, N, H |
| 571 | K | R, G, A, Q, N, H |
| 572 | G | R, N, A, K, Q, N, H |
| 573 | Q | R, D, G, A, K, N, H |
| 574 | V | R, I, G, A, K, Q, N, H |
| 575 | R | T, H, K, N, G, A, Q |
| 576 | I | R, H, K, N, G, A, Q |
| 577 | P | R, S, H, K, N, G, A, Q, T, |
| | | V, Y, F, L |
| 578 | V | R, H, K, N, G, A, Q |
| 579 | K | R, H, N, G, A, Q |
| 580 | F | R, V, H, K, N, G, A, Q |
| 581 | D | R, H, K, N, G, A, Q |
| 582 | V | R, G, H, K, N, G, A, Q |
| 583 | G | R, H, K, N, A, Q |
| 584 | R | G, K, H, K, N, A, Q |
| 585 | Q | R, G, Y, H, K, N, A |
| 586 | K | R, G, H, N, A, Q |
| 587 | G | R, H, K, NA, Q |
| 588 | T | R, G, K, H, N A, Q |
| 589 | L | R, G, H, K, N A, Q |
| 590 | Q | R, G, H, K, N, A |
| 591 | I | R, G, H, K, N, A, Q |
| 592 | G | R, H, K, N, A, Q |
| 593 | D | R, G, H, K, N, A, Q |
| 594 | R | G, H, K, N, A, Q |
| 595 | F | R, G, I, H, K, N, A, Q |
| 596 | C | R, G, M, H, K, N, A, Q |
| 597 | G | R, H, K, N, A, Q |
| 598 | Y | R, G, H, K, N, A, Q |
| 599 | D | A |
| 600 | Q | R, G, K, N, H, A, D, C, E, I, L, |
| | | M, F, P, S, T, W, Y, V |
| 601 | N | R, G, K, H, A, D, C, Q, E, I, L, |
| | | M, F, P, S, T, W, Y, V |
| 602 | Q | R, G, K, N, H, A, D, C, E, I, L, |
| | | M, F, P, S, T, W, Y, V |
| 603 | T | R, G, K, N, H, A, D, C, Q, E, I, |
| | | L, M, F, P, S, W, Y, V |
| 604 | A | R, G, K, N, H, D, C, Q, E, I, L, |
| | | M, F, P, S, T, W, Y, V |
| 605 | S | R, G, K, N, H, A, D, C, Q, E, I, |
| | | L, M, F, P, T, W, Y, V |
| 606 | H | R, G, K, N, A, D, C, Q, E, I, L, |
| | | M, F, P, S, T, W, Y, V |
| 607 | A | R, G, K, N, H, D, C, Q, E, I, L, |
| | | M, F, P, S, T, W, Y, V |
| 608 | Y | R, G, H, K, N, A, Q |
| 609 | S | R, G, H, K, N, A, Q |
| 610 | L | R, G, I, H, K, N, A, Q |
| 611 | W | R, G, H, K, N, A, Q |
| 612 | E | R, G, H, K, N, A, Q |
| 613 | V | R, G, H, K, N, A, Q |
| 614 | V | R, G, H, K, N, A, Q |
| 615 | K | R, G, E, H, N, A, Q |
| 616 | E | R, G, H, K, N, A, Q |
| 617 | G | R, H, K, N, A, Q |
| 618 | Q | R, G, T, H, K, N, A |
| 619 | Y | R, G, S, H, K, N, A, Q |
| 620 | H | R, G, K, N, A, Q |
| 621 | K | R, G, H, N, A, Q |
| 622 | E | R, G, H, K, N, A, Q |
| 623 | L | R, G, N, H, K, A, Q |
| 624 | G | R, H, K, N, A, Q |
| 625 | C | R, G, H, K, N, A, Q, A, Q |
| 626 | F | R, G, W, H, K, N, A, Q |
| 627 | V | R, G, H, K, N, A, Q |
| 628 | R | G, H, K, N, A, Q |
| 629 | F | R, G, L, H, K, N, A, Q |
| 630 | I | R, G, V, H, K, N, A, Q |
| 631 | S | R, G, E, H, K, N, A, Q |
| 632 | S | R, G, D, H, K, N, A, Q |
| 633 | G | R, H, K, N, A, Q |
| 634 | D | R, G, K, H, K, N, A, Q |
| 635 | I | R, G, H, K, N, A, Q |
| 636 | V | R, G, T, H, K, N, A, Q |
| 637 | S | R, G, H, K, N, A, Q |
| 638 | I | R, G, H, K, N, A, Q |
| 639 | T | R, G, H, K, N, A, Q |
| 640 | E | R, G, H, K, N, A, Q |
| 641 | N | R, G, H, K, A, Q |
| 642 | R | G, H, K, N, A, Q |
| 643 | G | R, H, K, N, A, Q |
| 644 | N | R, G, H, K, A, Q |
| 645 | Q | R, G, H, K, N, A |
| 646 | F | R, G, V, H, K, N, A, Q, Y |
| 647 | D | R, G, H, K, N, A, Q |
| 648 | Q | R, G, H, K, N, A, |
| 649 | L | R, G, H, K, N, A, Q |
| 650 | S | R, G, H, K, N, A, Q |
| 651 | Y | R, G, H, K, N, A, Q |
| 652 | E | R, G, D, H, K, N, A, Q |
| 653 | G | R, H, K, N, A, Q |
| 654 | L | R, G, H, K, N, A, Q |
| 655 | A | R, G, H, K, N, Q |
| 656 | Y | R, G, H, K, N, A, Q |
| 657 | P | R, G, H, K, N, A, Q |
| 658 | Q | R, G, H, K, N, A |
| 659 | Y | R, G, F, H, K, N, A, Q |
| 660 | A | R, G, H, K, N, Q |
| 661 | D | R, G, E, H, K, N, A, Q |
| 662 | W | R, G, H, K, N, A, Q |
| 663 | R | G, H, K, N, A, Q |
| 664 | K | R, G, H, N, A, Q |
| 665 | K | R, G, H, N, A, Q |
| 666 | A | R, G, H, K, N, Q |
| 667 | S | R, G, H, K, N, A, Q |
| 668 | K | R, G, H, N, A, Q |
| 669 | F | R, G, H, K, N, A, Q |
| 670 | V | R, G, L, H, K, N, A, Q |
| 671 | S | R, G, H, K, N, A, Q |
| 672 | L | R, G, S, H, K, N, A, Q |
| 673 | W | R, G, H, K, N, A, Q |
| 674 | Q | R, G, H, K, N, A |
| 675 | I | R, G, H, K, N, A, Q |
| 676 | T | R, G, H, K, N, A, Q |
| 677 | K | R, G, H, N, A, Q |
| 678 | K | R, G, H, N, A, Q |
| 679 | N | R, G, K, N, A, Q |
| 680 | K | R, G, H, N, A, Q |
| 681 | K | R, G, H, N, A, Q |
| 682 | K | R, G, H, N, A, Q |
| 683 | E | R, G, H, K, N, A, Q |
| 684 | I | R, G, D, H, K, N, A, Q |
| 685 | V | R, G, H, K, N, A, Q |
| 686 | T | R, G, H, K, N, A, Q |
| 687 | V | R, G, K, H, N, A, Q |
| 688 | E | R, G, H, K, N, A, Q |
| 689 | A | R, G, H, K, N, Q |
| 690 | K | R, G, H, N, A, Q |
| 691 | E | R, G, H, K, N, A, Q |
| 692 | K | R, G, H, N, A, Q |
| 693 | F | R, G, H, K, N, A, Q |
| 694 | D | R, G, K, H, N, A, Q |
| 695 | A | R, G, H, K, N, Q |
| 696 | I | R, G, H, K, N, A, Q |
| 697 | C | R, G, H, K, N, A, Q |
| 698 | K | R, G, W, H, N, A, Q |
| 699 | Y | R, G, H, K, N, A, Q |
| 700 | Q | R, G, H, K, N, A |
| 701 | P | R, G, H, K, N, A, Q |
| 702 | R | G, H, K, N, A, Q |
| 703 | L | R, G, H, K, N, A, Q |
| 704 | Y | R, G, H, K, N, A, Q |
| 705 | K | R, G, H, N, A, Q |
| 706 | F | R, G, W, H, K, N, A, Q |
| 707 | N | R, G, H, K, A, Q |
| 708 | K | R, G, H, N, A, Q |
| 709 | E | R, G, H, K, N, A, Q |
| 710 | Y | R, G, H, K, N, A, Q |
| 711 | A | R, G, H, K, N, Q |
| 712 | Y | R, G, H, K, N, A, Q |
| 713 | L | R, G, H, K, N, A, Q |
| 714 | L | R, G, H, K, N, A, Q |
| 715 | R | G, L, H, K, N, A, Q |
| 716 | D | R, G, H, K, N, A, Q |
| 717 | I | R, G, H, K, N, A, Q |
| 718 | V | R, G, M, H, K, N, A, Q |
| 719 | R | G, K, H, N, A, Q |
| 720 | G | R, H, K, N, A, Q |
| 721 | K | R, G, N, H, A, Q |
| 722 | S | R, G, H, K, N, A, Q |
| 723 | L | R, G, H, K, N, A, Q |
| 724 | V | R, G, H, K, N, A, Q |
| 725 | E | R, G, N, H, K, A, Q |
| 726 | L | R, G, H, K, N, A, Q |
| 727 | Q | R, G, H, K, N, A |
| 728 | Q | R, G, H, K, N, A |
| 729 | I | R, G, F, H, K, N, A, Q |
| 730 | R | G, H, K, N, A, Q |
| 731 | Q | R, G, A, H, K, N, A |
| 732 | E | R, G, H, K, N, A, Q |
| 733 | I | R, G, H, K, N, A, Q |
| 734 | F | R, G, H, K, N, A, Q |
| 735 | R | G, H, K, N, A, Q |
| 736 | F | R, G, H, K, N, A, Q |
| 737 | I | R, G, H, K, N, A, Q |
| 738 | E | R, G, H, K, N, A, Q |
| 739 | Q | R, G, H, K, N, A |
| 740 | D | R, G, H, K, N, A, Q |
| 741 | C | R, G, F, H, K, N, A, Q, S |
| 742 | G | R, H, K, N, A, Q |
| 743 | V | R, G, H, K, N, A, Q |
| 744 | T | R, G, H, K, N, A, Q |
| 745 | R | G, H, K, N, A, Q |
| 746 | L | R, G, H, K, N, A, Q |
| 747 | G | R, H, K, N, A, Q |
| 748 | S | R, G, H, K, N, A, Q |
| 749 | L | R, G, H, K, N, A, Q |
| 750 | S | R, G, H, K, N, A, Q |
| 751 | L | R, G, H, K, N, A, Q |
| 752 | S | R, G, H, K, N, A, Q |
| 753 | T | R, G, S, H, K, N, A, Q |
| 754 | L | R, G, H, K, N, A, Q |
| 755 | E | R, G, H, K, N, A, Q |
| 756 | T | R, G, H, K, N, A, Q |
| 757 | V | R, G, L, H, K, N, A, Q |
| 758 | K | R, G, H, N, A, Q |
| 759 | A | R, G, N, H, K, Q |
| 760 | V | R, G, H, K, N, A, Q |
| 761 | K | R, G, H, N, A, Q |
| 762 | G | R, S, H, K, N, A, Q |
| 763 | I | R, G, L, H, K, N, A, Q |
| 764 | I | R, G, H, K, N, A, Q |
| 765 | Y | R, G, S, H, K, N, A, Q |
| 766 | S | R, G, H, K, N, A, Q |
| 767 | Y | R, G, H, K, N, A, Q |
| 768 | F | R, G, H, K, N, A, Q |
| 769 | S | R, G, H, K, N, A, Q |
| 770 | T | R, G, L, H, K, N, A, Q |
| 771 | A | R, G, N, H, K, Q |
| 772 | L | R, G, H, K, N, A, Q |
| 773 | N | R, G, H, K, A, Q |
| 774 | A | R, G, N, H, K, Q |
| 775 | S | R, G, H, K, N, A, Q |
| 776 | K | R, G, H, N, A, Q |
| 777 | N | R, G, E, H, K, A, Q |
| 778 | N | R, G, H, K, A, Q |
| 779 | P | R, G, H, K, N, A, Q |
| 780 | I | R, G, H, K, N, A, Q |
| 781 | S | R, G, H, K, N, A, Q |
| 782 | D | R, G, H, K, N, A, Q |
| 783 | E | R, G, H, K, N, A, Q |
| 784 | Q | R, G, D, H, K, N, A |
| 785 | R | G, K, Q, H, N, A, Q |
| 786 | K | R, G, E, H, N, A, Q |
| 787 | E | R, G, H, K, N, A, Q |
| 788 | F | R, G, H, K, N, A, Q |
| 789 | D | R, G, H, K, N, A, Q |
| 790 | P | R, G, H, K, N, A, Q |
| 791 | E | R, G, H, K, N, A, Q |
| 792 | L | R, G, H, K, N, A, Q |
| 793 | F | R, G, H, K, N, A, Q |
| 794 | A | R, G, H, K, N, Q |
| 795 | L | R, G, H, K, N, A, Q |
| 796 | L | R, G, M, H, K, N, A, Q |
| 797 | E | R, G, V, H, K, N, A, Q |
| 798 | K | R, G, H, N, A, Q |
| 799 | L | R, G, I, H, K, N, A, Q |
| 800 | E | R, G, H, K, N, A, Q, S |
| 801 | L | R, G, H, K, N, A, Q |
| 802 | I | R, G, K, H, N, A, Q |
| 803 | R | G, H, K, N, A, Q |
| 804 | T | R, G, H, K, N, A, Q |
| 805 | R | G, N, H, K, A, Q |
| 806 | K | R, G, H, N, A, Q |
| 807 | K | R, G, H, N, A, Q |
| 808 | K | R, G, H, N, A, Q |
| 809 | Q | R, G, E, H, K, N, A, Q |
| 810 | K | R, G, H, N, A, Q |
| 811 | V | R, G, H, K, N, A, Q |
| 812 | E | R, G, S, H, K, N, A, Q |
| 813 | R | G, H, K, N, A, Q |
| 814 | I | R, G, H, K, N, A, Q |
| 815 | A | R, G, S, H, K, N, Q |
| 816 | N | R, G, S, H, K, A, Q |
| 817 | S | R, G, H, K, N, A, Q |
| 818 | L | R, G, H, K, N, A, Q |
| 819 | I | R, G, L, H, K, N, A, Q |
| 820 | Q | R, G, H, K, N, A |
| 821 | T | R, G, I, H, K, N, A, Q |
| 822 | C | R, G, A, H, K, N, A, Q |
| 823 | L | R, G, H, K, N, A, Q |
| 824 | E | R, G, H, K, N, A, Q |
| 825 | N | R, G, H, K, A, Q |
| 826 | N | R, G, H, K, A, Q |
| 827 | I | R, G, V, H, K, N, A, Q |
| 828 | K | R, G, H, N, A, Q |
| 829 | F | R, G, H, K, N, A, Q |
| 830 | I | R, G, H, K, N, A, Q |
| 831 | R | G, V, H, K, N, A, Q |
| 832 | G | R, V, H, K, N, A, Q |
| 833 | E | A |
| 834 | G | R, K, N, H, A, D, C, Q, E, I, |
| | | L, M, F, P, S, T, W, Y, V |
| 835 | D | R, G, K, N, H, A, C, Q, E, I, |
| | | L, M, F, P, S, T, W, Y, V |
| 836 | L | R, G, K, N, H, A, D, C, Q, E, |
| | | I, M, F, P, S, T, W, Y, V |
| 837 | S | R, G, K, N, H, A, D, C, Q, E, |
| | | I, L, M, F, P, T, W, Y, V |
| 838 | T | R, G, K, N, H, A, D, C, Q, E, |
| | | I, L, M, F, P, S, W, Y, V |
| 839 | T | R, G, K, N, H, A, D, C, Q, E, |
| | | I, L, M, F, P, S, W, Y, V |
| 840 | N | R, G, K, H, A, D, C, Q, E, I, |
| | | L, M, F, P, S, T, W, Y, V |
| 841 | N | R, G, K, H, A, D, C, Q, E, I, |
| | | L, M, F, P, S, T, W, Y, V |
| 842 | A | R, G, K, N, H, D, C, Q, E, I, |
| | | L, M, F, P, S, T, W, Y, V |
| 843 | T | R, G, K, N, H, A, D, C, Q, E, |
| | | I, L, M, F, P, S, W, Y, V |
| 844 | K | R, G, N, H, A, D, C, Q, E, I, |
| | | L, M, F, P, S, T, W, Y, V |
| 845 | K | R, G, N, H, A, D, C, Q, E, I, |
| | | L, M, F, P, S, T, W, Y, V |
| 846 | K | R, G, N, H, A, D, C, Q, E, I, |
| | | L, M, F, P, S, T, W, Y, V |
| 847 | A | R, G, K, N, H, D, C, Q, E, I, |
| | | L, M, F, P, S, T, W, Y, V |
| 848 | N | R, G, K, H, A, D, C, Q, E, I, |
| | | L, M, F, P, S, T, W, Y, V |
| 849 | S | R, G, K, N, H, A, D, C, Q, E, |
| | | I, L, M, F, P, T, W, Y, V |
| 850 | R | G, K, N, H, A, D, C, Q, E, |
| | | I, L, M, F, P, S, T, W, Y, V |
| 851 | S | R, G, K, N, H, A, D, C, Q, E, |
| | | I, L, M, F, P, T, W, Y, V |
| 852 | M | R, G, K, N, H, A, D, C, Q, E, |
| | | I, L, F, P, S, T, W, Y, V |
| 853 | D | R, G, K, N, H, A, C, Q, E, I, |
| | | L, M, F, P, S, T, W, Y, V |
| 854 | W | R, G, K, N, H, A, D, C, Q, E, |
| | | I, L, M, F, P, S, T, Y, V |
| 855 | L | R, G, K, N, H, A, D, C, Q, E, |
| | | I, M, F, P, S, T, W, Y, V |
| 856 | A | R, G, K, N, H, D, C, Q, E, I, |
| | | L, M, F, P, S, T, W, Y, V |
| 857 | R | G, K, N, H, A, D, C, Q, E, I, |
| | | L, M, F, P, S, T, W, Y, V |
| 858 | G | R, K, N, H, A, D, C, Q, E, I, |
| | | L, M, F, P, S, T, W, Y, V |
| 859 | V | R, G, K, N, H, A, D, C, Q, E, |
| | | I, L, M, F, P, S, T, W, Y |
| 860 | F | R, G, K, N, H, A, D, C, Q, E, |
| | | I, L, M, P, S, T, W, Y, V |
| 861 | N | R, G, K, H, A, D, C, Q, E, I, |
| | | L, M, F, P, S, T, W, Y, V |
| 862 | K | R, G, N, H, A, D, C, Q, E, I, |
| | | L, M, F, P, S, T, W, Y, V |
| 863 | I | R, G, K, N, H, A, D, C, Q, |
| | | E, L, M, F, P, S, T, W, Y, V |
| 864 | R | G, K, N, H, A, D, C, Q, E, I, |
| | | L, M, F, P, S, T, W, Y, V |
| 865 | Q | R, G, K, N, H, A, D, C, E, I, |
| | | L, M, F, P, S, T, W, Y, V |
| 866 | L | R, G, K, N, H, A, D, C, Q, E, |
| | | I, M, F, P, S, T, W, Y, V |
| 867 | A | R, G, K, N, H, D, C, Q, E, I, |
| | | L, M, F, P, S, T, W, Y, V |
| 868 | P | R, G, K, N, H, A, D, C, Q, E, |
| | | I, L, M, F, S, T, W, Y, V |
| 869 | M | R, G, K, N, H, A, D, C, Q, E, |
| | | I, L, F, P, S, T, W, Y, V |
| 870 | H | R, G, K, N, A, D, C, Q, E, I, |
| | | L, M, F, P, S, T, W, Y, V |
| 871 | N | R, G, K, H, A, D, C, Q, E, I, |
| | | L, M, F, P, S, T, W, Y, V |
| 872 | I | R, G, H, K, N, A, Q |
| 873 | T | R, G, H, K, N, A, Q |
| 874 | L | R, G, H, K, N, A, Q |
| 875 | F | R, G, H, K, N, A, Q |
| 876 | G | R, H, K, N, A, Q |
| 877 | C | R, G, K, N, H, A, D, Q, E, I, |
| | | L, M, F, P, S, T, W, Y, V |
| 878 | G | R, K, N, H, A, D, C, Q, E, I, |
| | | L, M, F, P, S, T, W, Y, V |
| 879 | S | R, G, K, N, H, A, D, C, Q, E, |
| | | I, L, M, F, P, T, W, Y, V |
| 880 | L | R, G, K, N, H, A, D, C, Q, E, |
| | | I, M, F, P, S, T, W, Y, V |
| 881 | Y | R, G, K, N, H, A, D, C, Q, E, |
| | | I, L, M, F, P, S, T, W, V |
| 882 | T | R, G, K, N, H, A, D, C, Q, E, |
| | | I, L, M, F, P, S, W, Y, V |
| 883 | S | R, G, K, N, H, A, D, C, Q, E, |
| | | I, L, M, F, P, T, W, Y, V |
| 884 | H | R, G, K, N, A, D, C, Q, E, I, |
| | | L, M, F, P, S, T, W, Y, V |
| 885 | Q | R, G, K, N, H, A, D, C, E, I, |
| | | L, M, F, P, S, T, W, Y, V |
| 886 | D | R, G, H, K, N, A, Q |
| 887 | P | R, G, H, K, N, A, Q |
| 888 | L | R, G, F, H, K, N, A, Q |
| 889 | V | R, G, H, K, N, A, Q |
| 890 | H | R, G, K, N, A, Q |
| 891 | R | G, H, K, N, A, Q |
| 892 | N | R, G, H, K, A, Q |
| 893 | P | R, G, H, K, N, A, Q |
| 894 | D | R, G, H, K, N, A, Q |
| 895 | K | R, G, H, N, A, Q |
| 896 | A | R, G, H, K, N, Q |
| 897 | M | R, G, K, H, N, A, Q |
| 898 | K | R, E, H, N, A, Q |
| 899 | C | R, G, A, H, K, N, Q |
| 900 | R | G, H, K, N, A, Q |
| 901 | W | R, G, F, Y, H, K, N, A, Q |
| 902 | A | R, G, H, K, N, Q |
| 903 | A | R, G, H, K, N, Q |
| 904 | I | R, G, V, H, K, N, A, Q |
| 905 | P | R, G, H, K, N, A, Q |
| 906 | V | R, G, P, H, K, N, A, Q |
| 907 | K | R, G, S, H, N, A, Q |
| 908 | D | R, G, H, K, N, A, Q |
| 909 | I | R, G, H, K, N, A, Q |
| 910 | G | R, K, H, K, N, A, Q |
| 911 | D | R, G, E, H, K, N, A, Q |
| 912 | W | R, G, Y, H, K, N, A, Q |
| 913 | V | R, G, H, K, N, A, Q |
| 914 | L | R, G, H, K, N, A, Q |
| 915 | R | G, H, K, N, A, Q |
| 916 | K | R, G, H, N, A, Q |
| 917 | L | R, G, F, H, K, N, A, Q |
| 918 | S | R, G, H, K, N, A, Q |
| 919 | Q | R, G, H, K, N, A |
| 920 | N | R, G, W, H, K, A, Q |
| 921 | L | R, G, H, K, N, A, Q |
| 922 | R | G, H, K, N, A, Q |
| 923 | A | R, G, H, K, N, Q |
| 924 | K | R, G, H, N, A, Q |
| 925 | N | R, G, T, H, K, A, Q, D, E |
| 926 | I | R, G, S, H, K, N, A, Q |
| 927 | G | R, T, H, K, N, A, Q |
| 928 | T | R, G, H, K, N, A, Q |
| 929 | G | R, H, K, N, A, Q |
| 930 | E | R, G, H, K, N, A, Q |
| 931 | Y | R, G, L, H, K, N, A, Q |
| 932 | Y | R, G, H, K, N, A, Q |
| 933 | H | R, G, K, N, A, Q |
| 934 | Q | R, G, E, H, K, N, A |
| 935 | G | R, A, H, K, N, A, Q |
| 936 | V | R, G, L, H, K, N, A, Q |
| 937 | K | R, G, H, N, A, Q |
| 938 | E | R, G, H, K, N, A, Q |
| 939 | F | R, G, H, K, N, A, Q |
| 940 | L | R, G, H, K, N, A, Q |
| 941 | S | R, G, H, K, N, A, Q |
| 942 | H | R, G, K, N, A, Q |
| 943 | Y | R, G, H, K, N, A, Q |
| 944 | E | R, G, H, K, N, A, Q |
| 945 | L | R, G, H, K, N, A, Q |
| 946 | Q | R, G, D, H, K, N, A |
| 947 | D | R, G, H, K, N, A, Q |
| 948 | L | R, G, D, H, K, N, A, Q |
| 949 | E | R, G, H, K, N, A, Q |
| 950 | E | R, G, L, H, K, N, A, Q |
| 951 | E | R, G, P, H, K, N, A, Q |
| 952 | L | R, G, K, H, K, N, A, Q |
| 953 | L | R, G, M, H, K, N, A, Q |
| 954 | K | R, G, H, N, A, Q |
| 955 | W | R, G, F, H, K, N, A, Q |
| 956 | R | G, H, K, N, A, Q |
| 957 | S | R, G, H, K, N, A, Q |
| 958 | D | R, G, L, H, K, N, A, Q |
| 959 | R | G, K, H, N, A, Q |
| 960 | K | R, G, H, K, N, A, Q |
| 961 | S | R, G, H, K, N, A, Q |
| 962 | N | R, G, H, K, A, Q |
| 963 | I | R, G, H, K, N, A, Q |
| 964 | P | R, G, H, K, N, A, Q |
| 965 | C | R, G, H, K, N, A, Q |
| 966 | W | R, G, H, K, N, A, Q |
| 967 | V | R, G, H, K, N, A, Q |
| 968 | L | R, G, H, K, N, A, Q |
| 969 | Q | R, G, H, K, N, A |
| 970 | N | R, G, H, K, A, Q |
| 971 | R | G, H, K, N, A, Q |
| 972 | L | G, I, H, K, N, A, Q |
| 973 | A | R, G, L, H, K, N, Q |
| 974 | E | R, G, H, K, N, A, Q |
| 975 | K | R, G, D, H, N, A, Q |
| 976 | L | R, G, H, K, N, A, Q |
| 977 | G | R, H, K, N, A, Q |
| 978 | N | R, G, H, K, A, Q |
| 979 | K | R, G, H, N, A, Q |
| 980 | E | R, G, H, K, N, A, Q |
| 981 | A | R, G, H, K, N, Q |
| 982 | V | R, G, H, K, N, A, Q |
| 983 | V | R, G, H, K, N, A, Q |
| 984 | Y | R, G, I, H, K, N, A, Q |
| 985 | I | R, G, H, K, N, A, Q |
| 986 | P | R, G, H, K, N, A, Q |
| 987 | V | R, G, H, K, N, A, Q |
| 988 | R | G, H, K, N, A, Q |
| 989 | G | R, H, K, N, A, Q |
| 990 | G | R, H, K, N, A, Q |
| 991 | R | G, H, K, N, A, Q |
| 992 | I | R, G, H, K, N, A, Q |
| 993 | Y | R, G, H, K, N, A, Q |
| 994 | F | R, G, L, H, K, N, A, Q |
| 995 | A | R, G, S, H, K, N, A, Q |
| 996 | T | R, G, H, K, N, A, Q |
| 997 | H | R, G, K, N, A, Q |
| 998 | K | R, G, P, H, N, A, Q |
| 999 | V | R, G, H, K, N, A, Q |
| 1000 | A | R, G, T, H, K, N, Q |
| 1001 | T | R, G, H, K, N, A, Q |
| 1002 | G | R, D, H, K, N, A, Q |
| 1003 | A | R, G, S, H, K, N, Q |
| 1004 | V | R, G, S, H, K, N, A, Q |
| 1005 | S | R, G, K, H, N, A, Q |
| 1006 | I | R, G, H, K, N, A, Q |
| 1007 | V | R, G, H, K, N, A, Q |
| 1008 | F | R, G, H, K, N, A, Q |
| 1009 | D | R, G, N, H, K, A, Q |
| 1010 | Q | R, G, H, K, N, A |
| 1011 | K | R, G, H, N, A, Q |
| 1012 | Q | R, G, E, H, K, N, A |
| 1013 | V | R, G, H, K, N, A, Q |
| 1014 | W | R, G, H, K, N, A, Q |
| 1015 | V | R, G, H, K, N, A, Q |
| 1016 | C | R, G, N, H, K, A, Q |
| 1017 | N | R, G, H, K, A, Q |
| 1018 | A | R, G, S, H, K, N, Q |
| 1019 | D | A |
| 1020 | H | R, G, K, N, A, Q |
| 1021 | V | R, G, H, K, N, A, Q |
| 1022 | A | R, G, H, K, N, Q, S |
| 1023 | A | R, G, H, K, N, Q |
| 1024 | A | R, G, V, H, K, N, Q |
| 1025 | N | R, G, H, K, A, Q |
| 1026 | I | R, G, H, K, N, A, Q, V |
| 1027 | A | R, G, V, H, K, N, Q |
| 1028 | L | R, G, H, K, N, A, Q |
| 1029 | T | R, G, H, K, N, A, Q |
| 1030 | V | R, G, H, K, N, A, Q |
| 1031 | K | R, G, H, N, A, Q |
| 1032 | G | R, H, K, N, A, Q |
| 1033 | I | R, G, H, K, N, A, Q |
| 1034 | G | R, H, K, N, A, Q |
| 1035 | E | R, G, I, H, K, N, A, Q |
| 1036 | Q | R, G, H, K, N, A |
| 1037 | S | R, G, H, K, N, A, Q |
| 1038 | S | R, G, E, H, K, N, A, Q |
| 1039 | D | R, G, H, K, N, A, Q |
| 1040 | E | R, G, H, K, N, A, Q |
| 1041 | E | R, G, H, K, N, A, Q |
| 1042 | N | R, G, H, K, A, Q |
| 1043 | P | R, G, H, K, N, A, Q |
| 1044 | D | R, G, K, H, N, A, Q |
| 1045 | G | R, H, K, N, A, Q |
| 1046 | S | R, G, K, H, K, N, A, Q |
| 1047 | R | G, K, H, K, N, A, Q |
| 1048 | I | R, G, H, K, N, A, Q |
| 1049 | K | R, G, H, N, A, Q |
| 1050 | L | R, G, H, K, N, A, Q |
| 1051 | Q | R, G, H, K, N, A, Q |
| 1052 | L | R, G, H, K, N, A, Q |
| 1053 | T | R, G, H, K, N, A, Q |
| 1054 | S | R, G, H, K, N, A, Q |

In some embodiments, the variant Cas12i2 domain of SEQ ID NO: 39 comprises the following mutations relative to SEQ ID NO: 1: D581R D911R I926R V1030G. In some embodiments, the variant Cas12i2 domain of SEQ ID NO: 40 comprises the following mutations relative to SEQ ID NO: 1: D581R 1926R V1030G. In some embodiments, the variant Cas12i2 domain of SEQ ID NO: 41 comprises the following mutations relative to SEQ ID NO: 1: D581R I926R V1030G S1046G. In some embodiments, the variant Cas12i2 domain of SEQ ID NO: 42 comprises the following mutations relative to SEQ ID NO: 1: D51R G624R F626R I926R V1030G E1035R S1046G. In some embodiments, the variant Cas12i2 domain of SEQ ID NO: 43 comprises the following mutations relative to SEQ ID NO: 1: D581R G624R F626R P868T I926R V1030G E1035R S51046G.

In some embodiments, the variant Cas12i2 domain comprises the amino acid substitutions listed in Table 3.

TABLE 3
Amino Acid Substitutions in Cas12i2 Domain Variants.
Substitutions Relative to SEQ ID NO: 1
D581R D911R I926R V1030G
D581R I926R V1030G
D581R I926R V1030G S1046G
D581R
L589R
E652R
T770R
Q820R
D835R
L836R
K862R
P868R
S879R
S883R
D911R
W912R
N925R
I926R
Q1010G
V1030G
D581R D911R I926R
L836R D911R
D581R L836R D911R I926R
D581R L836R S879R
D581R L836R D911R
D581R S879R
L836R D911R I926R
D581R L836R S879R D911R
L589R L836R S879R D911R
D581 D835G D911R I926R
D581R D911R I926R V1030G
D581R D911R I926R S1046G
D835G L836R D911R
L836R D911R V1030G
L836R D911R S1046G
D581R D835G L836R D911R I926R
D581R L836R D911R I926R V1030G
D581R L836R D911R I926R S1046G
D581R D835G S879R
L836R D911R I926R V1030G
D581R L836R S879R S1046G
D581R L836R S879R D911R S1046G
L836R D911R I926R S1046G
D581R P868R I926R V1030G
D581R P868R I926R V1030G S1046G
D581R P868R D911R 1926R V1030G
D581R D911R I926R V1030G S1046G
D581R P868R D911R I926R V1030G S1046G
D581K D911R I926R V1030G
D581N D911R I926R V1030G
D581H D911R I926R V1030G
D581R D911R I926K V1030G
D581R D911R I926N V1030G
D581R D911R I926H V1030G
D581R D911R I926R V1030K
D581R D911R I926R V1030N
D581R D911R I926R V1030H
D581R G587R D911R I926R V1030G
D581R L623R D911R I926R V1030G
D581R G624R D911R I926R V1030G
D581R F626R D911R I926R V1030G
D581R G633R D911R I926R V1030G
D581R L654R D911R I926R V1030G
D581R Q674R D911R I926R V1030G
D581R T676R D911R I926R V1030G
D581R K677R D911R I926R V1030G
D581R N679G D911R I926R V1030G
D581R A689G D911R I926R V1030G
D581R K692G D911R I926R V1030G
D581R Q727G D911R I926R V1030G
D581R Q728G D911R I926R V1030G
D581R Q739G D911R I926R V1030G
D581R L754G D911R I926R V1030G
D581R T756G D911R I926R V1030G
D581R G762R D911R I926R V1030G
D581R K776R D911R I926R V1030G
D581R L801R D911R I926R V1030G
D581R R803G D911R I926R V1030G
D581R S817G D911R I926R V1030G
D581R L823R D911R I926R V1030G
D581R N826R D911R I926R V1030G
D581R G834R D911R I926R V1030G
D581R S837G D911R I926R V1030G
D581R S837R D911R I926R V1030G
D581R T838R D911R I926R V1030G
D581R T839R D911R I926R V1030G
D581R H870R D911R I926R V1030G
D581R G876R D911R I926R V1030G
D581R G878R D911R I926R V1030G
D581R Y881R D911R I926R V1030G
D581R D886R D911R I926R V1030G
D581R P887R D911R I926R V1030G
D581R V889R D911R I926R V1030G
D581R D894R D911R I926R V1030G
D581R K895R D911R I926R V1030G
D581R D911R I926R G929R V1030G
D581R D911R I926R E930R V1030G
D581R D911R I926R L948R V1030G
D581R D911R I926R E949R V1030G
D581R D911R I926R E950R V1030G
D581R D911R I926R P964R V1030G
D581R D911R I926R K979R V1030G
D581R D911R I926R A1018R V1030G
D581R D911R I926R D1019R V1030G
D581R D911R I926R V1021R V1030G
D581R D911R I926R A1022R V1030G
D581R D911R I926R N1025R V1030G
D581R D911R I926R V1030R
D581R D911R I926R V1030G G1032R
D581R D911R I926R V1030G E1035R
D581R D911R I926R V1030G S1037R
D581R G834R D911R N925R I926R E930R V1030G E1035R
D581R G834R D911R N925R I926R V1030G
D581R G834R D911R I926R E930R V1030G
D581R G834R D911R I926R V1030G E1035R
D581R D911R N925R I926R V1030G E1035R
D581R G834R D911R N925R I926R E930R V1030R E1035R
D581R G834R D911R N925R I926R V1030R
D581R G834R D911R I926R E930R V1030R
D581R G834R D911R I926R V1030R E1035R
D581R D911R N925R I926R V1030R E1035R
D581R G624R G834R D911R N925R I926R E930R V1030G E1035R
D581R L623R G624R L654R L801R G834R P868R G876R S883R K895R D911R N925R I926R
E930R L948R K979R A1022R V1030G E1035R
D581R G587R L623R G624R F626R L654R Q674R K677R L801R G834R S837R T838R P868R
G876R Y881R S883R D886R D894R K895R D911R N925R I926R E930R L948R E949R K979R
A1022R V1030G E1035R
D581R G624R G834R N925R I926R E930R V1030G E1035R
D581R L623R G624R L654R L801R G834R P868R G876R S883R K895R N925R I926R E930R
L948R K979R A1022R V1030G E1035R
D581R G587R L623R G624R F626R L654R Q674R K677R L801R G834R S837R T838R P868R
G876R Y881R S883R D886R D894R K895R N925R I926R E930R L948R E949R K979R A1022R
V1030G E1035R
R575H
Q645R
D581R G587R D911R V1030G S1046G
D581R G624R D911R V1030G S1046G
D581R F626R D911R V1030G S1046G
D581R D911R V1030G E1035R S1046G
D581R G587R G624R D911R V1030G S1046G
D581R G587R F626R D911R V1030G S1046G
D581R G587R D911R V1030G E1035R S1046G
D581R G624R F626R D911R V1030G S1046G
D581R G624R D911R V1030G E1035R S1046G
D581R F626R D911R V1030G E1035R S1046G
D581R G587R G624R F626R D911R V1030G S1046G
D581R G587R G624R D911R V1030G E1035R S1046G
D581R G587R F626R D911R V1030G E1035R S1046G
D581R G624R F626R D911R V1030G E1035R S1046G
D581R G587R G624R F626R D911R V1030G E1035R S1046G
D581R G624R F626R I926R V1030G E1035R S1046G
D581R G624R F626R P868T I926R V1030G E1035R S1046G
D581K I926R V1030G S1046G G587R F626R
D581K I926R V1030G S1046G G587R G624R
D581R I926R V1030G S1046G G587R G624R F626R
D581R I926R V1030G S1046G G587R G624R F626R V967K
D581R I926R V1030G S1046G L855Q
D581R I926R V1030G S1046G H870S
D581R I926R V1030G S1046G A867Q
D581R I926R V1030G S1046G V859T
D581R I926R V1030G S1046G P868T
D581R I926R V1030G S1046G R857S
D581R I926R V1030G S1046G N871P
D581R I926R V1030G S1046G R850L
D581R I926R V1030G S1046G Q602L
D581R I926R V1030G S1046G M869L
D581R I926R V1030G S1046G L880W
D581R I926R V1030G S1046G M869W

Although the changes described herein may be one or more amino acid changes, changes to a Cas12i2 fusion protein may also be of a substantive nature, such as fusion of polypeptides as amino- and/or carboxyl-terminal extensions. For example, a Cas12i2 fusion protein may contain additional peptides, e.g., one or more peptides. Examples of additional peptides may include epitope peptides for labelling, such as a polyhistidine tag (His-tag), Myc, and FLAG. In some embodiments, a Cas12i2 fusion protein comprises: MKIEEGKGHHHHHH (SEQ ID NO: 66) or KIEEGKGHHHHHH (SEQ ID NO: 67). For example, in some embodiments, a Cas12i2 fusion protein comprises at least 80% (81%, 82%, 83%, 84%, 85%, 86%, 87%, 88%, 89%, 90%, 91%, 92%, 93%, 94%, 95%, 96%, 97%, 98%, 99%, or 100%) identity to SEQ ID NO: 68, SEQ ID NO: 69, SEQ ID NO: 73, or SEQ ID NO: 74. In some embodiments, a Cas12i2 fusion protein of any one of SEQ ID NOs: 45-52 is fused to a peptide sequence of SEQ ID NO: 66 or SEQ ID NO: 67. In some embodiments, a Cas12i2 fusion protein described herein can be fused to a detectable moiety such as a fluorescent protein (e.g., green fluorescent protein (GFP) or yellow fluorescent protein (YFP)).

In those embodiments where a tag is fused to a CRISPR nuclease (e.g., a Cas12i2 fusion protein), such tag may facilitate affinity-based or charge-based purification of the CRISPR nuclease (e.g., the Cas12i2 fusion protein), e.g., by liquid chromatography or bead separation utilizing an immobilized affinity or ion-exchange reagent. As a non-limiting example, a recombinant CRISPR nuclease of this disclosure comprises a polyhistidine (His) tag, and for purification is loaded onto a chromatography column comprising an immobilized metal ion (e.g. a Zn²⁺, Ni²⁺, Cu²⁺ ion chelated by a chelating ligand immobilized on the resin, which resin may be an individually prepared resin or a commercially available resin or ready to use column. Following the loading step, the column is optionally rinsed, e.g., using one or more suitable buffer solutions, and the His-tagged protein is then eluted using a suitable elution buffer. Alternatively, or additionally, if the recombinant CRISPR nuclease of this disclosure utilizes a FLAG-tag, such protein may be purified using immunoprecipitation methods known in the industry. Other suitable purification methods for tagged CRISPR nucleases or accessory proteins of this disclosure will be evident to those of skill in the art.

A nuclease described herein can be modified to have diminished nuclease activity, e.g., nuclease inactivation of at least 50%, at least 60%, at least 70%, at least 80%, at least 90%, at least 95%, at least 97%, or 100%, as compared to a reference nuclease. Nuclease activity can be diminished by several methods known in the art, e.g., introducing mutations into the RuvC domain (e.g., one or more catalytic residues of the RuvC domain). In a non-limiting example, a variant of SEQ ID NO: 1 comprising a mutation in residue D599, residue E833, and/or residue D1019 demonstrates diminished or no nuclease activity.

In some embodiments, the Cas12i2 fusion protein described herein can be self-inactivating. See, Epstein et al., “Engineering a Self-Inactivating CRISPR System for AAV Vectors,” Mol. Ther., 24 (2016): S50, which is incorporated by reference in its entirety.

Nucleic acid molecules encoding the Cas12i2 fusion protein described herein can further be codon-optimized. The nucleic acid can be codon-optimized for use in a particular host cell, such as a bacterial cell or a mammalian cell.

### Linkers

In some instances, a linker is a covalent linkage or connection between two or more components described herein. In some embodiments, the linker comprises a chemical linker. In some embodiments, a linker comprises a functional group pair. In some embodiments, a linker is a peptide linker. In some instances, the linker(s) is located N-terminal of the fusion domain. In some instances, the linker(s) is located C-terminal of the fusion domain. In some instances, a first linker is located N-terminal of the fusion domain and the second linker is located C-terminal of the fusion domain. In some embodiments, a first linker(s) is located C-terminal of a first fusion domain and a second linker is located N-terminal of a second fusion domain.

In some embodiments, a heterologous sequence comprises one or more linkers (e.g., peptide linkers) of at least 1, 2, 3, 4, 5, 6, 7, 8, 9, 10, 11, 12, 13, 14, 15, 16, 17, 18, 19, 20, 25, 30, 35, 40, 45, 50, or more amino acid residues. In some embodiments, the linker can be located N-terminal of a fusion domain. In certain embodiments, the linker can be located C-terminal of a fusion domain. The linker sequence may comprise any naturally occurring amino acid. In some embodiments, the linker comprises amino acids glycine and serine. In some embodiments, the linker comprises sets of glycine and serine repeats such as (G₄S)ₓ, where x is a positive integer between 0 and 15 (e.g., 1, 2, 3, 4, 5, 6, 7, 8, 9, 10, 11, 12, 13, 14, or 15). In some embodiments, the linker comprises an amino acid sequence of (GSG)ₓ, wherein X is an integer between 0 and 15 (e.g., 0, 1, 2, 3, 4, 5, 6, 7, 8, 9, 10, 11, 12, 13, 14, or 15). In some embodiments, the linker comprises an amino acid sequence of (GSSG)ₓ, wherein X is an integer between 0 and 15 (e.g., 0, 1, 2, 3, 4, 5, 6, 7, 8, 9, 10, 11, 12, 13, 14, or 15). In some embodiments, the linker comprises an amino acid sequence of (GSS)ₓ, wherein X is an integer between 0 and 15 (e.g., 0, 1, 2, 3, 4, 5, 6, 7, 8, 9, 10, 11, 12, 13, 14, or 15). In some embodiments, the linker comprises an amino acid sequence of GSSGSSGSSGSSGSS (SEQ ID NO: 44). In some embodiments, the linker can comprise the amino acid sequence of any of the following:

| Linker Amino Acid Sequence | SEQ ID NO |
|---|---|
| GGGGS | SEQ ID NO: 26 |
| GGGGSGGGGSGGGGSGGG | SEQ ID NO: 27 |
| GSGGGGSGGGGS | |
| GGGGSGGGGSGGGGS | SEQ ID NO: 28 |
| GSSG | SEQ ID NO: 29 |
| GSSGGSSG | SEQ ID NO: 30 |
| GSSGGSSGGSSG | SEQ ID NO: 31 |
| GSSGGSSGGSSGGSSG | SEQ ID NO: 32 |
| GSG | SEQ ID NO: 33 |
| GSGGSGGSGGSG | SEQ ID NO: 34 |
| GGGS | SEQ ID NO: 35 |
| GSSGSSGSSGSSGSS | SEQ ID NO: 44 |
| GSS | SEQ ID NO: 70 |
| GGSGGSGGSGGSGGS | SEQ ID NO: 71 |
| GGS | SEQ ID NO: 72 |

In some embodiments, the linker comprises the 16 residue “XTEN” linker, or a variant thereof (see, e.g., Schellenberger et al. (Nat. Biotechnol. 27: 1186-1190, 2009), the entirety of which is incorporated herein by reference.

In some embodiments, any peptide linker described herein may further comprise between 1-5 (e.g., 1, 2, 3, 4, or 5) amino acid residues N-terminal or C-terminal of the peptide linker. The 1-5 amino acids residues N-terminal or C-terminal of the peptide linker can comprise any naturally occurring or modified amino acid residue.

Also included within the scope of the invention are linkers described in WO2012/138475, incorporated herein by reference in its entirety.

### Targeting Moiety

In some embodiments, the composition described herein comprises a targeting moiety.

The targeting moiety may be substantially identical to a reference nucleic acid sequence if the targeting moiety comprises a sequence having least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the two nucleic acid molecules hybridize to each other under stringent conditions (e.g., within a range of medium to high stringency).

In some embodiments, the targeting moiety has at least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence.

### RNA Guide Sequence

In some embodiments, the targeting moiety comprises, or is, an RNA guide sequence. In some embodiments, the RNA guide sequence directs a Cas12i2 fusion protein described herein to a particular nucleic acid sequence. Those skilled in the art reading the below examples of particular kinds of RNA guide sequences will understand that, in some embodiments, an RNA guide sequence is site-specific. That is, in some embodiments, an RNA guide sequence associates specifically with one or more target nucleic acid sequences (e.g., specific DNA or genomic DNA sequences) and not to non-targeted nucleic acid sequences (e.g., non-specific DNA or random sequences).

In some embodiments, the composition as described herein comprises an RNA guide sequence that associates with a Cas12i2 domain of a Cas12i2 fusion protein described herein and directs a Cas12i2 fusion protein to a target nucleic acid sequence (e.g., DNA). The RNA guide sequence may associate with a nucleic acid sequence and alter functionality of a Cas12i2 fusion protein (e.g., alters affinity of the Cas12i2 fusion protein to a molecule, e.g., at least 10%, 15%, 20%, 25%, 30%, 35%, 40%, 45%, 50%, 55%, 60%, 65%, 70%, 75%, 80%, 85%, 90%, 95%, or more).

The RNA guide sequence may target (e.g., associate with, be directed to, contact, or bind) one or more nucleotides of a sequence, e.g., a site-specific sequence or a site-specific target. In some embodiments, a Cas12i2 domain (e.g., a Cas12i2 domain of a Cas12i2 fusion protein plus an RNA guide) is activated upon binding to a target nucleic acid, e.g., to a target strand of a target nucleic acid, wherein the target strand of the target nucleic acid has complementarity to a spacer sequence in the RNA guide.

In some embodiments, an RNA guide sequence comprises a spacer sequence. In some embodiments, the spacer sequence of the RNA guide sequence may be generally designed to have a length of between 15-35 nucleotides (e.g., 18, 19, 20, 21, 22, 23, 24, 25, 26, 27, 28, 29, or 30 nucleotides) and be complementary to a specific nucleic acid sequence. In some particular embodiments, the RNA guide sequence may be designed to be complementary to a specific DNA strand, e.g., of a genomic locus. In some embodiments, the spacer sequence is designed to be complementary to a specific DNA strand, e.g., of a genomic locus.

In certain embodiments, the RNA guide sequence includes, consists essentially of, or comprises a direct repeat sequence linked to a sequence or spacer sequence. In some embodiments, the RNA guide sequence includes a direct repeat sequence and a spacer sequence or a direct repeat-spacer-direct repeat sequence. In some embodiments, the RNA guide sequence includes a truncated direct repeat sequence and a spacer sequence, which is typical of processed or mature crRNA. In some embodiments, a nuclease forms a complex with the RNA guide sequence, and the RNA guide sequence directs the complex to associate with site-specific target nucleic acid that is complementary to at least a portion of the RNA guide sequence.

In some embodiments, the RNA guide sequence comprises a sequence, e.g., RNA sequence, at least 80%, at least 90%, at least 95%, at least 96%, at least 97%, at least 98%, at least 99% complementary to a target nucleic acid sequence. In some embodiments, the RNA guide sequence comprises a sequence at least 80%, at least 90%, at least 95%, at least 96%, at least 97%, at least 98%, at least 99% complementary to a DNA sequence. In some embodiments, the RNA guide sequence comprises a sequence at least 80%, at least 90%, at least 95%, at least 96%, at least 97%, at least 98%, at least 99% complementary to a target nucleic acid sequence. In some embodiments, the RNA guide sequence comprises a sequence at least 80%, at least 90%, at least 95%, at least 96%, at least 97%, at least 98%, at least 99% complementary to a genomic sequence. In some embodiments, the RNA guide sequence comprises a sequence complementary to or a sequence comprising at least 80%, at least 90%, at least 95%, at least 96%, at least 97%, at least 98%, at least 99% complementarity to a genomic sequence.

In some embodiments, a nuclease described herein includes one or more (e.g., two, three, four, five, six, seven, eight, or more) RNA guide sequences, e.g., RNA guides.

In some embodiments, the RNA guide has an architecture similar to, for example International Publication Nos. WO 2014/093622 and WO 2015/070083, the entire contents of each of which are incorporated herein by reference.

In some embodiments, an RNA guide sequence of the present invention comprises a direct repeat sequence having 80, 85, 90, 91, 92, 93, 94, 95, 96, 97, 98, 99 or 100% identity the direct repeat sequences of Table 4. In some embodiments, an RNA guide of the present invention comprises a direct repeat sequence having greater than 80, 85, 90, 91, 92, 93, 94, 95, 96, 97, 98, 99 or 100% identity to the direct repeat sequences of Table 4.

TABLE 4
Exemplary direct repeat sequences.

| Cas1212 | |
|---|---|
| SEQ ID NO | Direct Repeat Sequence |
| 1, 39-43, | GCAACACCUAAGAAAUCCGUCUUU |
| 45-52, | CAUUGACGGG (SEQ ID NO: 36) |
| 68, 69, | GUUGCAAAACCCAAGAAAUCCGUC |
| 73, 74 | UUUCAUUGACGG (SEQ ID NO: 37) |

In some embodiments, a Cas12i2 fusion protein and an RNA guide (e.g., an RNA guide comprising a direct repeat and a spacer) form a complex. In some embodiments, a Cas12i2 fusion protein and an RNA guide (e.g., an RNA guide comprising direct repeat-spacer-direct repeat sequence or pre-crRNA) form a complex. In some embodiments, the complex binds a target nucleic acid. In some embodiments, the Cas12i2 fusion protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 1, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 36 or SEQ ID NO: 37.

In some embodiments, the spacer of an RNA guide binds to a target nucleic acid, e.g., to the target strand (i.e., non-PAM strand) of a target nucleic acid, wherein the non-target strand (i.e., PAM strand) comprises a target sequence adjacent to a PAM sequence of any one of 5′-TTN-3′, 5′-TTH-3′, 5′-TTY-3′, or 5′-TTC-3′.

In some embodiments, the gRNA (e.g., a crRNA) comprises:

(SEQ ID NO: 38)
5′-AGAAAUCCGUCUUUCAUUGACGG[spacer]-3′.

In some embodiments, a Cas12i2 fusion protein and an RNA guide (e.g., an RNA guide comprising a direct repeat and a spacer) form a complex. In some embodiments, a Cas12i2 fusion protein and an RNA guide (e.g., an RNA guide comprising direct repeat-spacer-direct repeat sequence or pre-crRNA) form a complex. In some embodiments, the complex binds a target nucleic acid. In some embodiments, the Cas12i2 fusion protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 1, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 38.

In some embodiments, an RNA guide described herein comprises a uracil (U). In some embodiments, an RNA guide described herein comprises a thymine (T). In some embodiments, a direct repeat sequence of an RNA guide described herein comprises a uracil (U). In some embodiments, a direct repeat sequence of an RNA guide described herein comprises a thymine (T). Unless otherwise noted, all compositions and nucleases provided herein are made in reference to the active level of that composition or nuclease, and are exclusive of impurities, for example, residual solvents or by-products, which may be present in commercially available sources. Nuclease component weights are based on total active protein. All percentages and ratios are calculated by weight unless otherwise indicated. All percentages and ratios are calculated based on the total composition unless otherwise indicated. In the exemplified composition, the nuclease levels are expressed by pure enzyme by weight of the total composition and unless otherwise specified, the ingredients are expressed by weight of the total compositions.

### Modifications

The RNA guide sequence or any of the nucleic acid sequences encoding a Cas12i2 fusion protein described herein may include one or more covalent modifications with respect to a reference sequence, in particular the parent polyribonucleotide, which are included within the scope of this invention.

Exemplary modifications can include any modification to the sugar, the nucleobase, the internucleoside linkage (e.g. to a linking phosphate/to a phosphodiester linkage/to the phosphodiester backbone), and any combination thereof. Some of the exemplary modifications provided herein are described in detail below.

The RNA guide sequence or any of the nucleic acid sequences encoding components of a Cas12i2 fusion protein may include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O-(1-thiophosphate)-cytidine (a-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197). In some embodiments, the first isolated nucleic acid comprises messenger RNA (mRNA). In some embodiments, the mRNA comprises at least one nucleoside selected from the group consisting of pyridine-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotide (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADARI marks dsRNA as “self”. Cell Res. 25, 1283-1284, which is incorporated by reference in its entirety.

### Vectors

The present invention also provides a vector for expressing a Cas12i2 fusion protein described herein or nucleic acids encoding a Cas12i2 fusion protein described herein may be incorporated into a vector. In some embodiments, a vector of the invention includes a nucleotide sequence encoding a Cas12i2 fusion protein described herein. In some embodiments, a vector of the invention includes a nucleotide sequence encoding a Cas12i2 fusion protein described herein.

The present invention also provides a vector that may be used for preparation of a Cas12i2 fusion protein described herein or compositions comprising a Cas12i2 fusion protein described herein. In some embodiments, the invention includes the composition or vector described herein in a cell. In some embodiments, the invention includes a method of expressing a composition comprising a Cas12i2 fusion protein of the present invention, or vector or nucleic acid encoding the Cas12i2 fusion protein, in a cell. The method may comprise the steps of providing the Cas12i2 fusion protein, e.g., vector or nucleic acid, and delivering the Cas12i2 fusion protein to the cell.

Expression of natural or synthetic polynucleotides is typically achieved by operably linking a polynucleotide encoding the gene of interest, e.g., nucleotide sequence encoding a Cas12i2 fusion protein of the present invention, to a promoter and incorporating the construct into an expression vector. The expression vector is not particularly limited as long as it includes a polynucleotide encoding a Cas12i2 fusion protein of the present invention and can be suitable for replication and integration in eukaryotic cells.

Typical expression vectors include transcription and translation terminators, initiation sequences, and promoters useful for expression of the desired polynucleotide. For example, plasmid vectors carrying a recognition sequence for RNA polymerase (pSP64, pBluescript, etc.). may be used. Vectors including those derived from retroviruses such as lentivirus are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector.

Viral vector technology is well known in the art and described in a variety of virology and molecular biology manuals. Viruses which are useful as vectors include, but are not limited to phage viruses, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

The kind of the vector is not particularly limited, and a vector that can be expressed in host cells can be appropriately selected. To be more specific, depending on the kind of the host cell, a promoter sequence to ensure the expression of a nuclease of the present invention from a polynucleotide is appropriately selected, and this promoter sequence and the polynucleotide are inserted into any of various plasmids etc. for preparation of the expression vector.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. Depending on the promoter, it appears that individual elements can function either cooperatively or independently to activate transcription.

Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Examples of such a marker include a dihydrofolate reductase gene and a neomycin resistance gene for eukaryotic cell culture; and a tetracycline resistance gene and an ampicillin resistance gene for culture of E. coli and other bacteria. By use of such a selection marker, it can be confirmed whether the polynucleotide encoding a nuclease of the present invention has been transferred into the host cells and then expressed without fail.

The preparation method for recombinant expression vectors is not particularly limited, and examples thereof include methods using a plasmid, a phage or a cosmid.

### Cells

The Cas12i2 fusion protein described herein can be introduced into a variety of cells. In some embodiments, the cell is an isolated cell. In some embodiments, the cell is in cell culture. In some embodiments, the cell is ex vivo. In some embodiments, the cell is obtained from a living organism, and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the cell is an archaeal cell or derived from an archaeal cell.

In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T, MF7, K562, HeLa, and transgenic varieties thereof. Cell lines are available from a variety of sources known to those with skill in the art (see, e.g., the American Type Culture Collection (ATCC) (Manassas, Va.)). In some embodiments, a cell transfected with one or more nucleic acids (such as nuclease polypeptide encoding vector and RNA guide) is used to establish a new cell line comprising one or more vector-derived sequences to establish a new cell line comprising modification to the target nucleic acid or target locus. In some embodiments, the cell is an immortal or immortalized cell.

In some embodiments, the cell is a primary cell. In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a differentiated cell. For example, in some embodiments, the differentiated cell is a muscle cell (e.g., a myocyte), a fat cell (e.g., an adipocyte), a bone cell (e.g., an osteoblast, osteocyte, osteoclast), a blood cell (e.g., a monocyte, a lymphocyte, a neutrophil, an eosinophil, a basophil, a macrophage, a erythrocyte, or a platelet), a nerve cell (e.g., a neuron), an epithelial cell, an immune cell (e.g., a lymphocyte, a neutrophil, a monocyte, or a macrophage), a liver cell (e.g., a hepatocyte), a fibroblast, or a sex cell. In some embodiments, the cell is a terminally differentiated cell. For example, in some embodiments, the terminally differentiated cell is a neuronal cell, an adipocyte, a cardiomyocyte, a skeletal muscle cell, an epidermal cell, or a gut cell. In some embodiments, the cell is a mammalian cell, e.g., a human cell or a murine cell. In some embodiments, the murine cell is derived from a wild-type mouse, an immunosuppressed mouse, or a disease-specific mouse model.

### Production

In some embodiments, a Cas12i2 fusion protein of the present invention can be prepared by an in vitro coupled transcription-translation system. Bacteria that can be used for preparation of a Cas12i2 fusion protein of the present invention are not particularly limited as long as they can produce a Cas12i2 fusion protein of the present invention. Some non-limiting examples of the bacteria include E. coli cells described herein.

### Methods of Expression

The present invention includes a method for protein expression, comprising translating a Cas12i2 fusion protein described herein.

In some embodiments, a host cell described herein is used to express a Cas12i2 fusion protein. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of a Cas12i2 fusion protein. After expression of the Cas12i2 fusion protein, the host cells can be collected and Cas12i2 fusion protein purified from the cultures etc. according to conventional methods (for example, filtration, centrifugation, cell disruption, gel filtration chromatography, ion exchange chromatography, etc.).

In some embodiments, the methods for Cas12i2 fusion protein expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of a nuclease. In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids, about 1100 amino acids, about 1200 amino acids, about 1300 amino acids, about 1400 amino acids, about 1500 amino acids, about 1600 amino acids, about 1700 amino acids, about 1800 amino acids, about 1900 amino acids, about 2000 amino acids, or more of a Cas12i2 fusion protein.

A variety of methods can be used to determine the level of production of a Cas12i2 fusion protein in a host cell. Such methods include, but are not limited to, for example, methods that utilize either polyclonal or monoclonal antibodies specific for a Cas12i2 fusion protein. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of a Cas12i2 fusion protein in a cell, comprising providing a polyribonucleotide encoding the Cas12i2 fusion protein to a host cell wherein the polyribonucleotide encodes the Cas12i2 fusion protein, expressing the Cas12i2 fusion protein in the cell, and obtaining the Cas12i2 fusion protein from the cell.

### Delivery

Compositions described herein may be formulated, for example, including a carrier, such as a carrier and/or a polymeric carrier, e.g., a liposome, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding a Cas12i2 fusion protein, RNA guide, donor DNA, etc.), one or more transcripts thereof, and/or a pre-formed Cas12i2 fusion protein/RNA guide complex to a cell. Exemplary intracellular delivery methods, include, but are not limited to: viruses or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnectofection or magnet assisted transfection, particle bombardment; and hybrid methods, such as nucleofection. In some embodiments, the present application further provides cells produced by such methods, and organisms (such as animals, plants, or fungi) comprising or produced from such cells.

All references and publications cited herein are hereby incorporated by reference.

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1—Targeting of Mammalian Genes by Cas12i2 Fusion Proteins

This Example describes fusion protein activity (e.g., base editing or methylation) assessment on multiple targets using Cas12i2 fusion proteins introduced into mammalian cells by transient transfection.

The Cas12i2 fusion proteins described herein can be cloned into a pcda3.1 backbone (Invitrogen™). The plasmids can then be maxi-prepped and diluted. For RNA guide preparation, a dsDNA fragment encoding a crRNA can be derived by ultramers containing the target sequence scaffold, and the U6 promoter. Ultramers can be resuspended in Tris·HCl at a pH of 7.5. The amplification of the crRNA can be done using the aforementioned template, a forward primer, a reverse primer, NEB HiFi Polymerase, and water. Cycling conditions are: 1×(30 s at 98° C.), 30×(10 s at 98° C., 15 s at 67° C.), 1×(2 min at 72° C.). PCR products can be cleaned up with a 1.8×SPRI treatment and normalized to 25 ng/μL.

Approximately 16 hours prior to transfection, 25,000 HEK293T cells in DMEM/10% FBS+Pen/Strep can be plated into each well of a 96-well plate. On the day of transfection, the cells are 70-90% confluent. For each well to be transfected, a mixture of Lipofectamine™ 2000 and Opti-MEM™ can be prepared and then incubated at room temperature for 5-20 minutes (Solution 1). After incubation, the Lipofectamine™:OptiMEM™ mixture can be added to a separate mixture containing Cas12i2 plasmid and crRNA and water (Solution 2). In the case of negative controls, the crRNA is not included in Solution 2. The solution 1 and solution 2 mixtures can be mixed by pipetting up and down and then incubated at room temperature for 25 minutes. Following incubation, the Solution 1 and Solution 2 mixture can be added dropwise to each well of a 96 well plate containing the cells. 72 hours post transfection, cells can be trypsinized by adding 10 μL of TrypLE™ to the center of each well and incubated for approximately 5 minutes. 100 μL of D10 media can then be added to each well and mixed to resuspend cells. The cells can then be spun down, and the supernatant can be discarded. QuickExtract™ buffer can be added to the amount of the original cell suspension volume. Cells can be incubated at 65° C. for 15 minutes, 68° C. for 15 minutes, and 98° C. for 10 minutes.

Activity of a Cas12i2 fusion protein comprising a base editing domain can be monitored by next gen sequencing. Samples for Next Generation Sequencing can be prepared by two rounds of PCR. The first round (PCR1) is used to amplify specific genomic regions depending on the target. PCR1 products can be purified by column purification. Round 2 PCR (PCR2) can be done to add Illumina adapters and indexes. Reactions can then be pooled and purified by column purification. Sequencing runs can be done with a 150 cycle NextSeq v2.5 mid or high output kit.

Activity of a Cas12i2 fusion protein comprising a DNA methylation domain can be monitored, e.g., by methylation-specific PCR or whole-genome bisulfite sequencing.

### Example 2—Targeting of Mammalian Genes by Circularly Permuted Cas12i2 Polypeptides

This Example describes engineering and protein activity (e.g., indel activity) assessment of circularly permutated Cas12i2 polypeptides. The native amino and carboxy termini (residues 1 and 1,054) of the variant Cas12i2 polypeptide of SEQ ID NO: 40 were covalently linked with the following amino acid linker: GGSGGSGGSGGSGGS (SEQ ID NO: 71), and new N- and C-termini were introduced, thereby reorganizing the amino acid sequence of the protein. The positions of the new N- and C-termini relative to the amino acid positions of SEQ ID NO: 40 are shown in Table 5, and the sequences of the circularly permuted Cas12i2 polypeptides are shown in Table 6.

TABLE 5
N- and C-terminal Residues

| | New N terminus (previous | New C terminus (previous |
|---|---|---|
| SEQ ID NO | amino acid #) | amino acid #) |
| 40 | 1 | 1054 |
| 45 | 410 | 409 |
| 46 | 681 | 680 |
| 47 | 61 | 60 |
| 48 | 102 | 101 |
| 49 | 117 | 116 |
| 50 | 200 | 199 |
| 51 | 247 | 246 |
| 52 | 893 | 892 |

TABLE 6
Circular permutation variants and
corresponding nuclease sequences.

| SEQ ID NO | Nuclease sequence |
|---|---|
| 45 | MECSAQDILAAAKYNQQLDRYKSQKANPSV |
| | LGNQGFTWTNAVILPEKAQRNDRPNSLDLR |
| | IWLYLKLRHPDGRWKKHHIPFYDTRFFQEI |
| | YAAGNSPVDTCQFRTPRFGYHLPKLTDQTA |
| | IRVNKKHVKAAKTEARIRLAIQQGTLPVSN |
| | LKITEISATINSKGQVRIPVKFRVGRQKGT |
| | LQIGDRFCGYDQNQTASHAYSLWEVVKEGQ |
| | YHKELGCFVRFISSGDIVSITENRGNQFDQ |
| | LSYEGLAYPQYADWRKKASKFVSLWQITKK |
| | NKKKEIVTVEAKEKFDAICKYQPRLYKFNK |
| | EYAYLLRDIVRGKSLVELQQIRQEIFRFIE |
| | QDCGVTRLGSLSLSTLETVKAVKGIIYSYF |
| | STALNASKNNPISDEQRKEFDPELFALLEK |
| | LELIRTRKKKQKVERIANSLIQTCLENNIK |
| | FIRGEGDLSTINNATKKKANSRSMDWLARG |
| | VENKIRQLAPMHNITLFGCGSLYTSHQDPL |
| | VHRNPDKAMKCRWAAIPVKDIGDWVLRKLS |
| | QNLRAKNRGTGEYYHQGVKEFLSHYELQDL |
| | EEELLKWRSDRKSNIPCWVLQNRLAEKLGN |
| | KEAVVYIPVRGGRIYFATHKVATGAVSIVF |
| | DQKQVWVCNADHVAAANIALTGKGIGEQSS |
| | DEENPDGSRIKLQLTSGGSGGSGGSGGSGG |
| | SMSSAIKSYKSVLRPNERKNQLLKSTIQCL |
| | EDGSAFFFKMLQGLFGGITPEIVRFSTEQE |
| | KQQQDIALWCAVNWFRPVSQDSLTHTIASD |
| | NLVEKFEEYYGGTASDAIKQYFSASIGESY |
| | YWNDCRQQYYDLCRELGVEVSDLTHDLEIL |
| | CREKCLAVATESNQNNSIISVLFGTGEKED |
| | RSVKLRITKKILEAISNLKEIPKNVAPIQE |
| | IILNVAKATKETFRQVYAGNLGAPSTLEKF |
| | IAKDGQKEFDLKKLQTDLKKVIRGKSKERD |
| | WCCQEELRSYVEQNTIQYDLWAWGEMFNKA |
| | HTALKIKSTRNYNFAKQRLEQFKEIQSLNN |
| | LLVVKKLNDFFDSEFFSGEETYTICVHHLG |
| | GKDLSKLYKAWEDDPADPENAIVVLCDDLK |
| | NNFKKEPIRNILRYIFTIRQ |
| 46 | MKKEIVTVEAKEKFDAICKYQPRLYKFNKE |
| | YAYLLRDIVRGKSLVELQQIRQEIFRFIEQ |
| | DCGVTRLGSLSLSTLETVKAVKGIIYSYFS |
| | TALNASKNNPISDEQRKEFDPELFALLEKL |
| | ELIRTRKKKQKVERIANSLIQTCLENNIKF |
| | IRGEGDLSTINNATKKKANSRSMDWLARGV |
| | ENKIRQLAPMHNITLFGCGSLYTSHQDPLV |
| | HRNPDKAMKCRWAAIPVKDIGDWVLRKLSQ |
| | NLRAKNRGTGEYYHQGVKEFLSHYELQDLE |
| | EELLKWRSDRKSNIPCWVLQNRLAEKLGNK |
| | EAVVYIPVRGGRIYFATHKVATGAVSIVFD |
| | QKQVWVCNADHVAAANIALTGKGIGEQSSD |
| | EENPDGSRIKLQLTSGGSGGSGGSGGSGGS |
| | MSSAIKSYKSVLRPNERKNQLLKSTIQCLE |
| | DGSAFFFKMLQGLFGGITPEIVRESTEQEK |
| | QQQDIALWCAVNWFRPVSQDSLTHTIASDN |
| | LVEKFEEYYGGTASDAIKQYFSASIGESYY |
| | WNDCRQQYYDLCRELGVEVSDLTHDLEILC |
| | REKCLAVATESNQNNSIISVLFGTGEKEDR |
| | SVKLRITKKILEAISNLKEIPKNVAPIQEI |
| | ILNVAKATKETFRQVYAGNLGAPSTLEKFI |
| | AKDGQKEFDLKKLQTDLKKVIRGKSKERDW |
| | CCQEELRSYVEQNTIQYDLWAWGEMFNKAH |
| | TALKIKSTRNYNFAKQRLEQFKEIQSLNNL |
| | LVVKKLNDFFDSEFFSGEETYTICVHHLGG |
| | KDLSKLYKAWEDDPADPENAIVVLCDDLKN |
| | NFKKEPIRNILRYIFTIRQECSAQDILAAA |
| | KYNQQLDRYKSQKANPSVLGNQGFTWINAV |
| | ILPEKAQRNDRPNSLDLRIWLYLKLRHPDG |
| | RWKKHHIPFYDTRFFQEIYAAGNSPVDTCQ |
| | FRTPRFGYHLPKLTDQTAIRVNKKHVKAAK |
| | TEARIRLAIQQGTLPVSNLKITEISATINS |
| | KGQVRIPVKFRVGRQKGTLQIGDRFCGYDQ |
| | NQTASHAYSLWEVVKEGQYHKELGCFVRFI |
| | SSGDIVSITENRGNQFDQLSYEGLAYPQYA |
| | DWRKKASKFVSLWQITKKNK |
| 47 | MQQQDIALWCAVNWFRPVSQDSLTHTIASD |
| | NLVEKFEEYYGGTASDAIKQYFSASIGESY |
| | YWNDCRQQYYDLCRELGVEVSDLTHDLEIL |
| | CREKCLAVATESNQNNSIISVLFGTGEKED |
| | RSVKLRITKKILEAISNLKEIPKNVAPIQE |
| | IILNVAKATKETFRQVYAGNLGAPSTLEKF |
| | IAKDGQKEFDLKKLQTDLKKVIRGKSKERD |
| | WCCQEELRSYVEQNTIQYDLWAWGEMFNKA |
| | HTALKIKSTRNYNFAKQRLEQFKEIQSLNN |
| | LLVVKKLNDFFDSEFFSGEETYTICVHHLG |
| | GKDLSKLYKAWEDDPADPENAIVVLCDDLK |
| | NNFKKEPIRNILRYIFTIRQECSAQDILAA |
| | AKYNQQLDRYKSQKANPSVLGNQGFTWTNA |
| | VILPEKAQRNDRPNSLDLRIWLYLKLRHPD |
| | GRWKKHHIPFYDTRFFQEIYAAGNSPVDTC |
| | QFRTPRFGYHLPKLTDQTAIRVNKKHVKAA |
| | KTEARIRLAIQQGTLPVSNLKITEISATIN |
| | SKGQVRIPVKFRVGRQKGTLQIGDRFCGYD |
| | QNQTASHAYSLWEVVKEGQYHKELGCFVRF |
| | ISSGDIVSITENRGNQFDQLSYEGLAYPQY |
| | ADWRKKASKFVSLWQITKKNKKKEIVTVEA |
| | KEKFDAICKYQPRLYKFNKEYAYLLRDIVR |
| | GKSLVELQQIRQEIFRFIEQDCGVTRLGSL |
| | SLSTLETVKAVKGIIYSYFSTALNASKNNP |
| | ISDEQRKEFDPELFALLEKLELIRTRKKKQ |
| | KVERIANSLIQTCLENNIKFIRGEGDLSTI |
| | NNATKKKANSRSMDWLARGVFNKIRQLAPM |
| | HNITLFGCGSLYTSHQDPLVHRNPDKAMKC |
| | RWAAIPVKDIGDWVLRKLSQNLRAKNRGTG |
| | EYYHQGVKEFLSHYELQDLEEELLKWRSDR |
| | KSNIPCWVLQNRLAEKLGNKEAVVYIPVRG |
| | GRIYFATHKVATGAVSIVFDQKQVWVCNAD |
| | HVAAANIALTGKGIGEQSSDEENPDGSRIK |
| | LQLTSGGSGGSGGSGGSGGSMSSAIKSYKS |
| | VLRPNERKNQLLKSTIQCLEDGSAFFFKML |
| | QGLFGGITPEIVRFSTEQEK |
| 48 | MTASDAIKQYFSASIGESYYWNDCRQQYYD |
| | LCRELGVEVSDLTHDLEILCREKCLAVATE |
| | SNQNNSIISVLFGTGEKEDRSVKLRITKKI |
| | LEAISNLKEIPKNVAPIQEIILNVAKATKE |
| | TFRQVYAGNLGAPSTLEKFIAKDGQKEFDL |
| | KKLQTDLKKVIRGKSKERDWCCQEELRSYV |
| | EQNTIQYDLWAWGEMENKAHTALKIKSTRN |
| | YNFAKQRLEQFKEIQSLNNLLVVKKLNDFF |
| | DSEFFSGEETYTICVHHLGGKDLSKLYKAW |
| | EDDPADPENAIVVLCDDLKNNFKKEPIRNI |
| | LRYIFTIRQECSAQDILAAAKYNQQLDRYK |
| | SQKANPSVLGNQGFTWINAVILPEKAQRND |
| | RPNSLDLRIWLYLKLRHPDGRWKKHHIPFY |
| | DTRFFQEIYAAGNSPVDTCQFRTPRFGYHL |
| | PKLTDQTAIRVNKKHVKAAKTEARIRLAIQ |
| | QGTLPVSNLKITEISATINSKGQVRIPVKF |
| | RVGRQKGTLQIGDRFCGYDQNQTASHAYSL |
| | WEVVKEGQYHKELGCFVRFISSGDIVSITE |
| | NRGNQFDQLSYEGLAYPQYADWRKKASKFV |
| | SLWQITKKNKKKEIVTVEAKEKFDAICKYQ |
| | PRLYKFNKEYAYLLRDIVRGKSLVELQQIR |
| | QEIFRFIEQDCGVTRLGSLSLSTLETVKAV |
| | KGIIYSYFSTALNASKNNPISDEQRKEFDP |
| | ELFALLEKLELIRTRKKKQKVERIANSLIQ |
| | TCLENNIKFIRGEGDLSTINNATKKKANSR |
| | SMDWLARGVFNKIRQLAPMHNITLFGCGSL |
| | YTSHQDPLVHRNPDKAMKCRWAAIPVKDIG |
| | DWVLRKLSQNLRAKNRGTGEYYHQGVKEFL |
| | SHYELQDLEEELLKWRSDRKSNIPCWVLQN |
| | RLAEKLGNKEAVVYIPVRGGRIYFATHKVA |
| | TGAVSIVFDQKQVWVCNADHVAAANIALTG |
| | KGIGEQSSDEENPDGSRIKLQLTSGGSGGS |
| | GGSGGSGGSMSSAIKSYKSVLRPNERKNQL |
| | LKSTIQCLEDGSAFFFKMLQGLFGGITPEI |
| | VRFSTEQEKQQQDIALWCAVNWFRPVSQDS |
| | LTHTIASDNLVEKFEEYYGG |
| 49 | MESYYWNDCRQQYYDLCRELGVEVSDLTHD |
| | LEILCREKCLAVATESNQNNSIISVLFGTG |
| | EKEDRSVKLRITKKILEAISNLKEIPKNVA |
| | PIQEIILNVAKATKETFRQVYAGNLGAPST |
| | LEKFIAKDGQKEFDLKKLQTDLKKVIRGKS |
| | KERDWCCQEELRSYVEQNTIQYDLWAWGEM |
| | FNKAHTALKIKSTRNYNFAKQRLEQFKEIQ |
| | SLNNLLVVKKLNDFFDSEFFSGEETYTICV |
| | HHLGGKDLSKLYKAWEDDPADPENAIVVLC |
| | DDLKNNFKKEPIRNILRYIFTIRQECSAQD |
| | ILAAAKYNQQLDRYKSQKANPSVLGNQGFT |
| | WTNAVILPEKAQRNDRPNSLDLRIWLYLKL |
| | RHPDGRWKKHHIPFYDTRFFQEIYAAGNSP |
| | VDTCQFRTPRFGYHLPKLTDQTAIRVNKKH |
| | VKAAKTEARIRLAIQQGTLPVSNLKITEIS |
| | ATINSKGQVRIPVKFRVGRQKGTLQIGDRF |
| | CGYDQNQTASHAYSLWEVVKEGQYHKELGC |
| | FVRFISSGDIVSITENRGNQFDQLSYEGLA |
| | YPQYADWRKKASKFVSLWQITKKNKKKEIV |
| | TVEAKEKFDAICKYQPRLYKFNKEYAYLLR |
| | DIVRGKSLVELQQIRQEIFRFIEQDCGVTR |
| | LGSLSLSTLETVKAVKGIIYSYFSTALNAS |
| | KNNPISDEQRKEFDPELFALLEKLELIRTR |
| | KKKQKVERIANSLIQTCLENNIKFIRGEGD |
| | LSTINNATKKKANSRSMDWLARGVENKIRQ |
| | LAPMHNITLFGCGSLYTSHQDPLVHRNPDK |
| | AMKCRWAAIPVKDIGDWVLRKLSQNLRAKN |
| | RGTGEYYHQGVKEFLSHYELQDLEEELLKW |
| | RSDRKSNIPCWVLQNRLAEKLGNKEAVVYI |
| | PVRGGRIYFATHKVATGAVSIVFDQKQVWV |
| | CNADHVAAANIALTGKGIGEQSSDEENPDG |
| | SRIKLQLTSGGSGGSGGSGGSGGSMSSAIK |
| | SYKSVLRPNERKNQLLKSTIQCLEDGSAFF |
| | FKMLQGLFGGITPEIVRFSTEQEKQQQDIA |
| | LWCAVNWFRPVSQDSLTHTIASDNLVEKFE |
| | EYYGGTASDAIKQYFSASIG |
| 50 | MIPKNVAPIQEIILNVAKATKETFRQVYAG |
| | NLGAPSTLEKFIAKDGQKEFDLKKLQTDLK |
| | KVIRGKSKERDWCCQEELRSYVEQNTIQYD |
| | LWAWGEMENKAHTALKIKSTRNYNFAKQRL |
| | EQFKEIQSLNNLLVVKKLNDFFDSEFFSGE |
| | ETYTICVHHLGGKDLSKLYKAWEDDPADPE |
| | NAIVVLCDDLKNNFKKEPIRNILRYIFTIR |
| | QECSAQDILAAAKYNQQLDRYKSQKANPSV |
| | LGNQGFTWINAVILPEKAQRNDRPNSLDLR |
| | IWLYLKLRHPDGRWKKHHIPFYDTRFFQEI |
| | YAAGNSPVDTCQFRTPRFGYHLPKLTDQTA |
| | IRVNKKHVKAAKTEARIRLAIQQGTLPVSN |
| | LKITEISATINSKGQVRIPVKFRVGRQKGT |
| | LQIGDRFCGYDQNQTASHAYSLWEVVKEGQ |
| | YHKELGCFVRFISSGDIVSITENRGNQFDQ |
| | LSYEGLAYPQYADWRKKASKFVSLWQITKK |
| | NKKKEIVTVEAKEKFDAICKYQPRLYKFNK |
| | EYAYLLRDIVRGKSLVELQQIRQEIFRFIE |
| | QDCGVTRLGSLSLSTLETVKAVKGIIYSYF |
| | STALNASKNNPISDEQRKEFDPELFALLEK |
| | LELIRTRKKKQKVERIANSLIQTCLENNIK |
| | FIRGEGDLSTTNNATKKKANSRSMDWLARG |
| | VENKIRQLAPMHNITLFGCGSLYTSHQDPL |
| | VHRNPDKAMKCRWAAIPVKDIGDWVLRKLS |
| | QNLRAKNRGTGEYYHQGVKEFLSHYELQDL |
| | EEELLKWRSDRKSNIPCWVLQNRLAEKLGN |
| | KEAVVYIPVRGGRIYFATHKVATGAVSIVE |
| | DQKQVWVCNADHVAAANIALTGKGIGEQSS |
| | DEENPDGSRIKLQLTSGGSGGSGGSGGSGG |
| | SMSSAIKSYKSVLRPNERKNQLLKSTIQCL |
| | EDGSAFFFKMLQGLFGGITPEIVRESTEQE |
| | KQQQDIALWCAVNWFRPVSQDSLTHTIASD |
| | NLVEKFEEYYGGTASDAIKQYFSASIGESY |
| | YWNDCRQQYYDLCRELGVEVSDLTHDLEIL |
| | CREKCLAVATESNQNNSIISVLFGTGEKED |
| | RSVKLRITKKILEAISNLKE |
| 51 | MEFDLKKLQTDLKKVIRGKSKERDWCCQEE |
| | LRSYVEQNTIQYDLWAWGEMENKAHTALKI |
| | KSTRNYNFAKQRLEQFKEIQSLNNLLVVKK |
| | LNDFFDSEFFSGEETYTICVHHLGGKDLSK |
| | LYKAWEDDPADPENAIVVLCDDLKNNFKKE |
| | PIRNILRYIFTIRQECSAQDILAAAKYNQQ |
| | LDRYKSQKANPSVLGNQGFTWTNAVILPEK |
| | AQRNDRPNSLDLRIWLYLKLRHPDGRWKKH |
| | HIPFYDTRFFQEIYAAGNSPVDTCQFRTPR |
| | FGYHLPKLTDQTAIRVNKKHVKAAKTEARI |
| | RLAIQQGTLPVSNLKITEISATINSKGQVR |
| | IPVKFRVGRQKGTLQIGDRFCGYDQNQTAS |
| | HAYSLWEVVKEGQYHKELGCFVRFISSGDI |
| | VSITENRGNQFDQLSYEGLAYPQYADWRKK |
| | ASKFVSLWQITKKNKKKEIVTVEAKEKFDA |
| | ICKYQPRLYKENKEYAYLLRDIVRGKSLVE |
| | LQQIRQEIFRFIEQDCGVTRLGSLSLSTLE |
| | TVKAVKGIIYSYFSTALNASKNNPISDEQR |
| | KEFDPELFALLEKLELIRTRKKKQKVERIA |
| | NSLIQTCLENNIKFIRGEGDLSTINNATKK |
| | KANSRSMDWLARGVENKIRQLAPMHNITLF |
| | GCGSLYTSHQDPLVHRNPDKAMKCRWAAIP |
| | VKDIGDWVLRKLSQNLRAKNRGTGEYYHQG |
| | VKEFLSHYELQDLEEELLKWRSDRKSNIPC |
| | WVLQNRLAEKLGNKEAVVYIPVRGGRIYFA |
| | THKVATGAVSIVFDQKQVWVCNADHVAAAN |
| | IALTGKGIGEQSSDEENPDGSRIKLQLTSG |
| | GSGGSGGSGGSGGSMSSAIKSYKSVLRPNE |
| | RKNQLLKSTIQCLEDGSAFFFKMLQGLEGG |
| | ITPEIVRFSTEQEKQQQDIALWCAVNWFRP |
| | VSQDSLTHTIASDNLVEKFEEYYGGTASDA |
| | IKQYFSASIGESYYWNDCRQQYYDLCRELG |
| | VEVSDLTHDLEILCREKCLAVATESNQNNS |
| | IISVLFGTGEKEDRSVKLRITKKILEAISN |
| | LKEIPKNVAPIQEIILNVAKATKETFRQVY |
| | AGNLGAPSTLEKFIAKDGQK |
| 52 | MPDKAMKCRWAAIPVKDIGDWVLRKLSQNL |
| | RAKNRGTGEYYHQGVKEFLSHYELQDLEEE |
| | LLKWRSDRKSNIPCWVLQNRLAEKLGNKEA |
| | VVYIPVRGGRIYFATHKVATGAVSIVFDQK |
| | QVWVCNADHVAAANIALTGKGIGEQSSDEE |
| | NPDGSRIKLQLTSGGSGGSGGSGGSGGSMS |
| | SAIKSYKSVLRPNERKNQLLKSTIQCLEDG |
| | SAFFFKMLQGLFGGITPEIVRFSTEQEKQQ |
| | QDIALWCAVNWFRPVSQDSLTHTIASDNLV |
| | EKFEEYYGGTASDAIKQYFSASIGESYYWN |
| | DCRQQYYDLCRELGVEVSDLTHDLEILCRE |
| | KCLAVATESNQNNSIISVLFGTGEKEDRSV |
| | KLRITKKILEAISNLKEIPKNVAPIQEIIL |
| | NVAKATKETFRQVYAGNLGAPSTLEKFIAK |
| | DGQKEFDLKKLQTDLKKVIRGKSKERDWCC |
| | QEELRSYVEQNTIQYDLWAWGEMFNKAHTA |
| | LKIKSTRNYNFAKQRLEQFKEIQSLNNLLV |
| | VKKLNDFFDSEFFSGEETYTICVHHLGGKD |
| | LSKLYKAWEDDPADPENAIVVLCDDLKNNF |
| | KKEPIRNILRYIFTIRQECSAQDILAAAKY |
| | NQQLDRYKSQKANPSVLGNQGFTWINAVIL |
| | PEKAQRNDRPNSLDLRIWLYLKLRHPDGRW |
| | KKHHIPFYDTRFFQEIYAAGNSPVDTCQFR |
| | TPRFGYHLPKLTDQTAIRVNKKHVKAAKTE |
| | ARIRLAIQQGTLPVSNLKITEISATINSKG |
| | QVRIPVKFRVGRQKGTLQIGDRFCGYDQNQ |
| | TASHAYSLWEVVKEGQYHKELGCFVRFISS |
| | GDIVSITENRGNQFDQLSYEGLAYPQYADW |
| | RKKASKFVSLWQITKKNKKKEIVTVEAKEK |
| | FDAICKYQPRLYKFNKEYAYLLRDIVRGKS |
| | LVELQQIRQEIFRFIEQDCGVTRLGSLSLS |
| | TLETVKAVKGIIYSYFSTALNASKNNPISD |
| | EQRKEFDPELFALLEKLELIRTRKKKQKVE |
| | RIANSLIQTCLENNIKFIRGEGDLSTINNA |
| | TKKKANSRSMDWLARGVENKIRQLAPMHNI |
| | TLFGCGSLYTSHQDPLVHRN |

The variant Cas12i2 polypeptide of SEQ ID NO: 40 and the circularly permuted Cas12i2 polypeptides of SEQ ID NOs: 45-52 were cloned into a pcDNA3.1 backbone (Invitrogen™). RNA guides were cloned into a pUC19 backbone (New England Biolabs®). The plasmids were then maxi-prepped and diluted. The tested RNA guide and target sequences are shown in Table 7.

TABLE 7
Mammalian targets and corresponding crRNAs.

| Target | | Target |
|---|---|---|
| identifier | crRNA sequence | sequence |
| EMX1_T2 | AGACAUGUGUCCUC | GGATGGCG |
| | AGUGACACGGAUGG | ACTTCAGG |
| | CGACUUCAGGCACA | CACAGGAT |
| | GGAU | (SEQ ID |
| | (SEQ ID NO: 53) | NO: 54) |
| EMX1_T7 | AGACAUGUGUCCUC | AGCAAGGG |
| | AGUGACACAGCAAG | ACTATTCA |
| | GGACUAUUCAGGGA | GGGATGAA |
| | UGAA | (SEQ ID |
| | (SEQ ID NO: 55) | NO: 56) |
| EMX1_T8 | AGACAUGUGUCCUC | AAAATTGA |
| | AGUGACACAAAAUU | GCAATCTA |
| | GAGCAAUCUACCCU | CCCTGGTC |
| | GGUC | (SEQ ID |
| | (SEQ ID NO: 57) | NO: 58) |
| VEGFA_T5 | AGACAUGUGUCCUC | TTAAACTC |
| | AGUGACACUUAAAC | TCCATGGAC |
| | UCUCCAUGGACCAG | CAGGCTC |
| | GCUC | (SEQ ID |
| | (SEQ ID NO: 59) | NO: 60) |

Approximately 16 hours prior to transfection, HEK293T cells in DMEM/10% FBS+Pen/Strep were plated into each well of a 96-well plate. On the day of transfection, the cells were 70-90% confluent. For each well to be transfected, a mixture of Lipofectamine™ 2000 and Opti-MEM™ was prepared and then incubated at room temperature for 5-20 minutes (Solution 1). After incubation, the Lipofectamine™:OptiMEM™ mixture was added to a separate mixture containing Cas12i2 plasmid and RNA guide plasmid and water (Solution 2). In the case of negative controls, the crRNA was not included in Solution 2. The solution 1 and solution 2 mixtures were mixed by pipetting up and down and then incubated at room temperature for 25 minutes. Following incubation, Solution 1 and Solution 2 mixture were added dropwise to each well of a 96-well plate containing the cells. 72 hours post-transfection, cells were trypsinized by adding TrypLE™ to the center of each well and incubated for approximately 5 minutes. D10 media was then added to each well and mixed to resuspend cells. The cells were then spun down, and the supernatant was discarded. QuickExtract™ buffer was added to ⅕ the amount of the original cell suspension volume. Cells were incubated at 65° C. for 15 minutes, 68° C. for 15 minutes, and 98° C. for 10 minutes.

Samples for Next Generation Sequencing were prepared by two rounds of PCR. The first round (PCR1) was used to amplify specific genomic regions depending on the target. PCR1 products were purified by column purification. Round 2 PCR (PCR2) was done to add Illumina adapters and indexes. Reactions were then pooled and purified by column purification. Sequencing runs were done with a 150 cycle NextSeq v2.5 mid or high output kit.

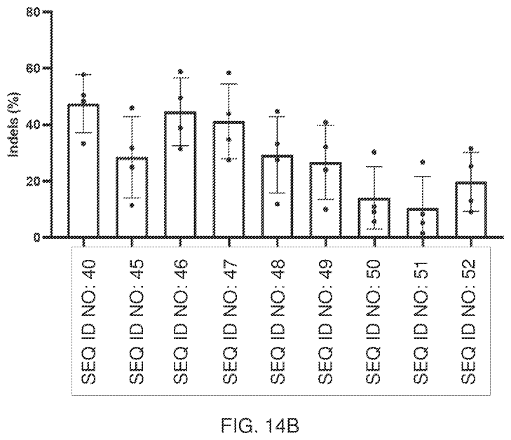

FIG. 14A and FIG. 14B show indel activity for variant Cas12i2 of SEQ ID NO: 40 and circularly permuted Cas12i2 polypeptides of SEQ ID NOs: 45-52. Each of the circularly permuted Cas12i2 polypeptides demonstrated indel activity at the tested mammalian targets. The circularly permuted Cas12i2 polypeptides of SEQ ID NO: 46 and SEQ ID NO: 47 demonstrated similar indel activity to that of the variant Cas12i2 polypeptide of SEQ ID NO: 40 (FIG. 14A and FIG. 14B).

This Example thus shows that circularly permuted Cas12i2 polypeptides function as nucleases. Therefore, the circularly permuted Cas12i2 polypeptides appeared to maintain domain structure despite rearrangement of the amino acid sequences.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

SEQUENCE LISTING

| | | Description or exemplary |
|---|---|---|
| SEQ ID NO | Sequence | uses |
| SEQ ID NO: 1 | MSSAIKSYKSVLRPNERKNQLLKSTIQCLEDGSAFF | Cas12i2 |
| | FKMLQGLFGGITPEIVRFSTEQEKQQQDIALWCAVN | |
| | WFRPVSQDSLTHTIASDNLVEKFEEYYGGTASDAIK | |
| | QYFSASIGESYYWNDCRQQYYDLCRELGVEVSDLTH | |
| | DLEILCREKCLAVATESNQNNSIISVLFGTGEKEDR | |
| | SVKLRITKKILEAISNLKEIPKNVAPIQEIILNVAK | |
| | ATKETFRQVYAGNLGAPSTLEKFIAKDGQKEFDLKK | |
| | LQTDLKKVIRGKSKERDWCCQEELRSYVEQNTIQYD | |
| | LWAWGEMENKAHTALKIKSTRNYNFAKQRLEQFKEI | |
| | QSLNNLLVVKKLNDFFDSEFFSGEETYTICVHHLGG | |
| | KDLSKLYKAWEDDPADPENAIVVLCDDLKNNFKKEP | |
| | IRNILRYIFTIRQECSAQDILAAAKYNQQLDRYKSQ | |
| | KANPSVLGNQGFTWTNAVILPEKAQRNDRPNSLDLR | |
| | IWLYLKLRHPDGRWKKHHIPFYDTRFFQEIYAAGNS | |
| | PVDTCQFRTPRFGYHLPKLTDQTAIRVNKKHVKAAK | |
| | TEARIRLAIQQGTLPVSNLKITEISATINSKGQVRI | |
| | PVKFDVGRQKGTLQIGDRFCGYDQNQTASHAYSLWE | |
| | VVKEGQYHKELGCFVRFISSGDIVSITENRGNQFDQ | |
| | LSYEGLAYPQYADWRKKASKFVSLWQITKKNKKKEI | |
| | VTVEAKEKFDAICKYQPRLYKFNKEYAYLLRDIVRG | |
| | KSLVELQQIRQEIFRFIEQDCGVTRLGSLSLSTLET | |
| | VKAVKGIIYSYFSTALNASKNNPISDEQRKEFDPEL | |
| | FALLEKLELIRTRKKKQKVERIANSLIQTCLENNIK | |
| | FIRGEGDLSTINNATKKKANSRSMDWLARGVENKIR | |
| | QLAPMHNITLFGCGSLYTSHQDPLVHRNPDKAMKCR | |
| | WAAIPVKDIGDWVLRKLSQNLRAKNIGTGEYYHQGV | |
| | KEFLSHYELQDLEEELLKWRSDRKSNIPCWVLQNRL | |
| | AEKLGNKEAVVYIPVRGGRIYFATHKVATGAVSIVF | |
| | DQKQVWVCNADHVAAANIALTVKGIGEQSSDEENPD | |
| | GSRIKLQLTS | |
| SEQ ID NO: 2 | SEFFSGEETYTICVHHL | PAM Distal Region of R-loop |
| | | 342-358 |
| SEQ ID NO: 3 | DPADPE | PAM Distal Region of R-loop |
| | | 373-378 |
| SEQ ID NO: 4 | RQECSA | PAM Distal Region of R-loop |
| | | 408-413 |
| SEQ ID NO: 5 | KKNKKKEIV | PAM Distal Region of R-loop |
| | | 677-685 |
| SEQ ID NO: 6 | VRGKSL | PAM Distal Region of R-loop |
| | | 718-723 |
| SEQ ID NO: 7 | ALNASKNNPISD | PAM Distal Region of R-loop |
| | | 771-782 |
| SEQ ID NO: 8 | LKWRSDRKSNIPC | PAM Distal Region of R-loop |
| | | 953-965 |
| SEQ ID NO: 9 | STEQEKQQQDI | PAM Proximal Region of R- |
| | | loop 55-65 |
| SEQ ID NO: 10 | YGGTASD | PAM Proximal Region of R- |
| | | loop 99-105 |
| SEQ ID NO: 11 | SASIGESYY | PAM Proximal Region of R- |
| | | loop 112-120 |
| SEQ ID NO: 12 | SNLKEIPKNVAP | PAM Proximal Region of R- |
| | | loop 195-206 |
| SEQ ID NO: 13 | KDGQKEFDL | PAM Proximal Region of R- |
| | | loop 241-250 |
| SEQ ID NO: 14 | GRQKGTLQIGDR | PAM Proximal Region of R- |
| | | loop 583-594 |
| SEQ ID NO: 15 | CGSLYTSHQDPLVHRNPDKAMKCRW | Positions to introduce an NLS |
| | | (e.g., which does not |
| | | necessarily interact with DNA) |
| | | 877-901 |
| SEQ ID NO: 16 | GTGEKED | Positions to introduce an NLS |
| | | (e.g., which does not |
| | | necessarily interact with DNA) |
| | | 173-179 |
| SEQ ID NO: 17 | KATKET | Positions to introduce an NLS |
| | | (not for interacting with DNA) |
| | | 216-221 |
| SEQ ID NO: 18 | SKERDWCC | Positions to introduce an NLS |
| | | (e.g., which does not |
| | | necessarily interact with DNA) |
| | | 265-272 |
| SEQ ID NO: 19 | RQECSA | Positions to introduce an NLS |
| | | (e.g., which does not |
| | | necessarily interact with DNA) |
| | | 408-413 |
| SEQ ID NO: 20 | AQRNDRPNSLDLR | Positions to introduce an NLS |
| | | (e.g., which does not |
| | | necessarily interact with DNA) |
| | | 456-468 |
| SEQ ID NO: 21 | HPDGRW | Positions to introduce an NLS |
| | | (e.g., which does not |
| | | necessarily interact with DNA) |
| | | 476-482 |
| SEQ ID NO: 22 | IYAAGNSPVDTCQFRT | Positions to introduce an NLS |
| | | (e.g., which does not |
| | | necessarily interact with DNA) |
| | | 498-513 |
| SEQ ID NO: 23 | VKEGQYHKELGC | Positions to introduce an NLS |
| | | (e.g., which does not |
| | | necessarily interact with DNA) |
| | | 614-625 |
| SEQ ID NO: 24 | GNKEAV | Positions to introduce an NLS |
| | | (e.g., which does not |
| | | necessarily interact with DNA) |
| | | 977-982 |
| SEQ ID NO: 25 | VFDQKQ | Positions to introduce an NLS |
| | | (e.g., which does not |
| | | necessarily interact with DNA) |
| | | 1007-1012 |
| SEQ ID NO: 26 | GGGGS | G4S |
| SEQ ID NO: 27 | GGGGGGGGSGGGGSGGGGSGGGGSGGGGS | (G4S)6 |
| SEQ ID NO: 28 | GGGGSGGGGSGGGGS | (G4S)4 |
| SEQ ID NO: 29 | GSSG | (G4S)3 |
| SEQ ID NO: 30 | GSSGGSSG | (GSSG)2 |
| SEQ ID NO: 31 | GSSGGSSGGSSG | (GSSG)3 |
| SEQ ID NO: 32 | GSSGGSSGGSSGGSSG | (GSSG)4 |
| SEQ ID NO: 33 | GSG | GSG |
| SEQ ID NO: 34 | GSGGSGGSGGSG | (GSG)4 |
| SEQ ID NO: 35 | GGGS | (G3S) |
| SEQ ID NO: 36 | GCAACACCUAAGAAAUCCGUCUUUCAUUGACGGG | Cas12i2 Direct Repeat |
| | | Sequence 1 |
| SEQ ID NO: 37 | GUUGCAAAACCCAAGAAAUCCGUCUUUCAUUGACGG | Cas12i2 Direct Repeat |
| | | Sequence 2 |
| SEQ ID NO: 38 | AGAAAUCCGUCUUUCAUUGACGG | Cas12i2 mature crRNA |
| SEQ ID NO: 39 | MSSAIKSYKSVLRPNERKNQLKSTIQCLEDGSAFFF | |
| | KMLQGLFGGITPEIVRFSTEQEKQQQDIALWCAVNW | |
| | FRPVSQDSLTHTIASDNLVEKFEEYYGGTASDAIKQ | |
| | YFSASIGESYYWNDCRQQYYDLCRELGVEVSDLTHD | |
| | LEILCREKCLAVATESNQNNSIISVLFGTGEKEDRS | |
| | VKLRITKKILEAISNLKEIPKNVAPIQEIILNVAKA | |
| | TKETFRQVYAGNLGAPSTLEKFIAKDGQKEFDLKKL | |
| | QTDLKKVIRGKSKERDWCCQEELRSYVEQNTIQYDL | |
| | WAWGEMENKAHTALKIKSTRNYNFAKQRLEQFKEIQ | |
| | SLNNLLVVKKLNDFFDSEFFSGEETYTICVHHLGGK | |
| | DLSKLYKAWEDDPADPENAIVVLCDDLKNNFKKEPI | |
| | RNILRYIFTIRQECSAQDILAAAKYNQQLDRYKSQK | |
| | ANPSVLGNQGFTWINAVILPEKAQRNDRPNSLDLRI | |
| | WLYLKLRHPDGRWKKHHIPFYDTRFFQEIYAAGNSP | |
| | VDTCQFRTPRFGYHLPKLTDQTAIRVNKKHVKAAKT | |
| | EARIRLAIQQGTLPVSNLKITEISATINSKGQVRIP | |
| | VKFRVGRQKGTLQIGDRFCGYDQNQTASHAYSLWEV | |
| | VKEGQYHKELGCFVRFISSGDIVSITENRGNQFDQL | |
| | SYEGLAYPQYADWRKKASKFVSLWQITKKNKKKEIV | |
| | TVEAKEKFDAICKYQPRLYKFNKEYAYLLRDIVRGK | |
| | SLVELQQIRQEIFRFIEQDCGVTRLGSLSLSTLETV | |
| | KAVKGIIYSYFSTALNASKNNPISDEQRKEFDPELF | |
| | ALLEKLELIRTRKKKQKVERIANSLIQTCLENNIKF | |
| | IRGEGDLSTTNNATKKKANSRSMDWLARGVENKIRQ | |
| | LAPMHNITLFGCGSLYTSHQDPLVHRNPDKAMKCRW | |
| | AAIPVKDIGRWVLRKLSQNLRAKNRGTGEYYHQGVK | |
| | EFLSHYELQDLEEELLKWRSDRKSNIPCWVLQNRLA | |
| | EKLGNKEAVVYIPVRGGRIYFATHKVATGAVSIVED | |
| | QKQVWVCNADHVAAANIALTGKGIGEQSSDEENPDG | |
| | SRIKLQLTS | |
| SEQ ID NO: 40 | MSSAIKSYKSVLRPNERKNQLLKSTIQCLEDGSAFF | |
| | FKMLQGLFGGITPEIVRESTEQEKQQQDIALWCAVN | |
| | WFRPVSQDSLTHTIASDNLVEKFEEYYGGTASDAIK | |
| | QYFSASIGESYYWNDCRQQYYDLCRELGVEVSDLTH | |
| | DLEILCREKCLAVATESNQNNSIISVLFGTGEKEDR | |
| | SVKLRITKKILEAISNLKEIPKNVAPIQEIILNVAK | |
| | ATKETFRQVYAGNLGAPSTLEKFIAKDGQKEFDLKK | |
| | LQTDLKKVIRGKSKERDWCCQEELRSYVEQNTIQYD | |
| | LWAWGEMENKAHTALKIKSTRNYNFAKQRLEQFKEI | |
| | QSLNNLLVVKKLNDFFDSEFFSGEETYTICVHHLGG | |
| | KDLSKLYKAWEDDPADPENAIVVLCDDLKNNFKKEP | |
| | IRNILRYIFTIRQECSAQDILAAAKYNQQLDRYKSQ | |
| | KANPSVLGNQGFTWTNAVILPEKAQRNDRPNSLDLR | |
| | IWLYLKLRHPDGRWKKHHIPFYDTRFFQEIYAAGNS | |
| | PVDTCQFRTPRFGYHLPKLTDQTAIRVNKKHVKAAK | |
| | TEARIRLAIQQGTLPVSNLKITEISATINSKGQVRI | |
| | PVKFRVGRQKGTLQIGDRFCGYDQNQTASHAYSLWE | |
| | VVKEGQYHKELGCFVRFISSGDIVSITENRGNQFDQ | |
| | LSYEGLAYPQYADWRKKASKFVSLWQITKKNKKKEI | |
| | VTVEAKEKFDAICKYQPRLYKFNKEYAYLLRDIVRG | |
| | KSLVELQQIRQEIFRFIEQDCGVTRLGSLSLSTLET | |
| | VKAVKGIIYSYFSTALNASKNNPISDEQRKEFDPEL | |
| | FALLEKLELIRTRKKKQKVERIANSLIQTCLENNIK | |
| | FIRGEGDLSTINNATKKKANSRSMDWLARGVENKIR | |
| | QLAPMHNITLFGCGSLYTSHQDPLVHRNPDKAMKCR | |
| | WAAIPVKDIGDWVLRKLSQNLRAKNRGTGEYYHQGV | |
| | KEFLSHYELQDLEEELLKWRSDRKSNIPCWVLQNRL | |
| | AEKLGNKEAVVYIPVRGGRIYFATHKVATGAVSIVF | |
| | DQKQVWVCNADHVAAANIALTGKGIGEQSSDEENPD | |
| | GSRIKLQLTS | |
| SEQ ID NO: 41 | MSSAIKSYKSVLRPNERKNQLLKSTIQCLEDGSAFF | |
| | FKMLQGLFGGITPEIVRFSTEQEKQQQDIALWCAVN | |
| | WFRPVSQDSLTHTIASDNLVEKFEEYYGGTASDAIK | |
| | QYFSASIGESYYWNDCRQQYYDLCRELGVEVSDLTH | |
| | DLEILCREKCLAVATESNQNNSIISVLFGTGEKEDR | |
| | SVKLRITKKILEAISNLKEIPKNVAPIQEIILNVAK | |
| | ATKETFRQVYAGNLGAPSTLEKFIAKDGQKEFDLKK | |
| | LQTDLKKVIRGKSKERDWCCQEELRSYVEQNTIQYD | |
| | LWAWGEMENKAHTALKIKSTRNYNFAKQRLEQFKEI | |
| | QSLNNLLVVKKLNDFFDSEFFSGEETYTICVHHLGG | |
| | KDLSKLYKAWEDDPADPENAIVVLCDDLKNNFKKEP | |
| | IRNILRYIFTIRQECSAQDILAAAKYNQQLDRYKSQ | |
| | KANPSVLGNQGFTWTNAVILPEKAQRNDRPNSLDLR | |
| | IWLYLKLRHPDGRWKKHHIPFYDTRFFQEIYAAGNS | |
| | PVDTCQFRTPRFGYHLPKLTDQTAIRVNKKHVKAAK | |
| | TEARIRLAIQQGTLPVSNLKITEISATINSKGQVRI | |
| | PVKFRVGRQKGTLQIGDRFCGYDQNQTASHAYSLWE | |
| | VVKEGQYHKELGCFVRFISSGDIVSITENRGNQFDQ | |
| | LSYEGLAYPQYADWRKKASKFVSLWQITKKNKKKEI | |
| | VTVEAKEKFDAICKYQPRLYKFNKEYAYLLRDIVRG | |
| | KSLVELQQIRQEIFRFIEQDCGVTRLGSLSLSTLET | |
| | VKAVKGIIYSYFSTALNASKNNPISDEQRKEFDPEL | |
| | FALLEKLELIRTRKKKQKVERIANSLIQTCLENNIK | |
| | FIRGEGDLSTTNNATKKKANSRSMDWLARGVENKIR | |
| | QLAPMHNITLFGCGSLYTSHQDPLVHRNPDKAMKCR | |
| | WAAIPVKDIGDWVLRKLSQNLRAKNRGTGEYYHQGV | |
| | KEFLSHYELQDLEEELLKWRSDRKSNIPCWVLQNRL | |
| | AEKLGNKEAVVYIPVRGGRIYFATHKVATGAVSIVF | |
| | DQKQVWVCNADHVAAANIALTGKGIGEQSSDEENPD | |
| | GGRIKLQLTS | |
| SEQ ID NO: 42 | MSSAIKSYKSVLRPNERKNQLLKSTIQCLEDGSAFF | |
| | FKMLQGLEGGITPEIVRESTEQEKQQQDIALWCAVN | |
| | WFRPVSQDSLTHTIASDNLVEKFEEYYGGTASDAIK | |
| | QYFSASIGESYYWNDCRQQYYDLCRELGVEVSDLTH | |
| | DLEILCREKCLAVATESNQNNSIISVLFGTGEKEDR | |
| | SVKLRITKKILEAISNLKEIPKNVAPIQEIILNVAK | |
| | ATKETFRQVYAGNLGAPSTLEKFIAKDGQKEFDLKK | |
| | LQTDLKKVIRGKSKERDWCCQEELRSYVEQNTIQYD | |
| | LWAWGEMFNKAHTALKIKSTRNYNFAKQRLEQFKEI | |
| | QSLNNLLVVKKLNDFFDSEFFSGEETYTICVHHLGG | |
| | KDLSKLYKAWEDDPADPENAIVVLCDDLKNNFKKEP | |
| | IRNILRYIFTIRQECSAQDILAAAKYNQQLDRYKSQ | |
| | KANPSVLGNQGFTWTNAVILPEKAQRNDRPNSLDLR | |
| | IWLYLKLRHPDGRWKKHHIPFYDTRFFQEIYAAGNS | |
| | PVDTCQFRTPRFGYHLPKLTDQTAIRVNKKHVKAAK | |
| | TEARIRLAIQQGTLPVSNLKITEISATINSKGQVRI | |
| | PVKFRVGRQKGTLQIGDRFCGYDQNQTASHAYSLWE | |
| | VVKEGQYHKELRCRVRFISSGDIVSITENRGNQFDQ | |
| | LSYEGLAYPQYADWRKKASKFVSLWQITKKNKKKEI | |
| | VTVEAKEKFDAICKYQPRLYKFNKEYAYLLRDIVRG | |
| | KSLVELQQIRQEIFRFIEQDCGVTRLGSLSLSTLET | |
| | VKAVKGIIYSYFSTALNASKNNPISDEQRKEFDPEL | |
| | FALLEKLELIRTRKKKQKVERIANSLIQTCLENNIK | |
| | FIRGEGDLSTTNNATKKKANSRSMDWLARGVENKIR | |
| | QLAPMHNITLFGCGSLYTSHQDPLVHRNPDKAMKCR | |
| | WAAIPVKDIGDWVLRKLSQNLRAKNRGTGEYYHQGV | |
| | KEFLSHYELQDLEEELLKWRSDRKSNIPCWVLQNRL | |
| | AEKLGNKEAVVYIPVRGGRIYFATHKVATGAVSIVF | |
| | DQKQVWVCNADHVAAANIALTGKGIGRQSSDEENPD | |
| | GGRIKLQLTS | |
| SEQ ID NO: 43 | MSSAIKSYKSVLRPNERKNQLKSTIQCLEDGSAFFF | |
| | KMLQGLFGGITPEIVRFSTEQEKQQQDIALWCAVNW | |
| | FRPVSQDSLTHTIASDNLVEKFEEYYGGTASDAIKQ | |
| | YFSASIGESYYWNDCRQQYYDLCRELGVEVSDLTHD | |
| | LEILCREKCLAVATESNQNNSIISVLFGTGEKEDRS | |
| | VKLRITKKILEAISNLKEIPKNVAPIQEIILNVAKA | |
| | TKETFRQVYAGNLGAPSTLEKFIAKDGQKEFDLKKL | |
| | QTDLKKVIRGKSKERDWCCQEELRSYVEQNTIQYDL | |
| | WAWGEMENKAHTALKIKSTRNYNFAKQRLEQFKEIQ | |
| | SLNNLLVVKKLNDFFDSEFFSGEETYTICVHHLGGK | |
| | DLSKLYKAWEDDPADPENAIVVLCDDLKNNFKKEPI | |
| | RNILRYIFTIRQECSAQDILAAAKYNQQLDRYKSQK | |
| | ANPSVLGNQGFTWTNAVILPEKAQRNDRPNSLDLRI | |
| | WLYLKLRHPDGRWKKHHIPFYDTRFFQEIYAAGNSP | |
| | VDTCQFRTPRFGYHLPKLTDQTAIRVNKKHVKAAKT | |
| | EARIRLAIQQGTLPVSNLKITEISATINSKGQVRIP | |
| | VKFRVGRQKGTLQIGDRFCGYDQNQTASHAYSLWEV | |
| | VKEGQYHKELGCFVRFISSGDIVSITENRGNQFDQL | |
| | SYEGLAYPQYADWRKKASKFVSLWQITKKNKKKEIV | |
| | TVEAKEKFDAICKYQPRLYKENKEYAYLLRDIVRGK | |
| | SLVELQQIRQEIFRFIEQDCGVTRLGSLSLSTLETV | |
| | KAVKGIIYSYFSTALNASKNNPISDEQRKEFDPELF | |
| | ALLEKLELIRTRKKKQKVERIANSLIQTCLENNIKF | |
| | IRGEGDLSTINNATKKKANSRSMDWLARGVENKIRQ | |
| | LAPMHNITLFGCGSLYTSHQDPLVHRNPDKAMKCRW | |
| | AAIPVKDIGRWVLRKLSQNLRAKNRGTGEYYHQGVK | |
| | EFLSHYELQDLEEELLKWRSDRKSNIPCWVLQNRLA | |
| | EKLGNKEAVVYIPVRGGRIYFATHKVATGAVSIVED | |
| | QKQVWVCNADHVAAANIALTGKGIGEQSSDEENPDG | |
| | SRIKLQLTS | |
| SEQ ID NO: 44 | GSSGSSGSSGSSGSS | (GSS)5 |
| SEQ ID NO: 45 | MECSAQDILAAAKYNQQLDRYKSQKANPSVLGNQGF | |
| | TWTNAVILPEKAQRNDRPNSLDLRIWLYLKLRHPDG | |
| | RWKKHHIPFYDTRFFQEIYAAGNSPVDTCQFRTPRF | |
| | GYHLPKLIDQTAIRVNKKHVKAAKTEARIRLAIQQG | |
| | TLPVSNLKITEISATINSKGQVRIPVKFRVGRQKGT | |
| | LQIGDRFCGYDQNQTASHAYSLWEVVKEGQYHKELG | |
| | CFVRFISSGDIVSITENRGNQFDQLSYEGLAYPQYA | |
| | DWRKKASKFVSLWQITKKNKKKEIVTVEAKEKFDAI | |
| | CKYQPRLYKFNKEYAYLLRDIVRGKSLVELQQIRQE | |
| | IFRFIEQDCGVTRLGSLSLSTLETVKAVKGIIYSYF | |
| | STALNASKNNPISDEQRKEFDPELFALLEKLELIRT | |
| | RKKKQKVERIANSLIQTCLENNIKFIRGEGDLSTIN | |
| | NATKKKANSRSMDWLARGVENKIRQLAPMHNITLFG | |
| | CGSLYTSHQDPLVHRNPDKAMKCRWAAIPVKDIGDW | |
| | VLRKLSQNLRAKNRGTGEYYHQGVKEFLSHYELQDL | |
| | EEELLKWRSDRKSNIPCWVLQNRLAEKLGNKEAVVY | |
| | IPVRGGRIYFATHKVATGAVSIVFDQKQVWVCNADH | |
| | VAAANIALIGKGIGEQSSDEENPDGSRIKLQLTSGG | |
| | SGGSGGSGGSGGSMSSAIKSYKSVLRPNERKNQLLK | |
| | STIQCLEDGSAFFFKMLQGLEGGITPEIVRESTEQE | |
| | KQQQDIALWCAVNWFRPVSQDSLTHTIASDNLVEKF | |
| | EEYYGGTASDAIKQYFSASIGESYYWNDCRQQYYDL | |
| | CRELGVEVSDLTHDLEILCREKCLAVATESNQNNSI | |
| | ISVLFGTGEKEDRSVKLRITKKILEAISNLKEIPKN | |
| | VAPIQEIILNVAKATKETFRQVYAGNLGAPSTLEKF | |
| | IAKDGQKEFDLKKLQTDLKKVIRGKSKERDWCCQEE | |
| | LRSYVEQNTIQYDLWAWGEMENKAHTALKIKSTRNY | |
| | NFAKQRLEQFKEIQSLNNLLVVKKLNDFFDSEFFSG | |
| | EETYTICVHHLGGKDLSKLYKAWEDDPADPENAIVV | |
| | LCDDLKNNFKKEPIRNILRYIFTIRQ | |
| SEQ ID NO: 46 | MKKEIVTVEAKEKFDAICKYQPRLYKFNKEYAYLLR | |
| | DIVRGKSLVELQQIRQEIFRFIEQDCGVTRLGSLSL | |
| | STLETVKAVKGIIYSYFSTALNASKNNPISDEQRKE | |
| | FDPELFALLEKLELIRTRKKKQKVERIANSLIQTCL | |
| | ENNIKFIRGEGDLSTINNATKKKANSRSMDWLARGV | |
| | FNKIRQLAPMHNITLFGCGSLYTSHQDPLVHRNPDK | |
| | AMKCRWAAIPVKDIGDWVLRKLSQNLRAKNRGTGEY | |
| | YHQGVKEFLSHYELQDLEEELLKWRSDRKSNIPCWV | |
| | LQNRLAEKLGNKEAVVYIPVRGGRIYFATHKVATGA | |
| | VSIVFDQKQVWVCNADHVAAANIALTGKGIGEQSSD | |
| | EENPDGSRIKLQLTSGGSGGSGGSGGSGGSMSSAIK | |
| | SYKSVLRPNERKNQLLKSTIQCLEDGSAFFFKMLQG | |
| | LFGGITPEIVRFSTEQEKQQQDIALWCAVNWFRPVS | |
| | QDSLTHTIASDNLVEKFEEYYGGTASDAIKQYFSAS | |
| | IGESYYWNDCRQQYYDLCRELGVEVSDLTHDLEILC | |
| | REKCLAVATESNQNNSIISVLFGTGEKEDRSVKLRI | |
| | TKKILEAISNLKEIPKNVAPIQEIILNVAKATKETF | |
| | RQVYAGNLGAPSTLEKFIAKDGQKEFDLKKLQTDLK | |
| | KVIRGKSKERDWCCQEELRSYVEQNTIQYDLWAWGE | |
| | MFNKAHTALKIKSTRNYNFAKQRLEQFKEIQSLNNL | |
| | LVVKKLNDFFDSEFFSGEETYTICVHHLGGKDLSKL | |
| | YKAWEDDPADPENAIVVLCDDLKNNFKKEPIRNILR | |
| | YIFTIRQECSAQDILAAAKYNQQLDRYKSQKANPSV | |
| | LGNQGFTWINAVILPEKAQRNDRPNSLDLRIWLYLK | |
| | LRHPDGRWKKHHIPFYDTRFFQEIYAAGNSPVDTCQ | |
| | FRTPRFGYHLPKLTDQTAIRVNKKHVKAAKTEARIR | |
| | LAIQQGTLPVSNLKITEISATINSKGQVRIPVKFRV | |
| | GRQKGTLQIGDRFCGYDQNQTASHAYSLWEVVKEGQ | |
| | YHKELGCFVRFISSGDIVSITENRGNQFDQLSYEGL | |
| | AYPQYADWRKKASKFVSLWQITKKNK | |
| SEQ ID NO: 47 | MQQQDIALWCAVNWFRPVSQDSLTHTIASDNLV | |
| | EKFEEYYGGTASDAIKQYFSASIGESYYWNDCR | |
| | QQYYDLCRELGVEVSDLTHDLEILCREKCLAVA | |
| | TESNQNNSIISVLFGTGEKEDRSVKLRITKKIL | |
| | EAISNLKEIPKNVAPIQEIILNVAKATKETFRQ | |
| | VYAGNLGAPSTLEKFIAKDGQKEFDLKKLQTDL | |
| | KKVIRGKSKERDWCCQEELRSYVEQNTIQYDLW | |
| | AWGEMFNKAHTALKIKSTRNYNFAKQRLEQFKE | |
| | IQSLNNLLVVKKLNDFFDSEFFSGEETYTICVH | |
| | HLGGKDLSKLYKAWEDDPADPENAIVVLCDDLK | |
| | NNFKKEPIRNILRYIFTIRQECSAQDILAAAKY | |
| | NQQLDRYKSQKANPSVLGNQGFTWTNAVILPEK | |
| | AQRNDRPNSLDLRIWLYLKLRHPDGRWKKHHIP | |
| | FYDTRFFQEIYAAGNSPVDTCQFRTPRFGYHLP | |
| | KLTDQTAIRVNKKHVKAAKTEARIRLAIQQGTL | |
| | PVSNLKITEISATINSKGQVRIPVKFRVGRQKG | |
| | TLQIGDRFCGYDQNQTASHAYSLWEVVKEGQYH | |
| | KELGCFVRFISSGDIVSITENRGNQFDQLSYEG | |
| | LAYPQYADWRKKASKFVSLWQITKKNKKKEIVT | |
| | VEAKEKFDAICKYQPRLYKFNKEYAYLLRDIVR | |
| | GKSLVELQQIRQEIFRFIEQDCGVTRLGSLSLS | |
| | TLETVKAVKGIIYSYFSTALNASKNNPISDEQR | |
| | KEFDPELFALLEKLELIRTRKKKQKVERIANSL | |
| | IQTCLENNIKFIRGEGDLSTINNATKKKANSRS | |
| | MDWLARGVENKIRQLAPMHNITLFGCGSLYTSH | |
| | QDPLVHRNPDKAMKCRWAAIPVKDIGDWVLRKL | |
| | SQNLRAKNRGTGEYYHQGVKEFLSHYELQDLEE | |
| | ELLKWRSDRKSNIPCWVLQNRLAEKLGNKEAVV | |
| | YIPVRGGRIYFATHKVATGAVSIVFDQKQVWVC | |
| | NADHVAAANIALTGKGIGEQSSDEENPDGSRIK | |
| | LQLTSGGSGGSGGSGGSGGSMSSAIKSYKSVLR | |
| | PNERKNQLLKSTIQCLEDGSAFFFKMLQGLFGG | |
| | ITPEIVRFSTEQEK | |
| SEQ ID NO: 48 | MTASDAIKQYFSASIGESYYWNDCRQQYYDLCR | |
| | ELGVEVSDLTHDLEILCREKCLAVATESNQNNS | |
| | IISVLFGTGEKEDRSVKLRITKKILEAISNLKE | |
| | IPKNVAPIQEIILNVAKATKETFRQVYAGNLGA | |
| | PSTLEKFIAKDGQKEFDLKKLQTDLKKVIRGKS | |
| | KERDWCCQEELRSYVEQNTIQYDLWAWGEMENK | |
| | AHTALKIKSTRNYNFAKQRLEQFKEIQSLNNLL | |
| | VVKKLNDFFDSEFFSGEETYTICVHHLGGKDLS | |
| | KLYKAWEDDPADPENAIVVLCDDLKNNFKKEPI | |
| | RNILRYIFTIRQECSAQDILAAAKYNQQLDRYK | |
| | SQKANPSVLGNQGFTWTNAVILPEKAQRNDRPN | |
| | SLDLRIWLYLKLRHPDGRWKKHHIPFYDIRFFQ | |
| | EIYAAGNSPVDTCQFRTPRFGYHLPKLIDQTAI | |
| | RVNKKHVKAAKTEARIRLAIQQGTLPVSNLKIT | |
| | EISATINSKGQVRIPVKFRVGRQKGTLQIGDRF | |
| | CGYDQNQTASHAYSLWEVVKEGQYHKELGCFVR | |
| | FISSGDIVSITENRGNQFDQLSYEGLAYPQYAD | |
| | WRKKASKFVSLWQITKKNKKKEIVTVEAKEKFD | |
| | AICKYQPRLYKFNKEYAYLLRDIVRGKSLVELQ | |
| | QIRQEIFRFIEQDCGVTRLGSLSLSTLETVKAV | |
| | KGIIYSYFSTALNASKNNPISDEQRKEFDPELF | |
| | ALLEKLELIRTRKKKQKVERIANSLIQTCLENN | |
| | IKFIRGEGDLSTINNATKKKANSRSMDWLARGV | |
| | FNKIRQLAPMHNITLFGCGSLYTSHQDPLVHRN | |
| | PDKAMKCRWAAIPVKDIGDWVLRKLSQNLRAKN | |
| | RGTGEYYHQGVKEFLSHYELQDLEEELLKWRSD | |
| | RKSNIPCWVLQNRLAEKLGNKEAVVYIPVRGGR | |
| | IYFATHKVATGAVSIVFDQKQVWVCNADHVAAA | |
| | NIALTGKGIGEQSSDEENPDGSRIKLQLTSGGS | |
| | GGSGGSGGSGGSMSSAIKSYKSVLRPNERKNQL | |
| | LKSTIQCLEDGSAFFFKMLQGLFGGITPEIVRF | |
| | STEQEKQQQDIALWCAVNWFRPVSQDSLTHTIA | |
| | SDNLVEKFEEYYGG | |
| SEQ ID NO: 49 | MESYYWNDCRQQYYDLCRELGVEVSDLTHDLEI | |
| | LCREKCLAVATESNQNNSIISVLFGTGEKEDRS | |
| | VKLRITKKILEAISNLKEIPKNVAPIQEIILNV | |
| | AKATKETFRQVYAGNLGAPSTLEKFIAKDGQKE | |
| | FDLKKLQTDLKKVIRGKSKERDWCCQEELRSYV | |
| | EQNTIQYDLWAWGEMENKAHTALKIKSTRNYNF | |
| | AKQRLEQFKEIQSLNNLLVVKKLNDFFDSEFFS | |
| | GEETYTICVHHLGGKDLSKLYKAWEDDPADPEN | |
| | AIVVLCDDLKNNFKKEPIRNILRYIFTIRQECS | |
| | AQDILAAAKYNQQLDRYKSQKANPSVLGNQGET | |
| | WINAVILPEKAQRNDRPNSLDLRIWLYLKLRHP | |
| | DGRWKKHHIPFYDTRFFQEIYAAGNSPVDTCQF | |
| | RTPRFGYHLPKLTDQTAIRVNKKHVKAAKTEAR | |
| | IRLAIQQGTLPVSNLKITEISATINSKGQVRIP | |
| | VKFRVGRQKGTLQIGDRFCGYDQNQTASHAYSL | |
| | WEVVKEGQYHKELGCFVRFISSGDIVSITENRG | |
| | NQFDQLSYEGLAYPQYADWRKKASKFVSLWQIT | |
| | KKNKKKEIVTVEAKEKFDAICKYQPRLYKENKE | |
| | YAYLLRDIVRGKSLVELQQIRQEIFRFIEQDCG | |
| | VTRLGSLSLSTLETVKAVKGIIYSYFSTALNAS | |
| | KNNPISDEQRKEFDPELFALLEKLELIRTRKKK | |
| | QKVERIANSLIQTCLENNIKFIRGEGDLSTINN | |
| | ATKKKANSRSMDWLARGVENKIRQLAPMHNITL | |
| | FGCGSLYTSHQDPLVHRNPDKAMKCRWAAIPVK | |
| | DIGDWVLRKLSQNLRAKNRGTGEYYHQGVKEFL | |
| | SHYELQDLEEELLKWRSDRKSNIPCWVLQNRLA | |
| | EKLGNKEAVVYIPVRGGRIYFATHKVATGAVSI | |
| | VFDQKQVWVCNADHVAAANIALTGKGIGEQSSD | |
| | EENPDGSRIKLQLTSGGSGGSGGSGGSGGSMSS | |
| | AIKSYKSVLRPNERKNQLLKSTIQCLEDGSAFF | |
| | FKMLQGLFGGITPEIVRFSTEQEKQQQDIALWC | |
| | AVNWFRPVSQDSLTHTIASDNLVEKFEEYYGGT | |
| | ASDAIKQYFSASIG | |
| SEQ ID NO: 50 | MIPKNVAPIQEIILNVAKATKETFRQVYAGNLG | |
| | APSTLEKFIAKDGQKEFDLKKLQTDLKKVIRGK | |
| | SKERDWCCQEELRSYVEQNTIQYDLWAWGEMEN | |
| | KAHTALKIKSTRNYNFAKQRLEQFKEIQSLNNL | |
| | LVVKKLNDFFDSEFFSGEETYTICVHHLGGKDL | |
| | SKLYKAWEDDPADPENAIVVLCDDLKNNFKKEP | |
| | IRNILRYIFTIRQECSAQDILAAAKYNQQLDRY | |
| | KSQKANPSVLGNQGFTWINAVILPEKAQRNDRP | |
| | NSLDLRIWLYLKLRHPDGRWKKHHIPFYDTRFF | |
| | QEIYAAGNSPVDTCQFRIPRFGYHLPKLIDQTA | |
| | IRVNKKHVKAAKTEARIRLAIQQGTLPVSNLKI | |
| | TEISATINSKGQVRIPVKFRVGRQKGTLQIGDR | |
| | FCGYDQNQTASHAYSLWEVVKEGQYHKELGCFV | |
| | RFISSGDIVSITENRGNQFDQLSYEGLAYPQYA | |
| | DWRKKASKFVSLWQITKKNKKKEIVTVEAKEKF | |
| | DAICKYQPRLYKFNKEYAYLLRDIVRGKSLVEL | |
| | QQIRQEIFRFIEQDCGVTRLGSLSLSTLETVKA | |
| | VKGIIYSYFSTALNASKNNPISDEQRKEFDPEL | |
| | FALLEKLELIRTRKKKQKVERIANSLIQTCLEN | |
| | NIKFIRGEGDLSTINNATKKKANSRSMDWLARG | |
| | VENKIRQLAPMHNITLFGCGSLYTSHQDPLVHR | |
| | NPDKAMKCRWAAIPVKDIGDWVLRKLSQNLRAK | |
| | NRGTGEYYHQGVKEFLSHYELQDLEEELLKWRS | |
| | DRKSNIPCWVLQNRLAEKLGNKEAVVYIPVRGG | |
| | RIYFATHKVATGAVSIVFDQKQVWVCNADHVAA | |
| | ANIALTGKGIGEQSSDEENPDGSRIKLQLTSGG | |
| | SGGSGGSGGSGGSMSSAIKSYKSVLRPNERKNQ | |
| | LLKSTIQCLEDGSAFFFKMLQGLEGGITPEIVR | |
| | FSTEQEKQQQDIALWCAVNWFRPVSQDSLTHTI | |
| | ASDNLVEKFEEYYGGTASDAIKQYFSASIGESY | |
| | YWNDCRQQYYDLCRELGVEVSDLTHDLEILCRE | |
| | KCLAVATESNQNNSIISVLFGTGEKEDRSVKLR | |
| | ITKKILEAISNLKE | |
| SEQ ID NO: 51 | MEFDLKKLQTDLKKVIRGKSKERDWCCQEELRS | |
| | YVEQNTIQYDLWAWGEMENKAHTALKIKSTRNY | |
| | NFAKQRLEQFKEIQSLNNLLVVKKLNDFFDSEF | |
| | FSGEETYTICVHHLGGKDLSKLYKAWEDDPADP | |
| | ENAIVVLCDDLKNNFKKEPIRNILRYIFTIRQE | |
| | CSAQDILAAAKYNQQLDRYKSQKANPSVLGNQG | |
| | FTWTNAVILPEKAQRNDRPNSLDLRIWLYLKLR | |
| | HPDGRWKKHHIPFYDTRFFQEIYAAGNSPVDTC | |
| | QFRTPRFGYHLPKLTDQTAIRVNKKHVKAAKTE | |
| | ARIRLAIQQGTLPVSNLKITEISATINSKGQVR | |
| | IPVKFRVGRQKGTLQIGDRFCGYDQNQTASHAY | |
| | SLWEVVKEGQYHKELGCFVRFISSGDIVSITEN | |
| | RGNQFDQLSYEGLAYPQYADWRKKASKFVSLWQ | |
| | ITKKNKKKEIVTVEAKEKFDAICKYQPRLYKFN | |
| | KEYAYLLRDIVRGKSLVELQQIRQEIFRFIEQD | |
| | CGVTRLGSLSLSTLETVKAVKGIIYSYFSTALN | |
| | ASKNNPISDEQRKEFDPELFALLEKLELIRTRK | |
| | KKQKVERIANSLIQTCLENNIKFIRGEGDLSTT | |
| | NNATKKKANSRSMDWLARGVENKIRQLAPMHNI | |
| | TLFGCGSLYTSHQDPLVHRNPDKAMKCRWAAIP | |
| | VKDIGDWVLRKLSQNLRAKNRGTGEYYHQGVKE | |
| | FLSHYELQDLEEELLKWRSDRKSNIPCWVLQNR | |
| | LAEKLGNKEAVVYIPVRGGRIYFATHKVATGAV | |
| | SIVFDQKQVWVCNADHVAAANIALIGKGIGEQS | |
| | SDEENPDGSRIKLQLTSGGSGGSGGSGGSGGSM | |
| | SSAIKSYKSVLRPNERKNQLLKSTIQCLEDGSA | |
| | FFFKMLQGLFGGITPEIVRESTEQEKQQQDIAL | |
| | WCAVNWFRPVSQDSLTHTIASDNLVEKFEEYYG | |
| | GTASDAIKQYFSASIGESYYWNDCRQQYYDLCR | |
| | ELGVEVSDLTHDLEILCREKCLAVATESNQNNS | |
| | IISVLFGTGEKEDRSVKLRITKKILEAISNLKE | |
| | IPKNVAPIQEIILNVAKATKETFRQVYAGNLGA | |
| | PSTLEKFIAKDGQK | |
| SEQ ID NO: 52 | MPDKAMKCRWAAIPVKDIGDWVLRKLSQNLRAK | |
| | NRGTGEYYHQGVKEFLSHYELQDLEEELLKWRS | |
| | DRKSNIPCWVLQNRLAEKLGNKEAVVYIPVRGG | |
| | RIYFATHKVATGAVSIVFDQKQVWVCNADHVAA | |
| | ANIALIGKGIGEQSSDEENPDGSRIKLQLTSGG | |
| | SGGSGGSGGSGGSMSSAIKSYKSVLRPNERKNQ | |
| | LLKSTIQCLEDGSAFFFKMLQGLFGGITPEIVR | |
| | FSTEQEKQQQDIALWCAVNWFRPVSQDSLTHTI | |
| | ASDNLVEKFEEYYGGTASDAIKQYFSASIGESY | |
| | YWNDCRQQYYDLCRELGVEVSDLTHDLEILCRE | |
| | KCLAVATESNQNNSIISVLFGTGEKEDRSVKLR | |
| | ITKKILEAISNLKEIPKNVAPIQEIILNVAKAT | |
| | KETFRQVYAGNLGAPSTLEKFIAKDGQKEFDLK | |
| | KLQTDLKKVIRGKSKERDWCCQEELRSYVEQNT | |
| | IQYDLWAWGEMFNKAHTALKIKSTRNYNFAKQR | |
| | LEQFKEIQSLNNLLVVKKLNDFFDSEFFSGEET | |
| | YTICVHHLGGKDLSKLYKAWEDDPADPENAIVV | |
| | LCDDLKNNFKKEPIRNILRYIFTIRQECSAQDI | |
| | LAAAKYNQQLDRYKSQKANPSVLGNQGFTWTNA | |
| | VILPEKAQRNDRPNSLDLRIWLYLKLRHPDGRW | |
| | KKHHIPFYDTRFFQEIYAAGNSPVDTCQFRTPR | |
| | FGYHLPKLTDQTAIRVNKKHVKAAKTEARIRLA | |
| | IQQGTLPVSNLKITEISATINSKGQVRIPVKFR | |
| | VGRQKGTLQIGDRFCGYDQNQTASHAYSLWEVV | |
| | KEGQYHKELGCFVRFISSGDIVSITENRGNQFD | |
| | QLSYEGLAYPQYADWRKKASKFVSLWQITKKNK | |
| | KKEIVTVEAKEKFDAICKYQPRLYKENKEYAYL | |
| | LRDIVRGKSLVELQQIRQEIFRFIEQDCGVTRL | |
| | GSLSLSTLETVKAVKGIIYSYFSTALNASKNNP | |
| | ISDEQRKEFDPELFALLEKLELIRTRKKKQKVE | |
| | RIANSLIQTCLENNIKFIRGEGDLSTINNATKK | |
| | KANSRSMDWLARGVENKIRQLAPMHNITLFGCG | |
| | SLYTSHQDPLVHRN | |
| SEQ ID NO: 53 | AGACAUGUGUCCUCAGUGACACGGAUGGCGACU | EMX1_T2 crRNA Sequence |
| | UCAGGCACAGGAU | |
| SEQ ID NO: 54 | GGATGGCGACTTCAGGCACAGGAT | EMX1_T2 Target Sequence |
| SEQ ID NO: 55 | AGACAUGUGUCCUCAGUGACACAGCAAGGGACU | EMX1_T7 crRNA Sequences |
| | AUUCAGGGAUGAA | |
| SEQ ID NO: 56 | AGCAAGGGACTATTCAGGGATGAA | EMX1_T7 Target Sequence |
| SEQ ID NO: 57 | AGACAUGUGUCCUCAGUGACACAAAAUUGAGCA | EMX1_T8 crRNA Sequences |
| | AUCUACCCUGGUC | |
| SEQ ID NO: 58 | AAAATTGAGCAATCTACCCTGGTC | EMX1-T8 Target Sequences |
| SEQ ID NO: 59 | AGACAUGUGUCCUCAGUGACACUUAAACUCUCC | VEGFA_T5 crRNA Sequence |
| | AUGGACCAGGCUC | |
| SEQ ID NO: 60 | TTAAACTCTCCATGGACCAGGCTC | VEGFA_T5 Target Sequence |
| SEQ ID NO: 61 | KRPAATKKAGQAKKKK | |
| SEQ ID NO: 62 | MKRTADGSEFESPKKKRKV | |
| SEQ ID NO: 63 | MKRTADGSEFESPKKKRKVE | |
| SEQ ID NO: 64 | KRTADGSEFESPKKKRKV | |
| SEQ ID NO: 65 | KRTADGSEFESPKKKRKVE | |
| SEQ ID NO: 66 | MKIEEGKGHHHHHH | |
| SEQ ID NO: 67 | KIEEGKGHHHHHH | |
| SEQ ID NO: 68 | MKIEEGKGHHHHHHMSSAIKSYKSVIRPNERKNQLL | |
| | KSTIQCLEDGSAFFFKMLQGLFGGITPEIVRESTEQ | |
| | EKQQQDIALWCAVNWFRPVSQDSLTHTIASDNLVEK | |
| | FEEYYGGTASDAIKQYFSASIGESYYWNDCRQQYYD | |
| | LCRELGVEVSDITHDLEILCREKCLAVATESNQNNS | |
| | IISVLFGIGEKEDRSVKLRITKKILEAISNLKEIPK | |
| | NVAPIQEIILNVAKATKETFRQVYAGNLGAPSTLEK | |
| | FIAKDGQKEFDLKKLQTDLKKVIRGKSKERDWCCQE | |
| | ELRSYVEQNTIQYDLWAWGEMENKAHTALKIKSTRN | |
| | YNFAKQRLEQFKEIQSLNNLLVVKKINDFEDSEFFS | |
| | GEETYTICVHHLGGKDLSKLYKAWEDDPADPENAIV | |
| | VLCDDLKNNEKKEPIRNILRYIFTIRQECSAQDILA | |
| | AAKYNQQLDRYKSQKANPSVLGNQGETWINAVILPE | |
| | KAQRNDRPNSLDLRIWLYLKLRHPDGRWKKHHIPFY | |
| | DTREFQEIYAAGNSPVDTCQFRIPREGYHLPKLIDQ | |
| | TAIRVNKKHVKAAKTEARIRLAIQQGTLPVSNLKIT | |
| | EISATINSKGQVRIPVKFRVGRQKGTLQIGDRFCGY | |
| | DQNQTASHAYSLWEVVKEGQYHKELGCFVRFISSGD | |
| | IVSITENRGNQFDQLSYEGLAYPQYADWRKKASKFV | |
| | SLWQITKKNKKKEIVIVEAKEKFDAICKYQPRLYKF | |
| | NKEYAYLLRDIVRGKSLVELQQIRQEIFRFIEQDCG | |
| | VTRLGSLSLSTLETVKAVKGIIYSYFSTALNASKNN | |
| | PISDEQRKEFDPELFALLEKLELIRTRKKKQKVERI | |
| | ANSLIQTCLENNIKFIRGEGDLSTINNATKKKANSR | |
| | SMDWLARGVENKIRQLAPMHNITLFGCGSLYTSHQD | |
| | PLVHRNPDKAMKCRWAAIPVKDIGDWVLRKLSQNER | |
| | AKNRGTGEYYHQGVKEFLSHYELQDLEEELLKWRSD | |
| | RKSNIPCWVLQNRLAEKLGNKEAVVYIPVRGGRIYF | |
| | ATHKVATGAVSIVEDQKQVWVCNADHVAAANIALTG | |
| | KGIGEQSSDEENPDGSRIKLQLTSGSKRTADGSEFE | |
| | SPKKKRKVE | |
| SEQ ID NO: 69 | MKIEEGKGHHHHHHMSSAIKSYKSVLRPNERKNQLL | |
| | KSTIQCLEDGSAFFFKMLQGLFGGITPEIVRFSTEQ | |
| | EKQQQDIALWCAVNWFRPVSQDSLTHTIASDNLVEK | |
| | FEEYYGGTASDAIKQYFSASIGESYYWNDCRQQYYD | |
| | LCRELGVEVSDITHDLEILCREKCLAVATESNQNNS | |
| | IISVLFGIGEKEDRSVKLRITKKILEAISNLKEIPK | |
| | NVAPIQEIILNVAKATKETFRQVYAGNLGAPSTLEK | |
| | FIAKDGQKEFDLKKLQTDLKKVIRGKSKERDWCCQE | |
| | ELRSYVEQNTIQYDLWAWGEMENKAHTALKIKSTRN | |
| | YNFAKQRLEQFKEIQSLNNLLVVKKINDFEDSEFFS | |
| | GEETYTICVHHLGGKDLSKLYKAWEDDPADPENAIV | |
| | VLCDDLKNNEKKEPIRNILRYIFTIRQECSAQDILA | |
| | AAKYNQQLDRYKSQKANPSVLGNQGFTWINAVILPE | |
| | KAQRNDRPNSLDLRIWLYLKLRHPDGRWKKHHIPFY | |
| | DTRFFQEIYAAGNSPVDTCQFRIPRFGYHLPKLIDQ | |
| | TAIRVNKKHVKAAKTEARIRLAIQQGTLPVSNLKIT | |
| | EISATINSKGQVRIPVKFRVGRQKGTLQIGDRFCGY | |
| | DQNQTASHAYSLWEVVKEGQYHKELGCFVRFISSGD | |
| | IVSITENRGNQFDQLSYEGLAYPQYADWRKKASKFV | |
| | SLWQITKKNKKKEIVIVEAKEKFDAICKYQPRLYKF | |
| | NKEYAYLLRDIVRGKSLVELQQIRQEIFRFIEQDCG | |
| | VTRLGSLSLSTLETVKAVKGIIYSYFSTALNASKNN | |
| | PISDEQRKEFDPELFALLEKLELIRTRKKKQKVERI | |
| | ANSLIQTCLENNIKFIRGEGDLSTINNATKKKANSR | |
| | SMDWLARGVENKIRQLAPMHNITLFGCGSLYTSHQD | |
| | PLVHRNPDKAMKCRWAAIPVKDIGDWVLRKLSQNER | |
| | AKNRGTGEYYHQGVKEFLSHYELQDLEEELLKWRSD | |
| | RKSNIPCWVLQNRLAEKLGNKEAVVYIPVRGGRIYF | |
| | ATHKVATGAVSIVEDQKQVWVCNADHVAAANIALTG | |
| | KGIGEQSSDEENPDGSRIKLQLTSGSKRTADGSEFE | |
| | SPKKKRKV | |
| SEQ ID NO: 70 | GSS | |
| SEQ ID NO: 71 | GGSGGSGGSGGSGGS | (GGS)5 |
| SEQ ID NO: 72 | GGS | |
| SEQ ID NO: 73 | MSSAIKSYKSVIRPNERKNQLLKSTIQCLEDGSAFF | |
| | FKMLQGLEGGITPEIVRESTEQEKQQQDIALWCAVN | |
| | WERPVSQDSLTHTIASDNLVEKFEEYYGGTASDAIK | |
| | QYFSASIGESYYWNDCRQQYYDLCRELGVEVSDLTH | |
| | DLEILCREKCLAVATESNQNNSIISVLEGTGEKEDR | |
| | SVKLRITKKILEAISNLKEIPKNVAPIQEIILNVAK | |
| | ATKETFRQVYAGNLGAPSTLEKFIAKDGQKEFDLKK | |
| | LQTDLKKVIRGKSKERDWCCQEELRSYVEQNTIQYD | |
| | LWAWGEMENKAHTALKIKSTRNYNFAKQRLEQFKEI | |
| | QSLNNLLVVKKLNDFEDSEFFSGEETYTICVHHLGG | |
| | KDLSKLYKAWEDDPADPENAIVVLCDDLKNNEKKEP | |
| | IRNILRYIFTIRQECSAQDILAAAKYNQQLDRYKSQ | |
| | KANPSVLGNQGETWINAVILPEKAQRNDRPNSLDER | |
| | IWLYLKLRHPDGRWKKHHIPFYDIRFFQEIYAAGNS | |
| | PVDTCQFRTPREGYHLPKLIDQTAIRVNKKHVKAAK | |
| | TEARIRLAIQQGTLPVSNLKITEISATINSKGQVRI | |
| | PVKFRVGRQKGTLQIGDRFCGYDQNQTASHAYSLWE | |
| | VVKEGQYHKELGCFVRFISSGDIVSITENRGNQFDQ | |
| | LSYEGLAYPQYADWRKKASKFVSLWQITKKNKKKEI | |
| | VTVEAKEKFDAICKYQPRLYKENKEYAYLLRDIVRG | |
| | KSLVELQQIRQEIFRFIEQDCGVTRLGSLSLSTLET | |
| | VKAVKGIIYSYFSTALNASKNNPISDEQRKEFDPEL | |
| | FALLEKLELIRTRKKKQKVERIANSLIQTCLENNIK | |
| | FIRGEGDLSTINNATKKKANSRSMDWLARGVENKIR | |
| | QLAPMHNITLFGCGSLYTSHQDPLVHRNPDKAMKCR | |
| | WAAIPVKDIGDWVLRKLSQNLRAKNRGTGEYYHQGV | |
| | KEFLSHYELQDLEEELLKWRSDRKSNIPCWVLQNRL | |
| | AEKLGNKEAVVYIPVRGGRIYFATHKVATGAVSIVE | |
| | DQKQVWVCNADHVAAANIALTGKGIGEQSSDEENPD | |
| | GSRIKLQLTSGSKRTADGSEFESPKKKRKVE | |
| SEQ ID NO: 74 | MSSAIKSYKSVLRPNERKNQLLKSTIQCLEDGSAFF | |
| | FKMLQGLEGGITPEIVRFSTEQEKQQQDIALWCAVN | |
| | WERPVSQDSLTHTIASDNLVEKFEEYYGGTASDAIK | |
| | QYFSASIGESYYWNDCRQQYYDLCRELGVEVSDITH | |
| | DLEILCREKCLAVATESNQNNSIISVLEGTGEKEDR | |
| | SVKLRITKKILEAISNLKEIPKNVAPIQEIILNVAK | |
| | ATKETFRQVYAGNLGAPSTLEKFIAKDGQKEFDLKK | |
| | LQTDLKKVIRGKSKERDWCCQEELRSYVEQNTIQYD | |
| | LWAWGEMENKAHTALKIKSTRNYNFAKQRLEQFKEI | |
| | QSLNNLLVVKKLNDFFDSEFFSGEETYTICVHHLGG | |
| | KDLSKLYKAWEDDPADPENAIVVLCDDLKNNFKKEP | |
| | IRNILRYIFTIRQECSAQDILAAAKYNQQLDRYKSQ | |
| | KANPSVLGNQGETWINAVILPEKAQRNDRPNSLDLR | |
| | IWLYLKLRHPDGRWKKHHIPFYDTRFFQEIYAAGNS | |
| | PVDICQFRTPREGYHLPKLIDQTAIRVNKKHVKAAK | |
| | TEARIRLAIQQGTLPVSNLKITEISATINSKGQVRI | |
| | PVKFRVGRQKGTLQIGDRFCGYDQNQTASHAYSLWE | |
| | VVKEGQYHKELGCFVRFISSGDIVSITENRGNQFDQ | |
| | LSYEGLAYPQYADWRKKASKFVSLWQITKKNKKKEI | |
| | VTVEAKEKFDAICKYQPRLYKENKEYAYLLRDIVRG | |
| | KSIVELQQIRQEIFRFIEQDCGVTRLGSLSLSTLET | |
| | VKAVKGIIYSYFSTALNASKNNPISDEQRKEFDPEL | |
| | FALLEKLELIRTRKKKQKVERIANSLIQTCLENNIK | |
| | FIRGEGDLSTINNATKKKANSRSMDWLARGVENKIR | |
| | QLAPMHNITLFGCGSLYTSHQDPLVHRNPDKAMKCR | |
| | WAAIPVKDIGDWVLRKLSQNLRAKNRGTGEYYHQGV | |
| | KEFLSHYELQDLEEELLKWRSDRKSNIPCWVLQNRL | |
| | AEKLGNKEAVVYIPVRGGRIYFATHKVATGAVSIVE | |
| | DQKQVWVCNADHVAAANIALTGKGIGEQSSDEENPD | |
| | GSRIKLQLTSGSKRTADGSEFESPKKKRKV | |

What is claimed is:

1. A Cas12i2 fusion protein comprising:
a) a first portion comprising amino acids 1-n of any one of SEQ ID NO: 1 or any one of SEQ ID NOs: 39-43, or a sequence having at least 80%, 85%, 90%, 95%, 96%, 97%, 98%, or 99% identity thereto;
b) a second portion comprising amino acids m-1054 of SEQ ID NO: 1 or any one of SEQ ID NOs: 39-43, or a sequence having at least 80%, 85%, 90%, 95%, 96%, 97%, 98%, or 99% identity thereto; and
c) a heterologous sequence disposed between the first portion and the second portion,
wherein n and m are each independently a number between:
i) 342-358 (e.g., 342, 343, 344, 345, 346, 347, 348, 349, 350, 351, 352, 353, 354, 355, 356, 357, or 358);
ii) 373-378 (e.g., 373, 374, 375, 376, 377, or 378);
iii) 408-413 (e.g., 408, 409, 410, 411, 412, or 413);
iv) 677-685 (e.g., 677, 678, 679, 680, 681, 682, 683, 684, or 685);
v) 718-723 (e.g., 718, 719, 720, 721, 722, or 723);
vi) 771-782 (e.g., 771, 772, 773, 774, 775, 776, 777, 778, 779, 780, 781, or 782);
vii) 953-965 (e.g., 953, 954, 955, 956, 957, 958, 959, 960, 961, 962, 963, 964, or 965);
viii) 55-65 (e.g., 55, 56, 57, 58, 59, 60, 61, 62, 63, 64, or 65);
ix) 99-105 (e.g., 99, 100, 101, 102, 103, 104, or 105);
x) 112-120 (e.g., 112, 113, 114, 115, 116, 117, 118, 119, or 120);
xi) 195-206 (e.g., 195, 196, 197, 198, 199, 200, 201, 202, 203, 204, 205, or 206);
xii) 241-250 (e.g., 241, 242, 243, 244, 245, 246, 247, 248, 249, or 250);
xiii) 583-594 (e.g., 583, 584, 585, 586, 587, 588, 589, 590, 591, 592, 593, or 594);
xiv) 877-901 (e.g., 877, 878, 879, 880, 881, 882, 883, 884, 885, 886, 887, 888, 889, 890, 891, 892, 893, 894, 895, 896, 897, 898, 899, 900, or 901);
xv) 173-179 (e.g., 173, 174, 175, 176, 177, 178, or 179);
xvi) 216-221 (e.g., 216, 217, 218, 219, 220, or 221);
xvii) 265-272 (e.g., 265, 266, 267, 268, 269, 270, 271, or 272);
xviii) 456-468 (e.g., 456, 457, 458, 459, 460, 461, 462, 463, 464, 465, 466, 467, or 468);
xix) 476-482 (e.g., 476, 477, 478, 479, 480, 481, or 482);
xx) 498-513 (e.g., 498, 499, 500, 501, 502, 503, 504, 505, 506, 507, 508, 509, 510, 511, 512, or 513);
xxi) 614-625 (e.g., 614, 615, 616, 617, 618, 619, 620, 621, 622, 623, 624, or 625);
xxii) 977-982 (e.g., 977, 978, 979, 980, 981, or 982); or
xxiii) 1007-1012 (e.g., 1007, 1008, 1009, 1010, 1011, or 1012).

2. The Cas12i2 fusion protein of claim 1, wherein n<m.

3. The Cas12i2 fusion protein of claim 1, wherein m=n+1.

4. The Cas12i2 fusion protein of any one of claims 1-3, wherein the Cas12i2 fusion protein is a Cas12i2 fusion protein as described herein.

5. The Cas12i2 fusion protein of any one of claims 1-4, wherein the heterologous sequence comprises a fusion domain (e.g., a base editing domain, a ssDNA binding domain, an NLS, a poly-basis domain, a restriction endonuclease, or a CRISPR nuclease).

6. The Cas12i2 fusion protein of any one of claims 1-5, wherein the heterologous sequence comprises at least one linker sequence.

7. The Cas12i2 fusion protein of any one of claims 1-6, wherein the heterologous sequence comprises a first linker (e.g., a first peptide linker) and a second linker (e.g., a second peptide linker).

8. The Cas12i2 fusion protein of claim 7, wherein the first linker and the second linker each independently comprise between 3 and 60 amino acid residues.

9. The Cas12i2 fusion protein of claim 7 or 8, wherein the first linker and the second linker each independently comprise one or more Gly residues and one or more Ser residues.

10. The Cas12i2 fusion protein of any one of claims 7-9, wherein the first linker and the second peptide linker each independently comprise (GSG)ₓ, (GGGS)ₓ, or (GSSG)ₓ, wherein x is an integer between 0 and 15 (e.g., 0, 1, 2, 3, 4, 5, 6, 7, 8, 9, 10, 11, 12, 13, 14, or 15).

11. The Cas12i2 fusion protein of any one of claims 7-10, wherein the first linker is N-terminal of the fusion domain and the second linker is C-terminal of the fusion domain.

12. The Cas12i2 fusion protein of any one of claims 7-11, wherein the first linker and the second linker are the same.

13. The Cas12i2 fusion protein of any one of claims 7-11, wherein the first linker and the second linker are different.

14. A Cas12i2 fusion protein comprising:
a) a Cas12i2 domain comprising an amino acid sequence of SEQ ID NO: 1 or any one of SEQ ID NOs: 39-43, or a sequence having at least 80%, 85%, 90%, 95%, 96%, 97%, 98%, or 99% identity thereto;
b) a first heterologous sequence disposed N-terminal of the Cas12i2 domain; and
c) a second heterologous sequence disposed C-terminal of the Cas12i2 domain,
wherein the first heterologous sequence comprises a dimerization domain, the second heterologous sequence comprises a dimerization domain, or the first heterologous sequence comprises a first dimerization domain and the second heterologous sequences comprises a second, compatible dimerization domain.

15. The Cas12i2 fusion protein of claim 14, wherein the heterologous sequence further comprises a fusion domain.

16. The Cas12i2 fusion protein of claim 14 or 15, wherein the Cas12i2 fusion protein is a Cas12i2 fusion protein as described herein (e.g., in section Fusion proteins with dimerization domains of the detailed description).

17. A Cas12i2 fusion protein comprising:
a) a Cas12i2 domain comprising an amino acid sequence of SEQ ID NO: 1 or any one of SEQ ID NOs: 39-43, or a sequence having at least 80%, 85%, 90%, 95%, 96%, 97%, 98%, or 99% identity thereto;
b) a first heterologous sequence disposed N-terminal of the Cas12i2 domain, wherein the first heterologous sequence comprises a first portion of a split fusion domain; and
c) a second heterologous sequence disposed C-terminal of the Cas12i2 domain, wherein the second heterologous sequence comprises a second portion of a split fusion domain, wherein the second portion of the split fusion domain can bind the first portion of the split fusion domain.

18. The Cas12i2 fusion protein of claim 17, wherein the Cas12i2 fusion protein is a Cas12i2 fusion protein as described herein (e.g., in section N-terminal and C-terminal split fusion).

19. An engineered, non-naturally occurring Cas12i2 fusion protein comprising:
a) a first portion comprising an amino acid sequence of an N-terminal portion of SEQ ID NO: 1 or any one of SEQ ID NOs: 39-43, or a sequence having at least 80%, 85%, 90%, 95%, 96%, 97%, 98%, or 99% identity thereto; and
b) a second portion comprising an amino acid sequence of a C-terminal portion of SEQ ID NO: 1 or any one of SEQ ID NOs: 39-43, or a sequence having at least 80%, 85%, 90%, 95%, 96%, 97%, 98%, or 99% identity thereto,
wherein the second portion is N-terminal of the first portion,
wherein the first portion and second portion together bind to an RNA guide comprising a direct repeat sequence and a spacer sequence.

20. The Cas12i2 fusion protein of claim 19, which is capable of specifically contacting a target nucleic acid.

21. The Cas12i2 fusion protein of claim 19, wherein the first portion and the second portion are linked by a heterologous sequence.

22. The Cas12i2 fusion protein of claim 21, wherein the heterologous sequence comprises one or more of:
a) a first linker (e.g., a first peptide linker);
b) a second linker (e.g., a second peptide linker); and
c) a fusion domain.

23. The Cas12i2 fusion protein of any one of claims 19-22, wherein the C-terminal most amino acid of the first portion is any amino acid residue of SEQ ID NO: 1 or any one of SEQ ID NOs: 39-43 chosen from residues:
a) 342-358 (e.g., residue 342, 343, 344, 345, 346, 347, 348, 349, 350, 351, 352, 353, 354, 355, 356, 357, or 358);
b) 373-378 (e.g., residue 373, 374, 375, 376, 377, or 378);
c) 408-413 (e.g., residue 408, 409, 410, 411, 412, or 413);
d) 677-685 (e.g., residue 677, 678, 679, 680, 681, 682, 683, 684, or 685);
e) 718-723 (e.g., residue 718, 719, 720, 721, 722, or 723);
f) 771-782 (e.g., residue 771, 772, 773, 774, 775, 776, 777, 778, 779, 780, 781, or 782);
g) 953-965 (e.g., residue 953, 954, 955, 956, 957, 958, 959, 960, 961, 962, 963, 964, or 965);
h) 55-65 (e.g., residue 55, 56, 57, 58, 59, 60, 61, 62, 63, 64, or 65);
i) 99-105 (e.g., residue 99, 100, 101, 102, 103, 104, or 105);
j) 112-120 (e.g., residue 112, 113, 114, 115, 116, 117, 118, 119, or 120);
k) 195-206 (e.g., residue 195, 196, 197, 198, 199, 200, 201, 202, 203, 204, 205, or 206);
l) 241-250 (e.g., residue 241, 242, 243, 244, 245, 246, 247, 248, 249, or 250);
m) 583-594 (e.g., residue 583, 584, 585, 586, 587, 588, 589, 590, 591, 592, 593, or 594);
n) 877-901 (e.g., residue 877, 878, 879, 880, 881, 882, 883, 884, 885, 886, 887, 888, 889, 890, 891, 892, 893, 894, 895, 896, 897, 898, 899, 900, or 901);
o) 173-179 (e.g., residue 173, 174, 175, 176, 177, 178, or 179);
p) 216-221 (e.g., residue 216, 217, 218, 219, 220, or 221);
q) 265-272 (e.g., residue 265, 266, 267, 268, 269, 270, 271, or 272);
r) 456-468 (e.g., residue 456, 457, 458, 459, 460, 461, 462, 463, 464, 465, 466, 467, or 468);
s) 476-482 (e.g., residue 476, 477, 478, 479, 480, 481, or 482);
t) 498-513 (e.g., residue 498, 499, 500, 501, 502, 503, 504, 505, 506, 507, 508, 509, 510, 511, 512, or 513);
u) 614-625 (e.g., residue 614, 615, 616, 617, 618, 619, 620, 621, 622, 623, 624, or 625);
v) 977-982 (e.g., residue 977, 978, 979, 980, 981, or 982);
w) 1007-1012 (e.g., residue 1007, 1008, 1009, 1010, 1011, or 1012);
x) 386-397 (e.g., residue 386, 387, 388, 389, 390, 391, 392, 393, 394, 395, 396, or 397); or
y) 831-844 (e.g., residue 831, 832, 833, 834, 835, 836, 837, 838, 839, 840, 841, 842, 843, or 844).

24. The Cas12i2 fusion protein of any previous claim, wherein the first portion further comprises a fusion domain, the second portion comprises a fusion domain, or the first portion and the second portion comprise a fusion domain.

25. The Cas12i2 fusion protein of claim 24, wherein the fusion domain comprises a FokI nuclease domain.

26. The Cas12i2 fusion protein of claim 25, wherein the FokI nuclease domain is a catalytically active FokI nuclease domain or a catalytically inactive FokI nuclease domain.

27. The Cas12i2 fusion protein of claim 26, wherein:
a) the first portion comprises a catalytically active FokI nuclease domain and the second portion comprises a catalytically inactive FokI nuclease domain; or
b) the first portion comprises a catalytically inactive FokI nuclease domain and the second portion comprises a catalytically active FokI nuclease domain.

28. The Cas12i2 fusion protein of any one of claims 19-27, wherein the Cas12i2 fusion protein comprises a catalytically inactive RuvC domain.

29. The Cas12i2 fusion protein of any one of claims 19-28, wherein the Cas12i2 fusion protein comprises nickase activity.

30. The Cas12i2 fusion protein of any previous claim, wherein the Cas12i2 fusion protein is capable of binding an RNA guide comprising a direct repeat sequence and a spacer sequence, wherein the spacer sequence is capable of hybridizing to a target nucleic acid.

31. The Cas12i2 fusion protein of any previous claim, wherein the fusion domain is chosen from a peptide tag, a fluorescent protein, a base-editing domain, a DNA methylation domain, a histone residue modification domain, a localization factor (e.g., an NLS), a transcription modification factor, a light-gated control factor, a chemically inducible factor, a chromatin visualization factor, a restriction endonuclease, or a CRISPR nuclease.

32. The Cas12i2 fusion protein of any of the previous claims, wherein the Cas12i2 fusion protein comprises a fusion domain having an amino acid sequence of SEQ ID NO: 66 or SEQ ID NO: 67, or a sequence having at least 80%, 85%, 90%, 95%, 96%, 97%, 98%, or 99% identity thereto.

33. The Cas12i2 fusion protein of claim 32, wherein the fusion domain is situated at the N-terminus or C-terminus of the Cas12i2 fusion protein.

34. The Cas12i2 fusion protein of any of the previous claims, wherein the fusion domain comprises an NLS.

35. The Cas12i2 fusion protein of claim 34, wherein the NLS comprises an amino acid sequence of any one of SEQ ID NOs: 61-65, or a sequence having at least 80%, 85%, 90%, 95%, 96%, 97%, 98%, or 99% identity thereto.

36. The Cas12i2 fusion protein of claim 32-35, wherein the Cas12i2 protein comprises an amino acid sequence of SEQ ID NO: 68, SEQ ID NO: 69, SEQ ID NO: 73, or SEQ ID NO: 74, or a sequence having at least 80%, 85%, 90%, 95%, 96%, 97%, 98%, or 99% identity thereto.

37. The Cas12i2 fusion protein of any previous claim, wherein the heterologous sequence is about 1-5, 10-20, 20-30, 30-40, 40-50, 50-100, 100-200, 200-300, 300-400, 400-500, 500-600, 600-700, 700-800, 800-900, 900-1100, 1100-1400, 1400-1600, 1600-1800, or 1800-2000 amino acids in length.

38. A system comprising:
(a) a Cas12i2 fusion protein of any of claims 1-37, and
(b) an RNA guide comprising a direct repeat sequence and a spacer sequence, wherein the spacer sequence is capable of hybridizing to a target nucleic acid, e.g., target strand (i.e., non-PAM strand) of a target nucleic acid.

39. A nucleic acid encoding the Cas12i2 fusion protein of any of claims 1-37 or the system of claim 38.

40. A composition comprising: a first nucleic acid encoding the Cas12i2 fusion protein of any of claims 1-37 and a second nucleic acid comprising or encoding an RNA guide comprising a direct repeat sequence and a spacer sequence, wherein the spacer sequence is capable of hybridizing to a target nucleic acid.

41. A vector comprising:
(a) the nucleic acid of claim 39; or
(b) one or both of the first nucleic acid and second nucleic acids of claim 40.

42. A cell (e.g., a eukaryotic cell or a prokaryotic cell) comprising the Cas12i2 fusion protein of any of claims 1-37 or the system of claim 38.

43. A method of contacting the Cas12i2 fusion protein of any one of claims 1-37 or the system of claim 38 with a target nucleic acid in a cell comprising:
(a) providing the Cas12i2 fusion protein or the system;
(b) providing an RNA guide comprising a direct repeat sequence and a spacer sequence, wherein the spacer sequence is capable of hybridizing to the target nucleic acid, wherein the Cas12i2 fusion protein is capable of binding to the RNA guide; and
(c) delivering (i) the Cas12i2 fusion protein or the system and (ii) the RNA guide to the cell,
wherein the cell comprises the target nucleic acid, wherein the Cas12i2 fusion protein binds to the RNA guide, and wherein the spacer sequence binds to the target nucleic acid.

44. The method of claim 43, wherein the target nucleic acid is a double-stranded DNA.

45. A method of modifying a target nucleic acid, the method comprising delivering to the target nucleic acid (i) a Cas12i2 fusion protein of any of claims 1-37 or the system of claim 38 and (ii) an RNA guide comprising a direct repeat sequence and a spacer sequence, wherein the spacer sequence is capable of hybridizing to the target nucleic acid, wherein the Cas12i2 fusion protein is capable of binding to the RNA guide, and wherein recognition of the target nucleic acid by the CRISPR-associated protein and RNA guide results in a modification of the target nucleic acid.

46. The method of claim 45, wherein the modification comprises DNA methylation, epigenetic modification, or DNA cleavage (e.g., single stranded cleavage, double stranded cleavage, or nicking).

47. The method of claim 45 or 46, wherein the target nucleic acid comprises a target strand and a non-target strand, and the system modifies the target strand.

48. The method of claim 45 or 46, wherein the target nucleic acid comprises a target strand and a non-target strand, and the system modifies the non-target strand.

49. The method of claim 45 or 46, wherein the target nucleic acid comprises a target strand and a non-target strand, and the system modifies the target strand and the non-target strand.
